(19) United States
(12) Patent Application Publication
(10) Pub. No.: US 2023/0235058 A1
(43) Pub. Date: Jul. 27, 2023

(54) BTLA ANTIBODIES

(71) Applicants: Oxford University Innovation Limited, Oxford (GB); MiroBio Limited, Oxford (GB)

(72) Inventors: Simon John DAVIS, Oxford, (GB); Richard John CORNALL, Oxford, (GB); Chirstopher Douglas PALUCH, Oxford, (GB)

(21) Appl. No.: 18/001,367

(22) PCT Filed: Jun. 11, 2021
(86) PCT No.: PCT/GB2021/051452

§ 371 Date: Dec. 9, 2022

(30) Foreign Application Priority Data
Jun. 11, 2020 (GB) 2008860.5

(51) Int. Cl. C07K 16/28; A61P 1/00; A61P 37/06

(52) U.S. Cl. CPC C07K 16/2818; A61P 1/00; A61P 37/06; A61K 2039/505

(57) ABSTRACT

This invention relates generally to antibodies that bind to human B and T lymphocyte attenuator (BTLA) and uses thereof. More specifically, the invention relates to agonistic antibodies that bind human BTLA and modulate its activity, and their use in treating inflammatory, autoimmune and proliferative diseases and disorders. Suitably, the antibodies also possess an Fc modification that enhances signalling through FcγR2B.

### FIELD OF THE INVENTION

This invention relates generally to antibodies, including antigen binding fragments, that bind to human B and T lymphocyte attenuator (BTLA) and to uses thereof. More specifically, the invention relates to agonistic antibodies that bind human BTLA and modulate its activity and have enhanced binding to and signaling through FcγR2B, and their use in treating inflammatory, autoimmune and proliferative diseases and disorders.

### BACKGROUND

The immune system must achieve a balance between the destruction of pathogens or dangerously mutated cells and tolerance of healthy self-tissue and innocuous commensals. To facilitate this balance the activity of immune cells is influenced by the integration of signals from multiple stimulatory and inhibitory receptors that attune cells to their environment. These surface-expressed receptors present attractive targets for the therapeutic modulation of immune responses. Many human diseases result from aberrant or unwanted activation of the immune system including autoimmune diseases, transplant rejection and graft-versus-host disease. Agonist agents capable of inducing signaling through inhibitory receptors could dampen these unwanted immune responses.

B and T lymphocyte attenuator (BTLA; also designated CD272) is an inhibitory member of the CD28 family of receptors that also includes CD28, CTLA-4, ICOS, and PD-1 (Watanabe et al., Nat Immunol. 4:670-679, 2003) It is widely expressed throughout the immune system on both myeloid and lymphoid cells (Han et al., J Immunol. 172:5931-9, 2004). Following engagement by its ligand herpesvirus entry mediator (HVEM), BTLA recruits the phosphatases SHP-1 and SHP-2 to its cytoplasmic domain (Sedy et al., Nat Immunol. 6:90-8, 2005), which in turn inhibit the signaling cascades of activating receptors. Mice lacking an intact BTLA gene show hyperproliferative B and T cell responses in vitro, higher titers to DNP-KLH post-immunization and an increased sensitivity to EAE (Watanabe et al, Nat. Immunol, 4:670-679, 2003). If observed until old age BTLA knock-out mice spontaneously develop autoantibodies, an autoimmune hepatitis like disease and inflammatory cell infiltrates in multiple organs (Oya et al., Arthritis Rheum 58: 2498-2510, 2008). This evidence indicates that the BTLA inhibitory receptor plays a crucial role in maintaining immune homeostasis and inhibiting autoimmunity. Furthermore, HVEM-BTLA signaling is involved in the regulation of mucosal inflammation and infection immunity (Shui et al., J Leukoc Biol. 89:517-523, 2011).

Therapeutic agents that are capable of modulating BTLA function to inhibit autoreactive lymphocytes in the context of autoimmune disorders would be highly desirable.

It has previously been shown that monoclonal antibodies binding to mouse BTLA can act as agonists, inducing signaling through the receptor to inhibit immune cell responses. In the presence of agonist anti-BTLA antibody (mAb), anti-CD3 and anti-CD28 activated T-cells show reduced IL-2 production and proliferation (Kreig et al., J. Immunol., 175, 6420-6472, 2005).

Furthermore, anti-mouse-BTLA agonist antibodies have been shown to ameliorate disease in murine models of graft-versus-host disease (Sakoda et al., Blood. 117:2506-2514; Albring et al., J Exp Med. 207:2551-9, 2010). Agonist antibodies targeting the human BTLA receptor have been shown to inhibit T cell responses ex-vivo (see Otsuki et al., Biochem Biophys Res Commun 344:1121-7, 2006; and WO2011/014438), but have not yet been translated to the clinic.

WO 2018/213113 (Eli Lilly & Co.) discloses certain antibodies to BTLA.

WO2020128446 (Oxford University Innovation Limited and MiroBio Limited), which published 25 Jun. 2020, discloses certain antibodies to BTLA.

In humans there is one inhibitory Fc gamma receptor (FcγR2B) whilst the other Fc gamma receptors all deliver immune activating signals (FcγR1A, FcγR2A, FcγR3A and FcγR3B). The important regulatory role of FcγR2B has been demonstrated through studies of FcγR2B knockout mice that have increased susceptibility to autoimmune disease (Nakamura et al. Journal of Experimental Medicine 191(5): 899-906, 2000). Furthermore, a polymorphism in the FcγR2B gene in humans is associated with risk of autoimmune disease, in particular systemic lupus erythematosus (Floto et al. Nature Medicine 11(10), 2005). FcγR2B is therefore considered to play a key role in controlling immune responses and is a promising target molecule for controlling autoimmune and inflammatory diseases.

Antibodies having an Fc with improved FcγR2B binding activity have been reported (Chu et al. Molecular Immunology 45(15): 3926-33, 2008). In this Document, FcγR2B-binding activity was improved by adding alterations such as S267E/L328F, G236D/S267E, and S239D/S267E to an antibody Fc region. Among them, the antibody introduced with the S267E/L328F mutation most strongly binds to FcγR2B and maintains the same level of binding to FcγR1A and FcγR2A (131H allotype) as that of a naturally-occurring IgG1. However, another report shows that this alteration enhances the binding to FcγR2A 131R several hundred times, to the same level of FcγR2B binding, which means the FcγR2B-binding selectivity is not improved in comparison with FcγR2A 131R (US Patent Publication No. 2009/0136485).

For a BTLA agonist antibody to be effective at suppressing immune responses without eliciting inflammatory FcγR signaling, the inventors propose adapting the antibody for selective Fc binding to FcγR2B. Molecules with more selective binding to FcγR2B would promote bidirectional inhibitory signalling through BTLA on the BTLA expressing cell and through FcγR2B on the FcγR2B expression cell, which would strengthen the immunosuppressive effect of the antibody. This would be desirable in a therapeutic antibody intended for the treatment of diseases of immune overactivation.

### BRIEF SUMMARY OF THE DISCLOSURE

The present invention relates to BTLA agonist antibodies, including antibody fragments thereof, having one or more desirable properties, including high binding affinities, high agonist potency, high agonist efficacy, good pharmacokinetics and low antigenicity in human subjects. In certain embodiments, such molecules also have increased binding to and thus drive signaling of FcγR2B yet possess in vivo half-lives sufficient for appropriate therapeutic use. The antibodies of the invention thus promote bidirectional inhibitory signalling through BTLA on the BTLA expressing cell and through FcγR2B on the FcγR2B expressing cell. In certain embodiments, such molecules have reduced binding to one or more activating Fcgamma receptors, such as FcγR2A or FcγR1A compared to a parent polypeptide. In certain embodiments, such molecules have an increased ratio of binding to FcγR2B/FcγR2A compared to a parent polypeptide. In certain embodiments, such molecules have an increased ratio of binding to FcγR2B/FcγR1A compared to a parent polypeptide. The invention also relates to use of the antibodies of the invention in the treatment of disease, such as autoimmune and/or inflammatory diseases.

According to a first aspect of the invention there is provided an isolated antibody that specifically binds to human BTLA, wherein said antibody comprises a heavy chain and a light chain, wherein said heavy chain comprises an Fc region that comprises a substitution that results in increased binding to FcγR2B compared to a parent molecule that lacks the substitution.

In some embodiments the antibody has an increased binding to FcγR2B compared to a parent molecule such that the value of [KD value of parent polypeptide for FcγR2B]/[KD value of variant polypeptide for FcγR2B] is greater than 1, such as greater than 1.1, 1.2, 1.3, 1.4, 1.5, 2, 2.5, 3, 5, 10, 15, 20, 25, 30, 35, 40, 45, 50, 60, 70, 80, 90, or 100.

In some embodiments, the antibody has selectivity for binding FcγR2B over FcγR2A.

In some embodiments, the antibody has enhanced FcγR2B binding activity and maintained or decreased binding activities towards FcγR2A (type R) and/or FcγR2A (type H) in comparison with a parent polypeptide. In some embodiments the value of [KD value of variant polypeptide for FcγR2A (type R)]/[KD value of variant polypeptide for FcγR2B] is 2 or more, such as 3, 4, 5, 6, 7, 8, 9, 10 or more. In some embodiments the value of [KD value of variant polypeptide for FcγR2A (type H)]/[KD value of variant polypeptide for FcγR2B] is 2 or more, such as 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, or 150 or more.

In some embodiments, the antibody has enhanced FcγR2B binding activity and maintained or decreased binding activities towards FcγR1A in comparison with a parent polypeptide. In some embodiments the value of [KD value of variant polypeptide for FcγR1A]/[KD value of variant polypeptide for FcγR2B] is 0.05 or more, such as at least 0.1, 0.2, 0.3, 0.4, 0.5, 1, 2, 3, 4, or more.

In some embodiments the antibody has reduced Fcγ1 binding activity in comparison with a parent polypeptide. In some embodiments the value of [KD value of variant polypeptide for FcγR1A]/[KD value of parent polypeptide for FcγR1A] is at least 10, 20, 50, 100, 200.

In some embodiments, the antibody binds a residue of human BTLA selected from:
(i) D52, P53, E55, E57, E83, Q86, E103, L106 and E92 (position according to SEQ ID NO:225).
(ii) Y39, K41, R42, Q43, E45 and S47 (position according to SEQ ID NO:225);
(iii) D35, T78, K81, S121 and L123 (position according to SEQ ID NO:225);
(iv) N65 and A64 (position according to SEQ ID NO:225); or
(v) H68 (position according to SEQ ID NO:225).

In some embodiments, the antibody comprises a heavy chain and a light chain, wherein said heavy chain comprises an Fc region that comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, or alanine (A) at position 297 (all numbering according to EU Index). Suitably the antibody that specifically binds to human BTLA is an agonistic antibody/antigen-binding fragment.

Suitably, the antibody is a human IgG1 or IgG4 with one or more amino acid substitutions selected from the group consisting of: hIgG1 G236D, hIgG1 G237D, hIgG1 P238D, hIgG1 D265A, hIgG1 S267E, hIgG1 P271G, hIgG1 A330R, hIgG1 K322A, hIgG1 N297A, hIgG4 P238D, hIgG4 G237D, hIgG4 P271G, hIgG4 S330R, hIgG4 F234A and hIgG4 L235A.

According to a variation of the first aspect of the invention there is provided an antibody that specifically binds to human BTLA, wherein said antibody comprises a heavy chain and a light chain, wherein said heavy chain comprises an Fc region that comprises an aspartic acid at position 238 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises an aspartic acid at position 237 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises an aspartic acid at position 236 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises an alanine at position 235 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises an alanine at position 234 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises a glycine at position 271 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises a glutamic acid at position 267 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises an alanine at position 265 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises an alanine at position 297 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises an alanine at position 322 (EU Index). Suitably the antibody that specifically binds to human BTLA is an agonistic antibody/antigen-binding fragment.

According to a variation of the first aspect of the invention there is provided an antibody that specifically binds to human BTLA, wherein said antibody comprises a heavy chain and a light chain, wherein said heavy chain comprises an Fc region that comprises an arginine at position 330 (EU Index). Suitably the antibody that specifically binds to human BTLA is an agonistic antibody/antigen-binding fragment. According to a variation of the first aspect of the invention there is provided an antibody that specifically binds to human BTLA, wherein said antibody comprises a heavy chain and a light chain, wherein said heavy chain comprises an Fc region that comprises an aspartic acid at position 237 (EU Index), an aspartic acid at position 238 (EU Index), a glycine at position 271 (EU Index) and an arginine at position 330 (EU Index). Suitably the antibody that specifically binds to human BTLA is an agonistic antibody/antigen-binding fragment.

In particular embodiments, the antibody has a heavy chain and/or light chain with at least one complementarity-determining region (CDR) as present in an antibody selected from the group consisting of: 6.2, 2.8.6, 3E8, 11.5.1, 12F11, 14D4, 15B6, 15C6, 16E1, 16F10, 16H2, 1H6, 21C7, 24H7, 26B1, 26F3, 27G9, 3A9, 4B1, 4D3, 4D5, 4E8, 4H4, 6G8, 7A1, 8B4, 8C4, and 831, as identified in Table 1 or Table 2 and described herein. Suitably, said antibody also comprises an Fc region that comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, and alanine (A) at position 297 (all numbering according to EU Index).

In additional embodiments, the antibody which binds human BTLA is selected from the group consisting of 6.2, 2.8.6, 3E8, or an antibody that competes for binding to human BTLA with any one of 6.2, 2.8.6 or 3E8, wherein the antibody specifically binds BTLA and induces signaling through the receptor. Said antibody also comprises an Fc region that comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, and alanine (A) at position 297 (all numbering according to EU Index).

According to a variation of the first aspect of the invention there is provided an isolated antibody that specifically binds to human BTLA, wherein said antibody comprises a heavy chain and a light chain comprising the HCDR1, HCDR2, HCDR3, LCDR1, LCDR2 and LCDR sequences disclosed in SEQ ID Nos: 1, 17, 3, 4, 12 and 6, respectively and an Fc region that comprises a substitution that results in increased binding to FcγR2B compared to the parent molecule that lacks the substitution. Suitably, the said antibody comprises the VH and VL sequences disclosed in SEQ ID Nos: 18 and 14, respectively. Suitably, the said antibody comprises a heavy chain and a light chain, wherein the heavy chain comprises the amino acid sequence disclosed in SEQ ID NO: 19 and/or the light chain comprises the amino acid sequence disclosed in SEQ ID NO: 16. In a particular embodiment said antibody comprises an Fc region that comprises an aspartic acid at position 236 (EU Index). Suitably the antibody is an agonistic antibody.

In a particular embodiment said antibody comprises an Fc region that comprises an aspartic acid at position 237 (EU Index). Suitably the antibody is an agonistic antibody.

In a particular embodiment said antibody comprises an Fc region that comprises an aspartic acid at position 238 (EU Index). Suitably the antibody is an agonistic antibody.

In a particular embodiment said antibody comprises an Fc region that comprises an alanine at position 235 (EU Index).

In a particular embodiment said antibody comprises an Fc region that comprises an alanine at position 234 (EU Index).

In a particular embodiment said antibody comprises an Fc region that comprises an alanine at position 265 (EU Index).

In a particular embodiment said antibody comprises an Fc region that comprises a glutamic acid at position 267 (EU Index).

In a particular embodiment said antibody comprises an Fc region that comprises a glycine at position 271 (EU Index).

In a particular embodiment said antibody comprises an Fc region that comprises an alanine at position 297 (EU Index).

In a particular embodiment said antibody comprises an Fc region that comprises an alanine at position 322 (EU Index).

In a particular embodiment said antibody comprises an Fc region that comprises an arginine at position 330 (EU Index).

In a particular embodiment said antibody comprises an Fc region that comprises an aspartic acid at position 237 (EU Index), an aspartic acid at position 238 (EU Index), a glycine at position 271 (EU Index) and an arginine at position 330 (EU Index).

In particular embodiments of the first aspect of the invention the antibody possesses increased binding to FcγR2B compared to the parent molecule that lacks the Fc region substitution, i.e. one or more of: hIgG1 G236D, hIgG1 G237D, hIgG1 P238D, hIgG1 D265A, hIgG1 S267E, hIgG1 P271G, hIgG1 A330R, hIgG1 K322A, hIgG1 N297A, hIgG4 P238D, hIgG4 G237D, hIgG4 P271G, hIgG4 S330R, hIgG4 F234A and hIgG4 L235A.

In particular embodiments of the first aspect of the invention the antibody possesses increased binding to FcγR2B and reduced binding to one or more activating Fcgamma receptors, such as FcγR2A or FcγR1A, compared to the parent molecule that lacks the Fc region substitution.

In particular embodiments of the first aspect of the invention the antibody possesses increased ratio of binding to FcγR2B/FcγR2A, compared to the parent molecule that lacks the Fc region substitution. Suitably, the increased ratio of binding FcγR2B/FcγR2A, is at least 1.1, 1.2, 1.3, 1.4, 1.5, 1.8, 2, 2.2, 2.5, 3, 3.5, 4, 5, 6, 7, 8, 9 or 10-fold compared to the parent molecule that lacks the Fc region substitution.

In particular embodiments of the first aspect of the invention the antibody possesses increased ratio of binding to FcγR2B/FcγR1A, compared to the parent molecule that lacks the Fc region substitution over the wild-type sequence. Suitably, the increased ratio of binding FcγR2B/FcγR1A, is at least 1.1, 1.2, 1.5, 2, 5, 10, 50, 100, 150, 200, 250-fold compared to the parent molecule that lacks the Fc region substitution.

By compared to the parent molecule that lacks the Fc region substitution we mean compared to the antibody molecule that has the same amino acid sequence other than the amino acid recited in the claim which represents the Fc substitution relative to wildtype Fc. For example, including any of the following substitutions: hIgG1 G236D, hIgG1 G237D, hIgG1 P238D, hIgG1 D265A, hIgG1 S267E, hIgG1 P271G, hIgG1 A330R, hIgG1 K322A, hIgG1 N297A, hIgG4 P238D, hIgG4 G237D, hIgG4 P271G, hIgG4 S330R, hIgG4 F234A and hIgG4 L235A. Thus, binding of the antibody molecule with or without the recited Fc substitution to FcγR2B can be measured and optionally binding of the antibody molecule with or without the recited Fc substitution to an activating Fcγ receptor, such as FcγR2A or FcγR1A can be measured. Thus, for example, if the binding against FcγR2B has increased by 1.5 fold compared to the parent molecule without the substitution then it displays 150% increased binding efficiency compared to the parent. If the binding against FcγR2A has decreased by 1.5 fold compared to the parent molecule without the substitution then it displays 67% binding efficiency compared to the parent. For this exemplar antibody molecule, the change in FcγR2B/FcγR2A binding ratio would be 150/67=2.24 fold. Any value over 1 shows that the molecule has enhanced selectivity for binding FcγR2B over FcγR2A compared to the parent molecule.

In particular embodiments of the first aspect of the invention, the antibody has an increased ratio of [KD value for binding of FcγR1A]/[KD value for binding of FcγR2B] compared to the parent molecule that lacks the Fc region substitution over the wild-type sequence. Suitably, the ratio of [KD value for binding of FcγR1A]/[KD value for binding of FcγR2B] for the variant molecule is at least 1.1, 1.2, 1.5, 2, 5, 10, 50, 100, 150, 200, 250, 300, 350, 400, 450, 500, 1000, 1500, 2000, 3000, 4000, 5000, 6000, 7000, 8000, 9000, or 10000 times the ratio of [KD value for binding of FcγR1A]/[KD value for binding of FcγR2B] for the parent molecule that lacks the Fc region substitution.

In particular embodiments of the first aspect of the invention, the antibody has an increased ratio of [KD value for binding of FcγR2A 131R]/[KD value for binding of FcγR2B] compared to the parent molecule that lacks the Fc region substitution over the wild-type sequence. Suitably, the ratio of [KD value for binding of FcγR2A 131R]/[KD value for binding of FcγR2B] for the variant molecule is at least 1.1, 1.2, 1.5, 2, 5, 10, 50, or 100 times the ratio of [KD value for binding of FcγR1A]/[KD value for binding of FcγR2B] for the parent molecule that lacks the Fc region substitution.

According to a second aspect of the invention there is provided an isolated nucleic acid comprising a nucleotide sequence that encodes a heavy chain polypeptide and/or a light chain polypeptide of the isolated antibody of the first aspect of the invention.

According to a third aspect of the invention there is provided a vector comprising the nucleic acid of the second aspect of the invention.

According to a fourth aspect of the invention there is provided a host cell comprising the nucleic acid sequence according to the second aspect of the invention or the vector according to the third aspect of the invention.

According to a fifth aspect of the invention there is provided a method of producing an antibody according to the first aspect of the invention, comprising the step of culturing the host cell of the fourth aspect of the invention under conditions for production of said antibody, and optionally isolating and/or purifying said antibody.

According to a sixth aspect of the invention there is provided a pharmaceutical composition comprising a pharmaceutically acceptable carrier and a therapeutically effective amount of the antibody of the first aspect of the invention, or that produced by the fifth aspect of the invention.

According to an seventh aspect of the invention there is provided a method of preparing a pharmaceutical composition, the method comprising formulating antibody in accordance with the first aspect of the invention, or one produced in accordance with the fifth aspect of the invention into a composition including at least one additional component. In a particular embodiment, the at least one additional component is a pharmaceutically acceptable excipient.

According to an eighth aspect of the invention there is provided a kit comprising an antibody in accordance with the first aspect of the invention or the pharmaceutical composition in accordance with the sixth aspect of the invention. Suitably, such a kit includes a package insert comprising instructions for use

According to a ninth aspect of the invention there is provided a method of treating a BTLA-related disease in a patient, comprising administering to the patient a therapeutically effective amount of the antibody of the first aspect of the invention or the pharmaceutical composition of the sixth aspect of the invention.

Suitably, the BTLA-related disease is an autoimmune or inflammatory disease.

### DETAILED DESCRIPTION

The inventors have identified particularly strong agonist antibodies to BTLA which are predicted to be more efficacious than current antibodies at suppressing T cell responses and thus be particularly useful in the treatment of immune mediated disorders. Such antibodies comprise at least one substitution at a location in the Fc portion of the molecule that selectively enhances binding to FcγR2B compared to a parent polypeptide. Suitably the antibody comprises a substitution at one or more of the following locations (EU Index positions): 234, 235, 236, 237, 238, 265, 267, 271, 297, 330 and 322. Suitably, the antibody is a human IgG1 or IgG4 with one or more amino acid substitutions selected from the group consisting of: hIgG1 G236D, hIgG1 G237D, hIgG1 P238D, hIgG1 D265A, hIgG1 S267E, hIgG1 P271G, hIgG1 A330R, hIgG1 K322A, hIgG1 N297A, hIgG4 P238D, hIgG4 G237D, hIgG4 P271G, hIgG4 S330R, hIgG4 F234A and hIgG4 L235A, all using EU Index numbering.

Modifications at one or more of the following positions: 236, 237, 238 and 267 (EU Index) are particularly suitable.

Combinations of Fc modifications are also suitable. In a particular embodiment, the set of modifications termed V9 is employed, wherein the antibody heavy chain comprises an Fc region that comprises an aspartic acid at position 237, an aspartic acid at position 238, a glycine at position 271, and an arginine at position 330 (numbering according to EU Index).

By introducing a P238D (EU Index) substitution into the Fc portion of the molecule (i.e. the antibody or antigen-binding fragment thereof), the molecule has enhanced binding to and signaling of FcγR2B but at a level that ensures that the antibody retains a sufficient in vivo half-life.

As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that wherever aspects are described herein with the language “comprising,” otherwise analogous aspects described in terms of “consisting of” and/or “consisting essentially of” are also provided.

It is to be understood that one, some, or all of the properties of the various embodiments described herein may be applied to any aspect unless the content clearly dictates otherwise. Furthermore, that the various embodiments may be combined to form other embodiments of the present invention. These and other aspects of the invention will become apparent to one of skill in the art. These and other embodiments of the invention are further described by the detailed description that follows.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of ordinary skill with a general dictionary of many of the terms used in this disclosure.

The term “about” as used herein refers to the usual error range for the respective value readily known to the skilled person in this technical field. Reference to “about” a value or parameter herein includes (and describes) embodiments that are directed to that value or parameter per se.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The carboxy-terminal portion of each chain defines a constant region primarily responsible for effector function. The amino-terminal portion of each chain defines a variable region responsible for binding to antigen. Kabat et al. (NIH Pub. No. 91/3242, p. 679-687; 1991) collected numerous primary sequences of the variable regions of heavy chains and light chains. Based on the degree of conservation of the sequences, they classified individual primary sequences into the CDR and the framework and made a list thereof (see Kabat et al., SEQUENCES OF IMMUNOLOGICAL INTEREST, 5th edition, NIH publication, No. 91-3242; 1991).

The identified CDRs of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). The numbering of amino acids in the variable domains is ordinal, based on the sequences provided in the sequence listing.

The numbering of amino acids in the constant domains, such as C_{L}, C_{H}1, C_{H}2, and C_{H}3 follow, unless otherwise indicated, the EU Index numbering disclosed in Kabat et al., (NIH Pub. No. 91/3242, p. 679-687; 1991), referred to herein as “EU Index”. For example, the EU Index is used to locate the substitution in the Fc region of the antibodies/antigen-binding fragments thereof of the invention. For example, glycine (G) to aspartic acid (D) at position 236 (identified as G236D) or proline (P) to aspartic acid (D) at position 238 (identified as P238D). Those skilled in the art of antibodies will appreciate that this numbering convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

The terms “B and T lymphocyte attenuator” and “BTLA” are used interchangeably and, unless the context dictates otherwise, with reference to either the protein or gene (or other nucleic acid encoding all or part of BTLA). The human BTLA sequences encompass all human isotype and variant forms. A representative example of full length human BTLA is disclosed in Genbank under accession number: AJ717664.1. Another representative polypeptide sequence of human BTLA is disclosed in SEQ ID NO: 225, which only differs from that in AJ717664.1 by two natural variant single nucleotide polymorphisms. Despite allelic variations, a human BTLA polypeptide sequence will typically have at least 90% sequence identity (such as at least 95%, 96%, 97%, 98%, 99% or 100%) to human BTLA in SEQ ID NO: 225.

A representative example of full length cynomolgus (cyno) BTLA is disclosed in Genbank under accession number: XP_005548224. A reference polypeptide sequence of cyno BTLA is disclosed in SEQ ID NO: 226. A cyno BTLA polypeptide sequence will typically have at least 90% sequence identity (such as at least 95%, 96%, 97%, 98%, 99% or 100%) to cyno BTLA as disclosed in SEQ ID NO: 226.

The term sequence identity is well known in the art. For the purposes of this invention, when determining whether a target sequence meets a defined limit (e.g. 90% identity), it is considered to meet the defined limit if it is identified as such using the BLAST (Basic local alignment search tool) algorithm (see Altschul et al. J Mol Biol 215:403-410, 1990) or Smith-Waterman algorithm (see Smith and Waterman. J Mol. Biol. 147:195-197, 1981).

### Antibodies (Including Antigen-Binding Fragments of Antibodies)

An antibody is an immunoglobulin molecule capable of specific binding to a target, such as a protein, polypeptide, peptide, carbohydrate, polynucleotide, lipid, or combinations of the foregoing through at least one antigen recognition site, located in the variable domain of the immunoglobulin molecule. In particular, as used herein, the term “antibody” encompasses intact polyclonal antibodies, intact monoclonal antibodies, multispecific antibodies such as bispecific antibodies generated from at least two intact antibodies, chimeric antibodies, humanised antibodies, human antibodies, any other modified immunoglobulin molecule and any fragments thereof comprising an antigen recognition site so long as the antibodies exhibit the desired biological activity. The antibody can be from any species. Suitably the antibody is a human antibody.

The term “antibody” as used herein, refers to an immunoglobulin molecule which specifically binds to an antigen and comprises an FcR binding site which may or may not be functional. The term embraces whole antibodies (such as IgG1, IgG4 and the like) and antigen binding fragments thereof.

As used herein, a BTLA agonist antibody refers to an antibody (including an antigen-binding fragment of an intact antibody) that binds to BTLA and enhances its coinhibitory signal to T and/or B cells.

The antigen-binding site refers to the part of a molecule that binds to all or part of the target antigen. In an antibody molecule it may be referred to as the antibody antigen-binding site and comprises the part of the antibody that specifically binds to all or part of the target antigen. Where an antigen is large, an antibody may only bind to a particular part of the antigen, which part is termed an epitope. An antibody antigen-binding site may be provided by one or more antibody variable domains. Preferably, an antibody antigen-binding site comprises an antibody light chain variable region (VL) and an antibody heavy chain variable region (VH).

The invention also encompasses antibody-fragments that comprise an antigen-binding site. Thus, the term “antigen-binding fragment thereof”, when referring to an antibody refers to antibody fragments, such as Fab, Fab′, F(ab′)2, diabodies, Fv fragments and single chain Fv (scFv) mutants that possess an antigen recognition site, and thus, the ability to bind to an antigen. Antigen-binding immunoglobulin (antibody) fragments are well known in the art. Such fragment need not have a functional Fc receptor binding site. In particular embodiments, the antigen-binding fragment thereof comprise an Fc portion with a substitution selected from one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, and alanine (A) at position 297 (all numbering according to EU Index). In a particular embodiment, the antigen-binding fragment thereof comprise an Fc portion with G236D (EU Index) substitution. In a particular embodiment, the antigen-binding fragment thereof comprise an Fc portion with P238D (EU Index) substitution. In a particular embodiment, the antigen-binding fragment thereof comprise an Fc portion with an aspartic acid at position 237 (EU Index), an aspartic acid at position 238 (EU Index), a glycine at position 271 (EU Index) and an arginine at position 330 (EU Index).

As used herein the terms “antibody fragment molecules of the invention”, “antibody fragment” and “antigen-binding fragment thereof”, are used interchangeably herein. Collectively an antibody or an antigen-binding fragment thereof may be referred to as an antigen-binding molecule.

The term “BTLA-binding molecule” as used herein, refers to both antibodies and binding fragments thereof capable of binding to BTLA.

There are five major classes (i.e., isotypes) of immunoglobulins: IgA, IgD, IgE, IgG, and IgM, and several of these may be further divided into subclasses (subtypes), e.g., IgG1, 1gG2, 1gG3, 1gG4, IgA1 and 1gA2. The heavy-chain constant regions that correspond to the different classes of immunoglobulins are called alpha, delta, epsilon, gamma, and mu, respectively. The subunit structures and three-dimensional configurations of different classes of immunoglobulins are well known. Unless dictated otherwise by contextual constraints the antibodies of the invention can be from one of these classes or subclasses of antibodies. Heavy-chain constant domains that correspond to the different classes of antibodies are typically denoted by the corresponding lower-case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

“Native antibodies” are usually heterotetrameric Y-shaped glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains. Each heavy chain comprises one variable domain (VH) and a constant region, which in the case of IgG, IgA, and IgD antibodies, comprises three domains termed C_{H}1, C_{H}2, and C_{H}3 (IgM and IgE have a fourth domain, C_{H}4). In IgG, IgA, and IgD classes the C_{H}1 and C_{H}2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues. The heavy chain variable region (VH) and light chain variable region (VL) can each be further subdivided into regions of hypervariability, termed CDRs, interspersed with regions that are more conserved, termed framework regions (FR). Each VH and VL, comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g. effector cells) and the first component (C1q) of the classical complement system.

The antibody of the invention may be from any animal species including murine, rat, human, or any other origin (including chimeric or humanised antibodies). In some embodiments, the antibody is monoclonal, e,g. a monoclonal antibody. In some embodiments, the antibody thereof is a human or humanised antibody or antigen-binding fragment thereof. A non-human antibody or antigen-binding fragment thereof may be humanised by recombinant methods to reduce its immunogenicity in man.

The term “monoclonal antibody” (“mAb”) as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody or fragment thereof, as not being a mixture of discrete antibodies or antigen-binding fragments. A mAb is typically highly specific, being directed against a single antigenic site/epitope, however a monoclonal antibody can also refer to a population of a substantially homogeneous bispecific antibody molecule.

A mAb may be produced by hybridoma, recombinant, transgenic or other techniques known to those skilled in the art. For example, a monoclonal antibody or antigen-binding fragment thereof in accordance with the present invention may be made by the hybridoma method first described by Kohler and Milstein (Nature 256:495, 1975) or may be made by recombinant DNA methods such as described in U.S. Pat. Nos. 4,816,567 and 6,331,415. The “monoclonal antibodies” may also be isolated from phage antibody libraries using the techniques described in Clackson et al., Nature 1991; 352:624-628 and Marks et al., J. Mol. Biol. 1991; 222:581-597, for example.

The term monoclonal may also be ascribed to an antigen-binding fragment of an antibody of the invention. It merely means that the molecule is produced or present in a single clonal form.

A “human” antibody (HumAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

Human antibodies can be prepared by administering an immunogen/antigen to a transgenic animal that has been modified to produce intact human antibodies or intact antibodies with human variable regions in response to antigenic challenge, but whose endogenous loci have been disabled, e.g., immunized xenomice (see, e.g., U.S. Pat. Nos. 6,075,181 and 6,150,584 regarding XENOMOUSE (trade mark) technology). See also, for example, Li et al., Proc. Natl. Acad. Sci. USA, 103:3557-3562 (2006) regarding human antibodies generated via a human B-cell hybridoma technology. Such animals typically contain all or a portion of the human immunoglobulin loci, which replace the endogenous immunoglobulin loci, or which are present extra chromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB™ technology; U.S. Pat. No. 7,041,870 describing K-M MOUSE™ technology, and U.S. Patent Application Publication No. US2007/0061900, describing VELOCIMOUSE™ technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol, 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991)) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26:265-268 (2006) (describing human-human hybridomas). Human hybridoma technology (Trioma technology) is also described in Vollmers and Brandlein, Histology and Histopathology, 20:927-937 (2005) and Vollmers and Brandlein, Methods and Findings in Experimental and Clinical Pharmacology, 27:185-91 (2005).

The terms “human” antibodies and “fully human” antibodies are used synonymously. This definition of a human antibody specifically excludes a humanised antibody comprising non-human antigen-binding residues.

As used herein, a “humanised antibody” refers to an antibody in which some, most or all of the amino acids outside the CDRs of a non-human antibody are replaced with corresponding amino acids derived from human immunoglobulins. In some embodiments, humanised antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanised antibody may comprise residues that are found neither in the recipient antibody nor in the imported CDR or framework sequences but are included to further refine and optimize antibody performance. In one embodiment of a humanised form of an Ab, some, most or all the amino acids outside the CDRs have been replaced with amino acids from human immunoglobulins, whereas some, most or all amino acids within one or more CDR regions are unchanged. Small additions, deletions, insertions, substitutions or modifications of amino acids are permissible provided they do not abrogate the ability of the antibody to bind to a particular antigen. A “humanised” antibody retains an antigenic specificity similar to that of the original antibody. In general, a humanised antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanised antibody optionally will also comprise at least a portion of an immunoglobulin constant region (Fc), typically that of a human immunoglobulin. For further details, see, e.g., Jones et al, Nature 321:522-525 (1986); Riechmann et al, Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. Suitably, the Fc will comprise the P238D substitution mutation (using “EU Index” numbering) to enhance the specificity for binding FcγR2B.

As used herein, Fc, Fc portion or Fc region, refers to the constant region of an antibody or antibody-like molecule excluding the first constant region immunoglobulin domain. Thus, Fc refers to the last two constant region immunoglobulin domains of IgA, IgD and IgG, and the last three constant region immunoglobulin domains of IgE, IgM, and the flexible hinge N-terminal to these domains. For IgG, Fc comprises immunoglobulin domains Cgamma2 and Cgamma3 (Cγ2 and Cγ3) and the hinge between Cgamma1 (Cγ1) and Cγ2. For IgA and IgM, Fc may include the J chain.

As used herein, an “engineered antibody” refers to an antibody, which may be a humanised antibody, wherein particular residues have been substituted for others so as to diminish an adverse effect or property. Such substitution could be within a CD domain. For example, as described herein (see Example 21), the CDRH2 of the humanised antibody 3E8 was modified with an N57Q substitution to remove deamidation potential, and/or a K63 S substitution to reduce predicted immunogenicity. The numbering in this instance is ordinal with reference to the provided sequence identifier.

A “chimeric antibody” refers to an antibody in which the variable regions are derived from one species and the constant regions are derived from another species, such as an antibody in which the variable regions are derived from a mouse antibody and the constant regions are derived from a human antibody or vice versa. The term also encompasses an antibody comprising a V region from one individual from one species (e.g., a first mouse) and a constant region from another individual from the same species (e.g., a second mouse). The term “antigen (Ag)” refers to the molecular entity used for immunization of an immunocompetent vertebrate to produce the antibody (Ab) that recognizes the Ag or to screen an expression library (e.g., phage, yeast or ribosome display library, among others). Herein, Ag is termed more broadly and is generally intended to include target molecules that are specifically recognized by the Ab, thus including portions or mimics of the molecule used in an immunization process for raising the Ab or in library screening for selecting the Ab.

A “bispecific” or “bifunctional” antibody is an artificial hybrid antibody having two different heavy/light chain pairs and two different binding sites. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Methods for making bispecific antibodies are within the purview of those skilled in the art. For example, bispecific antibodies can be produced by a variety of methods including fusion of hybridomas or linking of Fab′ fragments. See, e.g., Songsivilai, et al, (1990) Clin. Exp. Immunol. 79: 315-321, Kostelny, et al, (1992) J Immunol. 148:1547-1553. In addition, bispecific antibodies may be formed as “diabodies” (Holliger, et al, (1993) PNAS USA 90:6444-6448) or as “Janusins” (Traunecker, et al, (1991) EMBO J. 10:3655-3659 and Traunecker, et al, (1992) Int. J. Cancer Suppl. 7:51-52). Full length bispecific antibodies may be generated for example using Fab arm exchange (or half molecule exchange) between two monospecific bivalent antibodies by introducing substitutions at the heavy chain CH3 interface in each half molecule to favour heterodimer formation of two antibody half molecules having distinct specificity either in vitro in cell-free environment or using co-expression. The Fab arm exchange reaction is the result of a disulphide bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favour heterodimerization over homodimerization. The resulting product is a bispecific antibody having two Fab arms or half molecules which each bind a distinct epitope. The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A Y407V.

Bispecific antibodies may also be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. Another strategy for generating bispecific antibodies involves promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532.

Suitably, one of the two antibody half molecules in a bispecific molecule is an anti-BTLA antibody of the invention. Suitably the bispecific antibody comprises one binding arm that comprises a BTLA antigen binding region as disclosed herein and a second binding arm that comprises a binding region to another antigen (e.g. to a different BTLA antigen epitope or a completely different protein) and wherein the molecule comprises an Fc region that comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, and alanine (A) at position 297 (all numbering according to EU Index).

Generally, the term “epitope” refers to the area or region of an antigen to which an antibody specifically binds, i.e., an area or region in physical contact with the antibody. Thus, the term “epitope” refers to that portion of a molecule capable of being recognized by and bound by an antibody at one or more of the antibody's antigen-binding regions. Typically, an epitope is defined in the context of a molecular interaction between an “antibody, or antigen-binding portion thereof (Ab), and its corresponding antigen. Epitopes often consist of a surface grouping of molecules such as amino acids or sugar side chains and have specific three-dimensional structural characteristics as well as specific charge characteristics. In some embodiments, the epitope can be a protein epitope. Protein epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises non-contiguous polypeptides (or amino acids) within the antigenic protein to which an antibody specific to the epitope binds. The term “antigenic epitope” as used herein, is defined as a portion of an antigen to which an antibody can specifically bind as determined by any method well known in the art, for example, by conventional immunoassays.

An antibody that “specifically binds” to an epitope is a term well understood in the art, and methods to determine such specific binding are also well known in the art. A molecule is said to exhibit “specific binding” if it reacts or associates more frequently, more rapidly, with greater duration and/or with greater affinity with a particular cell, protein or substance than it does with alternative cells, proteins or substances.

A variety of assay formats may be used to select an antibody or peptide that specifically binds a molecule of interest. For example, solid-phase ELISA immunoassay, immunoprecipitation, Biacore™ (GE Healthcare, Piscataway, N.J.), KinExA, fluorescence-activated cell sorting (FACS), Octet™ (ForteBio, Inc., Menlo Park, Calif.) and Western blot analysis are among many assays that may be used to identify an antibody that specifically reacts with an antigen or a receptor, or ligand binding portion thereof, that specifically binds with a cognate ligand or binding partner. Typically, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background. Also, an antibody is said to “specifically bind” an antigen when the equilibrium dissociation constant (K_{D} or KD, as used interchangeably herein) is <7 nM.

In some embodiments, the present disclosure provides a chimeric antigen receptor comprising an antigen binding fragment of a BTLA binding antibody disclosed herein, a transmembrane domain, and an intracellular signaling domain. The term “Chimeric Antigen Receptor” (CAR), “artificial T cell receptor,” “chimeric T cell receptor,” or “chimeric immunoreceptor” as used herein refers to an engineered receptor, which grafts an arbitrary specificity onto an immune effector cell. CARs typically have an extracellular domain (ectodomain), which comprises an antigen-binding domain, a transmembrane domain, and an intracellular (endodomain) domain. The term “signaling domain” refers to the functional portion of a protein which acts by transmitting information within the cell to regulate cellular activity via defined signaling pathways by generating second messengers or functioning as effectors by responding to such messengers.

### Fcgamma Modifications

In humans, the FcγR1A (CD64A), FcγR2A (CD32A), FcγR2B (CD32B), FcγR3A (CD16A), and FcγR3B (CD16B) isoforms have been reported as the FcγR protein family, and different allotypes of these receptors have also been reported (Jefferis and Lund, Immunology Letters 82(1-2): 57-65, 2002). FcγR1A, FcγR2A, and FcγR3A are called activating FcγRs since they have immunologically active functions, and FcγR2B is called an inhibitory FcγR since it has immunosuppressive functions (Smith and Clatworthy, Nat Rev Immunol, 10(5), 328-343, 2010). In the literature, and herein, FcγR1A may also be referred to as FcγR1.

When activating FcγRs are triggered by binding to an antibody Fc region it leads to phosphorylation of immunoreceptor tyrosine-based activating motifs (ITAMs) contained in the intracellular domain or FcR common γ-chain (an interaction partner) and triggers an inflammatory immune response by initiating an activation signal cascade (Nimmerjahn and Ravetch, Nat Rev Immunol 8(1): 34-47, 2008). When the inhibitory receptor FcγR2B is triggered by binding to an antibody Fc region it leads to phosphorylation of immunoreceptor tyrosine-based inhibitory motifs (ITIMs) in the cytoplasmic tail, with subsequent recruitment of SH2-containing inositol polyphosphate 5-phosphatase (SHIP1), which in turn inhibits transduction of other activating signal cascades, and so suppresses the inflammatory immune response (Ravetch and Lanier, Science 290(5489): 84-89, 2000).

FcγR2B is the only FcγR expressed on B cells (Amigorena et al. European Journal of Immunology 19(8): 1379-85, 1989). Interaction of the antibody Fc region with FcγR2B has been reported to inhibit signaling through the B cell receptor, suppressing B cell proliferation and antibody production (Nimmerjahn and Ravetch, Advances in immunology 96: 179-204, 2007). In cell types expressing both activatory and inhibitory FcγR (such as macrophages, DCs, neutrophils, mast cells and basophils) the signalling threshold and outcome of FcγR engagement is determined by the balance of activating and inhibitory FcγR activation (Nimmerjahn and Ravetch, Science 310(5753): 1510-12, 2005).

The important regulatory role of FcγR2B has been demonstrated through studies of FcγR2B knockout mice that have increased susceptibility to autoimmune disease (Nakamura et al. Journal of Experimental Medicine 191(5): 899-906, 2000). Furthermore, a polymorphism in the FcγR2B gene in humans is associated with risk of autoimmune disease, in particular systemic lupus erythematosus (Floto et al. Nature Medicine 11(10), 2005). FcγR2B is therefore considered to play a key role in controlling immune responses and is a promising target molecule for controlling autoimmune and inflammatory diseases.

IgG1 and IgG4, the most commonly used antibody isotypes for commercially available antibody pharmaceuticals, are known to bind not only to FcγR2B but also strongly to activating FcγR (Bruhns et al. Blood 113(16): 3716-25, 2009). It may be possible to develop antibody pharmaceuticals having greater immunosuppressive properties compared with those of IgG1 or IgG4, by utilizing an Fc region with enhanced FcγR2B binding, or improved FcγR2B binding selectivity compared with activating FcγR.

Antibodies having an Fc with improved FcγR2B binding activity have been reported (Chu et al. Molecular Immunology 45(15): 3926-33, 2008). In this Document, FcγR2B-binding activity was improved by adding alterations such as S267E/L328F, G236D/S267E, and S239D/S267E to an antibody Fc region. Among them, the antibody introduced with the S267E/L328F mutation most strongly binds to FcγR2B and maintains the same level of binding to FcγR1A and FcγR2A (131H allotype) as that of a naturally-occurring IgG1. However, another report shows that this alteration enhances the binding to FcγR2A 131R several hundred times, to the same level of FcγR2B binding, which means the FcγR2B-binding selectivity is not improved in comparison with FcγR2A 131R (US Patent Publication No. 2009/0136485). In addition to its proinflammatory effects, antibody binding to FcγR2A can lead to activation of platelets resulting in thromboembolic events, as seen with the therapeutic antibody Bevacizumab (Meyer et al. Journal of Thrombosis and Haemostasis 7(1): 171-81, 2009; Scappaticci et al. Journal of the National Cancer Institute 99(16): 1232-39, 2007) and with antibodies targeting the CD40 ligand (Boumpas et al. Arthritis and rheumatism 48(3): 719-27, 2003; Robles-Carrillo et al. Journal of immunology (Baltimore, Md.: 1950) 185(3): 1577-83, 2010). Furthermore, antibodies with enhanced FcγR2A binding have been reported to enhance macrophage-mediated antibody dependent cellular phagocytosis (ADCP) (Richards et al. Molecular Cancer Therapeutics 7(8): 2517-27, 2008). When antibody's antigens are phagocytized by macrophages, antibodies themselves are also phagocytized at the same time. In that case, peptide fragments derived from those antibodies are also presented as an antigen and the antigenicity may become higher, thereby increasing the risk of production of antibodies against antibodies (anti-drug antibodies). More specifically, enhancing FcγR2A binding will increase the risk of production of antibodies against the antibodies, and this will remarkably decrease their value as pharmaceuticals. Therefore, antibodies with selective binding to FcγR2B and reduced binding to FcγR2A might be more effective immunosuppressives and also better tolerated therapeutics with a lower risk of inducing thromboembolic events and a lower immunogenicity.

Thus, for a BTLA agonist antibody to be effective at suppressing immune responses without eliciting inflammatory FcR signaling, the inventors propose adapting the antibody for selective Fc binding to FcγR2B.

Molecules with more selective binding to FcγR2B would promote bidirectional inhibitory signaling through BTLA on the BTLA expressing cell and through FcγR2B on the FcγR2B expression cell, which would strengthen the immunosuppressive effect of the antibody. This would be desirable in a therapeutic antibody intended for the treatment of diseases of immune overactivation.

However, very high affinity for FcγR2B can adversely impact antibody half-life due to turnover of the receptor in liver sinusoidal epithelial cells (Ganesan et al. The Journal of Immunology 189(10): 4981-88, 2012) as demonstrated by the FcγR2B enhanced IgG1 antibody XmAb7195 which binds to FcγR2B with a KD of 7.74 nM (Chu et al. Journal of Allergy and Clinical Immunology 129(4): 1102-15, 2012; https://linkinghub.elsevier.com/retrieve/pii/S0091674911018343 (May 13, 2020) and was reported by Xencor to have an average in vivo half-life of 3.9 days in a phase 1a trial (American Thoracic Society (ATS) 2016 International Conference in San Francisco, Calif.—A6476: Poster Board Number 407), compared to an average half-life of around 21 days for a wildtype IgG1 (Morell, Terry, and Waldmann. Journal of Clinical Investigation 49(4): 673-80, 1970; http://www.jci.org/articles/view/106279 (May 16, 2020)). Therefore, in the context of the present invention, whilst selectivity for FcγR2B and sufficient binding to support agonism might be desirable for a BTLA agonist antibody, excessively high affinity for FcγR2B might be undesirable in a therapeutic as the consequently shortened half-life would likely necessitate more frequent dosing.

Various mutations, including amino acid substitutions, can be incorporated into the heavy chain constant region of an antibody in order to modify signaling through one or more Fcγ receptors. WO2006/019447 (Xencor) discloses various Fc variant molecules (e.g. antibodies) with altered effector function through amino acid substitution in the Fe region.

The inventors have found that incorporation of P238D substitution mutation into a BTLA agonist antibody of the invention enhances the selectivity for binding to and signaling through FcγR2B without significantly diminishing the in vivo half-life of the antibody.

Whilst the Fc portion can accommodate other modifications (such as amino acid substitutions), in a particular embodiment the P238D modification is the only one introduced into the BTLA-binding molecules of the invention and relative to wild-type Ig Fc sequence.

In one embodiment, the antibody comprises an aspartic acid at position corresponding to position 238 of IgG1 (using EU Index). Suitably, the antibody of the invention comprises the hIgG1 constant region disclosed in SEQ ID NO: 227, or one with up to 5 amino acid modifications provided the P238D substitution is present.

In one embodiment, the antibody comprises an aspartic acid at position corresponding to position 238 of IgG4 (using EU Index). Suitably, the antibody of the invention comprises the hIgG4 constant region disclosed in SEQ ID NO: 235, or one with up to 5 amino acid modifications provided the P238D substitution is present.

The antibodies of the invention promote bidirectional inhibitory signaling through BTLA on the BTLA expressing cell and through FcγR2B on the FcγR2B expressing cell and possess in vivo half-lives sufficient for appropriate therapeutic use. Suitably the in vivo half-life is at least 5 days, such as at least 5, 6, 7, 8, 9, 10, 11, 12, 13, 14, 15, 16, 17, 18, 19, 20, 21, 22, 23, 24 or more days, in the human body. In a particular embodiment the in vivo half-life in a human is at least 10 days which will allow a suitable dosing regime, e.g. 3 weekly. Suitably the in vivo half-life is between about 10 and 30, such as between about 12 and 20 or 14 and 25 days.

In a particular embodiment of the invention the antibody of the invention exhibits an in vivo half-life within ±3 days of the half-life of a comparable control antibody that comprises a wild-type Fc region. The comparable control antibody being one that has the same heavy and light chain except for the Fc modification(s) that increases binding to FcγR2B, as described herein.

In a particular embodiment of the invention the antibody of the invention exhibits an in vivo half-life that retains at least 50%, such as at least 60%, at least 70%, at least 80% at least 90% of the half-life of a comparable control antibody that comprises a wild-type Fc region. The comparable control antibody being one that has the same heavy and light chain except for the Fc modification(s) that increases binding to FcγR2B, as described herein.

When the Fc modification that increases binding to FcγR2B is P238D substitution, in a particular embodiment the antibody of the invention exhibits an in vivo half-life within ±3 days of the half-life of a comparable control antibody that comprises an Fc region that comprises a proline at position 238 (EU Index).

In another particular embodiment, when the Fc modification that increases binding to FcγR2B is P238D substitution, the antibody of the invention exhibits an in vivo half-life that retains at least 50%, such as at least 60%, at least 70%, at least 80% at least 90% of the half-life of the parent antibody that comprises an Fc region that comprises a proline at position 238 (EU Index).

The longer the half-life the longer the period for which good receptor occupancy is achieved. This then means the longer the interval between doses, or in the alternative to a longer dose interval, a longer half-life would allow a lower dose to be given—which could be important if there are dose limiting toxicities at higher peak doses.

Producing an antibody with a long half-life may also have benefits such as reduced cost of goods, reduced treatment burden on the patient and increased patient compliance.

Suitably, the molecules of the invention are capable of a receptor occupancy >80% for at least 10, such as 14, 21, 28, 35, 42 or more days after a single dose of 10 mg/kg.

Suitably, the molecules of the invention are capable of being administered at a dose interval of 3 weeks, ideally 4 or more weeks, such as every 6 or 8 weeks.

According to a first aspect of the invention there is provided an antibody that specifically binds to human BTLA, wherein said antibody comprises a heavy chain and a light chain, wherein said heavy chain comprises an Fc region that comprises a substitution that results in increased binding to FcγR2B compared to the parent molecule that lacks the substitution. Suitably the antibody is an isolated antibody.

In some embodiments the antibody has an increased binding to FcγR2B compared to a parent molecule such that the value of [KD value of parent polypeptide for FcγR2B]/[KD value of variant polypeptide for FcγR2B] is greater than 1, such as greater than 1.1, 1.2, 1.3, 1.4, 1.5, 2, 2.5, 3, 5, 10, 15, 20, 25, 30, 35, 40, 45, 50, 60, 70, 80, 90, or 100.

In some embodiments, the antibody has selectivity for binding FcγR2B over FcγR2A.

In some embodiments, the antibody has enhanced FcγR2B binding activity and maintained or decreased binding activities towards FcγR2A (type R) and/or FcγR2A (type H) in comparison with a parent polypeptide. In some embodiments the value of [KD value of variant polypeptide for FcγR2A (type R)]/[KD value of variant polypeptide for FcγR2B] is 2 or more, such as 3, 4, 5, 6, 7, 8, 9, 10 or more. In some embodiments the value of [KD value of variant polypeptide for FcγR2A (type H)]/[KD value of variant polypeptide for FcγR2B] is 2 or more, such as 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, or 150 or more.

In some embodiments, the antibody has enhanced FcγR2B binding activity and maintained or decreased binding activities towards FcγR1A in comparison with a parent polypeptide. In some embodiments the value of [KD value of variant polypeptide for FcγR1A]/[KD value of variant polypeptide for FcγR2B] is 0.05 or more, such as at least 0.1, 0.2, 0.3, 0.4, 0.5, 1, 2, 3, 4, or more.

In some embodiments the antibody has reduced Fcγ1 binding activity in comparison with a parent polypeptide. In some embodiments the value of [KD value of variant polypeptide for FcγR1A]/[KD value of parent polypeptide for FcγR1A] is at least 10, 20, 50, 100, 200.

In some embodiments, the antibody binds a residue of human BTLA selected from: D52, P53, E55, E57, E83, Q86, E103, L106 and E92 (position according to SEQ ID NO:225). In some embodiments, the antibody binds a residue of human BTLA selected from: Y39, K41, R42, Q43, E45 and S47 (position according to SEQ ID NO:225). In some embodiments, the antibody binds a residue of human BTLA selected from: D35, T78, K81, S121 and L123 (position according to SEQ ID NO:225). In some embodiments, the antibody binds residue H68 of human BTLA (position according to SEQ ID NO:225). In some embodiments, the antibody binds a residue of human BTLA selected from: N65 and A64 (position according to SEQ ID NO:225).

In some embodiments, the antibody comprises a heavy chain and a light chain, wherein said heavy chain comprises an Fc region that comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, and alanine (A) at position 297 (all numbering according to EU Index). Suitably the antibody that specifically binds to human BTLA is an agonistic antibody/antigen-binding fragment.

Suitably, the antibody is a human IgG1 or IgG4 with one or more amino acid substitutions selected from the group consisting of: hIgG1 G236D, hIgG1 G237D, hIgG1 P238D, hIgG1 D265A, hIgG1 S267E, hIgG1 P271G, hIgG1 A330R, hIgG1 K322A, hIgG1 N297A, hIgG4 P238D, hIgG4 G237D, hIgG4 P271G, hIgG4 S330R, hIgG4 F234A and hIgG4 L235A. In particular embodiments the antibody that binds to human BTLA has a heavy chain and/or light chain with at least one CDR from an antibody selected from the group consisting of: 6.2, 2.8.6, 3E8,11.5.1, 12F11, 14D4, 15B6, 15C6, 16E1, 16F10, 16H2, 1H6, 21C7, 24H7, 26B1, 26F3, 27G9, 3A9, 4B1, 4D3, 4D5, 4E8, 4H4, 6G8, 7A1, 8B4, 8C4 and 831, as disclosed in Table 1 or Table 2 and described herein. In one embodiment, the antibody competes for binding to BTLA with its natural ligand HVEM. In another embodiment, the antibody does not interfere with binding of HVEM.

In particular embodiments, the isolated antibody which binds human BTLA is selected from the group consisting of 6.2, 2.8.6, 3E8, or an antibody that competes for binding to human BTLA with any one of 6.2, 2.8.6 or 3E8, wherein the antibody specifically binds BTLA and induces signaling through the receptor. Said antibody also comprises an Fc region that comprises an aspartic acid at position 238 (EU Index).

By an antibody selected from the group consisting of: 6.2, 2.8.6, 3E8, 11.5.1, 12F11, 14D4, 15B6, 15C6, 16E1, 16F10, 16H2, 1H6, 21C7, 24H7, 26B1, 26F3, 27G9, 3A9, 4B1, 4D3, 4D5, 4E8, 4H4, 6G8, 7A1, 8B4, 8C4 and 831, as disclosed in Table 1 and described herein, means any antibody or antigen-binding fragment thereof which comprises one or more, such as VH CDR 1, 2 and 3, or VL CDR 1, 2 and 3, or VH CDR 1, 2 and 3 and VL CDR 1, 2 and 3, from any of the antibodies disclosed in Tables 1 or 2 (whether murine, humanised or humanised/engineered).

According to a variation of the first aspect of the invention there is provided an isolated antibody that specifically binds human BTLA comprising at least one VH CDR that has an amino acid sequence as set forth in any of SEQ ID NO:1, SEQ ID NO: 2, SEQ ID NO:3, SEQ ID NO: 11 or SEQ ID NO: 17, with from 0 to 3 amino acid modifications, such as 0, 1, 2, or 3 amino acid modifications. In certain embodiments, the amino acid modifications include, but not limited to, amino acid substitution, addition, deletion, or chemical modification, without eliminating the antibody binding affinity or T-cell inhibitory effect of the modified amino acid sequence, as compared to the unmodified amino acid sequence.

According to another variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 1, CDRH2 has an amino acid sequence as set forth in SEQ ID NO: 2, 11 or 17, and CDRH3 has an amino acid sequence as set forth in SEQ ID NO: 3.

According to a variation of the first aspect of the invention there is provided an isolated antibody that specifically binds human BTLA comprising at least one VL CDR with an amino acid sequence as set forth in SEQ ID NO: 4, SEQ ID NO: 5, SEQ ID NO: 6 or SEQ ID NO: 12, with from 0 to 3 amino acid modifications.

According to another variation of the first aspect of the invention there is provided an isolated antibody that specifically binds human BTLA, comprising a heavy chain and a light chain, wherein the light chain comprises a light chain variable region comprising three CDRs: CDRL1, CDRL2 and CDRL3, wherein CDRL1 has an amino acid sequence as set forth in SEQ ID NO: 4, CDRL2 has an amino acid sequence as set forth in SEQ ID NO: 5 or 12, and CDRL3 has an amino acid sequence as set forth in SEQ ID NO: 6.

According to another variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 1, CDRH2 has an amino acid sequence as set forth in SEQ ID NO: 2, 11 or 17, and CDRH3 has an amino acid sequence as set forth in SEQ ID NO: 3, and the light chain comprises a light chain variable region comprising three CDRs: CDRL1, CDRL2 and CDRL3, wherein CDRL1 has an amino acid sequence as set forth in SEQ ID NO: 4, CDRL2 has an amino acid sequence as set forth in SEQ ID NO: 5 or 12, and CDRL3 has an amino acid sequence as set forth in SEQ ID NO: 6.

According to another variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 1, CDRH2 has an amino acid sequence as set forth in SEQ ID NO: 17, and CDRH3 has an amino acid sequence as set forth in SEQ ID NO: 3, and the light chain comprises a light chain variable region comprising three CDRs: CDRL1, CDRL2 and CDRL3, wherein CDRL1 has an amino acid sequence as set forth in SEQ ID NO: 4, CDRL2 has an amino acid sequence as set forth in SEQ ID NO: 12, and CDRL3 has an amino acid sequence as set forth in SEQ ID NO: 6; and wherein said heavy chain comprises an aspartic acid at position 238 (EU Index).

According to a variation of the first aspect of the invention there is provided an isolated antibody that specifically binds human BTLA comprising at least one VH CDR with an amino acid sequence as set forth in SEQ ID NO: 20, SEQ ID NO: 21 or SEQ ID NO: 22, with from 0 to 3 amino acid modifications.

According to a variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 20, CDRH2 has an amino acid sequence as set forth in SEQ ID NO: 21, and CDRH3 has an amino acid sequence as set forth in SEQ ID NO: 22.

According to a variation of the first aspect of the invention there is provided an isolated antibody that specifically binds human BTLA comprising at least one VL CDR with an amino acid sequence as set forth in SEQ ID NO: 23, SEQ ID NO: 24 or SEQ ID NO: 25, with from 0 to 3 amino acid modifications.

According to another variation of the first aspect of the invention there is provided an isolated antibody that specifically binds human BTLA, comprising a heavy chain and a light chain, wherein the light chain comprises a light chain variable region comprising three CDRs: CDRL1, CDRL2 and CDRL3, wherein CDRL1 has an amino acid sequence as set forth in SEQ ID NO: 23, CDRL2 has an amino acid sequence as set forth in SEQ ID NO: 24, and CDRL3 has an amino acid sequence as set forth in SEQ ID NO: 25.

According to another variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 20, CDRH2 has an amino acid sequence as set forth in SEQ ID NO: 21, and CDRH3 has an amino acid sequence as set forth in SEQ ID NO: 22, and the light chain comprises a light chain variable region comprising three CDRs: CDRL1, CDRL2 and CDRL3, wherein CDRL1 has an amino acid sequence as set forth in SEQ ID NO: 23, CDRL2 has an amino acid sequence as set forth in SEQ ID NO: 24, and CDRL3 has an amino acid sequence as set forth in SEQ ID NO: 25.

According to a variation of the first aspect of the invention there is provided an isolated antibody that specifically binds human BTLA comprising at least one VH CDR with an amino acid sequence as set forth in SEQ ID NO: 30, SEQ ID NO: 31, SEQ ID NO: 40, SEQ ID NO: 48 or SEQ ID NO: 32, with from 0 to 3 amino acid modifications.

According to a variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 30, CDRH2 has an amino acid sequence as set forth in SEQ ID NO: 31, 40 or 48, and CDRH3 has an amino acid sequence as set forth in SEQ ID NO: 32.

According to a variation of the first aspect of the invention there is provided an isolated antibody that specifically binds human BTLA comprising at least one VL CDR with an amino acid sequence as set forth in SEQ ID NO: 33, SEQ ID NO: 34 or SEQ ID NO: 35, with from 0 to 3 amino acid modifications.

According to another variation of the first aspect of the invention there is provided an isolated antibody thereof that specifically binds human BTLA, comprising a heavy chain and a light chain, wherein the light chain comprises a light chain variable region comprising three CDRs: CDRL1, CDRL2 and CDRL3, wherein CDRL1 has an amino acid sequence as set forth in SEQ ID NO: 33, CDRL2 has an amino acid sequence as set forth in SEQ ID NO: 34, and CDRL3 has an amino acid sequence as set forth in SEQ ID NO: 35.

According to another variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 30, CDRH2 has an amino acid sequence as set forth in SEQ ID NO: 31, 40 or 48, and CDRH3 has an amino acid sequence as set forth in SEQ ID NO: 32, and the light chain comprises a light chain variable region comprising three CDRs: CDRL1, CDRL2 and CDRL3, wherein CDRL1 has an amino acid sequence as set forth in SEQ ID NO: 33, CDRL2 has an amino acid sequence as set forth in SEQ ID NO:34, and CDRL3 has an amino acid sequence as set forth in SEQ ID NO:35.

In each of these aspects, the antibody has an Fc region which comprises at least one amino acid substitution that results in increased binding to FcγR2B compared to the parent molecule that lacks the substitution. In some embodiments, the antibody has selectivity for binding FcγR2B over FcγR2A compared to the parent molecule that lacks the substitution. In some embodiments, the antibody has selectivity for binding FcγR2B over FcγR1A compared to the parent molecule that lacks the substitution.

In a particular embodiment, the antibody comprises an Fc region which comprises an aspartic acid at position 238 (EU Index).

In a particular embodiment, the antibody comprises an Fc region which comprises an aspartic acid at position 237 (EU Index), an aspartic acid at position 238 (EU Index), a glycine at position 271 (EU Index) and an arginine at position 330 (EU Index).

According to another variation of the first aspect of the invention there is provided an isolated antibody that specifically binds to human BTLA, wherein said antibody comprises a heavy chain and a light chain, wherein: the heavy chain comprises an Fc region and a heavy chain variable region comprising three complementarity determining regions (CDRs): CDRH1, CDRH2 and CDRH3 and the light chain comprises a light chain variable region comprising three CDRs: CDRL1, CDRL2, and CDRL3, wherein (1) CDRH1, CDRH2, CDRH3 have an amino acid sequence as set forth in SEQ ID NO: 1, SEQ ID NO: 17, and SEQ ID NO: 3, respectively, with from 0 to 3 amino acid modification, and CDRL1, CDRL2, and CDRL3 have an amino acid sequence as set forth in SEQ ID NO: 4, SEQ ID NO: 12, and SEQ ID NO: 6, respectively, with from 0 to 3 amino acid modifications; or (2) CDRH1, CDRH2, CDRH3 have an amino acid sequence as set forth in SEQ ID NO: 20, SEQ ID NO: 21, and SEQ ID NO: 22, respectively, with from 0 to 3 amino acid modification, and CDRL1, CDRL2, and CDRL3 have an amino acid sequence as set forth in SEQ ID NO: 23, SEQ ID NO: 24, and SEQ ID NO: 25, respectively, with from 0 to 3 amino acid modifications; or (3) CDRH1, CDRH2, CDRH3 have an amino acid sequence as set forth in SEQ ID NO: 30, SEQ ID NO: 31, and SEQ ID NO: 32, respectively, with from 0 to 3 amino acid modification, and CDRL1, CDRL2, and CDRL3 have an amino acid sequence as set forth in SEQ ID NO: 33, SEQ ID NO: 34, and SEQ ID NO: 35, respectively, with from 0 to 3 amino acid modifications, and wherein the Fc region portion comprises an aspartic acid at position 238 (EU Index).

A typical antibody comprises 2 heavy chains and 2 light chains, wherein the paired heavy chains comprise the Fc region, thus as used herein a “heavy chain that comprises an Fc region” refers to the region on the H chain polypeptide which together with another H chain Fc region forms the functional Fc region.

According to another variation of the first aspect of the invention there is provided an isolated antibody that specifically binds human BTLA comprising at least one VH CDR with an amino acid sequence as set forth in (1) SEQ ID NO: 45, 46, or 47, with from 0 to 3 amino acid modifications; (2) SEQ ID NO: 53, 54, or 55, with from 0 to 3 amino acid modifications; (3) SEQ ID NO: 61, 62, or 63, with from 0 to 3 amino acid modifications; (4) SEQ ID NO: 61, 69, or 70, with from 0 to 3 amino acid modifications; (5) SEQ ID NO: 76, 77, or 78, with from 0 to 3 amino acid modifications; (6) SEQ ID NO: 45, 46, or 84, with from 0 to 3 amino acid modifications; (7) SEQ ID NO: 88, 89, or 90, with from 0 to 3 amino acid modifications; (8) SEQ ID NO: 95, 96, or 97, with from 0 to 3 amino acid modifications; (9) SEQ ID NO: 103, 104, or 105, with from 0 to 3 amino acid modifications; (10) SEQ ID NO: 76, 111, or 112, with from 0 to 3 amino acid modifications; (11) SEQ ID NO: 118, 119, or 120, with from 0 to 3 amino acid modifications; (12) SEQ ID NO: 126, 127, or 128, with from 0 to 3 amino acid modifications; (13) SEQ ID NO: 133, 134, or 135, with from 0 to 3 amino acid modifications; (14) SEQ ID NO: 103, 134, or 139, with from 0 to 3 amino acid modifications; (15) SEQ ID NO: 143, 144, or 145, with from 0 to 3 amino acid modifications; (16) SEQ ID NO: 151, 152, or 153, with from 0 to 3 amino acid modifications; (17) SEQ ID NO: 159, 160, or 161, with from 0 to 3 amino acid modifications; (18) SEQ ID NO: 167, 168, or 169, with from 0 to 3 amino acid modifications; (19) SEQ ID NO: 45, 46, or 177, with from 0 to 3 amino acid modifications; (20) SEQ ID NO: 181, 182, or 183, with from 0 to 3 amino acid modifications; (21) SEQ ID NO: 45, 191, or 192, with from 0 to 3 amino acid modifications; (22) SEQ ID NO: 196, 197, or 198, with from 0 to 3 amino acid modifications; (23) SEQ ID NO: 204, 205, or 206, with from 0 to 3 amino acid modifications; (24) SEQ ID NO: 212, 213, or 214, with from 0 to 3 amino acid modifications; (25) SEQ ID NO: 1, 2, or 3, with from 0 to 3 amino acid modifications; (26) SEQ ID NO: 20, 163, or 22, with from 0 to 3 amino acid modifications; (27) SEQ ID NO: 30, 48, or 32, with from 0 to 3 amino acid modifications; (28) SEQ ID NO: 1, 11, or 3, with from 0 to 3 amino acid modifications; (29) SEQ ID NO: 1, 17, or 3, with from 0 to 3 amino acid modifications; (30) SEQ ID NO: 20, 21, or 22, with from 0 to 3 amino acid modifications; (33) SEQ ID NO: 30, 31, or 32, with from 0 to 3 amino acid modifications; or (34) SEQ ID NO: 30, 40, or 32, with from 0 to 3 amino acid modifications. Said antibody also comprises an Fc region that comprises an aspartic acid at position 238 (EU Index).

According to a variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1, CDRH2, CDRH3 have an amino acid sequence as set forth in (1) SEQ ID NO: 45, 46, and 47, respectively; (2) SEQ ID NO: 53, 54, and 55, respectively; (3) SEQ ID NO: 61, 62, and 63, respectively; (4) SEQ ID NO: 61, 69, and 70, respectively; (5) SEQ ID NO: 76, 77, and 78, respectively; (6) SEQ ID NO: 45, 46, and 84, respectively; (7) SEQ ID NO: 88, 89, and 90, respectively; (8) SEQ ID NO: 95, 96, and 97, respectively; (9) SEQ ID NO: 103, 104, and 105, respectively; (10) SEQ ID NO: 76, 111, and 112, respectively; (11) SEQ ID NO: 118, 119, and 120, respectively; (12) SEQ ID NO: 126, 127, and 128, respectively; (13) SEQ ID NO: 133, 134, and 135, respectively; (14) SEQ ID NO: 103, 134, and 139, respectively; (15) SEQ ID NO: 143, 144, and 145, respectively; (16) SEQ ID NO: 151, 152, and 153, respectively; (17) SEQ ID NO: 159, 160, and 161, respectively; (18) SEQ ID NO: 167, 168, and 169, respectively; (19) SEQ ID NO: 45, 46, and 177, respectively; (20) SEQ ID NO: 181, 182, and 183, respectively; (21) SEQ ID NO: 45, 191, and 192, respectively; (22) SEQ ID NO: 196, 197, and 198, respectively; (23) SEQ ID NO: 204, 205, and 206, respectively; (24) SEQ ID NO: 212, 213, and 214, respectively; (25) SEQ ID NO: 1, 2, and 3, respectively; (26) SEQ ID NO: 20, 163, and 22, respectively; (27) SEQ ID NO: 30, 48, and 32, respectively; (28) SEQ ID NO: 1, 11, and 3, respectively; (29) SEQ ID NO: 1, 17, and 3, respectively; (30) SEQ ID NO: 20, 21, and 22, respectively; (33) SEQ ID NO: 30, 31, and 32, respectively; or (34) SEQ ID NO: 30, 40, and 32, respectively; wherein from 0 to 3 amino acid modifications can be present in any CDR/SEQ ID NO:. Said antibody also comprises an Fc region that comprises an aspartic acid at position 238 (EU Index).

According to a variation of the first aspect of the invention there is provided an isolated antibody that specifically binds human BTLA comprising at least one VL CDR with an amino acid sequence as set forth in (1) SEQ ID NO: 33, 34, or 35; (2) SEQ ID NO: 56, 57, or 58; (3) SEQ ID NO: 64, 65, or 66; (4) SEQ ID NO: 71, 72, or 73; (5) SEQ ID NO: 79, 80, or 81; (6) SEQ ID NO: 33, 34, or 85; (7) SEQ ID NO: 91, 65, or 92; (8) SEQ ID NO: 98, 99, or 100; (9) SEQ ID NO: 106, 107, or 108; (10) SEQ ID NO: 113, 114, or 115; (11) SEQ ID NO: 121, 122, or 123; (12) SEQ ID NO: 79, 129, or 130; (13) SEQ ID NO: 106, 107, or 136; (14) SEQ ID NO: 146, 147, or 148; (15) SEQ ID NO: 154, 155, or 156; (16) SEQ ID NO: 4, 12, or 164; (17) SEQ ID NO: 170, 171, or 172; (18) SEQ ID NO: 154, 155, or 178; (19) SEQ ID NO: 184, 185, or 186; (20) SEQ ID NO: 79, 80, or 189; (21) SEQ ID NO: 154, 155, or 193; (22) SEQ ID NO: 199, 200, or 201; (23) SEQ ID NO: 207, 208, or 209; (24) SEQ ID NO: 215, 34, or 216; (25) SEQ ID NO: 4, 5, or 6; (26) SEQ ID NO: 23, 176, or 25; (27) SEQ ID NO: 33, 34, or 35; (28) SEQ ID NO: 4, 12, or 6; (29) SEQ ID NO: 23, 24, or 25; or (30) SEQ ID NO: 33, 34, or 35; wherein from 0 to 3 amino acid modifications can be present in any CDR/SEQ ID NO:, and wherein said antibody also comprises an Fc region that comprises an aspartic acid at position 238 (EU Index).

According to another variation of the first aspect of the invention there is provided an isolated antibody that specifically binds human BTLA, comprising a heavy chain and a light chain, wherein the light chain comprises a light chain variable region comprising three CDRs: CDRL1, CDRL2 and CDRL3, wherein CDRL1, CDRL2, and CDRL3 have an amino acid sequence as set forth in (1) SEQ ID NO: 33, 34, and 35, respectively; (2) SEQ ID NO: 56, 57, and 58, respectively; (3) SEQ ID NO: 64, 65, and 66, respectively; (4) SEQ ID NO: 71, 72, and 73, respectively; (5) SEQ ID NO: 79, 80, and 81, respectively; (6) SEQ ID NO: 33, 34, and 85, respectively; (7) SEQ ID NO: 91, 65, and 92, respectively; (8) SEQ ID NO: 98, 99, and 100, respectively; (9) SEQ ID NO: 106, 107, and 108, respectively; (10) SEQ ID NO: 113, 114, and 115, respectively; (11) SEQ ID NO: 121, 122, and 123, respectively; (12) SEQ ID NO: 79, 129, and 130, respectively; (13) SEQ ID NO: 106, 107, and 136, respectively; (14) SEQ ID NO: 146, 147, and 148, respectively; (15) SEQ ID NO: 154, 155, and 156, respectively; (16) SEQ ID NO: 4, 12, and 164, respectively; (17) SEQ ID NO: 170, 171, and 172, respectively; (18) SEQ ID NO: 154, 155, and 178, respectively; (19) SEQ ID NO: 184, 185, and 186, respectively; (20) SEQ ID NO: 79, 80, and 189, respectively; (21) SEQ ID NO: 154, 155, and 193, respectively; (22) SEQ ID NO: 199, 200, and 201, respectively; (23) SEQ ID NO: 207, 208, and 209, respectively; (24) SEQ ID NO: 215, 34, and 216, respectively; (25) SEQ ID NO: 4, 5, and 6, respectively; (26) SEQ ID NO: 23, 176, and 25, respectively; (27) SEQ ID NO: 33, 34, and 35, respectively; (28) SEQ ID NO: 4, 5, and 6, respectively; (29) SEQ ID NO: 4, 12, and 6, respectively; (30) SEQ ID NO: 23, 24, and 25, respectively; or (31) SEQ ID NO: 33, 34, and 35, respectively; wherein from 0 to 3 amino acid modifications can be present in any CDR/SEQ ID NO:, and wherein said antibody also comprises an Fc region that comprises an aspartic acid at position 238 (EU Index)

According to another variation of the first aspect of the invention there is provided an isolated antibody that specifically binds human BTLA, comprising a heavy chain and a light chain, wherein the heavy chain comprises a heavy chain variable region comprising three CDRs: CDRH1, CDRH2 and CDRH3, and the light chain comprises a light chain variable region comprising three CDRs: CDRL1, CDRL2 and CDRL3, wherein (1) CDRH1, CDRH2, CDRH3 have an amino acid sequence as set forth in SEQ ID NO: 45, 46, and 47, respectively, and CDRL1, CDRL2, and CDRL3 have an amino acid sequence as set forth in SEQ ID NO: 33, 34, and 35, respectively; (2) CDRH1, CDRH2, CDRH3 have an amino acid sequence as set forth in SEQ ID NO: 53, 54, and 55, respectively, and CDRL1, CDRL2, and CDRL3 have an amino acid sequence as set forth in SEQ ID NO: 56, 57, and 58, respectively;, respectively; (3) CDRH1, CDRH2, CDRH3 have an amino acid sequence as set forth in SEQ ID NO: 61, 62, and 63, respectively, and CDRL1, CDRL2, and CDRL3 have an amino acid sequence as set forth in SEQ ID NO: 64, 65, and 66, respectively; (4) CDRH1, CDRH2, CDRH3 have an amino acid sequence as set forth in SEQ ID NO: 61, 69, and 70, respectively, and CDRL1, CDRL2, and CDRL3 have an amino acid sequence as set forth in SEQ ID NO: 71, 72, and 73, respectively; (5) CDRH1, CDRH2, CDRH3 have an amino acid sequence as set forth in SEQ ID NO: 76, 77, and 78, respectively, and CDRL1, CDRL2, and CDRL3 have an amino acid sequence as set forth in SEQ ID NO: 79, 80, and 81, respectively; (6) CDRH1, CDRH2, CDRH3 have an amino acid sequence as set forth in SEQ ID NO: 45, 46, and 84, respectively, and CDRL1, CDRL2, and CDRL3 have an amino acid sequence as set forth in SEQ ID NO: 33, 34, and 85, respectively; (7) CDRH1, CDRH2, CDRH3 have an amino acid sequence as set forth in SEQ ID NO: 88, 89, and 90, respectively, and CDRL1, CDRL2, and CDRL3 have an amino acid sequence as set forth in SEQ ID NO: 91, 65, and 92, respectively; (8) CDRH1, CDRH2, CDRH3 have an amino acid sequence as set forth in SEQ ID NO: 95, 96, and 97, respectively, and CDRL1, CDRL2, and CDRL3 have an amino acid sequence as set forth in SEQ ID NO: 98, 99, and 100, respectively; (9) CDRH1, CDRH2, CDRH3 have an amino acid sequence as set forth in SEQ ID NO: 103, 104, and 105, respectively, and CDRL1, CDRL2, and CDRL3 have an amino acid sequence as set forth in SEQ ID NO: 106, 107, and 108, respectively; (10) CDRH1, CDRH2, CDRH3 have an amino acid sequence as set forth in SEQ ID NO: 76, 111, and 112, respectively, respectively, and CDRL1, CDRL2, and CDRL3 have an amino acid sequence as set forth in SEQ ID NO: 113, 114, and 115, respectively; (11) CDRH1, CDRH2, CDRH3 have an amino acid sequence as set forth in SEQ ID NO: 118, 119, and 120, respectively, and CDRL1, CDRL2, and CDRL3 have an amino acid sequence as set forth in SEQ ID NO: 121, 122, and 123, respectively; (12) CDRH1, CDRH2, CDRH3 have an amino acid sequence as set forth in SEQ ID NO: 126, 127, and 128, respectively, and CDRL1, CDRL2, and CDRL3 have an amino acid sequence as set forth in SEQ ID NO: 79, 129, and 130, respectively; (13) CDRH1, CDRH2, CDRH3 have an amino acid sequence as set forth in SEQ ID NO: 133, 134, and 135, respectively, and CDRL1, CDRL2, and CDRL3 have an amino acid sequence as set forth in SEQ ID NO: 106, 107, and 136, respectively; (14) CDRH1, CDRH2, CDRH3 have an amino acid sequence as set forth in SEQ ID NO: 103, 134, and 139, respectively, and CDRL1, CDRL2, and CDRL3 have an amino acid sequence as set forth in SEQ ID NO: 106, 107, and 136, respectively; (15) CDRH1, CDRH2, CDRH3 have an amino acid sequence as set forth in SEQ ID NO: 143, 144, and 145, respectively, respectively, and CDRL1, CDRL2, and CDRL3 have an amino acid sequence as set forth in SEQ ID NO: 146, 147, and 148, respectively; (16) CDRH1, CDRH2, CDRH3 have an amino acid sequence as set forth in SEQ ID NO: 151, 152, and 153, respectively, respectively, and CDRL1, CDRL2, and CDRL3 have an amino acid sequence as set forth in SEQ ID NO: 154, 155, and 156, respectively; (17) CDRH1, CDRH2, CDRH3 have an amino acid sequence as set forth in SEQ ID NO: 159, 160, and 161, respectively, and CDRL1, CDRL2, and CDRL3 have an amino acid sequence as set forth in SEQ ID NO: 4, 12, and 164, respectively; (18) CDRH1, CDRH2, CDRH3 have an amino acid sequence as set forth in SEQ ID NO: 167, 168, and 169, respectively, and CDRL1, CDRL2, and CDRL3 have an amino acid sequence as set forth in SEQ ID NO: 170, 171, and 172, respectively; (19) CDRH1, CDRH2, CDRH3 have an amino acid sequence as set forth in SEQ ID NO: 45, SEQ ID NO: 46, and SEQ ID NO: 47, respectively, and CDRL1, CDRL2, and CDRL3 have an amino acid sequence as set forth in SEQ ID NO: 170, 171, and 172, respectively, respectively; (20) CDRH1, CDRH2, CDRH3 have an amino acid sequence as set forth in SEQ ID NO: 45, 46, and 177, and CDRL1, CDRL2, and CDRL3 have an amino acid sequence as set forth in SEQ ID NO: 154, 155, and 178, respectively; (21) CDRH1, CDRH2, CDRH3 have an amino acid sequence as set forth in SEQ ID NO: 181, 182, and 183, respectively, and CDRL1, CDRL2, and CDRL3 have an amino acid sequence as set forth in SEQ ID NO: 184, 185, and 186,; (22) CDRH1, CDRH2, CDRH3 have an amino acid sequence as set forth in SEQ ID NO: 76, 77, and 78, respectively, and CDRL1, CDRL2, and CDRL3 have an amino acid sequence as set forth in SEQ ID NO: 79, 80, and 189, respectively; (23) CDRH1, CDRH2, CDRH3 have an amino acid sequence as set forth in SEQ ID NO: 45, 191, and 192, respectively, and CDRL1, CDRL2, and CDRL3 have an amino acid sequence as set forth in SEQ ID NO: 154, 155, and 193, respectively; (24) CDRH1, CDRH2, CDRH3 have an amino acid sequence as set forth in SEQ ID NO: 196, 197, and 198, respectively, and CDRL1, CDRL2, and CDRL3 have an amino acid sequence as set forth in SEQ ID NO: 199, 200, and 201, respectively; (25) CDRH1, CDRH2, CDRH3 have an amino acid sequence as set forth in SEQ ID NO: 204, 205, and 206, respectively, and CDRL1, CDRL2, and CDRL3 have an amino acid sequence as set forth in SEQ ID NO: 207, 208, and 209, respectively; (26) CDRH1, CDRH2, CDRH3 have an amino acid sequence as set forth in SEQ ID NO: 212, 213, and 214, respectively, and CDRL1, CDRL2, and CDRL3 have an amino acid sequence as set forth in SEQ ID NO: 215, 34, and 216, respectively; (27) CDRH1, CDRH2, CDRH3 have an amino acid sequence as set forth in SEQ ID NO: 1, 2, and 3, respectively, respectively, and CDRL1, CDRL2, and CDRL3 have an amino acid sequence as set forth in SEQ ID NO: 4, 5, and 6, respectively; (28) CDRH1, CDRH2, CDRH3 have an amino acid sequence as set forth in SEQ ID NO: 20, 163, and 22, respectively, and CDRL1, CDRL2, and CDRL3 have an amino acid sequence as set forth in SEQ ID NO: 23, 176, and 25, respectively; (29) CDRH1, CDRH2, CDRH3 have an amino acid sequence as set forth in SEQ ID NO: 30, 48, and 32, respectively, and CDRL1, CDRL2, and CDRL3 have an amino acid sequence as set forth in SEQ ID NO: 33, 34, and 35, respectively; (30) CDRH1, CDRH2, CDRH3 have an amino acid sequence as set forth in SEQ ID NO: 1, 11, and 3, respectively, and CDRL1, CDRL2, and CDRL3 have an amino acid sequence as set forth in SEQ ID NO: 4, 12, and 6, respectively; (31) CDRH1, CDRH2, CDRH3 have an amino acid sequence as set forth in SEQ ID NO: 1, 11, and 3, respectively, and CDRL1, CDRL2, and CDRL3 have an amino acid sequence as set forth in SEQ ID NO: 4, 5, and 6, respectively; (32) CDRH1, CDRH2, CDRH3 have an amino acid sequence as set forth in SEQ ID NO: 1, 17, and 3, respectively, and CDRL1, CDRL2, and CDRL3 have an amino acid sequence as set forth in SEQ ID NO: 4, 12, and 6, respectively; (33) CDRH1, CDRH2, CDRH3 have an amino acid sequence as set forth in SEQ ID NO: 20, 21, and 22, respectively, and CDRL1, CDRL2, and CDRL3 have an amino acid sequence as set forth in SEQ ID NO: 23, 24, and 25, respectively; (34) CDRH1, CDRH2, CDRH3 have an amino acid sequence as set forth in SEQ ID NO: 30, 31, and 32, respectively, and CDRL1, CDRL2, and CDRL3 have an amino acid sequence as set forth in SEQ ID NO: 33, 34, and 35, respectively; or (35) CDRH1, CDRH2, CDRH3 have an amino acid sequence as set forth in SEQ ID NO: 30, 40, and 32, respectively, and CDRL1, CDRL2, and CDRL3 have an amino acid sequence as set forth in SEQ ID NO: 33, 34, and 35, respectively; wherein from 0 to 3 amino acid modifications can be present in any CDR/SEQ ID NO:, and wherein said antibody also comprises an Fc region that comprises an aspartic acid at position 238 (EU Index).

According to another variation of the first aspect of the invention there is provided an isolated antibody that specifically binds human BTLA, comprising: (1) at least one VH CDR with an amino acid sequence as set forth in SEQ ID NO: 45, 46, or 47, and at least one VL CDR with an amino acid sequence as set forth in SEQ ID NO: 33, 34, or 35; (2) at least one VH CDR with an amino acid sequence as set forth in SEQ ID NO: 53, 54, or 55, and at least one VL CDR with an amino acid sequence as set forth in SEQ ID NO: 56, 57, and 58; (3) at least one VH CDR with an amino acid sequence as set forth in SEQ ID NO: 61, 62, or 63, and at least one VL CDR with an amino acid sequence as set forth in SEQ ID NO: 64, 65, or 66; (4) at least one VH CDR with an amino acid sequence as set forth in SEQ ID NO: 61, 69, or 70, and at least one VL CDR with an amino acid sequence as set forth in SEQ ID NO: 71, 72, and 73; (5) at least one VH CDR with an amino acid sequence as set forth in SEQ ID NO: 76, 77, or 78, and at least one VL CDR with an amino acid sequence as set forth in SEQ ID NO: 79, 80, or 81 (6) at least one VH CDR with an amino acid sequence as set forth in SEQ ID NO: 45, 46, or 84, and at least one VL CDR with an amino acid sequence as set forth in SEQ ID NO: 33, 34, or 85; (7) at least one VH CDR with an amino acid sequence as set forth in SEQ ID NO: 88, 89, or 90, and at least one VL CDR with an amino acid sequence as set forth in SEQ ID NO: 91, 65, or 92; (8) at least one VH CDR with an amino acid sequence as set forth in SEQ ID NO: 95, 96, or 97, and at least one VL CDR with an amino acid sequence as set forth in SEQ ID NO: 98, 99, or 100; (9) at least one VH CDR with an amino acid sequence as set forth in SEQ ID NO: 103, 104, or 105, and at least one VL CDR with an amino acid sequence as set forth in SEQ ID NO: 106, 107, or 108; (10) at least one VH CDR with an amino acid sequence as set forth in SEQ ID NO: 76, 111, or 112, and at least one VL CDR with an amino acid sequence as set forth in SEQ ID NO: 113, 114, or 115; (11) at least one VH CDR with an amino acid sequence as set forth in SEQ ID NO: 118, 119, or 120, and at least one VL CDR with an amino acid sequence as set forth in SEQ ID NO: 121, 122, or 123; (12) at least one VH CDR with an amino acid sequence as set forth in SEQ ID NO: 126, 127, or 128, and at least one VL CDR with an amino acid sequence as set forth in SEQ ID NO: 79, 129, or 130; (13) at least one VH CDR with an amino acid sequence as set forth in SEQ ID NO: 133, 134, or 135, and at least one VL CDR with an amino acid sequence as set forth in SEQ ID NO: 106, 107, or 136; (14) at least one VH CDR with an amino acid sequence as set forth in SEQ ID NO: 103, 134, or 139, and at least one VL CDR with an amino acid sequence as set forth in SEQ ID NO: 106, 107, or 136; (15) at least one VH CDR with an amino acid sequence as set forth in SEQ ID NO: 143, 144, or 145, and at least one VL CDR with an amino acid sequence as set forth in S SEQ ID NO: 146, 147, or 148; (16) at least one VH CDR with an amino acid sequence as set forth in SEQ ID NO: 151, 152, or 153, and at least one VL CDR with an amino acid sequence as set forth in SEQ ID NO: 154, 155, or 156; (17) at least one VH CDR with an amino acid sequence as set forth in SEQ ID NO: 159, 160, or 161, and at least one VL CDR with an amino acid sequence as set forth in SEQ ID NO: 4, 12, or 164; (18) at least one VH CDR with an amino acid sequence as set forth in SEQ ID NO: 167, 168, or 169, and at least one VL CDR with an amino acid sequence as set forth in SEQ ID NO: 170, 171, or 172; (19) at least one VH CDR with an amino acid sequence as set forth in SEQ ID NO: 45, 46, or 47, and at least one VL CDR with an amino acid sequence as set forth in SEQ ID NO: 170, 171, or 172; (20) at least one VH CDR with an amino acid sequence as set forth in SEQ ID NO: 45, 46, or 177, and at least one VL CDR with an amino acid sequence as set forth in SEQ ID NO: 154, 155, or 178; (21) at least one VH CDR with an amino acid sequence as set forth in SEQ ID NO: 181, 182, or 183, and at least one VL CDR with an amino acid sequence as set forth in SEQ ID NO: 184, 185, or 186; (22) at least one VH CDR with an amino acid sequence as set forth in SEQ ID NO: 76, 77, or 78, and at least one VL CDR with an amino acid sequence as set forth in SEQ ID NO: 79, 80, or 189; (23) at least one VH CDR with an amino acid sequence as set forth in SEQ ID NO: 45, 191, or 192, and at least one VL CDR with an amino acid sequence as set forth in SEQ ID NO: 154, 155, or 193; (24) at least one VH CDR with an amino acid sequence as set forth in SEQ ID NO: 196, 197, or 198, and at least one VL CDR with an amino acid sequence as set forth in SEQ ID NO: 199, 200, or 201; (25) at least one VH CDR with an amino acid sequence as set forth in SEQ ID NO: 204, 205, or 206, and at least one VL CDR with an amino acid sequence as set forth in SEQ ID NO: 207, 208, or 209; (26) at least one VH CDR with an amino acid sequence as set forth in SEQ ID NO: 212, 213, or 214, and at least one VL CDR with an amino acid sequence as set forth in SEQ ID NO: 215, 34, or 216; (27) at least one VH CDR with an amino acid sequence as set forth in SEQ ID NO: 1, 2, or 3, and at least one VL CDR with an amino acid sequence as set forth in SEQ ID NO: 4, 5, or 6; (28) at least one VH CDR with an amino acid sequence as set forth in SEQ ID NO: 20, 163, or 22, and at least one VL CDR with an amino acid sequence as set forth in SEQ ID NO: 23, 176, or 25; (29) at least one VH CDR with an amino acid sequence as set forth in SEQ ID NO: 30, 48, or 32, and at least one VL CDR with an amino acid sequence as set forth in SEQ ID NO: 33, 34, or 35; (30) at least one VH CDR with an amino acid sequence as set forth in SEQ ID NO: 1, 11, or 3, and at least one VL CDR with an amino acid sequence as set forth in SEQ ID NO: 4, 12, or 6; (31) at least one VH CDR with an amino acid sequence as set forth in SEQ ID NO: 1, 11, or 3, and at least one VL CDR with an amino acid sequence as set forth in SEQ ID NO: 4, 5, or 6; (32) at least one VH CDR with an amino acid sequence as set forth in SEQ ID NO: 1, 17, or 3, and at least one VL CDR with an amino acid sequence as set forth in SEQ ID NO: 4, 12, or 6; (33) at least one VH CDR with an amino acid sequence as set forth in SEQ ID NO: 20, 21, or 22, and at least one VL CDR with an amino acid sequence as set forth in SEQ ID NO: 23, 24, or 25; (34) at least one VH CDR with an amino acid sequence as set forth in SEQ ID NO: 30, 31, or 32, and at least one VL CDR with an amino acid sequence as set forth in SEQ ID NO: 33, 34, or 35; or (35) at least one VH CDR with an amino acid sequence as set forth in SEQ ID NO: 30, 40, or 32, and at least one VL CDR with an amino acid sequence as set forth in SEQ ID NO: 33, 34, or 35; wherein from 0 to 3 amino acid modifications can be present in any CDR/SEQ ID NO:, and wherein said antibody also comprises an Fc region that comprises an aspartic acid at position 238 (EU Index).

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 7, 13 or 18, or a sequence with at least 90% sequence identity thereto.

In other embodiments, the heavy chain variable region comprises an amino acid sequence as set forth in SEQ ID NO: 7, 13 or 18, with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the light chain comprises a light chain variable region comprising an amino acid sequence as set forth in SEQ ID NO: 8 or 14, or a sequence with at least 90% sequence identity thereto.

In other embodiments, the light chain variable region comprises an amino acid sequence as set forth in SEQ ID NO: 8 or 14, with up to 10 modifications, such as 1, 2, 3, 4, 5, 6, 7, 8, 9 or 10 amino acid modifications therein.

In each of these embodiments, the antibody has an Fc region which comprises at least one amino acid substitution that results in increased binding to FcγR2B compared to the parent molecule that lacks the substitution. In some embodiments, the antibody has selectivity for binding FcγR2B over FcγR2A compared to the parent molecule that lacks the substitution.

In particular embodiments, the antibody comprises an Fc region comprises an aspartic acid at position 238 (EU Index).

According to another variation of the first aspect of the invention there is provided an isolated antibody that specifically binds human BTLA, comprising a heavy chain and a light chain, wherein the heavy chain comprises a heavy chain variable region comprising an amino acid sequence that has at least 90% sequence identity to the amino acid sequence as set forth in SEQ ID NO: 7, 13 or 18, and the light chain comprises a light chain variable region comprising an amino acid sequence that has at least 90% sequence identity to the amino acid sequence as set forth in SEQ ID NO: 8 or 14.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 18 or SEQ ID NO: 13, and the light chain comprises a light chain variable region comprising an amino acid sequence as set forth in SEQ ID NO: 14.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 7, and the light chain comprises a light chain variable region comprising an amino acid sequence as set forth in SEQ ID NO: 8.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 13, and the light chain comprises a light chain variable region comprising an amino acid sequence as set forth in SEQ ID NO: 14.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 13, and the light chain comprises a light chain variable region comprising an amino acid sequence as set forth in SEQ ID NO: 8.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 18, and the light chain comprises a light chain variable region comprising an amino acid sequence as set forth in SEQ ID NO: 14.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 9, 15 or 19, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 9, 15 or 19, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the light chain comprises an amino acid sequence as set forth in SEQ ID NO: 10, 16 or 29, or a sequence with at least 90% sequence identity thereto.

According to another variation of the first aspect of the invention there is provided an isolated antibody that specifically binds human BTLA, comprising a heavy chain and a light chain, wherein the light chain comprises an amino acid sequence as set forth in SEQ ID NO: 10, 16 or 29, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 9, or a sequence with at least 90% sequence identity thereto and the light chain comprises an amino acid sequence as set forth in SEQ ID NO: 10, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 9, or a sequence with up to 10 modifications, such as 1, 2, 3, 4, 5, 6, 7, 8, 9 or 10 amino acid modifications therein and wherein the light chain comprises an amino acid sequence as set forth in SEQ ID NO: 10, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 15, or a sequence with at least 90% sequence identity thereto and the light chain comprises an amino acid sequence as set forth in SEQ ID NO: 16, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 15, or a sequence with up to 10 modifications, such as 1, 2, 3, 4, 5, 6, 7, 8, 9 or 10 amino acid modifications therein and wherein the light chain comprises an amino acid sequence as set forth in SEQ ID NO: 16, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 15, or a sequence with at least 90% sequence identity thereto and the light chain comprises an amino acid sequence as set forth in SEQ ID NO: 10, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 15, or a sequence with up to 10 modifications, such as 1, 2, 3, 4, 5, 6, 7, 8, 9 or 10 amino acid modifications therein and wherein the light chain comprises an amino acid sequence as set forth in SEQ ID NO: 10, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 19, or a sequence with at least 90% sequence identity thereto and the light chain comprises an amino acid sequence as set forth in SEQ ID NO: 16, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 19, or a sequence with up to 10 modifications, such as 1, 2, 3, 4, 5, 6, 7, 8, 9 or 10 amino acid modifications therein and wherein the light chain comprises an amino acid sequence as set forth in SEQ ID NO: 16, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 26, or a sequence with at least 90% sequence identity thereto.

In other embodiments, the heavy chain variable region comprises an amino acid sequence as set forth in SEQ ID NO: 26, with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the light chain comprises a light chain variable region comprising an amino acid sequence as set forth in SEQ ID NO: 27, or a sequence with at least 90% sequence identity thereto.

In other embodiments, the light chain variable region comprises an amino acid sequence as set forth in SEQ ID NO: 27, with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises a heavy chain variable region comprising an amino acid sequence that has at least 90% sequence identity to the amino acid sequence as set forth in SEQ ID NO: 26, and the light chain comprises a light chain variable region comprising an amino acid sequence that has at least 90% sequence identity to the amino acid sequence as set forth in SEQ ID NO: 27.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 26, and the light chain comprises a light chain variable region comprising an amino acid sequence as set forth in SEQ ID NO: 27.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 28, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 28, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the light chain comprises an amino acid sequence as set forth in SEQ ID NO: 29, or a sequence with at least 90% sequence identity thereto.

According to another variation of the first aspect of the invention there is provided an isolated antibody that specifically binds human BTLA, comprising a heavy chain and a light chain, wherein the light chain comprises an amino acid sequence as set forth in SEQ ID NO: 29, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 28, or a sequence with at least 90% sequence identity thereto and the light chain comprises an amino acid sequence as set forth in SEQ ID NO: 29, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 28, or a sequence with up to 10 modifications, such as 1, 2, 3, 4, 5, 6, 7, 8, 9 or 10 amino acid modifications therein and wherein the light chain comprises an amino acid sequence as set forth in SEQ ID NO: 29, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 36 or 41, or a sequence with at least 90% sequence identity thereto.

In other embodiments, the heavy chain variable region comprises an amino acid sequence as set forth in SEQ ID NO: 36 or 41, with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the light chain comprises a light chain variable region comprising an amino acid sequence as set forth in SEQ ID NO: 37 or 43, or a sequence with at least 90% sequence identity thereto.

In other embodiments, the light chain variable region comprises an amino acid sequence as set forth in SEQ ID NO: 37 or 43, with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises a heavy chain variable region comprising an amino acid sequence that has at least 90% sequence identity to the amino acid sequence as set forth in SEQ ID NO: 36 or 41, and the light chain comprises a light chain variable region comprising an amino acid sequence that has at least 90% sequence identity to the amino acid sequence as set forth in SEQ ID NO: 37 or 43.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 36, and the light chain comprises a light chain variable region comprising an amino acid sequence as set forth in SEQ ID NO: 37.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 41, and the light chain comprises a light chain variable region comprising an amino acid sequence as set forth in SEQ ID NO: 37.

According to another variation of the first aspect of the invention there is provided an isolated antibody that specifically binds human BTLA, comprising a heavy chain and a light chain, wherein the heavy chain comprises a heavy chain variable region comprising an amino acid sequence as set forth in SEQ ID NO: 36, and the light chain comprises a light chain variable region comprising an amino acid sequence as set forth in SEQ ID NO: 43.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 38 or 42, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 38 or 42, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the light chain comprises an amino acid sequence as set forth in SEQ ID NO: 39 or 44, or a sequence with at least 90% sequence identity thereto.

According to another variation of the first aspect of the invention there is provided an isolated antibody that specifically binds human BTLA, comprising a heavy chain and a light chain, wherein the light chain comprises an amino acid sequence as set forth in SEQ ID NO: 39 or 44, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 38, or a sequence with at least 90% sequence identity thereto and the light chain comprises an amino acid sequence as set forth in SEQ ID NO: 39, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 38, or a sequence with up to 10 modifications, such as 1, 2, 3, 4, 5, 6, 7, 8, 9 or 10 amino acid modifications therein and wherein the light chain comprises an amino acid sequence as set forth in SEQ ID NO: 39, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 42, or a sequence with at least 90% sequence identity thereto and the light chain comprises an amino acid sequence as set forth in SEQ ID NO: 39, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 42, or a sequence with up to 10 modifications, such as 1, 2, 3, 4, 5, 6, 7, 8, 9 or 10 amino acid modifications therein and wherein the light chain comprises an amino acid sequence as set forth in SEQ ID NO: 39, or a sequence with up to 10 modifications, such as 1, 2, 3, 4, 5, 6, 7, 8, 9 or 10 amino acid modifications therein.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 38, or a sequence with at least 90% sequence identity thereto and the light chain comprises an amino acid sequence as set forth in SEQ ID NO: 44, or a sequence with at least 90% sequence identity thereto.

According to another variation of the first aspect of the invention there is provided an isolated antibody that specifically binds human BTLA, comprising a heavy chain and a light chain, wherein the heavy chain comprises an amino acid sequence as set forth in SEQ ID NO: 38, or a sequence with up to 10 modifications, such as 1, 2, 3, 4, 5, 6, 7, 8, 9 or 10 amino acid modifications therein and wherein the light chain comprises an amino acid sequence as set forth in SEQ ID NO: 44, or a sequence with up to 10 modifications, such as 1, 2, 3, 4, 5, 6, 7, 8, 9 or 10 amino acid modifications therein.

In other embodiments, the heavy chain variable region polypeptide has at least 92%, at least 95%, at least 97%, at least 98% or at least 99% identity with the sequence disclosed in SEQ ID NO: 18.

In other embodiments, the heavy chain variable region polypeptide has at least 92%, at least 95%, at least 97%, at least 98% or at least 99% identity with the sequence disclosed in SEQ ID NO: 26.

In other embodiments, the heavy chain variable region polypeptide has at least 92%, at least 95%, at least 97%, at least 98% or at least 99% identity with the sequence disclosed in SEQ ID NO: 36.

In other embodiments, the light chain variable region polypeptide has at least 92%, at least 95%, at least 97%, at least 98% or at least 99% identity with the sequence disclosed in SEQ ID NO: 14.

In other embodiments, the light chain variable region polypeptide has at least 92%, at least 95%, at least 97%, at least 98% or at least 99% identity with the sequence disclosed in SEQ ID NO: 27.

In other embodiments, the light chain variable region polypeptide has at least 92%, at least 95%, at least 97%, at least 98% or at least 99% identity with the sequence disclosed in SEQ ID NO: 43.

According to another variation of the first aspect of the invention there is provided an isolated antibody having primary VH domain and/or primary VL domain with at least one CDRH1, CDRH2, CDRH3, CDRL1, CDRL2, and CDRL3 of any antibody clone as set forth in Table 1 or Table 2. In certain embodiments, provided herein is an isolated antibody selected from the antibody clones as set forth in Table 1 or Table 2.

TABLE 1
Exemplary BTLA Agonistic Antibodies

| | | SEQ ID NOs | | | | | | | |
|---|---|---|---|---|---|---|---|---|---|
| Clone | Scheme | CDR H1 | CDR H2 | CDR H3 | CDR L1 | CDR L2 | CDR L3 | VH | VL |
| 10B1 | Kabat | 45 | 46 | 47 | 33 | 34 | 35 | 51 | 52 |
| 12F11 | Kabat | 53 | 54 | 55 | 56 | 57 | 58 | 59 | 60 |
| 14D4 | Kabat | 61 | 62 | 63 | 64 | 65 | 66 | 67 | 68 |
| 15B6 | Kabat | 61 | 69 | 70 | 71 | 72 | 73 | 74 | 75 |
| 15C6 | Kabat | 76 | 77 | 78 | 79 | 80 | 81 | 82 | 83 |
| 16E1 | Kabat | 45 | 46 | 84 | 33 | 34 | 85 | 86 | 87 |
| 16F10 | Kabat | 88 | 89 | 90 | 91 | 65 | 92 | 93 | 94 |
| 16H2 | Kabat | 95 | 96 | 97 | 98 | 99 | 100 | 101 | 102 |
| 1H6 | Kabat | 103 | 104 | 105 | 106 | 107 | 108 | 109 | 110 |
| 21C7 | Kabat | 76 | 111 | 112 | 113 | 114 | 115 | 116 | 117 |
| 24H7 | Kabat | 118 | 119 | 120 | 121 | 122 | 123 | 124 | 125 |
| 26B1 | Kabat | 126 | 127 | 128 | 79 | 129 | 130 | 131 | 132 |
| 26F3 | Kabat | 133 | 134 | 135 | 106 | 107 | 136 | 137 | 138 |
| 27G9 | Kabat | 103 | 134 | 139 | 106 | 107 | 136 | 141 | 138 |
| 3A9 | Kabat | 143 | 144 | 145 | 146 | 147 | 148 | 149 | 142 |
| 4B1 | Kabat | 151 | 152 | 153 | 154 | 155 | 156 | 157 | 158 |
| 4D3 | Kabat | 159 | 160 | 161 | 4 | 12 | 164 | 165 | 166 |
| 4D5 | Kabat | 167 | 168 | 169 | 170 | 171 | 172 | 173 | 174 |
| 4E8 | Kabat | 45 | 46 | 47 | 170 | 171 | 172 | 175 | 174 |
| 4H4 | Kabat | 45 | 46 | 177 | 154 | 155 | 178 | 179 | 180 |
| 6G8 | Kabat | 181 | 182 | 183 | 184 | 185 | 186 | 187 | 188 |
| 7A1 | Kabat | 76 | 77 | 78 | 79 | 80 | 189 | 82 | 190 |
| 8B4 | Kabat | 45 | 191 | 192 | 154 | 155 | 193 | 194 | 195 |
| 8C4 | Kabat | 196 | 197 | 198 | 199 | 200 | 201 | 202 | 203 |
| 11.5.1 | Kabat | 204 | 205 | 206 | 207 | 208 | 209 | 210 | 211 |
| 831 | Kabat | 212 | 213 | 214 | 215 | 34 | 216 | 217 | 218 |
| 6.2 | Kabat | 1 | 2 | 3 | 4 | 5 | 6 | 219 | 220 |
| 2.8.6 | Kabat | 20 | 163 | 22 | 23 | 176 | 25 | 221 | 222 |
| 3E8 | Kabat | 30 | 48 | 32 | 33 | 34 | 35 | 223 | 150 |

TABLE 2
Humanised and engineered antibodies

| | SEQ ID Nos. | | | | | | | | | |
|---|---|---|---|---|---|---|---|---|---|---|
| | | | | | | | | | Heavy | Light |
| Clone | CDR H1 | CDR H2 | CDR H3 | CDR L1 | CDR L2 | CDR L3 | VH | VL | chain | chain |
| humanised 6.2 | 1 | 2 | 3 | 4 | 5 | 6 | 7 | 8 | 9 | 10 |
| Engineered | 1 | 11 | 3 | 4 | 12 | 6 | 13 | 14 | 15 | 16 |
| humanised 6.2 | | | | | | | | | | |
| (Variant A) | | | | | | | | | | |
| Engineered | 1 | 11 | 3 | 4 | 5 | 6 | 13 | 8 | 15 | 10 |
| humanised 6.2 | | | | | | | | | | |
| (Variant B) | | | | | | | | | | |
| Engineered | 1 | 17 | 3 | 4 | 12 | 6 | 18 | 14 | 19 | 16 |
| humanised 6.2 | | | | | | | | | | |
| (Variant C) | | | | | | | | | | |
| Humanised | 20 | 21 | 22 | 23 | 24 | 25 | 26 | 27 | 28 | 29 |
| 2.8.6 | | | | | | | | | | |
| Humanised 3E8 | 30 | 31 | 32 | 33 | 34 | 35 | 36 | 37 | 38 | 39 |
| Engineered | 30 | 40 | 32 | 33 | 34 | 35 | 41 | 37 | 42 | 39 |
| humanised 3E8 | | | | | | | | | | |
| (Variant A) | | | | | | | | | | |
| Engineered | 30 | 31 | 32 | 33 | 34 | 35 | 36 | 43 | 38 | 44 |
| humanised 3E8 | | | | | | | | | | |
| (Variant B) | | | | | | | | | | |

In each of the first aspects of the invention, the antibody has an Fc region which comprises at least one amino acid substitution that results in increased binding to FcγR2B compared to the parent molecule that lacks the substitution and/or increased selectivity for binding FcγR2B over FcγR2A compared to the parent molecule that lacks the substitution. In some embodiments, the antibody has increased selectivity for binding FcγR2B over FcγR1A compared to the parent molecule that lacks the substitution.

In a particular embodiment, each of the antibodies according to the first aspect of the invention (which includes any variation of the first aspect) comprises an Fc region that comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, and alanine (A) at position 297 (all numbering according to EU Index)

In a particular embodiment, each of the antibodies according to the first aspect of the invention comprises an Fc with an aspartic acid at position 238 (EU Index).

In a particular embodiment, each of the antibodies according to the first aspect of the invention comprises an Fc with an aspartic acid at position 237 (EU Index).

In a particular embodiment, each of the antibodies according to the first aspect of the invention comprises an Fc with an aspartic acid at position 236 (EU Index).

In a particular embodiment, each of the antibodies according to the first aspect of the invention comprises an Fc with an alanine at position 235 (EU Index).

In a particular embodiment, each of the antibodies according to the first aspect of the invention comprises an Fc with an alanine at position 234 (EU Index).

In a particular embodiment, each of the antibodies according to the first aspect of the invention comprises an Fc with an alanine at position 265 (EU Index).

In a particular embodiment, each of the antibodies according to the first aspect of the invention comprises an Fc with a glutamic acid at position 267 (EU Index).

In a particular embodiment, each of the antibodies according to the first aspect of the invention comprises an Fc with a glycine at position 271 (EU Index).

In a particular embodiment, each of the antibodies according to the first aspect of the invention comprises an Fc with an alanine at position 297 (EU Index).

In a particular embodiment, each of the antibodies according to the first aspect of the invention comprises an Fc with an alanine at position 322 (EU Index).

In a particular embodiment, each of the antibodies according to the first aspect of the invention comprises an Fc with an arginine at position 330 (EU Index).

In a particular embodiment, each of the antibodies according to the first aspect of the invention comprises an Fc which comprises an aspartic acid at position 237 (EU Index), an aspartic acid at position 238 (EU Index), a glycine at position 271 (EU Index) and an arginine at position 330 (EU Index).

In particular embodiments, the antibody according to the first aspect of the invention comprises an Fc isotype with a substitution selected from the group consisting of: hIgG1 G236D, hIgG1 G237D, hIgG1 P238D, hIgG1 D265A, hIgG1 S267E, hIgG1 P271G, hIgG1 A330R, hIgG1 K322A, hIgG1 N297A, hIgG4 P238D, hIgG4 G237D, hIgG4 P271G, hIgG4 S330R, hIgG4 F234A and hIgG4 L235A.

In particular embodiments, the heavy chain or light chain also comprise a constant region. If the molecule is a full-length IgG-type antibody molecule, the heavy chain may comprise three constant domains. In certain embodiments the isolated antibody that specifically binds human BTLA exhibits a K_{D} for binding to human BTLA of at most about 10×10⁻⁹M. In certain embodiments the isolated antibody that specifically binds human BTLA exhibits a K_{D} for binding to human BTLA of at most about 4×10⁻⁹M. In certain embodiments the isolated antibody that specifically binds human BTLA exhibits a K_{D} for binding to human BTLA of at most about 1×10⁻⁹M.

In certain embodiments, an isolated antibody (e.g., humanised) of the invention binds human BTLA at 37° C. with a K_{D} of no more than about 10 nM (1×10⁻⁸M); suitably no more than about 1 nM; more suitably are embodiments in which the antibodies have K_{D} values at 37° C. of no more than about 500 pM (5×10⁻¹⁰ M), 200 pM, 100 pM, 50 pM, 20 pM, 10 pM, 5 pM or even 2 pM. The term “about”, as used in this context means +/−10%.

In certain embodiments, an isolated antibody (e.g., humanised) of the invention binds human BTLA at 37° C. with an on rate of at least 1.0×10⁵ (l/Ms). In certain embodiments, an isolated antibody (e.g., humanised) of the invention binds human BTLA at 37° C. with an on rate of at least 2.0×10⁵ (l/Ms), 3.0×10⁵ (l/Ms), 4.0×10⁵ (l/Ms), 5.0×10⁵ (l/Ms), 6.0×10⁵ (l/Ms), or 7.0×10⁵ (l/Ms).

In certain embodiments, an isolated antibody (e.g., humanised) of the invention binds human BTLA at 37° C. with an off rate of no more than or less than 1.0×10⁻³ (l/s). In certain embodiments, an isolated antibody (e.g., humanised) of the invention binds human BTLA at 37° C. with an off rate of no more than or less than 3.0×10⁻⁴ (l/s). In certain embodiments, an isolated antibody (e.g., humanised) of the invention binds human BTLA at 37° C. with an off rate of no more than or less than 2.0×10⁻⁴ (l/s), or 1.0×10⁻⁴ (l/s).

In particular embodiments of the first aspect of the invention, provided herein are isolated agonistic antibodies that specifically binds human B and T Lymphocyte Attenuator (BTLA) with a KD of less than 10 nM, as determined by surface plasmon resonance (SPR) at 37° C. using a method such as that described in Example 2, and wherein said antibody binds cynomolgus BTLA with a K_{D} of less than 20 nM, as determined by surface plasmon resonance (SPR) at 37° C. using a method such as that described in Example 2; does not inhibit binding of BTLA to herpes virus entry mediator (HVEM), as determined for example by surface plasmon resonance (SPR) using a method such as that described in Example 4; and inhibits proliferation of T cells in vitro, as determined for example by a mixed lymphocyte reaction assay using a method such as that described in Example 9. In some embodiments, said antibody binds human B and T Lymphocyte Attenuator (BTLA) with an on rate of at least 5.0×10⁵ (l/Ms) as determined by surface plasmon resonance (SPR) at 37° C. using a method such as that described in Example 2. In some embodiments, said antibody binds human B and T Lymphocyte Attenuator (BTLA) with an off rate of less than 3.0×10⁻⁴ (l/s) as determined by surface plasmon resonance (SPR) at 37° C. using a method such as that described in Example 2. In some embodiments, said antibody binds human B and T Lymphocyte Attenuator (BTLA) with an off rate from 3.0×10⁻⁴ (l/s) to 1.0×10⁻³ (l/s) as determined by surface plasmon resonance (SPR) at 37° C. using a method such as that described in Example 2. In some embodiments, the antibody binds a residue of human BTLA selected from: D52, P53, E55, E57, E83, Q86, E103, L106 and E92 as determined by x-ray crystallography or by flow cytometry of mutated receptors using a method such as that described in Example 5 (numbering here, e.g. D52, refers to the position in SEQ ID NO: 225). In some embodiments, the antibody binds a residue of human BTLA selected from: Y39, K41, R42, Q43, E45 and S47. In some embodiments, the antibody binds a residue of human BTLA selected from: D35, T78, K81, S121 and L123. In some embodiments, the antibody binds residue H68 of human BTLA. In some embodiments, the antibody binds a residue of human BTLA selected from: N65 and A64 (position according to SEQ ID NO:225).

Methods for characterizing the properties of an antibody of the invention are well known in the art. A suitable method for determining binding specificity using surface plasmon resonance (SPR) at 37° C. is described in Example 2. A suitable method for determining whether the tested antibody/fragment thereof inhibits binding of BTLA to herpes virus entry mediator (HVEM) is described in Example 4; this also employs surface plasmon resonance (SPR). A suitable method for determining whether the tested antibody/fragment thereof inhibits proliferation of T cells in vitro, is a mixed lymphocyte reaction assay such as that described in Example 9. Suitable methods for determining the site of binding of an antibody/fragment thereof to BTLA can utilise x-ray crystallography or flow cytometry of mutated receptors, such as by the method described in Example 5

In particular embodiments of the first aspect of the invention, provided herein are isolated agonistic antibodies that specifically binds human B and T Lymphocyte Attenuator (BTLA) with an on rate of at least 5.0×10⁵ (l/Ms), as determined by surface plasmon resonance (SPR) at 37° C. using a method such as that described in Example 2, wherein said antibody does not inhibit binding of BTLA to herpes virus entry mediator (HVEM) as determined for example by surface plasmon resonance (SPR) using a method such as that described in Example 4; and wherein said antibody inhibits proliferation of T cells in vitro, as determined for example by a mixed lymphocyte reaction assay using a method such as that described in Example 9. In some embodiments, said antibody binds human B and T Lymphocyte Attenuator (BTLA) with an off rate of less than 3.0×10⁻⁴ (l/s) as determined by surface plasmon resonance (SPR) at 37° C. using a method such as that described in Example 2. In some embodiments, said antibody binds human B and T Lymphocyte Attenuator (BTLA) with a K_{D} of less than 10 nM, as determined by surface plasmon resonance (SPR) at 37° C. using a method such as that described in Example 2. In some embodiments, said antibody binds cynomolgus BTLA with a K_{D} of less than 20 nM, as determined by surface plasmon resonance (SPR) at 37° C. using a method such as that described in Example 2. In some embodiments, the antibody binds a residue of human BTLA selected from: D52, P53, E55, E57, E83, Q86, E103, L106 and E92 as determined by x-ray crystallography or by flow cytometry of mutated receptors using a method such as that described in Example 5. In some embodiments, the antibody binds a residue of human BTLA selected from: Y39, K41, R42, Q43, E45 and S47. In some embodiments, the antibody binds a residue of human BTLA selected from: D35, T78, K81, S121 and L123. In some embodiments, the antibody binds residue H68 of human BTLA (position according to SEQ ID NO:225). In some embodiments, the antibody binds a residue of BTLA selected from: N65 and A64 (position according to SEQ ID NO:225).

In particular embodiments of the first aspect of the invention, provided herein are isolated agonistic antibody that specifically binds human B and T Lymphocyte Attenuator (BTLA) with an off rate from 3.0×10⁻⁴ (l/s) to 1.0×10⁻³ (l/s) as determined by surface plasmon resonance (SPR) at 37° C. using a method such as that described in Example 2, wherein said antibody does not inhibit binding of BTLA to herpes virus entry mediator (HVEM) as determined for example by surface plasmon resonance (SPR) using a method such as that described in Example 4; and wherein said antibody inhibits proliferation of T cells in vitro, as determined for example by a mixed lymphocyte reaction assay using a method such as that described in Example 9. In some embodiments, said antibody binds human B and T Lymphocyte Attenuator (BTLA) with a KD of less than 10 nM, as determined by surface plasmon resonance (SPR) at 37° C. using a method such as that described in Example 2. In some embodiments, said antibody binds cynomolgus BTLA with a K_{D} of less than 20 nM, as determined by surface plasmon resonance (SPR) at 37° C. using a method such as that described in Example 2. In some embodiments, said antibody binds human B and T Lymphocyte Attenuator (BTLA) with an on rate of at least 5.0×10⁵ (l/Ms) as determined by surface plasmon resonance (SPR) at 37° C. using a method such as that described in Example 2. In some embodiments, the antibody binds a residue of human BTLA selected from: D52, P53, E55, E57, E83, Q86, E103, L106 and E92 (positions according to SEQ ID NO:225) as determined by x-ray crystallography or by flow cytometry of mutated receptors using a method such as that described in Example 5. In some embodiments, the antibody binds a residue of human BTLA selected from: Y39, K41, R42, Q43, E45 and S47 (positions according to SEQ ID NO:225). In some embodiments, the antibody binds a residue of human BTLA selected from: D35, T78, K81, S121 and L123. In some embodiments, the antibody binds residue H68 of human BTLA (position according to SEQ ID NO:225). In some embodiments, the antibody binds a residue of human BTLA selected from: N65 and A64 (position according to SEQ ID NO:225).

In particular embodiments of the first aspect of the invention, provided herein are isolated agonistic antibodies that specifically binds human B and T Lymphocyte Attenuator (BTLA) with an off rate of less than 1.0×10⁻³ (l/s) and an on rate of at least 5.0×10⁵ (l/Ms), each as measured by surface plasmon resonance (SPR) at 37° C. using a method such as that described in Example 2, wherein said antibody does not inhibit binding of BTLA to herpes virus entry mediator (HVEM) as determined for example by surface plasmon resonance (SPR) using a method such as that described in Example 4; and wherein said antibody inhibits proliferation of T cells in vitro, as determined for example by a mixed lymphocyte reaction assay using a method such as that described in Example 9. In some embodiments, said antibody binds human B and T Lymphocyte Attenuator (BTLA) with a K_{D} of less than 10 nM, as determined by surface plasmon resonance (SPR) at 37° C. using a method such as that described in Example 2. In some embodiments, said antibody binds cynomolgus BTLA with a K_{D} of less than 20 nM, as determined by surface plasmon resonance (SPR) at 37° C. using a method such as that described in Example 2. In some embodiments, the antibody binds a residue of human BTLA selected from: D52, P53, E55, E57, E83, Q86, E103, L106 and E92 (position according to SEQ ID NO:225) as determined by x-ray crystallography or by flow cytometry of mutated receptors using a method such as that described in Example 5. In some embodiments, the antibody binds a residue of BTLA selected from: Y39, K41, R42, Q43, E45 and S47. In some embodiments, the antibody binds a residue of human BTLA selected from: D35, T78, K81, S121 and L123. In some embodiments, the antibody binds residue H68 of human BTLA. In some embodiments, the antibody binds a residue of human BTLA selected from: N65 and A64.

In particular embodiments of the first aspect of the invention, provided herein is an isolated agonistic antibody that specifically binds human B and T Lymphocyte Attenuator (BTLA) with a KD of less than 2 nM, as determined by surface plasmon resonance (SPR) at 37° C. using a method such as that described in Example 2, wherein said antibody inhibits binding of BTLA to herpes virus entry mediator (HVEM) as determined by surface plasmon resonance (SPR) using a method such as that described in Example 4; and inhibits proliferation of T cells in vitro, as determined for example by a mixed lymphocyte reaction assay using a method such as that described in Example 9. In some embodiments, said antibody binds human B and T Lymphocyte Attenuator (BTLA) with an on rate of less than 1.0×10⁶ (l/Ms), as determined by surface plasmon resonance (SPR) at 37° C. using a method such as that described in Example 2. In some embodiments, said antibody binds human B and T Lymphocyte Attenuator (BTLA) with an off rate of less than 1.0×10⁻³ (l/s), as determined by surface plasmon resonance (SPR) at 37° C. using a method such as that described in Example 2. In some embodiments, said antibody binds cynomolgus B and T Lymphocyte Attenuator (BTLA) with a K_{D} of less than 10 nM, as determined by surface plasmon resonance (SPR) at 37° C. using a method such as that described in Example 2. In some embodiments, the antibody binds a residue of human BTLA selected from: D52, P53, E55, E57, E83, Q86, E103, L106 and E92 (positions according to SEQ ID NO:225) as determined by x-ray crystallography or by flow cytometry of mutated receptors using a method such as that described in Example 5. In some embodiments, the antibody binds a residue of human BTLA selected from: Y39, K41, R42, Q43, E45 and S47 (position according to SEQ ID NO:225). In some embodiments, the antibody binds a residue of human BTLA selected from: D35, T78, K81, S121 and L123 (position according to SEQ ID NO:225). In some embodiments, the antibody binds residue H68 of BTLA (position according to SEQ ID NO:225). In some embodiments, the antibody binds a residue of human BTLA selected from: N65 and A64 (position according to SEQ ID NO:225).

In particular embodiments of the first aspect of the invention, provided herein is an isolated agonistic antibody that specifically binds human B and T Lymphocyte Attenuator (BTLA) with an on off rate of less than 1×10⁻³ (l/s) as determined by surface plasmon resonance (SPR) at 37° C. using a method such as that described in Example 2, wherein said antibody inhibits binding of BTLA to herpes virus entry mediator (HVEM) as determined by surface plasmon resonance (SPR) using a method such as that described in Example 4, and inhibits proliferation of T cells in vitro, as determined for example by a mixed lymphocyte reaction assay using a method such as that described in Example 9. In some embodiments, said antibody binds cynomolgus B and T Lymphocyte Attenuator (BTLA) with a K_{D} of less than 10 nM, as determined by surface plasmon resonance (SPR) at 37° C. using a method such as that described in Example 2. In some embodiments, said antibody binds human B and T Lymphocyte Attenuator (BTLA) with a KD of less than 2 nM, as determined by surface plasmon resonance (SPR) at 37° C. using a method such as that described in Example 2. In some embodiments, the antibody binds a residue of human BTLA selected from: D52, P53, E55, E57, E83, Q86, E103, L106 and E92 (positions according to SEQ ID NO:225) as determined by x-ray crystallography or by flow cytometry of mutated receptors using a method such as that described in Example 5. In some embodiments, the antibody binds a residue of human BTLA selected from: Y39, K41, R42, Q43, E45 and S47 (positions according to SEQ ID NO:225). In some embodiments, the antibody binds a residue of human BTLA selected from: D35, T78, K81, S121 and L123 (positions according to SEQ ID NO:225). In some embodiments, the antibody binds residue H68 of human BTLA (position according to SEQ ID NO:225). In some embodiments, the antibody binds a residue of human BTLA selected from: N65 and A64 (position according to SEQ ID NO:225).

In particular embodiments of the first aspect of the invention, provided herein is an isolated agonistic antibody that specifically binds human B and T Lymphocyte Attenuator (BTLA), wherein said antibody binds cynomolgus BTLA with a K_{D} of at least 5 nM, as determined by surface plasmon resonance (SPR) at 37° C. using a method such as that described in Example 2; and wherein said antibody inhibits binding of BTLA to herpes virus entry mediator (HVEM) as determined by surface plasmon resonance (SPR) using a method such as that described in Example 4; and inhibits proliferation of T cells in vitro, as determined for example by a mixed lymphocyte reaction assay using a method such as that described in Example 9. In some embodiments, the antibody binds a residue of human BTLA selected from: D52, P53, E55, E57, E83, Q86, E103, L106 and E92 (positions according to SEQ ID NO:225) as determined by x-ray crystallography or by flow cytometry of mutated receptors using a method such as that described in Example 5. In some embodiments, the antibody binds a residue of human BTLA selected from: Y39, K41, R42, Q43, E45 and S47 (positions according to SEQ ID NO:225). In some embodiments, the antibody binds a residue of human BTLA selected from: D35, T78, K81, S121 and L123 (positions according to SEQ ID NO:225). In some embodiments, the antibody binds residue H68 of human BTLA (position according to SEQ ID NO:225). In some embodiments, the antibody binds a residue of human BTLA selected from: N65 and A64 (position according to SEQ ID NO:225).

In particular embodiments of the first aspect of the invention, provided herein is an isolated agonistic antibody that specifically binds human B and T Lymphocyte Attenuator (BTLA), wherein said antibody binds cynomolgus BTLA with a K_{D} of at least than 50 nM, as determined by surface plasmon resonance (SPR) at 37° C. using a method such as that described in Example 2; and wherein said antibody does not inhibit binding of BTLA to herpes virus entry mediator (HVEM) as determined by surface plasmon resonance (SPR) using a method such as that described in Example 4; and inhibits proliferation of T cells in vitro, as determined for example by a mixed lymphocyte reaction assay using a method such as that described in Example 9. In some embodiments, the antibody binds a residue of human BTLA selected from: D52, P53, E55, E57, E83, Q86, E103, L106 and E92 (positions according to SEQ ID NO:225) as determined by x-ray crystallography or by flow cytometry of mutated receptors using a method such as that described in Example 5. In some embodiments, the antibody binds a residue of human BTLA selected from: Y39, K41, R42, Q43, E45 and S47 (positions according to SEQ ID NO:225). In some embodiments, the antibody binds a residue of human BTLA selected from: D35, T78, K81, S121 and L123 (positions according to SEQ ID NO:225). In some embodiments, the antibody binds residue H68 of human BTLA (position according to SEQ ID NO:225). In some embodiments, the antibody binds a residue of human BTLA selected from: N65 and A64 (position according to SEQ ID NO:225).

In particular embodiments of the first aspect of the invention, provided herein is an isolated agonistic antibody that specifically binds human B and T Lymphocyte Attenuator (BTLA) with a KD from 1400 nM to 3500 nM, as determined by surface plasmon resonance (SPR) at 37° C. using a method such as that described in Example 2; and wherein said antibody does not inhibit binding of BTLA to herpes virus entry mediator (HVEM) as determined by surface plasmon resonance (SPR) using a method such as that described in Example 4; and inhibits proliferation of T cells in in vitro, as determined for example by a mixed lymphocyte reaction assay using a method such as that described in Example 9. In some embodiments, said antibody binds human BTLA with an on rate of at least 2.0×10⁵ (l/Ms), as determined by surface plasmon resonance (SPR) at 37° C. using a method such as that described in Example 2. In some embodiments, said antibody binds human BTLA with an off rate of less than 10.0×10⁻¹ (l/s), as determined by surface plasmon resonance (SPR) at 37° C. using a method such as that described in Example 2. In some embodiments, the antibody binds a residue of human BTLA selected from: D52, P53, E55, E57, E83, Q86, E103, L106 and E92 (positions according to SEQ ID NO:225) as determined by x-ray crystallography or by flow cytometry of mutated receptors using a method such as that described in Example 5. In some embodiments, the antibody binds a residue of human BTLA selected from: Y39, K41, R42, Q43, E45 and S47 (positions according to SEQ ID NO:225). In some embodiments, the antibody binds a residue of human BTLA selected from: D35, T78, K81, S121 and L123 (positions according to SEQ ID NO:225). In some embodiments, the antibody binds residue H68 of human BTLA (position according to SEQ ID NO:225). In some embodiments, the antibody binds a residue of human BTLA selected from: N65 and A64 (position according to SEQ ID NO:225).

In particular embodiments of the first aspect of the invention, provided herein is an isolated agonistic antibody that specifically binds human B and T Lymphocyte Attenuator (BTLA) with an on rate from 1.7×10⁵ (l/Ms) to 2.5×10⁵ (l/Ms), as determined by surface plasmon resonance (SPR) at 37° C. using a method such as that described in Example 2; and wherein said antibody does not inhibit binding of BTLA to herpes virus entry mediator (HVEM) as determined by surface plasmon resonance (SPR) using a method such as that described in Example 4; and inhibits proliferation of T cells in in vitro, as determined for example by a mixed lymphocyte reaction assay using a method such as that described in Example 9. In some embodiments, said antibody binds human BTLA with an off rate of less than 3.0×10⁻¹ (l/s), as determined by surface plasmon resonance (SPR) at 37° C. using a method such as that described in Example 2. In some embodiments, said antibody binds human BTLA with an off rate from 3.0×10⁻¹ (l/s) to 5.0×10⁻¹ (l/s), as determined by surface plasmon resonance (SPR) at 37° C. using a method such as that described in Example 2. In some embodiments, said antibody binds human BTLA with a K_{D} of at least 150 nM, as determined by surface plasmon resonance (SPR) at 37° C. using a method such as that described in Example 2. In some embodiments, said antibody binds human BTLA with a KD from 150 nM to 1500 nM, as determined by surface plasmon resonance (SPR) at 37° C. using a method such as that described in Example 2. In some embodiments, said antibody binds to an epitope that blocks binding of 286 antibody. In some embodiments, the antibody binds a residue of human BTLA selected from: D52, P53, E55, E57, E83, Q86, E103, L106 and E92 as determined by x-ray crystallography or by flow cytometry of mutated receptors using a method such as that described in Example 5. In some embodiments, the antibody binds a residue of human BTLA selected from: Y39, K41, R42, Q43, E45 and S47 (positions according to SEQ ID NO:225). In some embodiments, the antibody binds a residue of human BTLA selected from: D35, T78, K81, S121 and L123 (positions according to SEQ ID NO:225). In some embodiments, the antibody binds residue H68 of human BTLA (position according to SEQ ID NO:225). In some embodiments, the antibody binds a residue of human BTLA selected from: N65 and A64 (position according to SEQ ID NO:225).

In particular embodiments of the first aspect of the invention, provided herein is an isolated agonistic antibody that specifically binds human B and T Lymphocyte Attenuator (BTLA) with a KD from 40 nM to 1200 nM, as determined by surface plasmon resonance (SPR) at 37° C. using a method such as that described in Example 2; and wherein said antibody does not inhibit binding of BTLA to herpes virus entry mediator (HVEM) as determined by surface plasmon resonance (SPR) using a method such as that described in Example 4; and inhibits proliferation of T cells in in vitro, as determined for example by a mixed lymphocyte reaction assay using a method such as that described in Example 9. In some embodiments, said antibody binds human BTLA with an on rate of at least 1.0×10⁵ (l/Ms), as determined by surface plasmon resonance (SPR) at 37° C. using a method such as that described in Example 2. In some embodiments, said antibody binds human BTLA with an on rate from 1.0×10⁵ (l/Ms) to 10×10⁵ (l/Ms), as determined by surface plasmon resonance (SPR) at 37° C. using a method such as that described in Example 2. In some embodiments, said antibody binds human BTLA with an off rate of less than 6.0×10⁻¹ (l/s), as determined by surface plasmon resonance (SPR) at 37° C. using a method such as that described in Example 2. In some embodiments, said antibody binds human BTLA with an off rate from 6.0×10⁻¹ (l/s) to 10.0×10⁻² (l/s), as determined by surface plasmon resonance (SPR) at 37° C. using a method such as that described in Example 2. In some embodiments, the antibody binds a residue of human BTLA selected from: D52, P53, E55, E57, E83, Q86, E103, L106 and E92 (positions according to SEQ ID NO:225) as determined by x-ray crystallography or by flow cytometry of mutated receptors using a method such as that described in Example 5. In some embodiments, the antibody binds a residue of human BTLA selected from: Y39, K41, R42, Q43, E45 and S47 (positions according to SEQ ID NO:225). In some embodiments, the antibody binds a residue of human BTLA selected from: D35, T78, K81, S121 and L123 (positions according to SEQ ID NO:225). In some embodiments, the antibody binds residue H68 of human BTLA (position according to SEQ ID NO:225). In some embodiments, the antibody binds a residue of human BTLA selected from: N65 and A64 (position according to SEQ ID NO:225).

In certain embodiments the isolated antibody of the invention that specifically binds human BTLA increases BTLA activity and/or signaling through the receptor.

As noted above, the term antibody when used in relation to the first aspect of the invention embraces whole antibodies as well as antigen-binding fragments thereof.

### Particular Fc Receptor Binding Embodiments

In certain embodiments of the invention, particularly when in accordance with the first aspect of the invention, the heavy chain comprises an Fc region that comprises a substitution that confers on the antibody molecule an increased binding to and thus enhanced signaling of FcγR2B. In certain embodiments, such molecules have reduced binding to one or more activating Fcgamma receptors, such as FcγR2A or FcγR1A compared to a parent polypeptide. In certain embodiments, such molecules have an increased ratio of binding to FcγR2B/FcγR2A compared to a parent polypeptide. In certain embodiments, such molecules have an increased ratio of binding to FcγR2B/FcγR1A compared to a parent polypeptide.

As noted above, in particular embodiments of the invention, particularly when in accordance with the first aspect of the invention, the antibody comprises a heavy chain and a light chain, wherein said heavy chain comprises an Fc region that comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, and alanine (A) at position 297 (all numbering according to EU Index). Suitably, the Fc region and thus the antibody itself is capable of binding to an Fcγ receptor.

In a particular embodiment, the Fc region binds to FcγR2B with a higher affinity relative to a comparable control antibody that comprises an Fc region that lacks the one or more Fc substitutions recited above. In particular embodiments, the antibody binds to FcγR2B with a dissociation constant (KD) of from about 5 μM to 0.1 μM, as determined by surface plasmon resonance (SPR). Suitably, the antibody binds to FcγR2B via its Fc region.

In particular embodiments, the antibody binds to FcγR2B with a K_{D} of at most 5 μM, as determined by surface plasmon resonance (SPR).

In particular embodiments, the antibody binds to FcγR2A (131R allotype) with a lower or equal affinity relative to a parental molecule. A parental molecule being the equivalent antibody that lacks the Fc substitution that confers on the antibody molecule an increased binding to and thus enhanced signaling of FcγR2B.

In particular embodiments, when the antibody comprises the P238D substitution the antibody binds to FcγR2A (131R allotype) with a lower or equal affinity relative to a comparable control antibody that comprises an Fc region that comprises a proline at position 238 (EU Index).

In particular embodiments, the antibody binds to FcγR2A (131R allotype) with a K_{D} of at least 20 μM, as determined by surface plasmon resonance (SPR).

In particular embodiments, the antibody binds to FcγR2A (131R allotype) with a KD of from about 25 μM to 35 μM, as determined by surface plasmon resonance (SPR).

In particular embodiments, the antibody binds to FcγR2A (131H allotype) with a lower or equal affinity relative to a parental molecule.

In particular embodiments, when the antibody comprises the P238D substitution the antibody binds to FcγR2A (131H allotype) with a lower or equal affinity relative to a comparable control antibody that comprises an Fc region that comprises a proline at position 238 (EU Index).

In particular embodiments, the antibody binds to FcγR2A (131H allotype) with a K_{D} of at least 50 μM, as determined by surface plasmon resonance (SPR).

In particular embodiments, the antibody possesses a [KD value of the antibody for FcγR2A (131R)/KD value of the antibody for FcγR2B] of 3 or more, such as at least 5. Suitably, as determined by surface plasmon resonance (SPR).

In particular embodiments, the antibody possesses a [KD value of the antibody for FcγR2A(131H)]/[KD value of the antibody for FcγR2B] of 10 or more, such as at least 15. Suitably, as determined by surface plasmon resonance (SPR).

In particular embodiments, the antibody possesses a [KD value of the antibody for FcγR2A (131R)/KD value of the antibody for FcγR2B] of 3 or more, such as at least 5 and/or a [KD value of the antibody for FcγR2A(131H)]/[KD value of the antibody for FcγR2B] of 10 or more, such as at least 15. Suitably, as determined by surface plasmon resonance (SPR).

Suitably, the antibody of the invention exhibits increased agonism of human BTLA expressed on the surface of a human immune cell, relative to a comparable control antibody/parental antibody, as measured by a BTLA agonist assay selected from a T cell activation assay such as that described in example 24, a mixed lymphocyte reaction such as that described in example 25 or a B cell activation assay such as that described in example 26.

Thus, if the antibody comprises the P238D substitution the antibody exhibits increased agonism of human BTLA expressed on the surface of a human immune cell, relative to a comparable control antibody that comprises an Fc region that comprises a proline at position 238, as measured by a BTLA agonist assay selected from a T cell activation assay such as that described in example 24, a mixed lymphocyte reaction such as that described in example 25 or a B cell activation assay such as that described in example 26.

In particular embodiments, the antibody of the invention is selected from the group consisting of: a human antibody, a humanised antibody, a chimeric antibody, a multispecific antibody (such as a bispecific antibody).

In particular embodiments, the antibody of the invention is an antigen-binding fragment antibody selected from the group consisting of: scFv, sc(Fv)², dsFv, Fab, Fab′, (Fab′)2 and diabody.

In particular embodiments, the heavy chain and light chain molecules that form the antigen-binding fragment are connected by a flexible linker. There are many commonly used flexible linkers and the choice of linker can be made by a person of skill in the art.

The peptide linker connecting scFv VH and VL domains joins the carboxyl terminus of one variable region domain to the amino terminus of another variable domain without significantly compromising the fidelity of the VH-VL pairing and antigen-binding sites. Peptide linkers can vary from 10 to 25 amino acids in length and are typically, but not always, composed of hydrophilic amino acids such as glycine (G) and serine (S). The linker can be one that is found in natural multi-domain proteins (e.g. see Argos P. J Mol Biol. 211:943-958, 1990; and. Heringa G. Protein Eng. 15:871-879, 2002), or adapted therefrom.

Commonly used flexible linkers have sequences consisting primarily of stretches of Gly and Ser residues (“GS” linker). An example of the most widely used flexible linker has the sequence of (Gly-Gly-Gly-Gly-Ser)ₙ (SEQ ID NO:232). By adjusting the copy number “n”, the length of this GS linker can be altered to achieve appropriate separation of the functional domains, or to maintain necessary inter-domain interactions. Generally, the GS linker with n=3 peptide is used as an scFv peptide linker (Leith et al., Int. J. Oncol. 24:765-771, 2004; Holiger et al. Proc. Natl. Acad. Sci. U.S.A. 90:6444-6448, 1993). This 15-amino acid linker sequence [designated as the (GGGGS)3 linker] is used in the Recombinant Phage Antibody System (RPAS kit) commercially available from Amersham. Several other linkers have also been used to create scFV molecules (e.g. KESGSVSSEQLAQFRSLD (SEQ ID NO: 233) and EGKSSGSGSESKST (SEQ ID NO: 234); Bird et al., Science 242:432-426, 1988).

### Epitope Binding

The inventors have mapped the epitopes on BTLA where the potent agonist and antibodies disclosed herein bind.

In particular embodiments, the antibody of the invention binds a residue of human BTLA selected from: D52, P53, E55, E57, E83, Q86, E103, L106, E92, Y39, K41, R42, Q43, E45, S47, D35, T78, K81, S121, L123, H68, N65, A64.

In particular embodiments, the antibody of the invention binds a residue of human BTLA selected from: D52, P53, E55, E57, E83, Q86, E103, L106, E92.

In particular embodiments, the antibody of the invention binds at least two residues of human BTLA selected from: D52, P53, E55, E57, E83, Q86, E103, L106, E92.

In particular embodiments, the antibody of the invention binds at least three residues of human BTLA selected from: D52, P53, E55, E57, E83, Q86, E103, L106 and E92.

In particular embodiments, the antibody of the invention binds at least five residues of human BTLA selected from: D52, P53, E55, E57, E83, Q86, E103, L106 and E92.

In particular embodiments, the antibody of the invention binds all of the residues of human BTLA selected from: D52, P53, E55, E57, E83, Q86, E103, L106 and E92.

In particular embodiments, the antibody of the invention binds a residue of human BTLA selected from: Y39, K41, R42, Q43, E45 and S47.

In a particular embodiment, the antibody of the invention binds at least two residues of human BTLA selected from: Y39, K41, R42, Q43, E45 and S47.

In particular embodiments, the antibody of the invention binds all of the residues of human BTLA selected from: Y39, K41, R42, Q43, E45 and S47.

In particular embodiments, the antibody of the invention binds a residue of human BTLA selected from: D35, T78, K81, S121 and L123.

In a particular embodiment, the antibody of the invention binds at least two residues of human BTLA selected from: D35, T78, K81, S121 and L123.

In particular embodiments, the antibody of the invention binds residue H68 of human BTLA

In particular embodiments, the antibody of the invention binds a residue of human BTLA selected from: N65 and A64.

In particular embodiments, the antibody of the invention binds both the N65 and A64 residues of human BTLA.

The numbering of the residues, such as K41 refers to the amino acid (K; lysine) at position 41; wherein the numbering refers to the position in human BTLA polypeptide as disclosed in SEQ ID NO: 225.

In particular embodiments, the antibody of the invention is an IgG1, IgG2 or IgG4 antibody. In particular embodiments, the antibody is a murine or human antibody.

In a particular embodiment, the antibody of the invention is a humanised antibody.

In a particular embodiment, the antibody of the invention is a fully human antibody.

In a particular embodiment, the antibody of the invention acts as an agonist inducing signaling through the BTLA receptor.

The antibodies (including antigen-binding fragments) of the invention are particularly potent agonists.

In a particular embodiment, the antibody of the invention has an EC50s of not more than 1 nM.

The agonist antibodies (e.g. full length/whole antibodies or antigen-binding fragments thereof) of the invention have particularly high efficacy.

In a particular embodiment the antibody of the invention inhibits T cell proliferation by at least 20%, suitably by at least 30%, more suitably by at least 40%.

In a particular embodiment the antibody of the invention inhibits T cell IFN-gamma production by at least 50%, suitably by at least 75%, more suitably by at least 95%, as measured for example by ELISA of supernatants in an in vitro mixed lymphocyte reaction.

In a particular embodiment the antibody of the invention inhibits T cell IL-2 production by at least 50%, suitably by at least 75%, more suitably by at least 95%, as measured for example by ELISA of supernatants in an in vitro mixed lymphocyte reaction.

In a particular embodiment the antibody of the invention inhibits T cell IL-17 production by at least 50%, suitably by at least 75%, more suitably by at least 95%, as measured for example by ELISA of supernatants in an in vitro mixed lymphocyte reaction.

In a particular embodiment the antibody of the invention reduces mortality in a murine GVHD model by at least 50%, suitably by at least 75%, more suitably by at least 95%, using a method such as that described in Example 12.

In a particular embodiment the antibody of the invention reduces weight loss in a murine T-cell colitis model by at least 50%, suitably by at least 75%, more suitably by at least 95%, using a method such as that described in Example 11.

In a particular embodiment the antibody of the invention reduces colon inflammation in a murine T-cell colitis model by at least 50%, suitably by at least 75%, more suitably by at least 95%, using a method such as that described in Example 11.

In certain aspects, the invention also relates to an isolated polypeptide comprising the VL domains or the VH domains of any of the antibodies described herein.

As noted herein, in particular embodiments the antibody that binds BTLA comprises a heavy chain and a light chain, wherein said heavy chain comprises an Fc region that comprises an aspartic acid at position 238 (EU Index).

### Nucleic Acid Molecules

The antibody of the invention will be encoded by nucleic acid. The antibody (including an antigen-binding fragment thereof) may be encoded by a single nucleic acid molecule or it may be encoded by two or more nucleic acid molecules. For example, as the antigen binding site is typically formed by the coming together of a heavy chain variable polypeptide region and a light chain variable polypeptide region, the two variable (heavy and light) polypeptide regions may be encoded by separate nucleic acid molecules. Alternatively, for example in the case of an ScFv, they may be encoded by the same nucleic acid molecule.

According to a second aspect of the invention there is provided one or more nucleic acid molecules that encode an antibody in accordance with the first aspect of the invention.

From the primary amino acid sequence of the polypeptide(s) encoding an antibody of the invention the person of skill in the art is able to determine suitable nucleotide sequence(s) that encodes the polypeptide(s) and, if desired, one that is codon-optimised (e.g. see Mauro and Chappell. Trends Mol Med. 20(11):604-613, 2014).

As used herein, when there is reference to a previous aspect of the invention, e.g. “in accordance with the first (or second etc.) aspect of the invention”, it is understood to also cover any recited variation of said aspect (e.g. variation of the first (or second etc.) aspect). Further, any embodiment that applies to a particular aspect of the invention also applies to any variation of that aspect, thus an embodiment that applies to the first aspect of the invention also applies to a variation of the first aspect of the invention.

According to a variation of the second aspect of the invention there is provided an isolated nucleic acid comprising a nucleotide sequence that encodes a heavy chain variable region polypeptide or a light chain variable region polypeptide of the invention. A heavy chain variable polypeptide or a light chain variable polypeptide of the invention refers to the individual polypeptide chains that include amino acids that make up part of the antigen-binding site. Of course, the said polypeptides may also comprise other domains such as constant domains, hinge regions, and an Fc region, such as one comprising one or more Fc receptor binding sites.

According to another variation of the second aspect of the invention there is provided an isolated nucleic acid which comprises one or more nucleotide sequence encoding polypeptides capable of forming an antibody of the invention. In particular embodiments, the said polypeptides may also comprise other domains such as constant domains, hinge regions, and an Fc region, such as one comprising one or more Fc receptor binding sites.

One of the nucleic acid molecules may encode just the polypeptide sequence that comprises the VL domain of the antibody or fragment thereof. One of the nucleic acid molecules may encode just the polypeptide sequence that comprises the VH domain of the antibody or fragment thereof. However, the nucleic acid molecule may also encode both VH and VL domain containing polypeptide sequences capable of forming the antibody (such as full length/whole antibody or an antigen-binding fragment thereof) of the invention.

The nucleic acid molecule(s) that encode the antibody of the invention, such as according to the first aspect of the invention, may be, or may be part of, a vector (such as a plasmid vector, cosmid vector or viral vector, or an artificial chromosome) that may comprise other functional regions (elements) such as one or more promoters, one or more origins or replication, one or more selectable marker(s), and one or more other elements typically found in expression vectors. The cloning and expression of nucleic acids that encode proteins, including antibodies, is well established and well within the skill of the person in the art.

According to a third aspect of the invention there is provided a vector comprising the nucleic acid of the second aspect of the invention. In particular embodiments, the vector is a plasmid vector, cosmid vector, viral vector, or an artificial chromosome.

The nucleic acids of the invention, including vector nucleic acids that comprise nucleotide sequences that encode the polypeptides capable of forming an antibody of the invention, may be in purified/isolated form.

Isolated/purified nucleic acids that encode an antibody of the invention will be free or substantially free of material with which they are naturally associated, such as other proteins or nucleic acids with which they are found in their natural environment, or the environment in which they are prepared (e.g. cell culture) when such preparation is by recombinant DNA technology practised in vitro or in vivo.

In particular embodiments, the nucleic acids of the invention are greater than 80%, such as greater than 90%, greater than 95%, greater than 97% and greater than 99% pure.

Thus, according to another variation of the third aspect of the invention there is provided a vector comprising a nucleic acid or nucleotide sequence that encodes a heavy chain variable polypeptide or a light chain variable polypeptide of the invention. In a particular embodiment, the vector comprises nucleic acid that encodes both the heavy and light chain variable regions. In particular embodiments, the said polypeptides may also comprise other domains such as constant domains, hinge regions, and an Fc region, such as one comprising one or more Fc receptor binding sites.

The nucleic acid and/or vector of the invention may be introduced into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid-based system. The plasmid system may be maintained episomally or may incorporated into the host cell or into an artificial chromosome. Incorporation may be either by random or targeted integration of one or more copies at single or multiple loci. For bacterial cells, suitable techniques may include calcium chloride transformation, electroporation and transfection using bacteriophage.

In one embodiment, the nucleic acid of the invention is integrated into the genome (e.g. chromosome) of the host cell. Integration may be promoted by inclusion of sequences that promote recombination with the genome, in accordance with standard techniques.

### Host Cells

A further aspect of the present invention provides a host cell containing nucleic acid as disclosed herein. Such a host cell may be in vitro and may be in culture.

The host cell can be from any species, such as a bacterium or yeast but suitably the host cell is a mammalian cell such as a human cell or rodent cell, for example a HEK293T cell or CHO-K1 cell.

Thus, according to a fourth aspect of the invention there is provided a host cell comprising the nucleic acid sequence according to the second aspect of the invention or the vector according to third aspect of the invention.

The host cell can be treated so as to cause or allow expression of the protein of the invention from the nucleic acid, e.g. by culturing host cells under conditions for expression of the encoding nucleic acid. The purification of the expressed product may be achieved by methods known to one of skill in the art.

Thus, the nucleic acids of the invention, including vector nucleic acids that comprise nucleotide sequences that encode the polypeptides capable of forming the antibodies of the invention, may be present in an isolated host cell. The host cell is typically part of a clonal population of host cells. As used herein, reference to a host cell also encompasses a clonal population of said cell. A clonal population is one that has been grown from a single parent host cell. The host cell can be from any suitable organism. Suitable host cells include bacterial, fungal or mammalian cells.

The host cell may serve to assist in amplifying the vector nucleic acid (such as with a plasmid) or it may serve as the biological factory to express the polypeptide(s) of the invention that form the BTLA antibody of the invention. A suitable host for amplifying the vector nucleic acid could be a bacterial or fungal cell, such as an Escherichia coli cell or Saccharomyces cerevisiae cell. A suitable host for expressing the proteins of the invention (i.e. the polypeptides making up the human BTLA-binding antibody of the invention would be a mammalian cell such as a HEK293T or CHO-K1 cell. In a particular embodiment, the host cell is a mammalian cell, such as a HEK293T or CHO-K1 cell.

A variety of host-expression vector systems may be utilized to express a BTLA-binding molecule as described herein (see e.g. U.S. Pat. No. 5,807,715). For example, mammalian cells such as Chinese hamster ovary cells (CHO), in conjunction with a vector such as the major intermediate early gene promoter element from human cytomegalovirus is an effective expression system for CEA proteins (Foecking et al., Gene, 45:101 (1986); and Cockett et al., Bio/Technology, 8:2 (1990)). Different host cells have characteristic and specific mechanisms for the post-translational processing and modification of proteins and gene products. Appropriate cell lines or host systems can be chosen to ensure the correct modification and processing of the protein of the disclosure. To this end, eukaryotic host cells which possess the cellular machinery for proper processing of the primary transcript, glycosylation, and phosphorylation of the gene product may be used. Such mammalian host cells include but are not limited to CHO, HEK, VERY, BHK, Hela, COS, MDCK, 293, 3T3, W138, BT483, Hs578T, HTB2, BT2O and T47D, NS0, CRL7O3O and HsS78Bst cells.

### Antibody Production

According to a fifth aspect of the invention there is provided a method of producing an antibody according to the first aspect of the invention, comprising the step of culturing the host cell of the fourth aspect of the invention under conditions for production of said antibody, and optionally isolating and/or purifying said antibody.

According to a variation of the fifth aspect of the invention there is provided a method of producing an antibody that binds to human BTLA, comprising the step of culturing the host cell that comprises nucleic acid encoding the polypeptide(s) that form the antibody that binds to human BTLA under conditions for production of said antibody, optionally further comprising isolating/purifying said antibody.

By isolated/purified we mean that the antibody of the invention, or polypeptides that make up these molecules, will be free or substantially free of material with which they are naturally associated, such as other proteins or nucleic acids with which they are found in their natural environment, or the environment in which they are prepared (e.g. cell culture) when such preparation is by recombinant DNA technology practised in vitro or in vivo.

According to a variation of the fifth of the invention there is provided a method for preparing an antibody that specifically binds human BTLA, the method comprising the steps of:

a) providing a host cell comprising one or more nucleic acid molecules encoding one or more polypeptides that comprise the amino acid sequence of a heavy chain variable domain and/or a light chain variable domain which when expressed are capable of combining to create a human BTLA-binding molecule;
b) culturing the host cell expressing the encoded amino acid sequence(s); and c) isolating the antibody molecule.

The one or more nucleic acid molecules are those describe above that encode for one or more polypeptides capable of forming an antibody of the invention that specifically binds human BTLA.

In a particular embodiment, the antibody comprises: i) a heavy chain variable region comprising three CDRs: CDRH1, CDRH2 and CDRH3, wherein CDRH1 has an amino acid sequence as set forth in SEQ ID NO: 1, CDRH2 has an amino acid sequence as set forth in SEQ ID NO: 17, and CDRH3 has an amino acid sequence as set forth in SEQ ID NO: 3; and

ii) a light chain variable region comprising three CDRs: CDRL1, CDRL2 and CDRL3, wherein CDRL1 has an amino acid sequence as set forth in SEQ ID NO: 4, CDRL2 has an amino acid sequence as set forth in SEQ ID NO: 12, and CDRL3 has an amino acid sequence as set forth in SEQ ID NO: 6.

In a particular embodiment, the antibody comprises: i) a heavy chain variable region comprising three CDRs: CDRH1, CDRH2 and CDRH3, wherein CDRH1 has an amino acid sequence as set forth in SEQ ID NO: 20, CDRH2 has an amino acid sequence as set forth in SEQ ID NO: 21, and CDRH3 has an amino acid sequence as set forth in SEQ ID NO: 22; and

ii) a light chain variable region comprising three CDRs: CDRL1, CDRL2 and CDRL3, wherein CDRL1 has an amino acid sequence as set forth in SEQ ID NO: 23, CDRL2 has an amino acid sequence as set forth in SEQ ID NO: 24, and CDRL3 has an amino acid sequence as set forth in SEQ ID NO: 25.

In a particular embodiment, the antibody comprises: i) a heavy chain variable region comprising three CDRs: CDRH1, CDRH2 and CDRH3, wherein CDRH1 has an amino acid sequence as set forth in SEQ ID NO: 30, CDRH2 has an amino acid sequence as set forth in SEQ ID NO: 31, and CDRH3 has an amino acid sequence as set forth in SEQ ID NO: 32; and

ii) a light chain variable region comprising three CDRs: CDRL1, CDRL2 and CDRL3, wherein CDRL1 has an amino acid sequence as set forth in SEQ ID NO: 33, CDRL2 has an amino acid sequence as set forth in SEQ ID NO: 34, and CDRL3 has an amino acid sequence as set forth in SEQ ID NO: 35.

In a particular embodiment, the antibody comprises:

i) a heavy chain variable region comprising an amino acid sequence disclosed in SEQ ID NO: 18, or a sequence with at least 90% sequence identity thereto; and
ii) a light chain variable region comprising an amino acid sequence disclosed in SEQ ID NO:14, or a sequence with at least 90% sequence identity thereto.

In a particular embodiment, the antibody comprises:

i) a heavy chain variable region comprising an amino acid sequence disclosed in SEQ ID NO: 26, or a sequence with at least 90% sequence identity thereto; and
ii) a light chain variable region comprising an amino acid sequence disclosed in SEQ ID NO:27, or a sequence with at least 90% sequence identity thereto.

In a particular embodiment, the antibody comprises:

i) a heavy chain variable region comprising an amino acid sequence disclosed in SEQ ID NO: 36, or a sequence with at least 90% sequence identity thereto; and
ii) a light chain variable region comprising an amino acid sequence disclosed in SEQ ID NO:43, or a sequence with at least 90% sequence identity thereto.

Conditions for the production of the antibody of the invention and purification of said molecules are well-known in the art. One way of attending to this is to prepare a clonal population of cells capable of expressing the antibody or fragment thereof of the invention and culturing these in a suitable growth medium for a period of time and at a temperature conducive to allow for expansion/growth of the cell population and expression of the protein(s) of interest. If the protein(s) of interest (e.g. antibody of invention) is expressed within the host cells then the cells may be lysed (e.g. using a mild detergent or sonication) to release the contents of the cell (and thus the protein of interest) into the surrounding medium (which could be the culture medium or another medium that the cells have been reconstituted in) and this medium is then subjected to purification processes. If the protein(s) of interest (e.g. antibody of invention) is secreted into the growth medium, then the medium is subjected to purification processes. Antibody purification typically involves isolation of antibody from, for example the medium or from the culture supernatant of a hybridoma cell line using well-established methods typically involving chromatography (e.g., using affinity chromatography, anionic and/or cationic exchange chromatography, size-exclusion chromatography or other separation techniques) to separate the protein of interest from unwanted host-derived proteins and other cellular contaminants (e.g. nucleic acids, carbohydrates etc.). The purified proteins may also be subjected to a virus inactivation step. Finally, the purified protein of interest may, for example, be lyophilised or formulated ready for storage, shipment and subsequent use. Preferably the protein of interest (e.g. whole antibody or antigen-binding fragment thereof of the invention) will be substantially free from contaminating proteins which were originally present in the culture medium following expression or cell-lysis.

In certain embodiments, the antibody of the invention will be at least 90%, at least 95%, at least 96%, at least 97%, at least 98% or at least 99% pure.

The proteins of the invention (e.g. whole antibody or antigen-binding fragment thereof of the invention) can be formulated into a suitable composition.

### Compositions

While the BTLA-binding molecule (antibody of the invention) may be administered alone, in certain embodiments administration is of a pharmaceutical composition wherein the BTLA-binding molecule is formulated with at least one pharmaceutically-acceptable excipient. The excipient may be a suitable pharmaceutical carrier solute. Such carriers are well known in the art and include phosphate buffered saline solutions, water, liposomes, various types of wetting agents, sterile solutions, etc. Compositions comprising such carriers can be formulated by well-known conventional methods. These pharmaceutical compositions can be administered to the subject at a suitable dose. The dosage regimen will be determined by the attending physician and clinical factors.

According to a sixth aspect of the invention there is provided a pharmaceutical composition comprising a pharmaceutically acceptable excipient and a therapeutically effective amount of the antibody of the first aspect of the invention, or that produced by the fifth aspect of the invention. In a particular embodiment, the composition comprises phosphate buffered saline.

A “pharmaceutical composition” refers to a preparation which is in such form as to permit the biological activity of the active ingredient to be effective, and which contains no additional components which are unacceptably toxic to a subject to which the formulation would be administered. The pharmaceutical composition will include one or more pharmaceutically acceptable excipients. The term excipient in this context refers to any additive, such as fillers, solubilisers, carriers, vehicles, additives and the like.

The pharmaceutical compositions can comprise one or more pharmaceutically acceptable excipients, including, e.g., water, ion exchangers, proteins, buffer substances, and salts. Preservatives and other additives can also be present. The excipient can be a solvent or dispersion medium. Suitable formulations for use in therapeutic methods disclosed herein are described in Remington's Pharmaceutical Sciences 16th edition, Osol, A. Ed. (1980).

“Pharmaceutically acceptable” excipients are those which can reasonably be administered to a subject mammal to provide an effective dose of the active ingredient employed. Pharmaceutical compositions of the invention are prepared for storage by mixing the composition with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable excipients are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized HER2 antibody formulations are described in WO 97/04801.

The pharmaceutical compositions to be used for in vivo administration must be sterile. This can be readily accomplished by filtration through sterile filtration membranes.

The route of administration of the BTLA binding moiety molecule, e.g., an antibody, or antigen-binding fragment thereof can be, for example, oral, parenteral, by inhalation or topical. The term parenteral as used herein includes, e.g., intravenous, intraarterial, intraperitoneal, intramuscular, subcutaneous, rectal, or vaginal administration.

Pharmaceutical compositions for parenteral administration include sterile aqueous or non-aqueous solutions, and suspensions. Examples of non-aqueous solvents are propylene glycol, polyethylene glycol, and injectable organic esters such as ethyl oleate. Aqueous carriers include water, aqueous solutions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the composition might comprise proteinaceous carriers, like, e.g., serum albumin or immunoglobulin, in certain embodiments of human origin. For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. As noted above, these are all referred to herein as excipients.

Compositions for injection can be administered with medical devices known in the art. For example, with a hypodermic needle. Needleless injection devices, such as those disclosed in U.S. Pat. Nos. 6,620,135 and 5,312,335 could also be utilised.

Pharmaceutical compositions for oral administration may be in tablet, capsule, powder, liquid or semi-solid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included as required.

An antibody of the present invention may be formulated in liquid, semi-solid or solid forms depending on the physicochemical properties of the molecule and the route of delivery. Formulations may include excipients, or combinations of excipients, for example: sugars, amino acids and surfactants. Liquid formulations may include a wide range of antibody concentrations and pH. Solid formulations may be produced by lyophilisation, spray drying, or drying by supercritical fluid technology, for example.

The pharmaceutical composition can be administered as a single dose, multiple doses or over an established period of time in an infusion. Dosage regimens also can be adjusted to provide the optimum desired response (e.g., a therapeutic or prophylactic response). In particular, parenteral formulations can be a single bolus dose, an infusion or a loading bolus dose followed with one or more maintenance doses. These compositions can be administered at specific fixed or variable intervals, e.g., once a day, or on an “as needed” basis.

### Dosages

The amount of the BTLA-binding molecule, or the pharmaceutical formulation containing such molecule, which will be therapeutically effective can be determined by standard clinical techniques, such as through dose ranging clinical trials. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula:

Dose (ml.)=[patient weight (kg)×dose level (mg/kg)/drug concentration (mg/mL)]

Therapeutically effective doses of the pharmaceutical compositions for the treatment of BTLA-related diseases or disorders, as discussed herein, will vary depending upon many different factors, including means of administration, target site, physiological state of the patient, weight or patient, sex of patient, age of patient, whether the patient is human or an animal, other medications administered, and whether treatment is prophylactic or therapeutic. The therapeutically effective dose is likely to have been determined from clinical trials and is something that the attending physician can determine using treatment guidelines. Usually, the patient is a human, but non-human mammals can also be treated. Treatment dosages can be titrated using routine methods known to those of skill in the art to optimize safety and efficacy.

In various embodiments, the BTLA-binding molecule is administered at a concentration of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg.

A pharmaceutical composition of the invention may be administered alone or in combination with other treatments, either simultaneously or sequentially dependent upon the condition to be treated. Such combination would likely be with other immunosuppressives such as one selected from: corticosteroids, cyclosporine, azathioprine, sulfasalazine, methotrexate, mycophenolate, tacrolimus and fingolimod, or other biologics such as infliximab, adalimumab, ustekinumab, tocilizumab and rituximab.

According to a seventh aspect of the invention there is provided a method of preparing a pharmaceutical composition, the method comprising formulating an antibody in accordance with the first aspect of the invention, or one produced in accordance with the fifth aspect of the invention into a composition including at least one additional component. In a particular embodiment, the at least one additional component is a pharmaceutically acceptable excipient.

### Kits

Further, the product (e.g. BTLA binding molecule or a pharmaceutical composition thereof) can be packaged and sold in the form of a kit. Such articles of manufacture can have labels or package inserts indicating instructions about the product and the appropriate use of the product for the treatment of a subject suffering from or predisposed to a disease or disorder.

Thus, according to an eighth aspect of the invention there is provided a kit comprising an antibody in accordance with the first aspect of the invention or the pharmaceutical composition in accordance with the sixth aspect of the invention. Suitably, such a kit includes a package insert comprising instructions for use.

### Therapy/Medical Uses

An antibody of the invention or a pharmaceutical composition comprising said antibody or antigen-binding fragment thereof may be used in therapy, typically as a medicament.

In certain embodiments, an antibody of the invention or a pharmaceutical composition comprising said antibody may be used for treating or preventing any disease or condition in a subject in need thereof.

BTLA is involved in down-regulating immune responses and there are many diseases or conditions that could be treated by suppressing host T-cells and/or B-cells (e.g. see Crawford & Wherry. Editorial: Therapeutic potential of targeting BTLA. J Leukocyte Biol. 86:5-8, 2009). Diseases or conditions that could benefit from treatment with an anti-BTLA agonist are referred to herein as “BTLA-related diseases”. BTLA-related diseases include inflammatory or autoimmune diseases, and disorders of excessive immune cell proliferation.

Specific BTLA-related diseases that can be treated with the BTLA-binding molecules of the invention include: Addison's disease, allergy, alopecia areata, amyotrophic lateral sclerosis, ankylosing spondylitis, anti-phospholipid syndrome, asthma (including allergic asthma), autoimmune haemolytic anaemia, autoimmune hepatitis, autoimmune pancreatitis, autoimmune polyendocrine syndrome, Behcet's disease, bullous pemphigoid, cerebral malaria, chronic inflammatory demyelinating polyneuropathy, coeliac disease, Crohn's disease, Cushing's Syndrome, dermatomyositis, diabetes mellitus type 1, eosinophilic granulomatosis with polyangiitis, graft versus host disease, Graves' disease, Guillain-Barre syndrome, Hashimoto's thyroiditis, Hidradenitis Suppurativa, inflammatory fibrosis (e.g., scleroderma, lung fibrosis, and cirrhosis), juvenile arthritis, Kawasaki disease, leukemia, lymphoma, lymphoproliferative disorders, multiple sclerosis, myasthenia gravis, myeloma, neuromyelitis optica, pemphigus, polymyositis, primary biliary cholangitis, primary sclerosing cholangitis, psoriasis, psoriatic arthritis, rheumatoid arthritis, sarcoidosis, Sjögren's syndrome, systemic lupus erythematosus, Takayasu's arteritis, temporal arteritis, transplant rejection, transverse myelitis, ulcerative colitis, uveitis, vasculitis, vitiligo and Vogt-Koyanagi-Harada Disease.

In particular embodiments, the disease to be treated is selected from the group consisting of: Crohn's disease, ulcerative colitis, rheumatoid arthritis, systemic lupus erythematosus, psoriasis, graft versus host disease, transplant rejection, multiple sclerosis, vasculitis, Sjögren's syndrome, Behcet's disease, uveitis, diabetes mellitus type 1, Hashimoto's thyroiditis, primary sclerosing cholangitis, myasthenia gravis.

In particular embodiments, the disorder of excessive immune cell proliferation is selected from lymphoma, leukemia, systemic mastocytosis, myeloma, or a lymphoproliferative disorder.

According to a ninth aspect of the invention there is provided an antibody in accordance with the first aspect of the invention or the pharmaceutical composition in accordance with the sixth aspect of the invention for use in therapy.

In a particular embodiment, the therapy is treatment or prevention of a BTLA-related disease.

In a particular embodiment, the BTLA-related disease is one caused by decreased expression and/or activity of BTLA in a subject. In particular, any disease or disorder characterised by the presence or activity of T or B cells can be treated with a BTLA agonist antibody of the invention.

In one embodiment, the BTLA-related disease is an inflammatory disease (such as rheumatoid arthritis), an autoimmune disease or disorder (such as graft versus host) or a proliferative disease or disorder (such as cancer).

In a particular embodiment, the therapy is treatment or prevention of inflammatory or autoimmune diseases, and disorders of excessive immune cell proliferation.

According to a variation of the ninth aspect of the invention there is provided a method of treating a patient in need thereof, comprising administering to the patient an antibody (or BTLA binding molecule) in accordance with the first aspect of the invention or the pharmaceutical composition in accordance with the sixth aspect of the invention. In a particular embodiment the patient in need of treatment, or to be treated, has (or is suffering from) a BTLA-related disease. In a particular embodiment, the patient in need of treatment, or to be treated, has (or is suffering from) an inflammatory disease, an autoimmune disease, or a disorder of excessive immune cell proliferation.

In a particular embodiment, the antibody in accordance with the first aspect of the invention or the pharmaceutical composition in accordance with the sixth aspect of the invention is administered to a patient in need thereof in a pharmaceutically acceptable amount.

In a variation of this ninth aspect of the invention there is provided an antibody (or BTLA binding molecule) in accordance with the first aspect of the invention or the pharmaceutical composition in accordance with the sixth aspect of the invention for use in a method of treating a patient in need thereof. In a particular embodiment, the method is for treating or preventing a BTLA-related disease. In particular embodiments, the method is for treating or preventing inflammatory or autoimmune diseases, and disorders of excessive immune cell proliferation.

In a further variation of this aspect there is provided use of an antibody in accordance with the first aspect of the invention or the pharmaceutical composition in accordance with the sixth aspect of the invention in the manufacture of a medicament for the treatment of a patient in need thereof.

In one embodiment, the therapy is for treating a BTLA-related disease. Suitably, the BTLA-related disease is an inflammatory disease (such as asthma), an autoimmune disease or disorder (such as rheumatoid arthritis) or an immunoproliferative disease or disorder (such as lymphoma).

In particular embodiments, the antibody of the invention or a pharmaceutical composition comprising said antibody is used to suppress T-cells and/or B-cells.

In particular embodiments, the antibody of the invention or a pharmaceutical composition comprising said antibody is used for treating or preventing a disease or condition in a subject in need thereof selected from the group consisting of: Addison's disease, allergy, alopecia areata, amyotrophic lateral sclerosis, ankylosing spondylitis, anti-phospholipid syndrome, asthma (including allergic asthma), autoimmune haemolytic anaemia, autoimmune hepatitis, autoimmune pancreatitis, autoimmune polyendocrine syndrome, Behcet's disease, bullous pemphigoid, cerebral malaria, chronic inflammatory demyelinating polyneuropathy, coeliac disease, Crohn's disease, Cushing's Syndrome, dermatomyositis, diabetes mellitus type 1, eosinophilic granulomatosis with polyangiitis, graft versus host disease (GVHD), Graves' disease, Guillain-Barre syndrome, Hashimoto's thyroiditis, Hidradenitis Suppurativa, inflammatory fibrosis (e.g., scleroderma, lung fibrosis, and cirrhosis), juvenile arthritis, Kawasaki disease, leukemia, lymphoma, lymphoproliferative disorders, multiple sclerosis (MS), myasthenia gravis, myeloma, neuromyelitis optica, pemphigus, polymyositis, primary biliary cholangitis, primary sclerosing cholangitis, psoriasis, psoriatic arthritis, rheumatoid arthritis, sarcoidosis, Sjögren's syndrome, systemic lupus erythematosus, Takayasu's arteritis, temporal arteritis, transplant rejection, transverse myelitis, ulcerative colitis, uveitis, vasculitis, vitiligo and Vogt-Koyanagi-Harada Disease.

In particular embodiments, the antibody of the invention or a pharmaceutical composition comprising said antibody is used for treating or preventing a disease or condition in a subject in need thereof selected from the group consisting of: Crohn's disease, ulcerative colitis, rheumatoid arthritis, systemic lupus erythematosus, psoriasis, graft versus host disease, transplant rejection, multiple sclerosis, vasculitis, Sjögren's syndrome, Behcet's disease, uveitis, diabetes mellitus type 1, Hashimoto's thyroiditis, primary sclerosing cholangitis, myasthenia gravis. In one embodiment, the immunoproliferative disease is cancer. Suitably the cancer is a leukemia or a lymphoma.

In another embodiment, the antibody of the invention or a pharmaceutical composition comprising said antibody is for use in the prevention or treatment of transplant rejection.

In another embodiment, the invention relates to the prevention or treatment of graft versus host disease.

In another embodiment, the antibody of the invention or a pharmaceutical composition comprising said antibody is for use in the treatment of rheumatoid arthritis.

In other embodiments, the antibody of the invention or a pharmaceutical composition comprising said antibody is for use in the treatment of diabetes, such as type 1 diabetes.

In another embodiment, the antibody of the invention or a pharmaceutical composition comprising said antibody is for use in the treatment of psoriasis.

In another embodiment, the antibody of the invention or a pharmaceutical composition comprising said antibody is for use in the treatment of multiple sclerosis.

In another embodiment, the antibody of the invention or a pharmaceutical composition comprising said antibody is for use in the treatment of colitis.

The term “effective amount” or “therapeutically effective amount” refers to a dosage or an amount of a drug that is sufficient to ameliorate the symptoms in a patient or to achieve a desired biological outcome, e.g., with cancer, an increased death of tumour cells, reduced tumour size, increased progression free survival or overall survival etc. As disclosed elsewhere herein, the effective amount will typically be assessed through extensive human clinical studies.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The invention will now be further described with reference to the following non-limiting Examples and accompanying Figures.

### BRIEF DESCRIPTION OF THE FIGURES

FIG. 1

Binding of antibodies to human and cynomolgus BTLA in soluble and cell expressed forms. (a) Surface plasmon resonance (SPR) binding curves for soluble monomeric human BTLA extracellular domain injected at increasing concentrations over immobilized anti-BTLA antibody; graphs show SPR signal after reference and blank subtraction. (b) Association and dissociation rates for binding to human or cynomolgus BTLA as calculated by curve fitting using BiaEvaluation software. (c) Binding of antibody 2.8.6, compared to isotype control antibody, to a human BTLA or cynomolgus BTLA expressing Jurkat cell line (data points represent mean+/−SD of triplicate wells at each antibody concentration). (d) EC50s for antibody binding to transfected cell lines, as calculated by non-linear curve fitting using GraphPad Prism software

FIG. 2

(a) Blockade of ligand binding by anti-BTLA antibodies was assessed by SPR. Human BTLA extracellular domain was immobilized on the sensor chip. Human HVEM was injected to confirm binding, then allowed to fully dissociate. A saturating concentration of anti-BTLA antibody was then injected, followed immediately by a second injection of HVEM. (b) Equilibrium binding of HVEM after injection of antibody was expressed as a percent of HVEM binding prior to antibody injection. Saturation of BTLA with clone 11.5.1, but not with clone 2.8.6, blocked subsequent binding of ligand.

FIG. 3

Epitope mapping of anti-BTLA antibodies. (a) HEK293T cells transfected with BTLA constructs in a bicistronic vector also expressing GFP were stained with Pacific Blue conjugated anti-BTLA antibody. Clone 11.5.1 binds to cells transfected with wild-type receptor (left) but not to cells transfected with BTLA having a Y39R mutation (right). (b) Binding to each BTLA mutant construct was expressed as a percentage of binding to wild-type BTLA for clones 2.8.6 and 11.5.1. (c) Mutations Y39R and K41E which selectively eliminate binding of clone 11.5.1 were mapped onto the crystal structure of human BTLA (black residues). Residues critical for binding of the ligand HVEM are highlighted in grey.

FIG. 4

(a) The crystal structure of human BTLA extracellular domain in complex with the Fab′ fragment of clone 2.8.6. Residues on BTLA which are buried at the interface are highlighted in black. (b) The epitope of antibody 2.8.6 is shown (black residues) in relation to the HVEM binding site (grey residues).

FIG. 5

(a) Strategy for creation of a chimeric BTLA gene in humanised-BTLA mice. A section of human genomic DNA from the beginning of exon 2 to the end of exon 3 was inserted into the mouse locus replacing the mouse sequence from the beginning of exon 2 to the end of exon 4. The sequences at the exon-intron junction at the beginning of mouse exon 2 and end of mouse exon 4 were left intact to ensure proper splicing.

FIG. 6

(a) Protocol for T cell transfer assay to assess anti-BTLA antibodies in vivo. A mixture of humanised and wild-type OVA specific CD4 T cells was injected into recipient mice. The next day mice were immunised with ovalbumin in Alum to activate the transferred cells and 24 hours later were dosed with anti-human-BTLA antibody or isotype control. Eight days after initial cell transfer the ratio of humanised to wild-type cells in the transferred population in the spleen was assessed by flow cytometry. (b) Clone 11.5.1 and to a lesser extent 2.8.6 both reduced expansion of the humanised cells relative to the wild-type. Graph shows pooled data from two (for 11.5.1) or three (for 2.8.6) repeat experiments. Each data point represents an individual recipient mouse.

FIG. 7

Effect of anti-BTLA clone 2.8.6 on CD4 T-cell proliferation in a mixed lymphocyte reaction in vitro. T cells from humanised C57BL/6 mice were stained with CellTraceViolet and added to Mitomycin C treated Balb/c stimulator cells in the presence of anti-BTLA antibody or isotype control. After 96 hours, proliferation of humanised CD4 cells was assessed and normalised to proliferation in the absence of antibody. Clone 2.8.6 inhibited proliferation of humanised cells with an IC50 of 0.029 nM and had a maximal effect of 42% inhibition of proliferation. Data points represent mean+/−SD of triplicate wells at each antibody concentration and are representative of 5 independent experiments.

FIG. 8

(a) Effect of clone 2.8.6 in a T cell colitis model. RAG knockout recipient mice were injected with CD45RBhiCD25-CD4+ T cells from humanised BTLA mice and treated with 200 μg 2.8.6 or isotype control antibody on days 7, 21 and 35. Isotype control treated mice progressively lost weight from 3 weeks onwards, whilst 2.8.6 treated mice were spared. (b) 8 weeks after cell transfer colons were processed to extract lamina propria lymphocytes and the total number of inflammatory cells extracted per colon was calculated. Isotype control treated mice had significantly more infiltrating immune cells than 2.8.6 treated mice. (c) Colon weight to length ratios were calculated as a marker of inflammation and thickening. 2.8.6 treatment prevented the increase in weight to length ratio seen in isotype control treated mice.

FIG. 9

(a) Effect of BTLA antibodies in a parent-to-F1 model of GVHD. C57BL/6 splenocytes and bone marrow cells from humanised-BTLA mice were injected into CB6F1 recipient mice, which were then treated with anti-BTLA antibody or isotype control. Untreated mice developed clinical GVHD with progressive weight loss, dermatitis and diarrhea and were culled when they reached pre-specified humane endpoints. 2.8.6 and 11.5.1 antibody treated mice were relatively spared, with survival comparable to control mice reconstituted with syngeneic cells. (b) 5 weeks after cell transfer mice were culled and colon weight to length ratio was calculated as a marker of gut inflammation. 2.8.6 and 11.5.1 treatment prevented the colon thickening seen in untreated mice.

FIG. 10

(a) Effect of D265A mutated clone 11.5.1 in a T cell transfer assay in vivo. This mutated antibody, which does not bind Fc receptors, no longer inhibited proliferation of humanised BTLA cells, instead lead to enhanced proliferation due to receptor blockade. (b) The D265A mutated 11.5.1 antibody no longer inhibited T cell proliferation in a mixed lymphocyte reaction.

FIG. 11

Anti-BTLA antibodies do not fix complement. Splenocytes from humanised BTLA mice were incubated with 10% rabbit complement for 1 hour at 37° C. in the presence of 20 μg/ml BTLA antibody, isotype control or positive control (a depleting CD20 antibody). Anti-CD20 antibody depleted the majority of B cells confirming the activity of the rabbit complement, but BTLA antibodies did not deplete either B or T cells, even though both these populations stain positive for BTLA.

FIG. 12

Anti-BTLA antibodies do not cause antibody-dependent-cell-mediated cytotoxicity. Splenocytes from humanised BTLA mice were incubated for 24 hours at 37° C. in the presence of 20 μg/ml BTLA antibody, isotype control or positive control (a depleting CD20 antibody). Anti-CD20 antibody depleted the majority of B cells by inducing ADCC by effector cells in the mixture, but BTLA antibodies did not deplete either B or T cells, even though both these populations stain positive for BTLA.

FIG. 13

Anti-BTLA antibodies do not deplete B or T cells in vivo. Humanised BTLA mice were injected with 200 μg of 2.8.6 antibody. At 24 hours spleens and bone marrow were collected and cell populations assessed by flow cytometry. 2.8.6 did not deplete B or T cells in the spleen or affect the frequency of different B cell precursor populations in the bone marrow (n=3 mice per group).

FIG. 14

BTLA expression levels on B cells or CD4⁺ T cells from humanised mice following 6 days of in vivo incubation with antibodies 2.8.6 or 11.5.1, compared to BTLA expression on cells from mice injected with isotype control antibody (n=5 mice per group).

FIG. 15

Agonist effect of BTLA antibodies in a reporter assay is dependent on Fc receptor binding and isotypes with greater FcγR2B binding are more effective agonists. A Jurkat T cell line expressing GFP under the control of NFkB-responsive transcriptional elements was transfected with human BTLA and stimulated by co-culture with a BW5147 cell line expressing an anti-CD3 ScFv construct on its surface. NFkB signaling was detected by measuring the GFP geomean by flow cytometry after 24 hours of culture. The inhibitory effect of adding BTLA agonist antibodies of different isotypes to the culture was assessed in the condition where the BW5147 cell line was also transfected to express hFcγR2B (a) or in the condition where no Fc receptors were present (b). Data points are the mean+/−SD of triplicate wells at each antibody concentration, and are representative of 3 independent experiments.

FIG. 16

Humanised anti-BTLA agonist antibodies 2.8.6, 6.2_varC and 3E8 expressed on a P238D isotype have greater efficacy and potency in a reporter assay, compared to an Fc fusion protein of BTLA's ligand HVEM or the prior art BTLA agonist 22B3. A Jurkat T cell line expressing GFP under the control of NFkB-responsive transcriptional elements was transfected with human BTLA and stimulated by co-culture with a BW5147 cell line expressing an anti-CD3 ScFv construct and hFcγR2B on its surface. NFkB signaling was detected by measuring the GFP geomean by flow cytometry after 24 hours of culture. The inhibitory effect of BTLA agonist antibodies added to the co-culture was assessed. Data points are the mean+/−SD of triplicate wells at each antibody concentration and are representative of 3 independent experiments.

FIG. 17

Humanised anti-BTLA 2.8.6 inhibits CD4 T cell proliferation in a mixed leukocyte reaction. Purified primary human T cells from a blood bank donor were stained with a cell proliferation tracking dye and co-cultured for 5 days with allogeneic monocyte derived dendritic cells from a different donor in a 4:1 ratio, in the presence of BTLA agonist antibodies or hIgG1 P238D isotype control. Cell populations were identified by flow cytometry and proliferation was assessed by dilution of the tracking dye. CD4 proliferation in the presence of BTLA antibody was normalized to the proliferation in the presence of the equivalent concentration of isotype control. Data was collated from 6 independent experiments with different donor pairs. 2.8.6 significantly inhibited CD4 T cell proliferation as a P238D isotype but not in other isotype formats. The prior art molecule 22B3 had no significant effect on CD4 proliferation.

FIG. 18

Humanised anti-BTLA agonist antibodies 2.8.6, 6.2 varC and 3E8 expressed on a P238D isotype inhibit primary B cell activation in response to the TLR9 agonist ODN2006. Primary human B cells were isolated from healthy donor PBMCs and stimulated with 0.01 μM ODN2006 in the presence or absence of different doses of P238D isotype control antibody or selected BTLA agonist antibodies. After 5 days, IL-10 concentration in the supernatant was assessed by ELISA. Bars represent the mean+/−SD of triplicate wells at each antibody concentration, and are representative of 3 independent experiments.

FIG. 19

Humanised anti-BTLA agonist antibodies 2.8.6, 6.2 varC and 3E8 expressed on a P238D isotype significantly reduce weight loss in a xenogeneic graft vs host disease model. Irradiated NSG mice were reconstituted IV with 10 million human PBMCs on day 0 and then treated IP on day 1 with 10 mg/kg BTLA antibody or P238D isotype control. Mice were weighed regularly and weight is plotted relative to starting weight (n=9 mice per group, data points represent mean+/−SD).

### EXAMPLES

In the examples that follow it is shown that antibodies such as 11.5.1 and 2.8.6 bind to human BTLA with high affinity. Using transgenic mice expressing the human receptor it is shown that, following binding to BTLA, these antibodies inhibit T cell responses in vitro and in vivo and are able to ameliorate disease in murine models of inflammatory bowel disease and graft-versus-host disease. Whilst these agonist effects are dependent on Fc-receptor binding, the antibodies do not cause depletion of BTLA expressing cells via cytotoxicity and do not induce receptor down-modulation. Introduction of the P238D modification in the heavy chain greatly enhances the agonist signaling of FcγR2B and increases the ratio of signaling of FcγR2B over FcγR2A. Such dual BTLA and FcγR2B agonist antibodies are expected to be of therapeutic utility, particularly in autoimmune and inflammatory disease settings.

### Example 1. Generation and Sequencing of Anti-BTLA Antibodies

Antibodies recognizing the human immune cell receptor BTLA were generated by BioGenes GmbH via immunizing mice with the extracellular region of human BTLA (BTLA^{K31-R151}). Splenocytes from immunized mice were fused with Sp2/0-Ag14 myeloma cells and resulting hybridomas selected for reactivity with human BTLA by ELISA of supernatants, in conjunction with dilution cloning. Antibodies were isotyped from hybridoma supernatant using a Rapid Mouse Isotyping Kit (RayBiotech). The antibodies produced by clones 2.8.6 and 11.5.1 were both found to be IgG1k.

To sequence the immunoglobulin variable domains, RNA was extracted from hybridomas using TRIzol Reagent (ThermoFisher) as per the manufacturer's instructions. RNA was reverse transcribed to produce cDNA using primers specific for the first constant domain of the heavy chain or for the constant domain of the light chain, and Super Script II Reverse Transcriptase (Invitrogen) as per manufacturer's instructions.

PCR was then performed using primers targeting conserved regions of the immunoglobulin locus as previously described (Tiller et al., J Immunol Methods. 350:183-193, 2009) and PCR products were sequenced. In some cases, identification of functional light chain was complicated by abundant non-functional kappa light chain cDNA from the fusion myeloma cell line, and to resolve this a previously described technique was employed, adding excess primer specific for the non-functional chain CDR3 to force truncation of the aberrant chain product (Yuan et al. J Immunol Methods. 294:39553-61, 2005).

Variable domain sequences were assessed using the NCBI IgBlast tool to determine the location of the CDRs.

### Example 2. Binding to Soluble Human and Cynomolgus BTLA

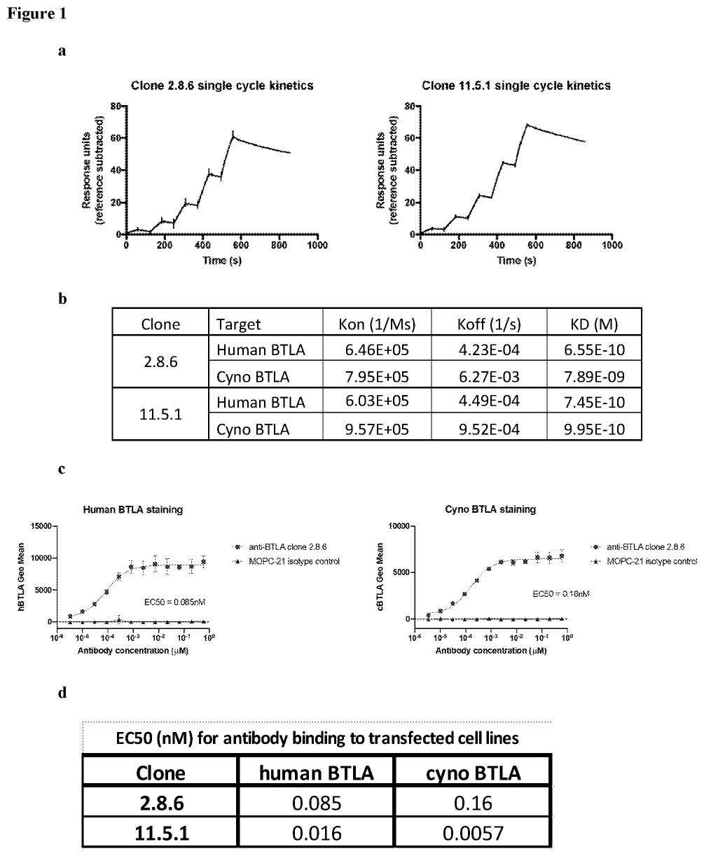

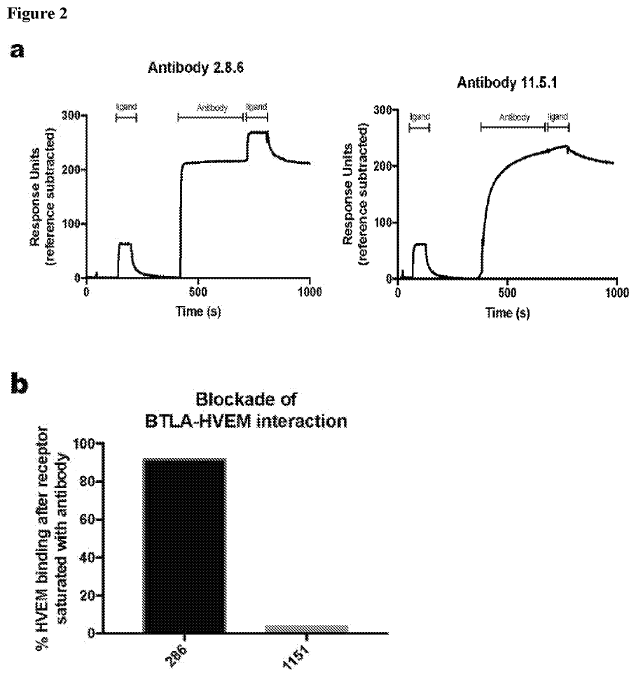

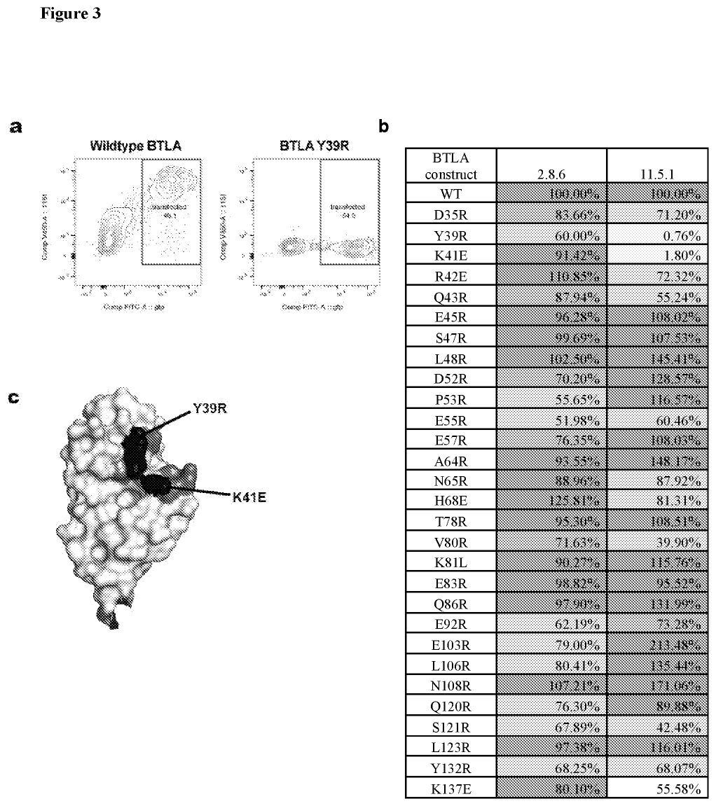

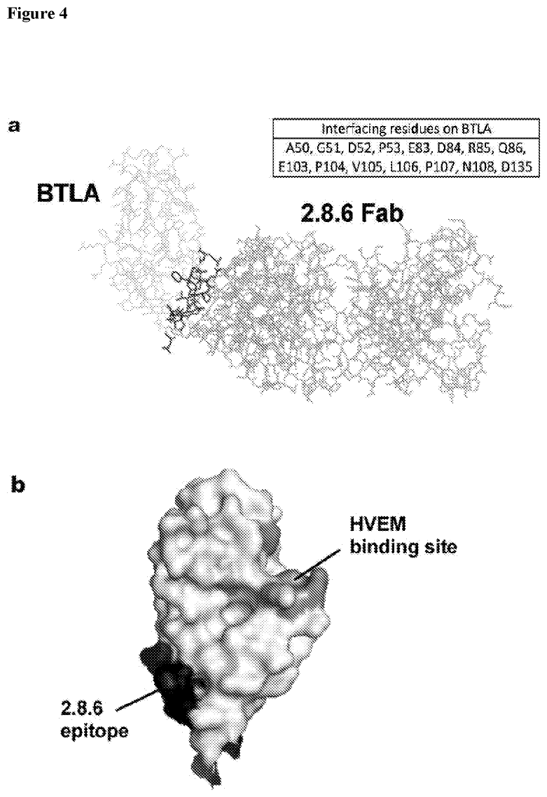

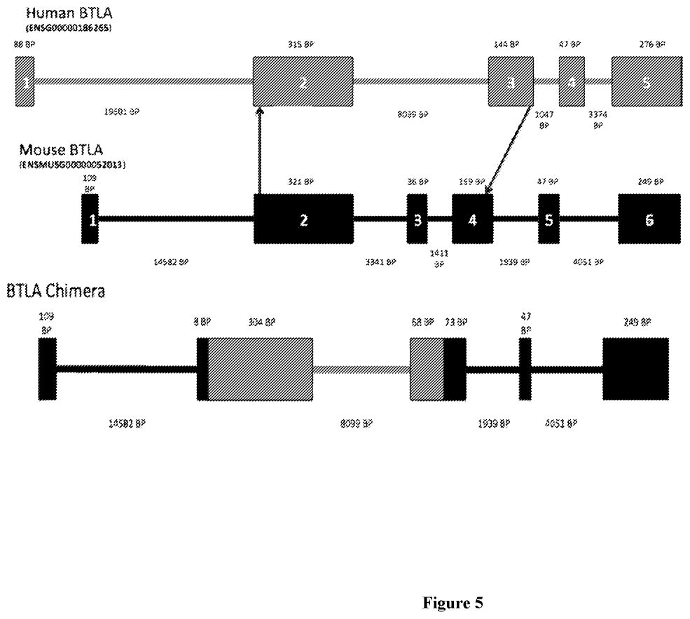

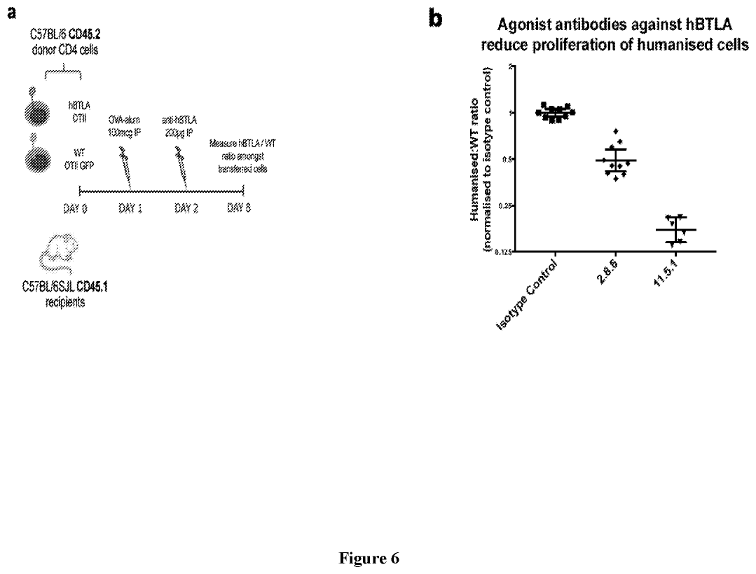

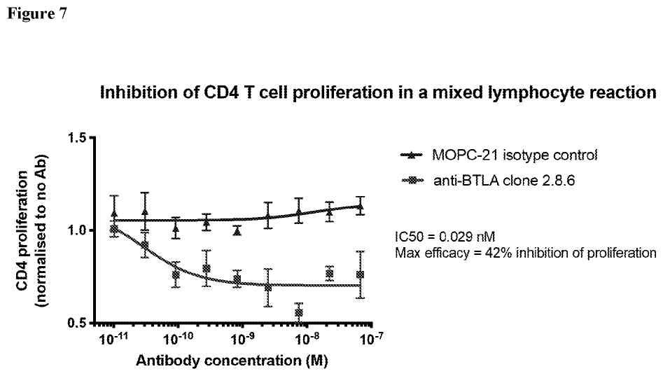

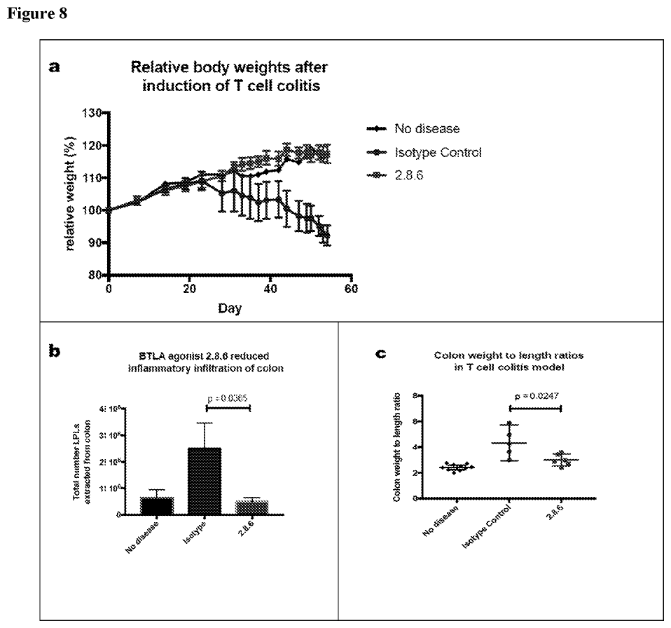

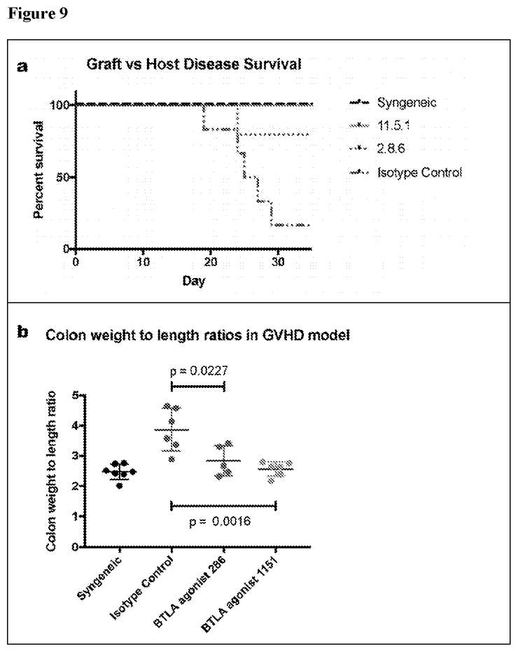

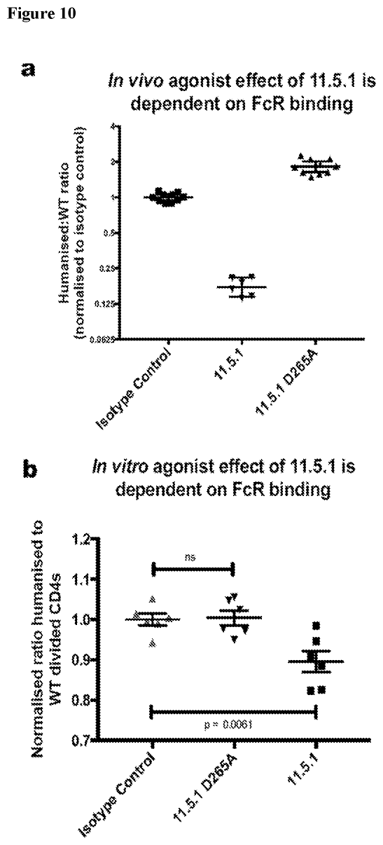

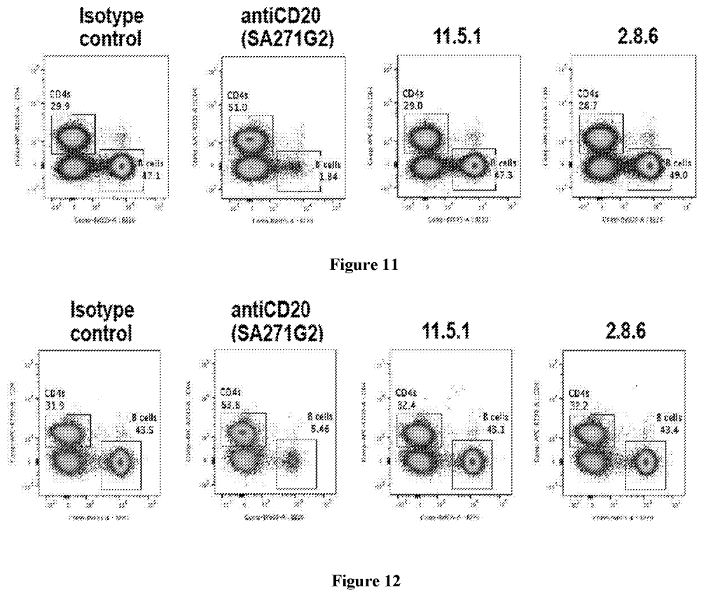

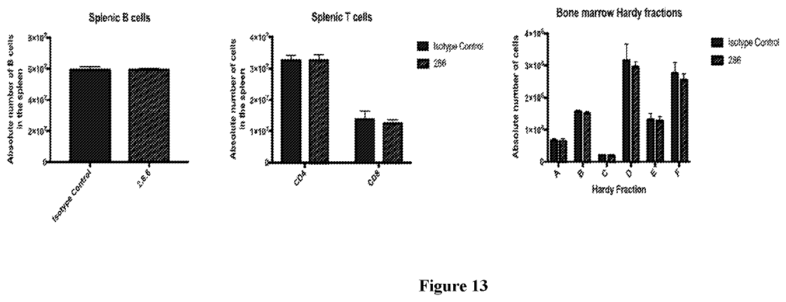

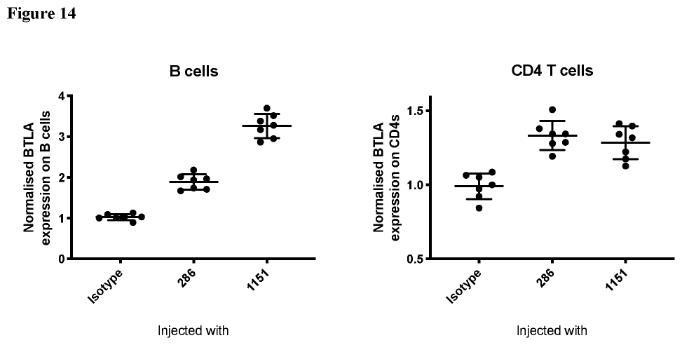

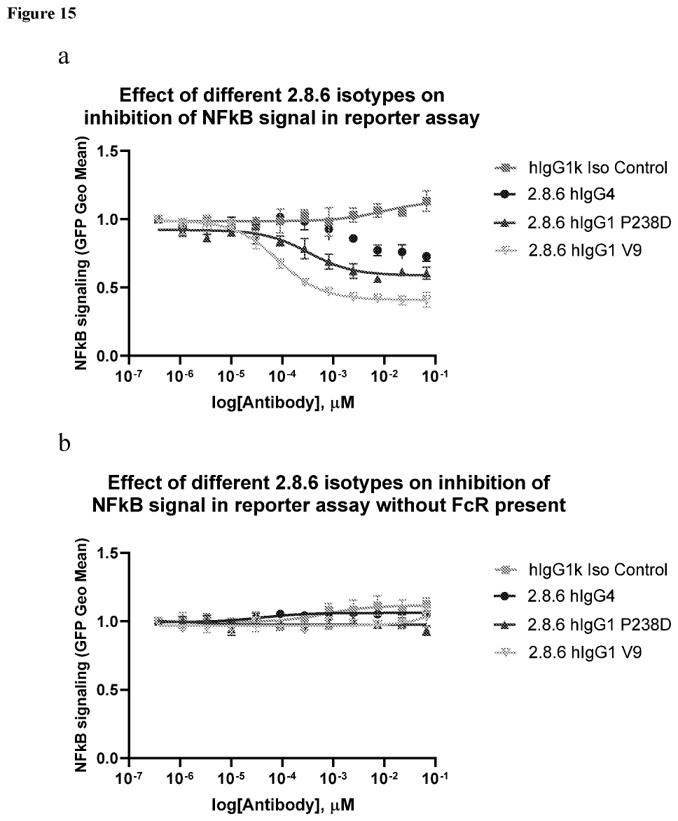

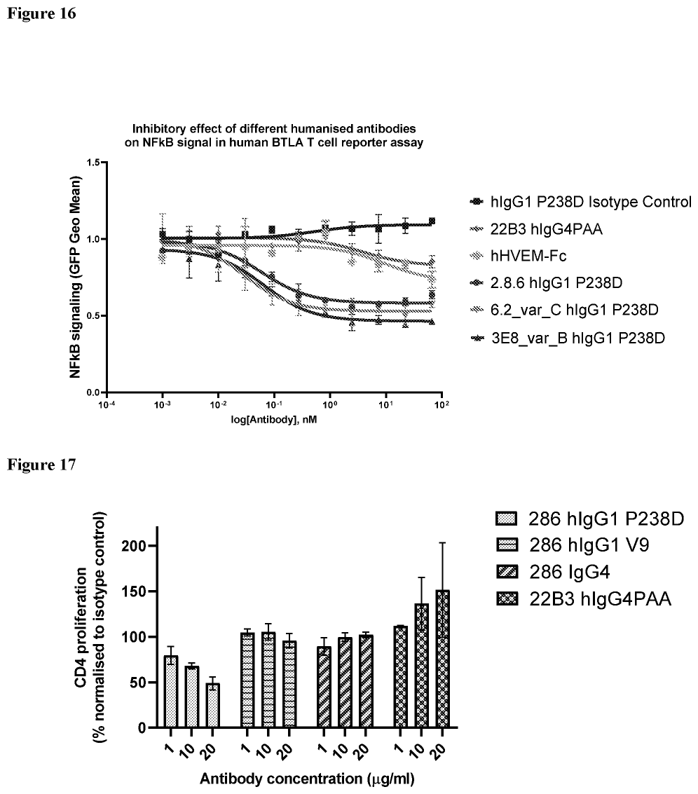

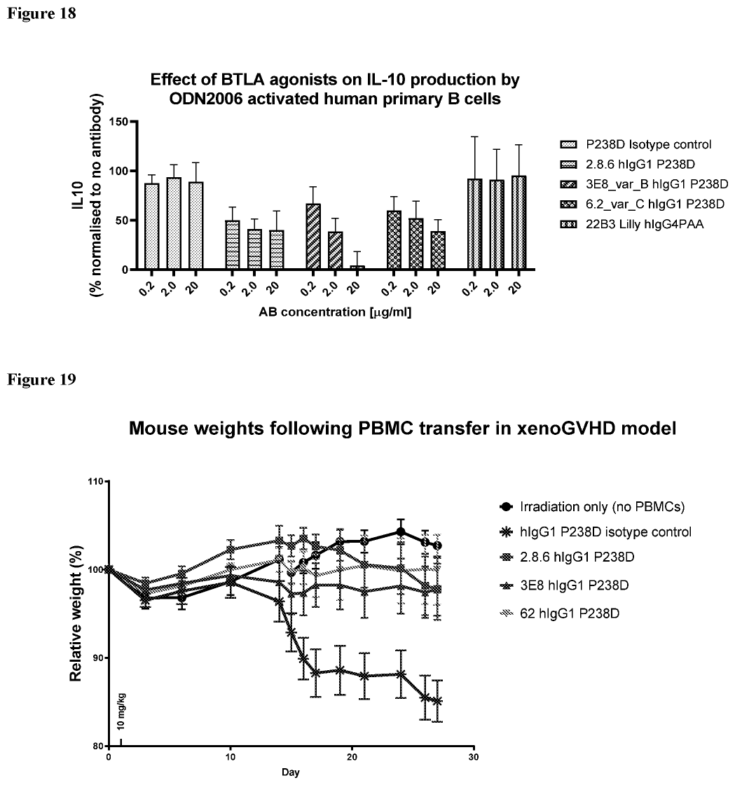

The binding affinity and kinetics of the BTLA agonist antibodies of the present invention (2.8.6 and 11.5.1) to human or cynomolgus BTLA were determined by surface plasmon resonance using the Biacore T200 (GE Healthcare). Mouse antibody capture kit (GE Healthcare) was used to coat a Series S CMS Sensor Chip (GE Healthcare) with polyclonal anti-mouse IgG. Anti-BTLA antibody was then captured onto the biosensor surface and a negative control antibody (clone Mopc21; Biolegend) captured in the reference channel. Various concentrations of monomeric soluble human BTLA extracellular domain (BTLA^{K31-R151}) (from SEQ ID NO: 225) or soluble cynomolgus macaque BTLA extracellular domain (BTLA^{K31-R151}) (from SEQ ID NO: 226) were then injected over the immobilized antibodies in the buffer 10 mM Hepes, 150 mM NaCl, 0.005% v/v Surfactant P20, pH 7.4 (HBS-P) at 37° C., in a single cycle kinetics analysis (FIG. 1a). Association and dissociation rates were fitted using BiaEvaluation Software (GE Healthcare) after reference and blank subtractions, and dissociation constants were calculated (FIG. 1b). Clone 2.8.6 bound human BTLA with a KD of 0.65 nM and cynomolgus BTLA with a KD of 7.89 nM. Clone 11.5.1 bound human BTLA with a KD of 0.75 nM and cynomolgus BTLA with a KD of 0.99 nM. In a separate experiment against human BTLA only, Clone 2.8.6 bound human BTLA with a KD of 0.37 nM and Clone 11.5.1 bound human BTLA with a KD of 0.53 nM.

### Example 3. Binding to BTLA on Cells

The ability of the BTLA agonist antibodies of the present invention (2.8.6 and 11.5.1) to bind to human or cynomolgus BTLA expressed on the cell surface was assessed by flow cytometry. A lentiviral transfection system was used to express full length human or cynomolgus BTLA in a Jurkat T cell line. 1×10⁵ cells per well were plated in 96 well U-bottom plates. BTLA antibody binding versus mIgG1 isotype control (clone MOPC-21, Biolegend #400165) was assessed at twelve concentrations by 1 in 3 serial dilution in FACS buffer (PBS, 2% FCS, 0.05% sodium azide), starting at a concentration of 90 μg/ml. Non-specific antibody binding was prevented by addition of Fc block (Biolegend #101319). Antibodies were incubated with cells for 30 minutes on ice, then cells were washed twice with FACS buffer prior to staining with an AF647 conjugated anti-mIgG1 secondary antibody (Biolegend #406618). Secondary antibody was incubated for 30 minutes on ice, then cells were washed and resuspended in FACS buffer for analysis on a flow cytometer. The geometric mean fluorescent intensity of secondary antibody was plotted for each concentration and the EC50 for receptor binding calculated by non-linear curve fitting using GraphPad Prism software. Clone 11.5.1 bound to human BTLA expressing cells with an EC50 of 0.016 nM and cynomolgus BTLA expressing cells with an EC50 of 0.0057 nM. Clone 2.8.6 bound to human BTLA expressing cells with an EC50 of 0.085 nM and cynomolgus BTLA expressing cells with an EC50 of 0.16 nM (FIG. 1c-d).

### Example 4. Competition with the Natural Ligand HVEM for Binding to BTLA

The ability of the BTLA agonist antibodies of the present invention (2.8.6 and 11.5.1) to block natural ligand binding to BTLA was assessed by surface plasmon resonance using the Biacore T200 (GE Healthcare). Human BTLA extracellular domain (BTLA^{31K-151R}) was covalently coupled to a CMS Sensor chip using amine coupling. Human HVEM extracellular domain, fused to mouse IgG1 Fc, was then injected over the immobilized hBTLA in HBS-P buffer at 37° C., and allowed to fully dissociate. A saturating amount of anti-BTLA antibody (2.8.6 or 11.5.1) was then injected, followed immediately by a second injection of human HVEM-mFc at the same concentration as the initial injection (FIG. 2A). Equilibrium HVEM binding (in Resonance Units) after saturation of BTLA with antibody was expressed as a percentage of binding prior to antibody injection (FIG. 2B). If HVEM binding following saturation with antibody was >90% of the binding prior to antibody injection, then the antibody was considered non-blocking.

### Example 5. Binding Epitope of Antibody 11.5.1 on Human BTLA

The functional epitope of the antibody 11.5.1 on human BTLA was determined by flow cytometry assessment of binding to a panel of single residue mutants of the receptor expressed on the cell surface. Constructs encoding the human extracellular region of BTLA with the transmembrane and intracellular regions of murine CD28 were cloned into the bi-cistronic mammalian expression vector pGFP2-n2 (BioSignal Packard Ltd), which also encodes GFP. Mutant constructs varying by one amino acid were prepared using the “drastic” mutagenesis approach (Davis et al. Proc Natl Acad Sci USA. 95, 5490-4 (1998)). Plasmids (2 μg/well) were transfected into HEK-293T cells in 6 well plates using Genejuice transfection reagent (Novagen; 6 μl/well). Mock and no-transfection controls were included with each experiment. Cells were harvested at 48 hours and stained with fluorochrome-conjugated anti-BTLA antibody at 10 μg/ml, alongside a Live/Dead marker, in PBS, 0.05% azide, 2% FCS (FACS buffer) for 1 h at 4° C. Cells were washed, pelleted and resuspended in 200 μl FACS buffer before being analysed on a BD FACSCanto flow cytometer. GFP-positive (transfected) viable cells were gated and analysed for binding of anti-BTLA antibodies (an example of the binding analysis for clone 11.5.1 is shown in FIG. 3a). For each mutant the Geo-mean of anti-BTLA antibody binding to transfected cells was expressed as a percentage of binding to the wild-type receptor (FIG. 3b). A panel of anti-BTLA antibodies was assessed and any mutation that eliminated binding of all antibodies was excluded from the analysis, on the assumption that such mutations lead to drastic changes in protein folding or expression rather than indicating an antibody epitope. The mutations Y39R and K41E completely abolish binding of antibody 11.5.1 whilst leaving binding of 2.8.6 unaffected. These mutations are mapped onto the human BTLA crystal structure (Compaan et al., J Biol Chem. 280:39553-61, 2005) in FIG. 3c (black residues), indicating the binding epitope of 11.5.1. Residues required for HVEM binding (Gln37, Arg42, Pro59, His127; from patent publication number WO2017004213) are also mapped onto the structure in grey demonstrating that 11.5.1 binds to an epitope very close to the HVEM binding site.

### Example 6. Crystal Structure of the Fab′ Fragment of 2.8.6 in Complex with Human BTLA

The structural epitope of antibody 2.8.6 on human BTLA was determined by solving the crystal structure of antibody Fab in complex with human BTLA extracellular domain. The heavy and light variable domains of antibody 2.8.6 were cloned into the pOPINVH and pOPINVL expression vectors (Addgene), which encode the first constant domain of the mouse IgG1 heavy chain (with a 6×Histidine tag) and the constant domain of the mouse Ig kappa chain, respectively. These vectors were transiently co-transfected into HEK293T cells to produce the Fab′ fragment of anti-BTLA 2.8.6, which was purified by Ni-NTA purification. Human BTLA Ig-V set domain (BTLA^{S33-D135}) was cloned into the pGMT7 vector and expressed in BL21(DE3)pLysS E. coli cells (Novagen) to produce inclusion bodies. The inclusion bodies were isolated from the cell pellet by sonication and washed repeatedly with a wash solution containing 0.5% Triton X-100. The purified BTLA inclusion bodies were solubilized in a denaturant solution containing 6 M guanidine hydrochloride. The solubilized protein solution was diluted slowly in refolding buffer [0.1 M Tris-HCl (pH 8.0), 0.6 M L-arginine, 2 mM ethylenediaminetetraacetic acid, 3.73 mM cystamine, and 6.73 mM cysteamine] to a final protein concentration of 1-2 μM and then stirred for 48 h at 4° C. The refolded mixture of BTLA was then concentrated with a VIVA FLOW50 system (Sartorius). BTLA was purified by gel filtration on a Superdex 75 column (GE Healthcare).

The purified BTLA and Fab′ were mixed and purified as a complex by size exclusion chromatography. The crystal suitable for data collection was obtained in 0.2 M calcium acetate, 0.1 M imidazole pH 8.0, 10% (w/v) PEG 8000 at 293° K by the hanging drop vapor-diffusion method. The final dataset was collected at the Photon Factory, and the structure was determined by molecular replacement using the structure of BTLA (PDB ID; 2AW2 chain A) and anti-PD1-Fab (PDB ID: SGGS chain C, D) as search probes.

The residues on BTLA at the interface with antibody 2.8.6 are A50, G51, D52, P53, E83, D84, R85, Q86, E103, P104, V105, L106, P107, N108, D135.

### Example 7. Development of Humanised BTLA Mice

To provide a platform to assess anti human-BTLA antibodies in mouse models, a knock-in strain of C57Bl/6 mice was developed expressing a chimeric form of BTLA with the human extracellular region and the murine transmembrane and signaling regions. A section of human genomic DNA from the beginning of exon 2 to the end of exon 3 was inserted into the mouse locus replacing the mouse sequence from the beginning of exon 2 to the end of exon 4. The sequences at the exon-intron junction at the beginning of mouse exon 2 and end of mouse exon 4 were left intact to ensure proper splicing (FIG. 5).

### Example 8. Inhibition of Antigen-Specific T Cell Proliferation In Vivo

The ability of the BTLA agonist antibodies of the present invention (2.8.6 and 11.5.1) to inhibit antigen specific T cell proliferation in vivo was assessed using a sensitive T-cell transfer assay (FIG. 6a). In this assay, 5×10⁵ T-cells, comprising a mixture of purified OTII (TCR transgenic) CD4⁺ T cells specific for ovalbumin (OVA) from mice expressing homozygous human BTLA (hBTLA), and from OT-II mice expressing the wild-type murine BTLA receptor (The Jackson Laboratory), were transferred into non-transgenic C57BL/6 recipients. The transferred cells were distinguished from host cells using the CD45.2 (versus CD45.1) allotypic marker. The wild-type donor cells also expressed green fluorescent protein under the control of the human ubiquitin C promoter to allow them to be distinguished from the humanised donor cells by flow cytometry. The day after T cell transfer, the recipient mice were immunised with 100 ovalbumin (Sigma-Aldrich) in 100 μl PBS mixed with 100 μl Imject Alum (ThermoFisher), to induce expansion of the T cells. On the second day, the mice were dosed with 200 μg of antibody, intraperitoneally. Eight days following the initial transfer of the T cells, the ratio of the humanised BTLA-expressing and wild-type OVA-specific T-cells in the spleen was determined by flow cytometry. In this way, it was possible to track the expansion or contraction of the humanised cells, which bind the anti-human BTLA antibodies, relative to the wild-type controls, which do not. Both antibodies 2.8.6 and 11.5.1 led to reduced expansion of the humanised BTLA cells relative to the wild-type controls indicating that they are inducing signaling through the inhibitory BTLA receptor, which leads to reduced T cell proliferation (FIG. 6b).

### Example 9. Inhibition of T Cell Proliferation in a Mixed Lymphocyte Reaction

The ability of the BTLA agonist antibodies of the present invention (2.8.6 and 11.5.1) to inhibit proliferation of primary T cells from the humanised mice in vitro was assessed using a mixed lymphocyte reaction (MLR). Splenocytes from Balb/c mice were treated with Mitomycin C for 30 mins at 37° C. then washed and used as stimulator cells. T cells were purified from the spleens of humanised BTLA mice, by negative selection using magnetic-activated cell sorting (Mojosort Mouse CD3 T cell isolation kit, Biolegend #480023), and stained with CellTrace Violet Cell Proliferation Kit (ThermoFisher) to use as responder cells. 4×10⁵ stimulator cells and 2×10⁵ responder cells per well were mixed in 96-well U-bottom plates with various concentrations of anti-BTLA or isotype control antibody (clone MOPC-21, Biolegend #400165). Serial 1 in 3 dilutions of antibody were assessed starting at a concentration of 1 μg/ml for a total of 10 concentrations. Polyclonal anti-mHVEM antibody (R&D systems #AF2516) was also added to all wells at 1 μg/ml to block any baseline signaling through the BTLA pathway and accentuate the effects of agonist antibodies. After 96 hours, dilution of CellTrace Violet in responder cells was assessed by flow cytometry as a marker of proliferation. Proliferation in the presence of anti-BTLA antibody or isotype control was compared to proliferation in the absence of antibody. CD4⁺ and CD8⁺ populations were gated out and analysed separately. Both antibodies 2.8.6 and 11.5.1 reduced proliferation of human-BTLA expressing T cells, indicating that they induce inhibitory signaling through the human BTLA receptor. Clone 2.8.6 inhibited CD4 T cells with an IC50 of 0.029 nM and had a maximal effect of 42% inhibition of proliferation (FIG. 7). Clone 11.5.1 inhibited CD4 T cells with an IC50 of 0.016 nM and had a maximal effect of 33% inhibition of proliferation.

### Example 10. Inhibition of NFkB Signalling in Human BTLA or Cynomolgus BTLA Transfected Jurkat T Cell Lines

The ability of the BTLA agonist antibodies of the present invention (2.8.6 and 11.5.1) to inhibit NFkB signalling was assessed using a BTLA transfected reporter T cell line. A Jurkat T cell line stably transfected with an expression cassette that includes NF-κB-responsive transcriptional elements upstream of a minimal CMV promoter (mCMV)-GFP cassette (Source BioSciences #TR850A-1) was used as a reporter cell line for NFkB signalling. A lentiviral transfection system was used to express full length human or cynomolgus BTLA in this reporter cell line. These cells were mixed with a stimulator cell line comprised of bw5147 cells expressing an anti-CD3 ScFv construct on their surface as described by Leitner et al. J Immunol Methods. 2010 Oct. 31; 362(1-2):131-41. The stimulator cell line was also transfected with murine FcγR2B to provide Fc receptors for presentation of the agonist BTLA antibodies. 5×10⁴ reporter cells per well were mixed in 96 well U-bottom plates with 5×10⁴ stimulator cells in the presence of various concentrations of BTLA antibody or isotype control (clone MOPC-21, Biolegend #400165). After 24 hours incubation at 37° C., cells were pelleted and stained for flow cytometry with a viability dye (Zombie Aqua, Biolegend #423101) and a mouse CD45 antibody (Pe-Cy7 conjugated clone 104, Biolegend #109830) to separate stimulator (murine) from responder (human) cells. Geometric mean of GFP expression was assessed for each antibody concentration and normalized to GFP expression in the absence of antibody. Clone 2.8.6 inhibited human BTLA transfected cells with an IC50 of 0.06 nM and cynomolgus BTLA transfected cells with an IC50 of 0.22 nM. Clone 11.5.1 inhibited human BTLA transfected cells with an IC50 of 0.033 nM and cynomolgus BTLA transfected cells with an IC50 of 0.14 nM.

### Example 11. Treatment of a T Cell Driven Mouse Model of Colitis by Antibody 2.8.6

The ability of the BTLA agonist antibody 2.8.6 to ameliorate a T cell driven model of colitis was assessed using the humanised mice. This T cell transfer model has previously been described as a murine model of inflammatory bowel disease (Ostanin et al., Am J Physiol Gastrointest Liver Physiol. 296:G135-46, 2009). CD45RB^{hi}CD25-CD4+ T cells sorted from spleens and lymph nodes of humanised BTLA mice were injected intraperitoneally into Rag1 KO recipients, (Rag1^{tm1Mom}; The Jackson Laboratory), at a dose of 5×10⁵ cells per mouse. The transferred T cells cause an inflammatory colitis that develops after approximately 3 weeks and leads to diarrhea and weight loss. Rag1 KO cagemates that did not receive transferred T cells serve as non-diseased controls. On days 7, 21 and 35 after T cell transfer the recipient mice were injected intraperitoneally with 200 μg of 2.8.6 or isotype control antibody. All mice were weighed regularly, and at 8 weeks colons were weighed and measured and inflammatory infiltration assessed by histology, as well as by cell counting and flow cytometry of extracted lamina propria leucocytes. Antibody 2.8.6 prevented weight loss (FIG. 8a) and significantly reduced inflammatory infiltration of colons (FIG. 8b). Colon inflammation in diseased mice led to an increased colon weight:length ratio that was not seen in 2.8.6 treated mice (FIG. 8c).

### Example 12. Treatment of a Mouse Model of Graft-Versus-Host Disease (GVHD)

The effects of the anti-BTLA agonist antibodies were assessed in a non-lethal parent-into-F1 model of GVHD. Bone marrow cells (BMCs) and splenocytes were harvested from humanised BTLA donor mice (C57BL/6 background; H2B). 2×10⁷ BMCs and 107 splenocytes were injected intravenously into CB6F1 (H2B^{/d}) recipients that had been lethally irradiated with 9 Gy total body irradiation. Irradiated CB6F1 mice reconstituted with syngeneic BMCs and splenocytes served as non-diseased controls. On the day of immune cell transfer mice were injected intraperitoneally with 200 μg anti-BTLA antibody or isotype control. Mice were weighed regularly and GVHD was monitored by calculating relative loss of body weight and by clinical observation. Mice were culled 5 weeks after immune cell transfer or when they reached a humane endpoint (which included >20% weight loss relative to starting weight in the first 14 days, or >15% weight loss at any other time). At the time of death colons were weighed and measured and a colon weight:length ratio calculated as a marker of colon inflammation, which is a prominent clinical feature of GVHD. Both antibodies 2.8.6 and 11.5.1 significantly reduced weight loss, leading to increased survival (FIG. 9a) and prevented colon inflammation (FIG. 9b).

### Example 13. Agonist Activity of Antibody 11.5.1 is Dependent on Fc Receptor Binding

Antibody 11.5.1 was recombinantly expressed as a mIgG1k containing a D265A mutation which has previously been described as significantly reducing Fc receptor binding (Clynes et al., Nat Med. 6:443-446, 2000). This mutated antibody was assessed in the T cell transfer assay described in Example 8. The parental 11.5.1 antibody inhibited proliferation of humanised T cells as its net effect is agonism of the BTLA receptor. The FcR-null D265A mutation, however, led to enhanced proliferation of humanised T cells suggesting that the FcR-null mutation removes the antibody's agonistic effect, leaving only the effect of receptor blockade (FIG. 10a).

The D265A mutated 11.5.1 antibody was also assessed in the in vitro MLR assay described in Example 9. Again, the parental 11.5.1 antibody inhibited proliferation of humanised T cells as its net effect is agonism of the BTLA receptor. The FcR-null D265A mutation removes the antibody's agonistic effect, so this antibody showed no effect in this assay (FIG. 10b). The FcR null 11.5.1 antibody did not enhance proliferation of humanised cells in this assay as HVEM was blocked (by the addition of polyclonal anti-HVEM antibody) so there was no baseline signaling through the pathway to be blocked by the BTLA blocking antibody.

### Example 14. Antibodies 2.8.6 and 11.5.1 do not Fix Complement In Vitro

Splenocytes from humanised mice were incubated with 10% baby rabbit complement (BioRad) and anti-BTLA antibodies (or an isotype control or a positive control depleting anti-CD20 antibody; clone SA271G2 from Biolegend) at 20 μg/ml for 15 min at 37° C. Whilst anti-CD20 antibody depleted the majority of B220⁺ B cells, anti-BTLA antibodies did not deplete either B220⁺ or CD4⁺ cells (FIG. 11), even though both these populations stain positively for BTLA.

### Example 15. Antibodies 2.8.6 and 11.5.1 do not Induce ADCC In Vitro

Whole splenocytes (including myeloid effector cells) from humanised mice were incubated with anti-BTLA antibodies (or isotype control or depleting anti-CD20 antibody SA271G2) at 20 μg/ml for 24 hours at 37° C. Whilst anti-CD20 antibody depleted the majority of B220⁺ cells, anti-BTLA antibodies did not deplete either B220⁺ or CD4⁺ cells (FIG. 12), even though both these populations stain positively for BTLA.

### Example 16. Antibodies 2.8.6 and 11.5.1 do not Deplete BTLA Expressing Cells In Vivo

Humanised BTLA mice were injected intraperitoneally with 200 μg anti-BTLA antibody or isotype control. At 24 hours spleens were harvested and the frequency of different cell populations identified by flow cytometry. Anti-BTLA antibody had no effect on the frequency or absolute number of B or T cells in the spleen or on the number of B cell precursors in the bone marrow (FIG. 13).

### Example 17. Antibodies 2.8.6 and 11.5.1 Stabilize Expression of BTLA on Immune Cells In Vivo

Humanised mice were injected intraperitoneally with 10 mg/kg of antibody 2.8.6 or 11.5.1. Six days after injection mice were humanely sacrificed and spleens harvested and processed to single cell suspension for assessment by flow cytometry. Cells were stained with a cocktail of antibodies to identify immune cell subsets and with fluorescently conjugated anti-BTLA antibody that had a non-competing epitope with the antibody that had been injected. The geometric mean of BTLA staining following in vivo incubation with anti-BTLA antibody was normalized to the geometric mean of BTLA staining (using the same staining antibody) following incubation with isotype control. BTLA expression was significantly higher on B cells and CD4 T cells from mice that had been injected with either clone 2.8.6 or 11.5.1, compared to mice that had been injected with isotype control (FIG. 14). This suggests that clones 2.8.6 and 11.5.1 stabilise expression of BTLA on the cell surface in vivo, rather than inducing receptor down-modulation, as has been observed with other BTLA antibodies in the prior art (M.-L. del Rio et al./Immunobiology 215 (2010) 570-578). For the purposes of immunosuppression an agonist antibody that stabilizes expression of the receptor presents the benefit of enabling prolonged high levels of inhibitory signaling through the pathway compared to a downmodulating antibody.

### Example 18. Tolerability and Side Effects in Animal Models

There were no tolerability issues or side effects noted in any animal studies with antibodies 2.8.6 or 11.5.1.

### Example 19. Characterisation of Exemplary BTLA Antibodies

Described in this example is characterisation of exemplary mIgG1 BTLA antibodies provided herein in addition to 2.8.6 and 11.5.1. Various clones listed in Tables 1 and 2 were evaluated for their binding affinity to BTLA and inhibition efficiency of lymphocytes (Table 3).

TABLE 1
Exemplary BTLA Agonistic Antibodies

| | | SEQ ID NOs | | | | | | | |
|---|---|---|---|---|---|---|---|---|---|
| Clone | Scheme | CDR H1 | CDR H2 | CDR H3 | CDR L1 | CDR L2 | CDR L3 | VH | VL |
| 10B1 | Kabat | 45 | 46 | 47 | 33 | 34 | 35 | 51 | 52 |
| 12F11 | Kabat | 53 | 54 | 55 | 56 | 57 | 58 | 59 | 60 |
| 14D4 | Kabat | 61 | 62 | 63 | 64 | 65 | 66 | 67 | 68 |
| 15B6 | Kabat | 61 | 69 | 70 | 71 | 72 | 73 | 74 | 75 |
| 15C6 | Kabat | 76 | 77 | 78 | 79 | 80 | 81 | 82 | 83 |
| 16E1 | Kabat | 45 | 46 | 84 | 33 | 34 | 85 | 86 | 87 |
| 16F10 | Kabat | 88 | 89 | 90 | 91 | 65 | 92 | 93 | 94 |
| 16H2 | Kabat | 95 | 96 | 97 | 98 | 99 | 100 | 101 | 102 |
| 1H6 | Kabat | 103 | 104 | 105 | 106 | 107 | 108 | 109 | 110 |
| 21C7 | Kabat | 76 | 111 | 112 | 113 | 114 | 115 | 116 | 117 |
| 24H7 | Kabat | 118 | 119 | 120 | 121 | 122 | 123 | 124 | 125 |
| 26B1 | Kabat | 126 | 127 | 128 | 79 | 129 | 130 | 131 | 132 |
| 26F3 | Kabat | 133 | 134 | 135 | 106 | 107 | 136 | 137 | 138 |
| 27G9 | Kabat | 103 | 134 | 139 | 106 | 107 | 136 | 141 | 138 |
| 3A9 | Kabat | 143 | 144 | 145 | 146 | 147 | 148 | 149 | 142 |
| 4B1 | Kabat | 151 | 152 | 153 | 154 | 155 | 156 | 157 | 158 |
| 4D3 | Kabat | 159 | 160 | 161 | 4 | 12 | 164 | 165 | 166 |
| 4D5 | Kabat | 167 | 168 | 169 | 170 | 171 | 172 | 173 | 174 |
| 4E8 | Kabat | 45 | 46 | 47 | 170 | 171 | 172 | 175 | 174 |
| 4H4 | Kabat | 45 | 46 | 177 | 154 | 155 | 178 | 179 | 180 |
| 6G8 | Kabat | 181 | 182 | 183 | 184 | 185 | 186 | 187 | 188 |
| 7A1 | Kabat | 76 | 77 | 78 | 79 | 80 | 189 | 82 | 190 |
| 8B4 | Kabat | 45 | 191 | 192 | 154 | 155 | 193 | 194 | 195 |
| 8C4 | Kabat | 196 | 197 | 198 | 199 | 200 | 201 | 202 | 203 |
| 11.5.1 | Kabat | 204 | 205 | 206 | 207 | 208 | 209 | 210 | 211 |
| 831 | Kabat | 212 | 213 | 214 | 215 | 34 | 216 | 217 | 218 |
| 6.2 | Kabat | 1 | 2 | 3 | 4 | 5 | 6 | 219 | 220 |
| 2.8.6 | Kabat | 20 | 163 | 22 | 23 | 176 | 25 | 221 | 222 |
| 3E8 | Kabat | 30 | 48 | 32 | 33 | 34 | 35 | 223 | 150 |

TABLE 2
Humanised and engineered antibodies

| | SEQ ID Nos. | | | | | | | | | |
|---|---|---|---|---|---|---|---|---|---|---|
| | | | | | | | | | Heavy | Light |
| Clone | CDR H1 | CDR H2 | CDR H3 | CDR L1 | CDR L2 | CDR L3 | VH | VL | chain | chain |
| humanised 6.2 | 1 | 2 | 3 | 4 | 5 | 6 | 7 | 8 | 9 | 10 |
| Engineered | 1 | 11 | 3 | 4 | 12 | 6 | 13 | 14 | 15 | 16 |
| humanised 6.2 | | | | | | | | | | |
| (Variant A) | | | | | | | | | | |
| Engineered | 1 | 11 | 3 | 4 | 5 | 6 | 13 | 8 | 15 | 10 |
| humanised 6.2 | | | | | | | | | | |
| (Variant B) | | | | | | | | | | |
| Engineered | 1 | 17 | 3 | 4 | 12 | 6 | 18 | 14 | 19 | 16 |
| humanised 6.2 | | | | | | | | | | |
| (Variant C) | | | | | | | | | | |
| Humanised | 20 | 21 | 22 | 23 | 24 | 25 | 26 | 27 | 28 | 29 |
| 2.8.6 | | | | | | | | | | |
| Humanised 3E8 | 30 | 31 | 32 | 33 | 34 | 35 | 36 | 37 | 38 | 39 |
| Engineered | 30 | 40 | 32 | 33 | 34 | 35 | 41 | 37 | 42 | 39 |
| humanised 3E8 | | | | | | | | | | |
| (Variant A) | | | | | | | | | | |
| Engineered | 30 | 31 | 32 | 33 | 34 | 35 | 36 | 43 | 38 | 44 |
| humanised 3E8 | | | | | | | | | | |
| (Variant B) | | | | | | | | | | |

For each antibody, the association rate (“on rate”) and dissociation rate (“off rate”) for binding human BTLA, and KD for binding human or cynomolgus BTLA were measured according to the method described in Example 2, fitting curves for injection of BTLA extracellular domain at a single concentration. Inhibition efficiency of individual antibodies on T cells was also evaluated at a single concentration of 10 μg/ml. MLR assay was performed for each individual antibody according to the method as described in Example 9 (two biological repeats as shown in Table 4); anti-CD3 assay was performed according to the method described below (two biological repeats, Table 4); and inhibition of NFkB signalling in human BTLA transfected Jurkat T cell line by each antibody was determined according to the method as described in Example 10 (Table 4). The average inhibition of T cells relative to isotype control in various in vitro stimulation assays for each exemplary antibody was calculated as a mean of the percentage inhibition of all assay results (Table 3 and Table 4).

TABLE 3
Characterisation of binding affinity and inhibitory effect of exemplary antibodies

| | | Human | Human | Human | Cyno | Average | |
|---|---|---|---|---|---|---|---|
| | | BTLA | BTLA | BTLA | BTLA | inhibitory | |
| | Ligand | On rate | Off rate | KD | KD | effect | |
| Clone | Blocking | (1/Ms) | (1/s) | (nM) | (nM) | in vitro | Epitope |
| 2.8.6 | No | 6.46E+05 | 4.23E−04 | 0.65 | 7.89 | 39% | 1 |
| 24H7 | No | 2.43E+05 | 1.60E−04 | 0.66 | — | 30% | 4 |
| 11.5.1 | Yes | 6.03E+05 | 4.49E−04 | 0.75 | 0.99 | 30% | 2 |
| 14D4 | Yes | 2.54E+05 | 3.77E−04 | 1.49 | 1.83 | 33% | 2 |
| 6.2 | No | 6.30E+05 | 1.07E−03 | 1.70 | 9.71 | 35% | 1 |
| 4B1 | No | 5.77E+05 | 1.85E−03 | 3.21 | — | 29% | 4 |
| 8B4 | No | 5.38E+05 | 4.40E−03 | 8.17 | — | 29% | 4 |
| 16H2 | No | 3.97E+05 | 3.27E−03 | 8.25 | 160.1 | 34% | 1 |
| 1H6 | Yes | 7.72E+05 | 6.90E−03 | 8.94 | 6.08 | 31% | 2 |
| 8C4 | Yes | 3.63E+05 | 5.76E−03 | 15.89 | 161.48 | 19% | 2 |
| 26B1 | Yes | 3.23E+05 | 9.70E−03 | 30.03 | 167.66 | 21% | 3 |
| 7A1 | No | 4.13E+05 | 1.66E−02 | 40.17 | — | 24% | 1 |
| 21C7 | No | 9.30E+05 | 4.06E−02 | 43.65 | — | 18% | 5 |
| 16F10 | No | 5.81E+05 | 2.83E−02 | 48.78 | — | — | 1 |
| 6G8 | No | 3.18E+05 | 1.67E−02 | 52.42 | — | — | 1 |
| 3E8 | No | 5.43E+05 | 6.08E−02 | 111.98 | 607.46 | 41% | 1 |
| 4E8 | No | 1.75E+05 | 3.14E−02 | 180.00 | — | — | 1 |
| 27G9 | Yes | 1.92E+05 | 8.38E−02 | 436.86 | 653.63 | 16% | 2 |
| 15C6 | No | 1.93E+05 | 1.38E−01 | 718.44 | — | — | 1 |
| 12F11 | No | 2.15E+05 | 1.55E−01 | 722.33 | — | 24% | 1 |
| 10B1 | No | 4.22E+05 | 5.21E−01 | 1233.36 | — | 21% | 1 |
| 15B6 | No | 4.47E+05 | 5.76E−01 | 1287.18 | — | 14% | 1 |
| 4D3 | No | 1.52E+05 | 2.51E−01 | 1651.32 | — | — | 1 |
| 4H4 | No | 2.03E+05 | 3.47E−01 | 1708.23 | — | 26% | 4 |
| 26F3 | Yes | 9.21E+05 | 2.02E+00 | 2195.81 | 809.75 | 9% | 2 |
| 16E1 | No | 7.30E+05 | 2.13E+00 | 2923.69 | — | 15% | 1 |
| 4D5 | No | 2.70E+05 | 7.90E−01 | 2929.18 | — | — | 1 |
| 3A9 | No | 4.06E+05 | 1.63E+00 | 4006.90 | — | 19% | 1 |

TABLE 4
Inhibitory effect assay results of exemplary antibodies

| | | | | | | | T cell | |
|---|---|---|---|---|---|---|---|---|
| | MLR | | AntiCD3/CD28 | | AntiCD3/CD28 | | reporter | |
| | (CD4 T cell proliferation) | | (CD4 T cell proliferation) | | (CD69+ CD4 T cells) | | (NFκB | |
| Clone | repeat 1 | repeat 2 | repeat 1 | repeat 2 | repeat 1 | repeat 2 | signaling) | Average |
| 2.8.6 | 30% | 36% | 23% | 35% | 58% | 67% | 22% | 39% |
| 24H7 | 23% | 31% | 13% | 23% | 52% | 44% | 22% | 30% |
| 6.2 | 31% | 35% | 19% | 21% | 53% | 61% | 26% | 35% |
| 11.5.1 | 23% | 18% | 21% | 28% | 50% | 47% | 19% | 30% |
| 11.5.1 D265A | −3% | 1% | −3% | −9% | −47% | −26% | −13% | −14% |
| 4B1 | 33% | 30% | 14% | 18% | 47% | 41% | 23% | 29% |
| 14D4 | 39% | 26% | 24% | 29% | 43% | 52% | 16% | 33% |
| 831 | 25% | 34% | 10% | 8% | 50% | 53% | 24% | 29% |
| 16H2 | 40% | 26% | 11% | 23% | 51% | 60% | 29% | 34% |
| 1H6 | 31% | 16% | 26% | 19% | 47% | 53% | 26% | 31% |
| 8B4 | 33% | 23% | 20% | 4% | 51% | 47% | 24% | 29% |
| 21C7 | 8% | 17% | 10% | −4% | 39% | 35% | 23% | 18% |
| 3E8 | 43% | 35% | 27% | 35% | 52% | 64% | 30% | 41% |
| 7A1 | 23% | 29% | 14% | 17% | 28% | 38% | 20% | 24% |
| 26B1 | 12% | 10% | 11% | 19% | 35% | 30% | 29% | 21% |
| 8C4 | 42% | −2% | 12% | 4% | 29% | 29% | 21% | 19% |
| 27G9 | 9% | 8% | 10% | 13% | 24% | 22% | 24% | 16% |
| 12F11 | 28% | 23% | 5% | 9% | 30% | 40% | 30% | 24% |
| 15C6 | 19% | 8% | 2% | −2% | 12% | 19% | 9% | 10% |
| 26F3 | 9% | −5% | 4% | 0% | 19% | 17% | 20% | 9% |
| 4D3 | 12% | 9% | −4% | −2% | 6% | 2% | 26% | 7% |
| 10B1 | 16% | 25% | 8% | 14% | 24% | 36% | 27% | 21% |
| 16E1 | 33% | 8% | 4% | 8% | 9% | 23% | 22% | 15% |
| 15B6 | 7% | 13% | 9% | 16% | 13% | 20% | 21% | 14% |
| 3A9 | 7% | 24% | 9% | 9% | 22% | 34% | 27% | 19% |
| 4H4 | 10% | 17% | 14% | 22% | 43% | 52% | 25% | 26% |
| No antibody | 3% | −3% | 1% | −6% | 2% | −9% | 2% | −1% |

The ability of the BTLA agonist antibodies to inhibit anti-CD3 and anti-CD28 induced T cell activation was assessed as follows. Splenocytes from humanised BTLA mice were processed to single cell suspension and treated with ACK buffer to lyse red blood cells. Cells were stained with CFSE (Biolegend Cat #423801) to enable tracking of cell proliferation. 2×10⁵ cells per well were plated in 96 well U-bottom plates with soluble anti-CD3 antibody (clone 145.2C11; Biolegend #100339) and anti-CD28 (clone 37.51; Biolegend #102115) each at a concentration of 50 ng/ml, and soluble anti-BTLA antibody or isotype control at a concentration of 10 μg/ml. After 72 hours cells were analysed by flow cytometry to assess proliferation (“antiCD3/CD28 (CD4 T cell proliferation)”) and T cell activation by staining of surface expressed activation markers (“antiCD3/CD28 (CD69+CD4 T cells)”). For each BTLA antibody the percentage inhibition compared to isotype control antibody was calculated.

Further, for each BTLA antibody, their ligand blocking capability, e.g., competition with HVEM for binding to BTLA, was assessed according to the method as described in Example 4, and the results are presented as “Yes” for more than 90% inhibition of HVEM-BTLA binding, and “No” for less than 10% inhibition of HVEM-BTLA binding. Functional epitope of each BTLA antibody was also determined according to the method as described in Example 5. The “epitope” column in Table 3 summarizes the epitope group that each individual BTLA antibody binds to. Antibodies 2.8.6, 6.2, 831, 16H2, 7A1, 16F10, 6G8, 3E8, 4E8, 15C6, 12F11, 10B1, 15B6, 4D3, 16E1, 4D5 and 3A9 all bind to a first epitope (named “epitope 1” in the table) comprising at least one critical residue selected from the list: D52, P53, E55, E57, E83, Q86, E103, L106 and E92 (position according to SEQ ID NO:225). Antibodies binding to epitope 1 do not compete with the ligand HVEM for binding to BTLA. Antibodies 11.5.1, 14D4, 1H6, 8C4, 27G9, 26F3 all bind to a different second epitope (“epitope 2”) comprising at least one critical residue selected from the list: Y39, K41, R42, Q43, E45 and S47. Antibodies binding to epitope 2 do compete with the ligand HVEM for binding to BTLA. Antibody 26B1 binds to a third epitope (“epitope 3”) comprising at least one critical residue selected from the list: D35, T78, K81, S121 and L123. Antibodies binding to epitope 3 do compete with the ligand HVEM for binding to BTLA.

Antibodies 24H7, 4B1, 8B4, 4H4 all bind to a different fourth epitope (“epitope 4”) comprising the critical residue H68. Antibodies binding to epitope 4 do not compete with the ligand HVEM for binding to BTLA. Antibody 21C7 binds to a different fifth epitope (“epitope 5”) comprising at least one critical residue selected from the list: N65 and A64. Antibodies binding to epitope 5 do not compete with the ligand HVEM for binding to BTLA.

### Example 20. Humanisation and CDR Engineering of BTLA Antibodies 6.2, 2.8.2 and 3E8

Antibody 2.8.6 was humanised by CDR grafting on to homologous human germline framework regions (See SEQ ID NO: 26, 27). IGHV2-5*08 was used for the heavy chain and IGKV3-11*01 for the light chain. After humanisation, binding to BTLA was assessed by SPR. Humanised 2.8.6 bound to monomeric BTLA with a K_{D} of 0.73 nM.

The variable domains of 6.2 and 3E8 were humanised by germlining to homologous human germline framework regions (Seq ID No. 7, 8 and 36, 37). For 3E8 the acceptor frameworks selected were VH1-1-08 and JH6 for the heavy chain and VK3-L6 and JK2 for the light chain. For 6.2 the acceptor frameworks selected were VH3-3-21 and JH6 for the heavy chain and VK2-A19 and JK4 for the light chain.

It is sometimes possible to substitute certain residues in the CDRs or variable domain framework regions of an antibody to remove undesirable characteristics without significantly impacting target binding. The CDRH2 of the humanised antibody 6.2 was modified with N56Q alone (SEQ ID NO: 17) or N56Q and D54E substitutions (Seq ID NO: 11) to remove deamidation potential and isomerisation potential respectively. The CDRL2 of humanized 6.2 was modified with a D61E substitution to reduce predicted immunogenicity as determined by Lonza's Epibase analysis (Seq ID NO: 12). Outside of the CDRs, an S77T substitution was introduced into the heavy variable framework region of humanized 6.2 to reduce predicted immunogenicity and a Q51K substitution was introduced into the light variable framework region to reduce immunogenicity. Three engineered variants of humanized 6.2 containing different combinations of these substitutions were created (Engineered humanized 6.2 “Variant A”, “Variant B” and “Variant C”). Table 2 describes the constituent CDRs and variable domains for each of these variants. An engineered variant of antibody 6.2 containing a CDRH2 with just the N56Q and not the D54E substitution (e.g. engineered humanised 6.2 variant C) is not disclosed in PCT/GB2019/053569.

Similarly, the CDRH2 of the humanised antibody 3E8 was modified with an N57Q substitution to remove deamidation potential and a K63S substitution to reduce predicted immunogenicity (Seq ID No. 40). Outside of the CDRs, G42D and A61S substitutions were introduced into the light chain variable framework of 3E8, to reduce predicted immunogenicity. Furthermore, P15L and P81A substitutions were introduced into the light chain variable framework to revert these positions to the murine sequence instead of introducing prolines that can have an impact on the local conformation. The sequence of the engineered 3E8 light chain variable domain contain all four of these substitutions is given in Seq ID No. 43. Table 2 describes the constituent CDRs and variable domains for engineered variants of humanized 3E8.

### Example 21. Binding of Humanised Anti-BTLA Antibodies to Soluble Human and Cynomolgus BTLA

The binding affinity and kinetics of humanised BTLA agonist antibodies to human or cynomolgus BTLA were determined by surface plasmon resonance using the Biacore 8K (GE Healthcare). Human antibody capture kit (GE Healthcare cat #29234600) was used to coat a Series S CMS Sensor Chip (GE Healthcare) with polyclonal anti-human IgG. Anti-BTLA antibody was then captured onto the biosensor surface and a negative control antibody (human IgG1k isotype control; Sino Biological cat #HG1K) captured in the reference channel. Various concentrations of monomeric soluble human BTLA extracellular domain (BTLA^{K31-R151}, produced recombinantly in house) or soluble cynomolgus macaque BTLA extracellular domain (BTLA^{K31-R151}, produced recombinantly in house) were then injected over the immobilized antibodies in the buffer HBS-EP (GE Healthcare, cat #BR100669), pH 7.4 (HBS-P) at 37° C., in a single cycle kinetics analysis. For human BTLA concentrations from 673 nM to 164 pM in serial four-fold dilutions were used. For cyno BTLA concentration from 1351 nM to 330 pM in serial four-fold dilutions were used. Association and dissociation rates were fitted using BiaEvaluation Software (GE Healthcare) after reference and blank subtractions, and dissociation constants were calculated (Table 5). Humanised 2.8.6 binds human BTLA with a KD of 2.33 nM and cynomolgus BTLA with a KD of 147 nM. Humanised 3E8 variant B (3E8_var_B) binds human BTLA with a KD of 141 nM and cynomolgus BTLA with a KD of 1520 nM. The humanised 6.2 variant A, which contains both D54E and N56Q substitutions in its CDRH2 to remove isomerisation and deamidation potential respectively, binds to human BTLA with a KD of 10.9 nM and cynomolgus BTLA with a KD of 695 nM. This binding represents a significant reduction in affinity from the parent clone 6.2 antibody, which binds to human BTLA with a KD of 1.7 nM, and cynomolgus BTLA with a KD of 9.71 nM (Table 5). A humanised variant of 6.2 that contains just the N56Q substitution but not the D54E substitution in CDRH2, termed Humanised 6.2 variant C (or 6.2_var_C), binds human BTLA with a KD of 1.25 nM and cynomolgus BTLA with a KD of 15.4 nM therefore retaining affinity much closer to the parent clone.

TABLE 5
Binding kinetics and affinity for antibodies binding to soluble human or
cynomolgus BTLA, as determined by surface plasmon resonance at 37° C.

| | Human BTLA | | | Cyno BTLA | | |
|---|---|---|---|---|---|---|
| Antibody | ka (1/Ms) | kd (1/s) | KD (M) | ka (1/Ms) | kd (1/s) | KD (M) |
| Humanised | 6.73 × 10⁵ | 1.57 × 10⁻³ | 2.33 × 10⁻⁹ | 1.7 × 10⁵ | 2.5 × 10⁻² | 1.47 × 10⁻⁷ |
| 2.8.6 | | | | | | |
| Humanised | 6.94 × 10⁵ | 7.56 × 10⁻³ | 1.09 × 10⁻⁸ | 7.29 × 10⁴ | 5.07 × 10⁻² | 6.95 × 10⁻⁷ |
| 6.2_var_A | | | | | | |
| Humanised | 1.06 × 10⁶ | 1.33 × 10⁻³ | 1.25 × 10⁻⁹ | 2.29 × 10⁵ | 3.52 × 10⁻³ | 1.54 × 10⁻⁸ |
| 6.2_var_C | | | | | | |
| Humanised | 8.65 × 10⁵ | 1.22 × 10⁻¹ | 1.41 × 10⁻⁷ | 2.87 × 10⁵ | 4.31 × 10⁻¹ | 1.52 × 10⁻⁶ |
| 3E8_var_B | | | | | | |

### Example 22. Binding of Humanised Anti-BTLA Antibodies to BTLA on Cells

The ability of the BTLA agonist antibodies of the present invention to bind to human or cynomolgus BTLA expressed on the cell surface was assessed by flow cytometry. A lentiviral transfection system was used to express full length human or cynomolgus BTLA in a Jurkat T cell line. 1×10⁵ cells per well were plated in 96 well U-bottom plates. BTLA antibody binding versus hIgG1k P238D isotype control (clone MOPC-21, produced recombinantly by Absolute Antibody; Heavy chain SEQ ID NO: 230, light chain SEQ ID NO: 231) was assessed at twelve concentrations by 1 in 3 serial dilution in FACS buffer (PBS, 2% FCS, 0.05% sodium azide), starting at a concentration of 30 μg/ml. Non-specific antibody binding was prevented by addition of Fc block (Biolegend #101319). Antibodies were incubated with cells for 60 minutes on ice, then cells were washed twice with FACS buffer prior to staining with an AF647 conjugated anti-hIgG secondary antibody (Clone HP6017; BioLegend cat #409320). Secondary antibody was incubated for 30 minutes on ice, then cells were washed and resuspended in FACS buffer for analysis on a flow cytometer. The geometric mean fluorescent intensity of secondary antibody was plotted for each concentration and the EC50 for receptor binding calculated by non-linear curve fitting using GraphPad Prism software. Humanised 2.8.6 binds to human BTLA expressing cells with an EC50 of 0.066 nM (FIG. 15a) and cynomolgus BTLA expressing cells with an EC50 of 0.854 nM (FIG. 15b). Humanised 6.2_var_C binds to human BTLA expressing cells with an EC50 of 0.062 nM and cynomolgus BTLA expressing cells with an EC50 of 0.148 nM. Humanised 3E8_var_B binds to human BTLA expressing cells with an EC50 of 0.177 nM and cynomolgus BTLA expressing cells with an EC50 of 15.6 nM.

### Example 23. Binding Affinities of Fc Variant Antibodies to Human Fc Receptors

In Example 13 it was demonstrated surprisingly that the agonist function of BTLA antibodies may be dependent on Fc receptor engagement by the Fc portion of the antibody. In humans there is one inhibitory Fc gamma receptor (FcγR2B) whilst the other Fc gamma receptors all deliver immune activating signals (FcγR1A, FcγR2A, FcγR3A and FcγR3B). For a BTLA agonist antibody to be effective at suppressing immune responses without eliciting inflammatory FcR signalling we propose it might require selective Fc binding to FcγR2B. Furthermore, selective binding to FcγR2B would promote bidirectional inhibitory signalling through BTLA on the BTLA expressing cell and through FcγR2B on the FcγR2B expression cell, which would strengthen the immunosuppressive effect of the antibody. This would be desirable in a therapeutic antibody intended for the treatment of diseases of immune overactivation. Conversely, very high affinity for FcγR2B can adversely impact antibody half-life due to turnover of the receptor in liver sinusoidal epithelial cells (Ganesan et al. The Journal of Immunology 189(10): 4981-88, 2012) as demonstrated by the FcγR2B enhanced IgG1 antibody XmAb7195 which binds to FcγR2B with a KD of 7.74 nM (Chu et al. Journal of Allergy and Clinical Immunology 129(4): 1102-15, 2012; https://linkinghub.elsevier.com/retrieve/pii/S0091674911018343 (May 13, 2020) and was reported by Xencor to have an average in vivo half-life of 3.9 days in a phase 1a trial (American Thoracic Society (ATS) 2016 International Conference in San Francisco, Calif.—A6476: Poster Board Number 407), compared to an average half-life of around 21 days for a wildtype IgG1 (Morell, Terry, and Waldmann. Journal of Clinical Investigation 49(4): 673-80, 1970; http://www.jci.org/articles/view/106279 (May 16, 2020)). Therefore, whilst selectivity for FcγR2B and sufficient binding to support agonism might be desirable for a BTLA agonist antibody, excessively high affinity for FcγR2B might be undesirable in a therapeutic as the consequently shortened half-life would likely necessitate more frequent dosing.

A range of Fc mutated antibody variants were recombinantly produced (containing the variable domains of humanised 2.8.6) and their binding to the different human Fc gamma receptors assessed by surface plasmon resonance (at 37° C. in buffer HBS-EP+ at pH7.4). Fc variants were recombinantly produced on either a hIgG1 or a hIgG4 backbone with substitutions known to impact FcR binding or likely to do so based on their position in the Fc-FcR binding interface (hIgG1 G236D, hIgG1 G237D, hIgG1 P238D, hIgG1 D265A, hIgG1 S267E, hIgG1 P271G, hIgG1 A330R, hIgG1 K322A, hIgG1 N297A, hIgG4 P238D, hIgG4 G237D, hIgG4 P271G, hIgG4 S330R, hIgG4 F234A, hIgG4 L235A). These mutations were assessed as single substitutions or in combinations. Variants containing sections of sequence switched from hIgG2 as described by Armour et al. (Molecular Immunology 40(9): 585-93, 2003) were also assessed (termed delta b, delta c, delta ab and delta ac). The binding of mIgG1 and mIgG1 D265A to human FcRs was also assessed.

For the low affinity FcγRs (FcγR2A, FcγR2B, FcγR3A and FcγR3B) the interactions were assessed by surface plasmon resonance with the recombinantly expressed FcRs (extracellular domains only) as analyte. Briefly, recombinant human BTLA extracellular domain (BTLA^{K31-R151}) was covalently immobilised to both flow cells of all channels of a CMS Series S sensor chip using the GE Healthcare Amine coupling kit. The 2.8.6 Fc variant to be assessed was then captured (approx. 500-1000 response units) in flow cell 2 of each channel. Steady state affinity analysis was then performed by injecting varying concentrations of FcR in multiple cycles and measuring equilibrium binding. Double referencing was used (subtracting the signal in the reference Fc1 and also subtracting the signal from a blank zero concentration injection). KDs were calculated from the Langmuir curves (plotting equilibrium binding against analyte concentration to determine the concentration required for half maximal binding).

For the high affinity FcR interactions (FcγR1A, and also FcRn assessed at pH6.0) the binding was assessed in a kinetic analysis with antibody as analyte. Briefly, biotinylated FcR (Sino Biological, cat #10256-H085-B for FcγR1A or cat #CT009-H08H-B for FcRn) was captured in flow cell 2 on a streptavidin chip (Series S Sensor Chip SA-BR-1005-31) as per the provided protocol. Reference flow cell 1 was left empty in all channels. Purified antibody was then injected at a single concentration and on/off rates calculated by curve fitting on BiaEvaluation software. FcRn interaction at pH6.0 does not cause inflammatory signalling but is required for maintained antibody half-life in vivo and so this interaction is desirable for a therapeutic antibody. IgG Fc has two binding sites for FcRn so this assessment performed with FcRn immobilised at high density provides an avidity estimate for the interaction rather than a true KD.

The KD values for each of the Fc variants binding to each of the human Fc receptors where they were assessed are provided in Table 6. The presence of the P238D mutation significantly enhanced selectively for FcγR2B (by slightly increasing affinity to FcγR2B whilst drastically reducing affinity to other FcγRs). A previously described combination of mutations including P238D (P238D G237D P271G A330R), termed V9 (Mimoto et al. Protein Engineering, Design and Selection 26(10): 589-98, 2013), significantly increased binding affinity to FcγR2B but also retained significant binding to the 131R polymorphic variant of FcγR2A. The same effect of increasing FcγR2B selectivity was seen when the P238D single or combination substitutions were introduced into a hIgG4 backbone.

TABLE 6
Binding affinities (KDs) for Fc variants binding to human FcRs as assessed
by SPR at 37 C. n/a = not assessed, NB = no binding detected.

| | KD (μM) | | | | | | | FcRn pH6 |
|---|---|---|---|---|---|---|---|---|
| | | FcγR2A | FcγR2A | | FcγR3A | FcγR3A | | (avidity |
| | FcγR1A | 131R | 131H | FcγR2B | 158F | 158V | FcγR3B | in nM) |
| hIgG1 | 0.00375 | 1.57 | 1.98 | 8.65 | 9.37 | 2.78 | 22.9 | 4.58 |
| hIgG4 | 0.026 | 4.08 | 8.89 | 6.39 | 228 | 89.3 | 1100 | 13 |
| hIgG1 P238D | 0.465 | 28.9 | 76.5 | 4.78 | 2480 | 7010 | 1580 | 1.43 |
| hIgG1 P238D | 0.697 | 1.84 | 26.2 | 0.173 | 216 | 321 | 6090 | 1.98 |
| G237D P271G | | | | | | | | |
| A330R (V9) | | | | | | | | |
| hIgG4PAA | 1.34 | 21.4 | 39.1 | 41 | NB | 15 | NB | n/a |
| hIgG4 P238D | n/a | 47.9 | 312 | 17.5 | NB | NB | NB | n/a |
| hIgG4 P238D | n/a | 2.01 | 30.5 | 0.574 | 933 | NB | NB | n/a |
| G237D P271G | | | | | | | | |
| S330R | | | | | | | | |
| hIgG1 D265A | 0.497 | 48.3 | 40.1 | 193 | 1490 | NB | 5200 | 12 |
| hIgG1 D265A | NB | 91.4 | NB | 748 | NB | NB | NB | 14 |
| G236D | | | | | | | | |
| hIgG1 D265A | n/a | 67 | 39.4 | 347 | NB | NB | 9100 | n/a |
| A330R | | | | | | | | |
| hIgG1 D265A | 0.415 | 26.7 | 173 | 157 | NB | NB | NB | 11.9 |
| S267E | | | | | | | | |
| hIgG1 D265A | NB | 61.8 | NB | 113 | NB | 1650 | NB | 18.7 |
| G236D S267E | | | | | | | | |
| hIgG1 D265A | NB | 91.8 | 91.3 | 398 | NB | NB | NB | 14 |
| G236D A330R | | | | | | | | |
| hIgG1 D265A | n/a | 22 | 113 | 80.1 | NB | NB | NB | n/a |
| S267E A330R | | | | | | | | |
| hIgG1 D265A | NB | 54.4 | NB | 104 | NB | NB | NB | n/a |
| G236D S267E | | | | | | | | |
| A330R | | | | | | | | |
| hIgG1 D265A | n/a | 348 | 4590 | 684 | NB | NB | NB | n/a |
| P238D | | | | | | | | |
| hIgG4 D265A | n/a | 191 | 280 | 1000 | NB | NB | NB | n/a |
| hIgG4 D265A | n/a | 680 | NB | NB | NB | NB | NB | n/a |
| P238D | | | | | | | | |
| hIgG4 D265A | n/a | 167 | 2430 | 114 | NB | NB | NB | n/a |
| G236D S267E | | | | | | | | |
| A330R | | | | | | | | |
| hIgG1 delta ab | NB | 9.56 | 4.86 | 82.5 | 722 | 494 | 2820 | 14.8 |
| hIgG1 delta ab | NB | — | 771 | 876 | NB | NB | NB | n/a |
| P238D | | | | | | | | |
| hIgG1 delta ac | 2.85 | 79.3 | 82.2 | 131 | 344 | 96.4 | NB | 11.9 |
| hIgG1 delta ac | NB | 399 | NB | 1170 | NB | 1410 | NB | 14.2 |
| P238D | | | | | | | | |
| hIgG4 delta b | n/a | 6.2 | 5.67 | 46.2 | 1860 | 350 | 3240 | 12.1 |
| hIgG4 delta b | n/a | 994 | NB | NB | NB | NB | NB | 17.3 |
| P238D | | | | | | | | |
| hIgG4 delta c | NB | 53.5 | 73.7 | 64.4 | 697 | 127 | 2290 | 14.5 |
| hIgG4 delta c | NB | 502 | NB | 826 | NB | 4460 | NB | 18.7 |
| P238D | | | | | | | | |
| hIgG1 K322A | 0.0052 | n/a | 4.6 | 11 | 5.75 | 2.18 | 11.9 | n/a |
| hIgG1 N297A | 8.7 | 447 | 2150 | 1080 | NB | NB | NB | 9.41 |
| mouse IgG1 | NB | 35.1 | 648 | 2510 | NB | NB | NB | 18.1 |
| D265A | | | | | | | | |
| mouse IgG1 | NB | 0.127 | 2.3 | 5.77 | 273 | 1860 | NB | n/a |

### Example 24. Inhibition of T Cell Activation by Humanised BTLA Agonists in an NFkB Reporter Assay is Dependent on Fc Receptor Binding

BTLA is an inhibitory receptor expressed on T cells and so agonist antibodies against BTLA might be expected to inhibit T cell activation by inducing inhibitory signalling through the receptor. The ability of selected humanised BTLA agonist antibodies to inhibit T cell activation was assessed using a BTLA transfected reporter T cell line. A Jurkat T cell line stably transfected with an expression cassette that includes NF-κB-responsive transcriptional elements upstream of a minimal CMV promoter (mCMV)-GFP cassette (Source BioSciences #TR850A-1) was used as a reporter cell line for NFkB signalling. A lentiviral transfection system was used to express full length human BTLA in this reporter cell line. These cells were mixed with a stimulator cell line comprised of bw5147 cells expressing an anti-CD3 ScFv construct on their surface as described by Leitner et al. (J Immunol Methods. 362(1-2):131-41, 2010). The stimulator cell line was also transfected with human FcγR2B to provide Fc receptors for presentation of the agonist BTLA antibodies. 5×10⁴ reporter cells per well were mixed in 96 well U-bottom plates with 5×10⁴ stimulator cells in the presence of various concentrations of BTLA antibody or hIgG1k isotype control antibody (Sino Biologicals cat #HG1K). After 24 hours incubation at 37° C., cells were pelleted and stained for flow cytometry with a viability dye (Zombie Aqua, Biolegend #423101) and a mouse CD45 antibody (Pe-Cy7 conjugated clone 104, Biolegend #109830) to separate stimulator (murine) from responder (human) cells. Geometric mean of GFP expression was assessed for each antibody concentration and normalized to GFP expression in the absence of antibody.

Humanised 2.8.6 was tested on a hIgG4 isotype, as well as a hIgG1 P238D isotype and a hIgG1 V9 (P238D G237D P271G A330R) isotype. 2.8.6 hIgG1 P238D led to more effective inhibition of NFkB signal than the 2.8.6 hIgG4, and 2.8.6 hIgG1 V9 led to more effective inhibition still (FIG. 15a). Therefore, in conditions where FcγR2B is the only Fc receptor present increasing affinity for FcγR2B confers superior agonistic activity upon BTLA agonist antibodies. When the same antibodies were tested in a modified version of the assay in which the stimulator cells do not express FcγR2B no inhibitory effect of any antibody was seen, confirming that the antibody agonism of BTLA is dependent on FcR engagement by the antibodies (FIG. 15b). The mouse IgG1 parent antibodies of 2.8.6, 6.2 and 3E8 were also able to inhibit T cell activation in the reporter assay when human FcγR2B was expressed on the stimulator cells, in fitting with the cross-reactivity between mIgG1 and hFcγR2B observed in Example 23.

Humanised 2.8.6, 6.2_var_C and 3E8_var_B were all produced on a hIgG1 P238D isotype and compared in the T cell reporter assay described above. They were also compared against the prior art BTLA agonist 22B3 (expressed on a hIgG4PAA isotype) as described in WO 2018/213113 and a fusion protein of the natural BTLA ligand HVEM fused to a mIgG1 Fc region (hHVEM-mFc, produced recombinantly in house; hHVEM-mFc fusion protein including signal peptide and C-terminal His-tag has the sequence disclosed in SEQ ID NO: 229). All three of the humanised P238D variant antibodies demonstrated significantly greater inhibition of NFkB signal compared to 22B3 or hHVEM-mFc (FIG. 16a). 3E8_var_B inhibited NFkB signal by up to 54% with an IC50 of 65 pM. 6.2_var_C inhibited NFkB signal by up to 47% with an IC50 of 28 pM. 2.8.6 inhibited by up to 42% with an IC50 of 59 pM. 22B3 inhibited NFkB signal by up to 18% with an IC50 of 3.8 nM. hHVEM-mFc inhibited NFkB signal by up to 27% with an IC50 of 9.6 nM. Therefore, in conditions where FcγR2B is the only Fc receptor present humanised 2.8.6 hIgG1 P238D, 6.2_var_C hIgG1 P238 and 3E8 hIgG1 P238D are all significantly more efficacious and potent agonists of BTLA than the prior art antibody 22B3 hIgG4PAA and deliver a stronger signal than the endogenous ligand HVEM as an Fc fusion protein.

### Example 25. Inhibition of Primary Human T Cell Proliferation in a Mixed Lymphocyte Reaction by Humanised BTLA Agonists

The ability of selected BTLA agonist antibodies to inhibit human T cell proliferation was assessed in the context of a mixed lymphocyte reaction (MLR). Briefly, human primary T cells were isolated from healthy donor peripheral blood mononuclear cells (PBMCs) using human Pan T cell isolation kit (Miltenyi Biotec cat #130-096-535) and stained with a cell proliferation tracking dye, Tag-it Violet (Biolegend cat #425101). Allogeneic monocyte-derived dendritic cells (DC) were generated by culturing CD14+ monocytes isolated from PBMCs using a CD14+ isolation kit (Miltenyi Biotec cat #130-050-201). CD14+ monocytes were treated with human recombinant IL-4 (Peprotech cat #200-04) and GM-CSF (Biolegend cat #572904) for 7 days. DC maturation was then induced by adding human recombinant TNF-α (Biolegend cat #717904) for an additional 2 days. Mature dendritic cells express both activating and inhibitory FcγRs (Guilliams et al. Nature Reviews Immunology 14(2): 94-108, 2014. http://www.nature.com/articles/nri3582 (May 18, 2020)).

MLR was then performed by co-culturing 1×10⁵ total T cells with allogeneic mature DCs at a ratio of 4:1 (T:DC) in flat-bottom 96-well plates. T cells and DCs were incubated for 5 days with no antibody or in the presence of different doses of BTLA agonist antibody (2.8.6 hIgG1 P238D, 2.8.6 hIgG1 V9, 2.8.6 IgG4), a hIgG1k isotype control antibody (Sino Biologicals cat #HG1K), or the prior art BTLA agonist 22B3 hIgG4PAA. After 5 days T cell proliferation was evaluated by flow cytometry. T cells were harvested and stained with anti-CD3 antibody (PerCP/Cy5.5 conjugated clone OKT3, Biolegend cat #317336), anti-CD4 antibody (BB515 conjugated clone RPA-T4, BD Horizon cat #564419), anti-CD8 antibody (BV510 conjugated clone SK1, BD Horizon cat #563919) together with a viability dye (Zombie NIR, Biolegend cat #423105) and acquired on a BD FACSCelesta instrument. CD4 proliferation (measured as the percentage of CTV low cells) in the presence of antibody was normalised to the average proliferation in the absence of antibody. FIG. 17 shows data combined from 6 separate MLRs with different PBMC donors. Antibody 2.8.6 on the hIgG1 P238D isotype significantly inhibited CD4 T cell proliferation with an average inhibitory effect of 51% at 10 μg/ml. Antibody 2.8.6 on either the hIgG1 V9 isotype or the hIgG4 isotype had no inhibitory effect. Therefore, unexpectedly, in conditions where multiple Fc receptors are present the hIgG1 P238D isotype, which selectively binds to FcγR2B, confers superior agonistic activity onto BTLA agonists than other isotypes tested. The ineffectiveness in this setting of the hIgG1 V9 isotype, which binds FcγR2B with a ˜30-fold higher affinity than the P238D isotype, might be due to activatory signalling through FcγR2A(131R) to which it also retains significant binding. Alternatively, the ineffectiveness of the hIgG1 V9 isotype might be due to the stable formation of cis interactions between antibody, BTLA and FcγR2B on the same cell surface (for example on dendritic cells which express both receptors), which might not induce signalling but would block the formation of productive trans interactions between antibody, BTLA and FcγR2B on different cells. The lower affinity of the P238D isotype for FcγR2B might mean that if these cis interactions form, they are shorter lived and do not completely block trans interactions.

The 22B3 hIgG4PAA also had no inhibitory effect in the mixed lymphocyte reaction, and in fact trended towards increasing proliferation of CD4 T cells, which could be explained by the antibody blocking the natural inhibitory signalling through BTLA by interfering with its interaction with the ligand HVEM. The antibodies 6.2, 3E8 and 286 bind to an epitope on BTLA that does not overlap with the HVEM binding interface and so these antibodies do not block the BTLA-HVEM interaction (Example 19).

### Example 26. Inhibition of Primary Human B Cell Activation by BTLA Agonists

The ability of BTLA agonist antibodies to inhibit primary human B cell activation was evaluated. B cells express high levels of both BTLA and FcγR2B.

Human primary B cells were isolated from healthy donor peripheral blood mononuclear cells using human B cell isolation kit (Miltenyi Biotec cat #130-050-301) and stained with a cell proliferation tracking dye, Tag-it Violet™ (Biolegend cat #425101). 1×10⁵ B cells per well of a 96 well flat bottom plate were then stimulated with 0.01 μM of the TLR9 agonist ODN2006 (InvivoGen cat #tlrl-2006-1), in the presence or absence of different doses of isotype control antibody or selected BTLA agonist antibodies. BTLA agonist 2.8.6, 6.2_var_C and 3E8_var_B (all hIgG1 P238D isotype) were tested and compared against the prior art BTLA agonist 22B3 hIgG4PAA. A recombinant HVEM fusion protein (hHVEM-mFc, produced in house) was used as a positive control. After 5 days of incubation at 37° C., B cells were harvested and stained with anti-CD20 antibody (PE-CF594 conjugated clone 2H7, BD Horizon #562295) together with a viability dye (Zombie NIR, Biolegend #423105) to evaluate their proliferation by flow cytometry. In addition, culture supernatant was collected to assess by ELISA the production of IL-6 (rndsystems cat #DY206) and IL-10 (rndsystems cat #DY217B).

Following procedures essentially as described above, BTLA agonist antibodies were able to inhibit B cell proliferation as efficiently as the hHVEM-mFc positive control. In addition, all three antibody variants demonstrated significantly greater inhibition of B cell proliferation compared to 22B3. Furthermore, the P238D BTLA agonists impaired the production of IL-10 (FIG. 18) and IL-6 by activated B cells. Consistent with the proliferation data the ability of P238D BTLA antibodies to inhibit IL-10 and IL-6 production was greater compared to the 22B3 antibody.

### Example 27. Treatment of a Xenogeneic Model of Graft-Versus-Host Disease (GVHD)

Prevention of human PBMC-driven graft vs. host disease (GvHD) was determined in vivo. Briefly, female NSG mice (JAX Labs, Stock #05557), approximately 8-10 weeks old (n=10 mice per treatment group) were irradiated with 2.4Gy total body irradiation. Human peripheral blood mononuclear cells (PBMCs) were isolated from a leukopak (a HemaCare product ordered via Tissue Solutions) and resuspended at 50×10⁶ cells per ml of PBS. Mice are injected with 200 μl cell suspension (10×10⁶ PBMCs) intravenously (IV) by tail injection 1 day after irradiation. The following day mice are treated with 10 mg/kg of test antibody by intraperitoneal injection. Mice are weighed regularly and euthanised when they have lost 15% body weight or after 28 days. At study termination infiltration of human PBMCs into lung, liver and spleen is quantified by flow cytometry using markers for hCD45, hCD4, hCD8, hCD20, hCD25 and FOXP3.

Following procedures as described above humanised 2.8.6 hIgG1 P238D, 6.2_var_C hIgG1 P238D and 3E8_var_B hIgG1 P238D all significantly reduced weight loss compared to hIgG1 P238D isotype control (FIG. 19) and led to a significant reduction in infiltrating human immune cells in lung, liver and spleen. A trend to increased frequency of regulatory T cells was also observed with all three BTLA agonists.

### Example 28. In Vivo Half-Life of P238D Mutated hIgG1 Antibody in Cynomolgus Macaques and Prediction of Human Half Life

The in vivo half-life of 6.2_var_C on a hIgG1 P238D isotype in cynomolgus macaques was evaluated. 2 female macaques were injected IV with 3 mg/kg of the antibody and 2 female macaques were injected with 10 mg/kg of the antibody. Macaques were bled before antibody injection, and at 1 hour, 6 hours, 24 hours, 48 hours, 72 hours, 168 hours, 240 hours, 336 hours, 432 hours and 504 hours after antibody injection. The concentration of 6.2_var_C in serum at each of these time points was assessed by target capture ELISA. A 96 well microplate (Thermoscientific Cat #439454) was coated overnight at 4° C. with 100 μl of human BTLA extracellular domain at 1 ug/ml in PBS. The plate was then washed 3 times with wash buffer (PBS with 0.05% Tween 20 (ThermoScientific Cat #28320)), and wells were blocked for 1 hour at room temperature with 300 μl SuperBlock buffer (Thermoscientific Cat #37515), followed by again washing 3 times with wash buffer. 100 μl of serum samples diluted in ELISA buffer (PBS, 1% Bovine Serum Albumin, 0.05% Tween 20) were then added and incubated for 1 hour at room temperature. An 11-point standard curve of 6.2_var_C at known concentrations in ELISA buffer was performed in duplicate and duplicate wells containing only ELISA buffer used as blanks. Following incubation, wells were washed 3 times with wash buffer, then HRP-conjugated anti-human detection antibody (Abcam Cat #ab98624) diluted 1 in 20,000 in ELISA buffer was added and incubated for 1 hour at room temperature. Wells were again washed 3 times with wash buffer then 100 μL of Ultra TMB-ELISA Substrate Solution (ThermoScientific Cat #34028) was added per well. Incubated for 90 seconds with a covering of foil to ensure the plate was not in direct light then 50 μL of stop solution (ThermoScientific Cat #SSO4) added per well. Absorbance at 450 nm then read on a Thermo MultiSkan FC. Concentrations interpolated from standard curve using GraphPad Prism software.

Using the serum antibody concentrations at each time point, pharmacokinetics in each monkey was fitted with a 2-compartment model (Dirks et al. Clin. Pharmacokinet 49(10):633-659, 2010). The average terminal half-life in macaques was calculated as 5.4 days (130 hours). The model parameters (the volumes of distribution V1 and V2, clearance C1 and inter-compartmental exchange coefficient Q) were then scaled to human using allometric scaling. With allometric scaling the parameter₁ for a species with a body weight BW₁ is estimated from the parameter₂ from another species with body weight BW₂ with the equation:

$${parameter}_{1} = {parameter}_{2} \cdot {( \frac{B {W}_{1}}{B {W}_{2}} )}^{β}$$

where β is the scaling coefficient for the given parameter. This approach is well documented and has been shown to provide adequate predictions in human from preclinical species (Dong et al Clin Pharmacokinet, 50(2):131-142, 2011) and (Wang et al. Biopharmaceutics & drug disposition, 31:253-263, 2010).

For humans, a body weight of 70 kg was assumed. For cynomolgus monkeys, a reference body weight of 3 kg was used. Theoretical scaling exponents for large molecules were used: β=1 for V1 and V2, β3=0.75 for C1 (as described in Kleiber et al. Hilgardia 6(11): 315-333. 1932) and β=2/3 for Q. For the scaling of the inter-compartmental exchange coefficient Q, it was assumed that the rate of exchange of the compound depended on the surface area of the vascular endothelium. This assumption was based on the implementation of the inter-compartmental exchange which is written as:

Q·(cₚ−cₜ)=P·S(cₚ−cₜ)

where cₚ−cₜ is the concentration difference across the vascular boundary, P is the vascular permeability coefficient with units (m/s) and S is the surface area in units (m²) of the vasculature that is involved in the exchange. It was assumed that the vascular permeability P is a property of the molecule, and that it is independent of the species. The only difference between species is the vascular surface which is scaled with a coefficient of 2/3 with body weight. With these arguments, the assumed scaling value for Q is 2/3.

The predicted terminal half-life in human was then computed from the scaled parameters using a 2-compartment model. The average predicted half-life in humans was calculated as 12.5 days (300 hours).

### Certain Embodiments of the Invention

1. An isolated antibody that specifically binds to human BTLA, wherein said antibody comprises a heavy chain and a light chain, wherein said heavy chain comprises an Fc region that comprises a substitution that results in increased binding to FcγR2B compared to a parent molecule that lacks the substitution.
2. The antibody according to embodiment 1, wherein the antibody has selectivity for binding FcγR2B over FcγR2A compared to a parent molecule that lacks the substitution.
3. The antibody according to embodiment 1 or 2, wherein the antibody has:
(i) enhanced FcγR2B binding activity and maintained or decreased binding activities towards FcγR2A (type R) and/or FcγR2A (type H) in comparison with a parent polypeptide; and/or
(ii) a value of [KD value of polypeptide variant for FcγR2A (type R)]/[KD value of polypeptide variant for FcγR2B] of 2 or more, such as 3, 4, 5, 6, 7, 8, 9, 10 or more; and/or
(iii) a value of [KD value of polypeptide variant for FcγR2A (type H)]/[KD value of polypeptide variant for FcγR2B] of 2 or more, such as 3, 4, 5, 6, 7, 8, 9, 10 or more; and/or
(iv) enhanced FcγR2B binding activity and maintained or decreased binding activities towards FcγR1A in comparison with a parent polypeptide; and/or
(v) a value of [KD value of polypeptide variant for FcγR1A]/[KD value of polypeptide variant for FcγR2B] of 2 or more, such as 3, 4, 5, 6, 7, 8, 9, 10 or more.
4. The antibody of any of embodiments 1 to 3, wherein the antibody binds a residue of human BTLA selected from:
(i) D52, P53, E55, E57, E83, Q86, E103, L106 and E92 (position according to SEQ ID NO:225); or
(ii) Y39, K41, R42, Q43, E45 and S47; or
(iii) D35, T78, K81, S121 and L123; or
(iv) H68; or
(v) N65 and A64;
   wherein each position is in relation to the amino acid sequence disclosed in SEQ ID NO:225.
   5. An antibody that specifically binds to human BTLA, wherein said antibody comprises a heavy chain and a light chain, wherein said heavy chain comprises an Fc region that comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index).
   6. Then antibody of embodiment 5, wherein said heavy chain comprises an Fc region that comprises an aspartic acid at position 238 (EU Index).
   7. The antibody of any one of the preceding embodiments, which is an agonistic antibody.
   8. The antibody of embodiments 6, wherein said antibody binds to FcγR2B with a higher affinity relative to a comparable control antibody that comprises an Fc region that comprises a proline at position 238 (EU Index).
   9. The antibody of any one of the preceding embodiments, wherein said antibody binds to FcγR2B with an affinity of from about 5 μM to 0.1 μM, as determined by surface plasmon resonance (SPR).
   10. The antibody of any one of the preceding embodiments, wherein said antibody binds to FcγR2B with an affinity of at most 5 μM, as determined by surface plasmon resonance (SPR).
   11. The antibody of any one of embodiments 6 to 10, wherein said antibody binds to FcγR2A (131R allotype) with a lower or equal affinity relative to a comparable control antibody that comprises an Fc region that comprises a proline at position 238 (EU Index).
   12. The antibody of any one of the preceding embodiments, wherein said antibody binds to FcγR2A (131R allotype) with a K_{D} of at least 20 μM, as determined by surface plasmon resonance (SPR).
   13. The antibody of any one of embodiments 6 to 12, wherein said antibody binds to FcγR2A (131H allotype) with a lower or equal affinity relative to a comparable control antibody that comprises an Fc region that comprises a proline at position 238 (EU Index).
   14. The antibody of any preceding embodiment, wherein said antibody binds to FcγR2A (131H allotype) with a K_{D} of at least 50 μM, as determined by surface plasmon resonance (SPR).
   15. The antibody of any one of the preceding embodiments, wherein said antibody exhibits increased agonism of human BTLA expressed on the surface of a human immune cell as measured by a BTLA agonist assay selected from a T cell activation assay such as that described in example 24, a mixed lymphocyte reaction such as that described in example 25 or a B cell activation assay such as that described in example 26.
   16. An isolated antibody that specifically binds to human BTLA, wherein said antibody comprises a heavy chain and a light chain, wherein: the heavy chain comprises an Fc region and a heavy chain variable region comprising three complementarity determining regions (CDRs): CDRH1, CDRH2 and CDRH3 and the light chain comprises a light chain variable region comprising three CDRs: CDRL1, CDRL2, and CDRL3, wherein (1) CDRH1, CDRH2, CDRH3 have an amino acid sequence as set forth in SEQ ID NO: 1, SEQ ID NO: 17, and SEQ ID NO: 3, respectively, with from 0 to 3 amino acid modification, and CDRL1, CDRL2, and CDRL3 have an amino acid sequence as set forth in SEQ ID NO: 4, SEQ ID NO: 12, and SEQ ID NO: 6, respectively, with from 0 to 3 amino acid modifications; or (2) CDRH1, CDRH2, CDRH3 have an amino acid sequence as set forth in SEQ ID NO: 20, SEQ ID NO: 21, and SEQ ID NO: 22, respectively, with from 0 to 3 amino acid modification, and CDRL1, CDRL2, and CDRL3 have an amino acid sequence as set forth in SEQ ID NO: 23, SEQ ID NO: 24, and SEQ ID NO: 25, respectively, with from 0 to 3 amino acid modifications; or (3) CDRH1, CDRH2, CDRH3 have an amino acid sequence as set forth in SEQ ID NO: 30, SEQ ID NO: 31, and SEQ ID NO: 32, respectively, with from 0 to 3 amino acid modification, and CDRL1, CDRL2, and CDRL3 have an amino acid sequence as set forth in SEQ ID NO: 33, SEQ ID NO: 34, and SEQ ID NO: 35, respectively, with from 0 to 3 amino acid modifications, and wherein the Fc region comprises an aspartic acid at position 238 (EU Index).
   17. An isolated antibody that specifically binds BTLA, comprising a heavy chain and a light chain, wherein (1) the heavy chain comprises a heavy chain variable region comprising an amino acid sequence as set forth in SEQ ID NO: 18, or a sequence with at least 90% identity thereto and an Fc region comprising an aspartic acid at position 238 (EU Index) and the light chain comprises a light chain variable region comprising an amino acid sequence as set forth in SEQ ID NO: 14, or a sequence with at least 90% identity thereto; or (2) the heavy chain comprises a heavy chain variable region comprising an amino acid sequence as set forth in SEQ ID NO: 26, or a sequence with at least 90% identity thereto and an Fc region comprising an aspartic acid at position 238 (EU Index) and the light chain comprises a light chain variable region comprising an amino acid sequence as set forth in SEQ ID NO: 27, or a sequence with at least 90% identity thereto; or (3) the heavy chain comprises a heavy chain variable region comprising an amino acid sequence as set forth in SEQ ID NO: 36, or a sequence with at least 90% identity thereto and an Fc region comprising an aspartic acid at position 238 (EU Index) and the light chain comprises a light chain variable region comprising an amino acid sequence as set forth in SEQ ID NO: 43, or a sequence with at least 90% identity thereto.
   18. An isolated antibody that specifically binds BTLA, comprising a heavy chain and a light chain, wherein (1) the heavy chain comprises an amino acid sequence as set forth in SEQ ID NO: 19, or a sequence with at least 90% sequence identity thereto, and the light chain comprises an amino acid sequence as set forth in SEQ ID NO: 16, or a sequence with at least 90% identity thereto; (2) the heavy chain comprises an amino acid sequence as set forth in SEQ ID NO: 28, or a sequence with at least 90% sequence identity thereto, and the light chain comprises an amino acid sequence as set forth in SEQ ID NO: 29, or a sequence with at least 90% identity thereto; or (3) the heavy chain comprises an amino acid sequence as set forth in SEQ ID NO: 38, or a sequence with at least 90% sequence identity thereto, and the light chain comprises an amino acid sequence as set forth in SEQ ID NO: 44, or a sequence with at least 90% identity thereto; and wherein for each of 1) (2) and (3) the heavy chain comprises an aspartic acid at position 238 (EU Index).
   19. The antibody of any one of the preceding embodiments, which is an IgG1, IgG2 or IgG4 antibody.
   20. The antibody of any one of the preceding embodiments, which is selected from the group consisting of: a human antibody, a humanised antibody, a chimeric antibody and a multispecific antibody (such as a bispecific antibody).
   21. The antibody of any one of the preceding embodiments, which is monoclonal.
   22. The antibody of any one of the preceding embodiments, wherein said antibody agonizes human BTLA expressed on the surface of an immune cell, wherein said immune cell is optionally a T cell.
   23. The antibody of any one of the preceding embodiments, wherein binding of said antibody to human BTLA expressed on the surface of an immune cell decreases proliferation of said cell relative to a comparable immune cell not bound by said antibody, and wherein said cell is optionally a T cell.
   24. The antibody of embodiment 23, wherein said decrease in cell proliferation is at least about 10%, 15%, 20%, 25%, 30%, 40%, or 50%.
   25. The antibody of embodiment 23, wherein said decrease in cell proliferation is from about 10% to 50%, 10% to 40%, 10% to 30%, 10% to 20%, 10% to 15%, 20% to 50%, 20% to 40%, or 20% to 30%.
   26. The antibody of any one of the preceding embodiments, wherein said antibody comprises a domain that binds to an Fc receptor.
   27. The antibody of any one of the preceding embodiments, wherein said Fc receptor is expressed on the surface of an immune cell.
   28. The antibody of embodiment 27, wherein said immune cell is an antigen presenting cell.
   29. The antibody of embodiment 28, wherein said antigen presenting cell is a dendritic cell, macrophage, monocyte, or neutrophil.
   30. The antibody of any one of the preceding embodiments, wherein said antibody binds to human BTLA expressed on the surface of a T cell.
   31. The antibody of any one of embodiments 26 to 30, wherein said Fc receptor is FcγR2B.
   32. The antibody of any one of the preceding embodiments, wherein binding of said antibody to human BTLA expressed on the surface of an immune cell decreases NFκB signaling of said immune cell relative to a comparable immune cell not bound by said antibody, and wherein said immune cell is optionally a T cell.
   33. The antibody of embodiment 32, wherein said decrease in NFκB signaling of said immune cell is measured by an assay described in Example 10.
   33. The antibody of embodiment 32 or 33, wherein said decrease in NFκB signaling of said immune cell is at least about 10%, 15%, 20%, 25%, 30%, or 40%.
   34. The antibody of embodiment 32 or 33, wherein said decrease in NFκB signaling of said immune cell is from about 10% to 40%, 10% to 30%, 10% to 20%, 20% to 40%, or 20% to 30%.
   35. The antibody of any one of the preceding embodiments, wherein binding of said antibody to human BTLA expressed on the surface of an immune cell decreases dephosphorylation of a cytoplasmic domain of said human BTLA.
   36. The antibody of embodiment 35, wherein said dephosphorylation is mediated by CD45 expressed on the surface of said immune cell.
   37. The antibody of any one of the preceding embodiments, wherein said antibody specifically binds human B and T Lymphocyte Attenuator (BTLA) with a K_{D} of less than 10 nM, each as determined by surface plasmon resonance (SPR) at 37° C., and wherein said antibody binds cynomolgus BTLA with a K_{D} of less than 20 nM, as determined by surface plasmon resonance (SPR) at 37° C.; does not inhibit binding of BTLA to herpes virus entry mediator (HVEM); and inhibits proliferation of T cells in vitro, as determined by a mixed lymphocyte reaction assay.
   38. The antibody of embodiment 37, wherein said antibody binds human B and T Lymphocyte Attenuator (BTLA) with an on rate of at least 5.0×10⁵ (l/Ms) at 37° C.
   39. The antibody of embodiment 37 or 38, wherein said antibody binds human B and T Lymphocyte Attenuator (BTLA) with an off rate of less than 3.0×10⁻⁴ (l/s) at 37° C.
   40. The antibody of any one of embodiments 37 to 39, wherein said antibody binds human B and T Lymphocyte Attenuator (BTLA) with an off rate from 3.0×10⁻⁴ (l/s) to 1.0×10⁻³ (l/s).
   41. The antibody of any one of the preceding embodiments, wherein said antibody specifically binds human B and T Lymphocyte Attenuator (BTLA) with an on rate of at least 5.0×10⁵ (l/Ms), as determined by surface plasmon resonance (SPR) at 37° C., wherein said antibody does not inhibit binding of BTLA to herpes virus entry mediator (HVEM); and wherein said antibody inhibits proliferation of T cells in vitro, as determined by a mixed lymphocyte reaction assay.
   42. The antibody of any one of the preceding embodiments, wherein said antibody binds human B and T Lymphocyte Attenuator (BTLA) with a K_{D} of less than 10 nM, as determined by surface plasmon resonance (SPR) at 37° C.
   43. The antibody of any one of the preceding embodiments, wherein said antibody binds cynomolgus BTLA with a K_{D} of less than 20 nM, as determined by surface plasmon resonance (SPR) at 37° C.
   44. The antibody of any one of the preceding embodiments, wherein said antibody specifically binds human B and T Lymphocyte Attenuator (BTLA) with an off rate from 3.0×10⁻⁴ (l/Ms) to 1.0×10⁻³ (l/Ms) as measured by surface plasmon resonance (SPR) at 37° C., wherein said antibody does not inhibit binding of BTLA to herpes virus entry mediator (HVEM); and wherein said antibody inhibits proliferation of T cells in vitro, as determined by a mixed lymphocyte reaction assay.
   45. The antibody of any one of the preceding embodiments, wherein said antibody specifically binds human B and T Lymphocyte Attenuator (BTLA) with an off rate of less than 1.0×10⁻³ (l/Ms) and an on rate of at least 5.0×10⁵ (l/Ms), each as measured by surface plasmon resonance (SPR) at 37° C., wherein said antibody does not inhibit binding of BTLA to herpes virus entry mediator (HVEM); and wherein said antibody inhibits proliferation of T cells in vitro, as determined by a mixed lymphocyte reaction assay.
   46. The antibody of any one of the preceding embodiments, wherein said antibody specifically binds human B and T Lymphocyte Attenuator (BTLA) with a K_{D} of less than 2 nM, as determined by surface plasmon resonance (SPR) at 37° C., wherein said antibody inhibits binding of BTLA to herpes virus entry mediator (HVEM); and inhibits proliferation of T cells in vitro, as determined by a mixed lymphocyte reaction assay.
   47. The antibody of any one of the preceding embodiments, wherein said antibody specifically binds human B and T Lymphocyte Attenuator (BTLA), wherein said antibody binds cynomolgus BTLA with a K_{D} of at least 5 nM, as determined by surface plasmon resonance (SPR) at 37° C.; and wherein said antibody inhibits binding of BTLA to herpes virus entry mediator (HVEM); and inhibits proliferation of T cells in vitro, as determined by a mixed lymphocyte reaction assay.
   48. The antibody of any one of the preceding embodiments, wherein said antibody specifically binds human B and T Lymphocyte Attenuator (BTLA), wherein said antibody binds cynomolgus BTLA with a K_{D} of at least than 50 nM, as determined by surface plasmon resonance (SPR) at 37° C.; and wherein said antibody does not inhibit binding of BTLA to herpes virus entry mediator (HVEM); and inhibits proliferation of T cells in vitro, as determined by a mixed lymphocyte reaction assay.
   49. The antibody of any one of the preceding embodiments, which has an in vivo half-life of at least 7 days in the human body.
   50. A nucleic acid which comprises one or more nucleotide sequences encoding polypeptides capable of forming an antibody of any of embodiments 1 to 49.
   51. An expression vector comprising the nucleic acid molecule of embodiment 50.
   52. A host cell comprising the nucleic acid sequence of embodiment 50 or 51.
   53. A method of producing an antibody (or BTLA binding molecule) that binds to BTLA, comprising the step of culturing the host cell of embodiment 52 under conditions for production of said antibody, optionally further comprising isolating and/or purifying said antibody.
   54. A method for preparing a human antibody (or BTLA binding molecule) that specifically binds BTLA, the method comprising the steps of:
(i) providing a host cell comprising one or more nucleic acid molecules encoding the amino acid sequence of a heavy chain and a light chain which when expressed are capable of combining to create an antibody of any one of embodiments 1 to 49;
(ii) culturing the host cell expressing the encoded amino acid sequence; and
(iii) isolating the antibody.
   55. A pharmaceutical composition comprising a therapeutically effective amount of the antibody of any one of embodiments 1 to 49 and at least one pharmaceutically acceptable excipient.
   56. An antibody in accordance with any one of embodiments 1 to 49, or the pharmaceutical composition in accordance with embodiment 55, for use in therapy.
   57. An antibody in accordance with any one of embodiments 1 to 49, or the pharmaceutical composition in accordance with embodiment 55, for use in the treatment or prevention of inflammatory or autoimmune diseases, and disorders of excessive immune cell proliferation.
   58. The antibody for use according to embodiment 56, wherein the inflammatory or autoimmune disease is selected from Addison's disease, allergy, alopecia areata, amyotrophic lateral sclerosis, ankylosing spondylitis, anti-phospholipid syndrome, asthma (including allergic asthma), autoimmune haemolytic anaemia, autoimmune hepatitis, autoimmune pancreatitis, autoimmune polyendocrine syndrome, Behcet's disease, bullous pemphigoid, cerebral malaria, chronic inflammatory demyelinating polyneuropathy, coeliac disease, Crohn's disease, Cushing's Syndrome, dermatomyositis, diabetes mellitus type 1, eosinophilic granulomatosis with polyangiitis, graft versus host disease, Graves' disease, Guillain-Barre syndrome, Hashimoto's thyroiditis, Hidradenitis Suppurativa, inflammatory fibrosis (e.g., scleroderma, lung fibrosis, and cirrhosis), juvenile arthritis, Kawasaki disease, leukemia, lymphoma, lymphoproliferative disorders, multiple sclerosis, myasthenia gravis, myeloma, neuromyelitis optica, pemphigus, polymyositis, primary biliary cholangitis, primary sclerosing cholangitis, psoriasis, psoriatic arthritis, rheumatoid arthritis, sarcoidosis, Sjögren's syndrome, systemic lupus erythematosus, Takayasu's arteritis, temporal arteritis, transplant rejection, transverse myelitis, ulcerative colitis, uveitis, vasculitis, vitiligo and Vogt-Koyanagi-Harada Disease.
   59. The antibody for use according to embodiment 57, wherein the disorder of excessive immune cell proliferation is selected from lymphoma, leukemia, systemic mastocytosis, myeloma, or a lymphoproliferative disorder.
   60. An isolated antibody that specifically binds B and T lymphocyte attenuator (BTLA), comprising a heavy chain and a light chain, wherein the heavy chain comprises a heavy chain variable region comprising three CDRs: CDRH1, CDRH2 and CDRH3, wherein (i) CDRH1, CDRH2, CDRH3 have an amino acid sequence as set forth in SEQ ID NO: 1, SEQ ID NO: 17, and SEQ ID NO: 3, respectively, and wherein the light chain comprises a light chain variable region comprising three CDRs: CDRL1, CDRL2 and CDRL3, wherein CDRL1 has an amino acid sequence as set forth in SEQ ID NO: 4, CDRL2 has an amino acid sequence as set forth in SEQ ID NO: 12, and CDRL3 has an amino acid sequence as set forth in SEQ ID NO: 6; and wherein said heavy chain comprises an aspartic acid at position 238 (EU Index).
   61. An isolated antibody that specifically binds BTLA, comprising a heavy chain and a light chain, wherein the heavy chain comprises a heavy chain variable region comprising an amino acid sequence as set forth in SEQ ID NO: 18, and wherein the heavy chain comprises an aspartic acid at position 238 (EU Index).
   62. The isolated antibody according to embodiment 61, wherein the light chain comprises a light chain variable region comprising an amino acid sequence as set forth in SEQ ID NO: 14, or a sequence with at least 90% identity thereto.
   63. The isolated antibody according to any one of embodiments 60 to 62, wherein the heavy chain comprises the amino acid sequence as set forth in SEQ ID NO: 19 and the light chain comprises an amino acid sequence as set forth in SEQ ID NO: 16.
   64. An isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 20, CDRH2 has an amino acid sequence as set forth in SEQ ID NO: 21, and CDRH3 has an amino acid sequence as set forth in SEQ ID NO: 22, and the light chain comprises a light chain variable region comprising three CDRs: CDRL1, CDRL2 and CDRL3, wherein CDRL1 has an amino acid sequence as set forth in SEQ ID NO: 23, CDRL2 has an amino acid sequence as set forth in SEQ ID NO: 24, and CDRL3 has an amino acid sequence as set forth in SEQ ID NO: 25; and wherein said heavy chain comprises an aspartic acid at position 238 (EU Index).
   65. The isolated antibody according to embodiment 64, wherein the heavy chain comprises the amino acid sequence as set forth in SEQ ID NO: 28 and the light chain comprises an amino acid sequence as set forth in SEQ ID NO: 29.
   66. An isolated antibody that specifically binds human BTLA, comprising a heavy chain and a light chain, wherein the heavy chain comprises a heavy chain variable region comprising three CDRs: CDRH1, CDRH2 and CDRH3, wherein CDRH1 has an amino acid sequence as set forth in SEQ ID NO: 30, CDRH2 has an amino acid sequence as set forth in SEQ ID NO: 31, and CDRH3 has an amino acid sequence as set forth in SEQ ID NO: 32, and the light chain comprises a light chain variable region comprising three CDRs: CDRL1, CDRL2 and CDRL3, wherein CDRL1 has an amino acid sequence as set forth in SEQ ID NO: 33, CDRL2 has an amino acid sequence as set forth in SEQ ID NO: 34, and CDRL3 has an amino acid sequence as set forth in SEQ ID NO: 35; and wherein said heavy chain comprises an aspartic acid at position 238 (EU Index).
   67. The isolated antibody according to embodiment 66, wherein the heavy chain comprises the amino acid sequence as set forth in SEQ ID NO: 38 and the light chain comprises an amino acid sequence as set forth in SEQ ID NO: 39.
   68. The antibody of any one of embodiments 60 to 67 or 82-84, which is an IgG1, IgG2 or IgG4 antibody.
   69. The antibody of any one of embodiments 60 to 68 or 82 to 84, which is selected from the group consisting of: a human antibody, a humanised antibody, a chimeric antibody and a multispecific antibody (such as a bispecific antibody).
   70. The antibody of any one of embodiments 60 to 69 or 82 to 84, which is an antigen-binding fragment moiety selected from the group consisting of: scFv, sc(Fv)2, dsFv, Fab, Fab′, (Fab′)2 and a diabody.
   71. The antibody of any one of embodiments 60 to 70 or 82 to 84, which is monoclonal.
   72. The antibody of any one of embodiments 60 to 71 or 82 to 84, wherein said antibody agonizes human BTLA expressed on the surface of an immune cell, wherein said immune cell is optionally a T cell.
   73. The antibody of any one of embodiments 60 to 72 or 82 to 84, wherein binding of said antibody to human BTLA expressed on the surface of an immune cell decreases proliferation of said cell relative to a comparable immune cell not bound by said antibody, and wherein said cell is optionally a T cell.
   74. An isolated nucleic acid which comprises one or more nucleotide sequences encoding polypeptides capable of forming an antibody in any of embodiments 60 to 73 or 82 to 84.
   75. A host cell comprising the nucleic acid sequence according to embodiment 74.
   76. A method of producing an antibody that binds to BTLA, comprising the step of culturing the host cell of embodiment 75, under conditions for production of said antibody, optionally further comprising isolating and/or purifying said antibody.
   77. A method for preparing a human antibody that specifically binds BTLA, the method comprising the steps of:
i) providing a host cell comprising one or more nucleic acid molecules encoding the amino acid sequence of a heavy chain and a light chain which when expressed are capable of combining to create an antibody of any of embodiments 60 to 73 or 82 to 84;
ii) culturing the host cell expressing the encoded amino acid sequence; and
iii) isolating the antibody.
   78. A pharmaceutical composition comprising a therapeutically effective amount of the antibody of any of embodiments 60 to 73 or 82 to 84 and at least one pharmaceutically acceptable excipient.
   79. An antibody in accordance with any one of embodiments 60 to 73 or 82 to 84, or the pharmaceutical composition in accordance with embodiment 78, for use in therapy.
   80. An antibody in accordance with any one of embodiments 60 to 73 or 82 to 84, or the pharmaceutical composition in accordance with embodiment 78, for use in the treatment or prevention of inflammatory or autoimmune diseases, and disorders of excessive immune cell proliferation
   81. The antibody for use according to claim 80, wherein the inflammatory or autoimmune disease is selected from Addison's disease, allergy, alopecia areata, amyotrophic lateral sclerosis, ankylosing spondylitis, anti-phospholipid syndrome, asthma (including allergic asthma), autoimmune haemolytic anaemia, autoimmune hepatitis, autoimmune pancreatitis, autoimmune polyendocrine syndrome, Behcet's disease, bullous pemphigoid, cerebral malaria, chronic inflammatory demyelinating polyneuropathy, coeliac disease, Crohn's disease, Cushing's Syndrome, dermatomyositis, diabetes mellitus type 1, eosinophilic granulomatosis with polyangiitis, graft versus host disease, Graves' disease, Guillain-Barre syndrome, Hashimoto's thyroiditis, Hidradenitis Suppurativa, inflammatory fibrosis (e.g., scleroderma, lung fibrosis, and cirrhosis), juvenile arthritis, Kawasaki disease, leukemia, lymphoma, lymphoproliferative disorders, multiple sclerosis, myasthenia gravis, myeloma, neuromyelitis optica, pemphigus, polymyositis, primary biliary cholangitis, primary sclerosing cholangitis, psoriasis, psoriatic arthritis, rheumatoid arthritis, sarcoidosis, Sjögren's syndrome, systemic lupus erythematosus, Takayasu's arteritis, temporal arteritis, transplant rejection, transverse myelitis, ulcerative colitis, uveitis, vasculitis, vitiligo and Vogt-Koyanagi-Harada Disease.
   82. An isolated antibody that specifically binds BTLA, comprising a heavy chain and a light chain, wherein (1) the heavy chain comprises a heavy chain variable region comprising an amino acid sequence as set forth in SEQ ID NO: 18, or a sequence with at least 90% identity thereto and the light chain comprises a light chain variable region comprising an amino acid sequence as set forth in SEQ ID NO: 14, or a sequence with at least 90% identity thereto.
   83. An isolated antibody that specifically binds BTLA, comprising a heavy chain and a light chain, wherein (1) the heavy chain comprises an amino acid sequence as set forth in SEQ ID NO: 19, or a sequence with at least 90% sequence identity thereto and light chain comprises an amino acid sequence as set forth in SEQ ID NO: 16, or a sequence with at least 90% identity thereto.
   84. An isolated human antibody that specifically binds B and T lymphocyte attenuator (BTLA), comprising a heavy chain and a light chain, wherein
   (a) the heavy chain comprises a heavy chain variable region comprising three CDRs: CDRH1, CDRH2 and CDRH3, wherein CDRH1, CDRH2, CDRH3 have an amino acid sequence as set forth in SEQ ID NO: 1, SEQ ID NO: 17, and SEQ ID NO: 3, respectively, and wherein the light chain comprises a light chain variable region comprising three CDRs: CDRL1, CDRL2 and CDRL3, wherein CDRL1 has an amino acid sequence as set forth in SEQ ID NO: 4, CDRL2 has an amino acid sequence as set forth in SEQ ID NO: 12, and CDRL3 has an amino acid sequence as set forth in SEQ ID NO: 6; and/or
   (b) the heavy chain comprises a heavy chain variable region comprising an amino acid sequence as set forth in SEQ ID NO: 18; or a sequence with at least 90% identity thereto; and/or (c) the light chain comprises a light chain variable region comprising an amino acid sequence as set forth in SEQ ID NO: 14, or a sequence with at least 90% identity thereto;
   optionally wherein the antibody is an IgG1, IgG2 or IgG4 antibody.

### Sequences:

TABLE 7
Exemplary CDR Sequences

| SEQ | | SEQ | |
|---|---|---|---|
| ID | Amino Acid | ID | Amino Acid |
| NOs | Sequences | NOs | Sequences |
| 45 | SYGIS | 136 | WQGTHFPQT |
| 46 | EIYPRSGNTY | 139 | TYYGSSQYYF |
| | YNEKFKG | | DY |
| 47 | NYGSSYPFAY | 143 | DYYIN |
| 33 | SASSSVSSSY | 144 | RIYPGSGNTY |
| | LH | | YNEKFKG |
| 34 | RTSNLAS | 145 | GYGNSDY |
| 35 | QQWSGYPFT | 146 | RASQSIGTRI |
| | | | H |
| 53 | DYYMN | 147 | YASESIS |
| 54 | DINPNNGGTS | 148 | QQSNSWPYT |
| | YNQKFKG | | |
| 55 | WRQLRSDY | 30 | SYAIR |
| 56 | LASQTIGTWL | 48 | EIYPRSGNTY |
| | A | | YNENFKG |
| 57 | AATSLAD | 32 | SGGASYTMDY |
| 58 | QQLYSTPLT | 151 | SYGLI |
| 61 | SYWMH | 152 | EIYPRSGSTY |
| | | | YNEWFKG |
| 62 | MIHPNNGIPN | 153 | RRGTGDGFDY |
| | YNEKFKS | | |
| 63 | EGYYGSEGYF | 154 | SASQGISNYL |
| | DV | | N |
| 64 | SASSSISYIH | 155 | YTSSLHS |
| 65 | DTSKLAS | 156 | QQYIELPFT |
| 66 | HQRSTYPYT | 159 | DYYMH |
| 69 | MIHPNSGSTN | 160 | YIYPNNGGNG |
| | YNEKFKS | | YNQKFKG |
| 70 | KRGGLGDY | 161 | GDYYGSLRLT |
| | | | FAY |
| 71 | RASKSVSTSG | 4 | KSSQSLLYSS |
| | YSYMH | | NQKNYLA |
| 72 | LASNLES | 12 | WASTRES |
| 73 | QHSRELPYT | 164 | QQYYSYPLT |
| 76 | SSWMN | 167 | TYGVS |
| 77 | RIYPGDGDTN | 168 | WINTYSGVPT |
| | YNGKFKG | | YADDFKG |
| 78 | RGYGYLAY | 169 | VTTILHWYFD |
| | | | V |
| 79 | KASQDVSTAV | 170 | RASQEISGYL |
| | A | | S |
| 80 | SASYRYT | 171 | AASTLDS |
| 81 | QQHYSTPYT | 172 | LQYASYPFT |
| 84 | GYGSSYGFAY | 177 | RRGAGDGFDY |
| 85 | QQWSGYPWT | 178 | QQYSKLPFT |
| 88 | SGYYWN | 181 | DHTIH |
| 89 | YISYDGSNNY | 182 | YIYPRDGSTK |
| | NPSLKN | | YNEKFKG |
| 90 | IYGNYYAMDY | 183 | SNWNFDY |
| 91 | SASSSVSYMH | 184 | KASQDVGTAV |
| | | | A |
| 92 | QQWSSNPPT | 185 | WASTRRT |
| 95 | DYYMI | 186 | QQYSSYPLT |
| 96 | NINPNNGGTT | 189 | QQHYSTPWT |
| | YNQKFKG | | |
| 97 | GGLRPLYFDY | 191 | EIYPRSGTTY |
| | | | YNEKFKG |
| 98 | KASENVDTYV | 192 | RISSGSGVDY |
| | S | | |
| 99 | GASNRYT | 193 | QQYSELPWT |
| 100 | GQSYSYPLT | 196 | SGYDWH |
| 103 | NTYMH | 197 | YISYSGSTNY |
| | | | NPSLKS |
| 104 | RIDPANGNTK | 198 | GTPVVAEDYF |
| | YDPKFQG | | DY |
| 105 | TYYGSSQHYF | 199 | RSSTGAVTTS |
| | DY | | NYAN |
| 106 | KSSQSLLDSD | 200 | ATNNRAP |
| | GKTYLN | | |
| 107 | LVSKLDS | 201 | ALWYSNHLV |
| 108 | WQDTHFPQT | 20 | TYGVH |
| 111 | RIYPGDGDAN | 21 | VMWPGGRTSY |
| | YNGKFKG | | NPSLKS |
| 112 | EGHYYGSGYR | 22 | GDYEYDYYAM |
| | WYLDV | | DY |
| 113 | RASENIYSNL | 23 | RASSSVSYMH |
| | A | | |
| 114 | AATNLAD | 24 | ATSNRAT |
| 115 | QHFRGAPFT | 25 | HQWSSNPYT |
| 118 | DYEIH | 204 | SAYWN |
| 119 | PIDPDTGNTA | 205 | YISYSGSTYF |
| | YNQNLKG | | NPSLKS |
| 120 | GGYDSDWGFA | 206 | SHYYGYYFDY |
| | Y | | |
| 121 | RSSKSLLHSN | 207 | RASETIDSYG |
| | GNTFLF | | DSLMH |
| 163 | VMWPGGRTSY | 208 | RASNLES |
| | NPAPMS | | |
| 176 | ATSNLAS | 209 | QQTDEDPYT |
| 122 | RMSDLAS | 1 | SYGMS |
| 123 | MQHLEYPFT | 2 | SIRSDGNTYY |
| | | | PDSVKG |
| 126 | DYYLN | 3 | GGYYGSSPYY |
| 127 | LIDPYNGGSS | 5 | WASTRDS |
| | CNQKFKG | | |
| 128 | GNAMDY | 6 | QQYYNYLT |
| 129 | WASTRHT | 212 | SGYSWH |
| 130 | QQHYIIPYM | 213 | YIHYSGSTNY |
| | | | NPSLKS |
| 133 | NTYMY | 214 | GPHRYDGVWF |
| | | | AY |
| 134 | RIDPANGNTK | 215 | SASSSISSNY |
| | YAPKFQG | | LH |
| 135 | LYYGSSYDYF | 216 | QQGTNIPLT |
| | DY | | |
| 31 | EIYPRSGNTY | 40 | EIYPRSGQTY |
| | YAQKFQG | | YAQSFQG |
| 11 | SIRSEGQTYY | 17 | SIRSDGQTYY |
| | PDSVKG | | PDSVKG |

TABLE 8
Exemplary VH and VL Sequences

| SEQ ID | |
|---|---|
| NOs | Amino Acid Sequences |
| 51 | QVQLQQSGAELARPGASVKLSCKASGYTFTSYGISWVKQRTGQGLEWIGEIYP |
| | RSGNTYYNEKFKGKATLTADKSSSTAYMELRSLTSEDSAVYFCARNYGSSYPF |
| | AYWGQGTLVTVSA |
| 59 | EVQLQQSGPELVKPGASVKISCKASGYTFTDYYMNWVKQSHGKSLEWIGDINP |
| | NNGGTSYNQKFKGKATLTVDKSSSTAYMELRSLTSEDSAVYYCARWRQLRSD |
| | YWGQGTTLTVSS |
| 67 | QVQLQQPGAELVKPRASVKLSCKASGYTFTSYWMHWVKQRPGQGLEWIGMI |
| | HPNNGIPNYNEKFKSKATLTVDKSSTTAYMQLSSLTSEDSAVYHCAREGYYGS |
| | EGYFDVWGTGTTVTVSS |
| 74 | QVQLQQPGAELVKPGASVKLSCKASGYTFTSYWMHWVKQRPGQGLEWIGMI |
| | HPNSGSTNYNEKFKSKATLTVDKSSSTAYMQLSSLTSEDSAVYYCARKRGGLG |
| | DYWGQGTSVTVSS |
| 82 | QVQLQQSGPELVKPGASVKISCKASGYAFSSSWMNWVKQRPGKGLEWIGRIY |
| | PGDGDTNYNGKFKGKATLTADKSSSTAYMQLSSLTSEDSAVYFCARRGYGYL |
| | AYWGQGTLVTVSA |
| 86 | QVQLQQSGAELARPGASVKLSCKASGYTFTSYGISWVKQRTGQGLEWIGEIYP |
| | RSGNTYYNEKFKGKATLTADKSSSTAYMELRSLTSEDSAVYFCARGYGSSYGF |
| | AYWGQGTLVTVSA |
| 93 | DVQLQESGPGLVKPSQSLSLTCSVTGYSITSGYYWNWIRQFPGNKLEWMGYIS |
| | YDGSNNYNPSLKNRISITRDTSKNQFFLKLNSVTTEDTATYYCASIYGNYYAM |
| | DYWGQGTSVTVSS |
| 101 | EVQLQQSGPELVQPGASVKISCKASGYTFTDYYMIWVKQSHGKSLEWIGNINP |
| | NNGGTTYNQKFKGKATLTVDKSSSTAYMGLPSLTSEDSAVYYCARGGLRPLY |
| | FDYWGQGTTLTVSS |
| 109 | EVQLQQSVAELVRPGASVKLSCTASGFNIKNTYMHWVKQRPEQGLEWIGRIDP |
| | ANGNTKYDPKFQGKATITADTSSNTAYVQLSSLTSEDTAIYYCALTYYGSSQH |
| | YFDYWGQGTTLTVSS |
| 116 | QIQLQQSGPELVKPGASVKISCKASGYAFSSSWMNWVKKRPGKGLEWIGRIYP |
| | GDGDANYNGKFKGKATLTADKSSSTAYMQLSSLTSEDSAVYFCAGEGHYYGS |
| | GYRWYLDVWGTGTTVTVSS |
| 124 | QVQLQQSGAELVRPGASVTLSCKASGYTFTDYEIHWVKQTLVHGLEWIGPIDP |
| | DTGNTAYNQNLKGKAILTADKSSSTAYMELRSLTSEDSAVYYCTRGGYDSDW |
| | GFAYWGQGTLVTVSA |
| 131 | EVQLQQSGPVLVKPGASVKMSCKASGYTFTDYYLNWVKQSHGKSLEWIGLID |
| | PYNGGSSCNQKFKGKATLTVDKSSSTAYMDLNSLTSEDSAVYYCARGNAMD |
| | YWGQGTSVTVSS |
| 137 | EVQLQQSVAELVRPGASVKLSCTASGFNIKNTYMYWVKQRPEQGLEWIGRIDP |
| | ANGNTKYAPKFQGKATITADTSSNTAYLQLSSLTSEDTAIYYCALLYYGSSYD |
| | YFDYWGQGTTLTVSS |
| 141 | EVQLQQSVAELVRPGASVKLSCTASGFNIKNTYMHWVKQRPEQGLEWIGRIDP |
| | ANGNTKYAPKFQGKATITADTSSNTAYLQLSSLTSEDTAIYYCALTYYGSSQY |
| | YFDYWGQGTTLTVSS |
| 149 | QVQLKQSGAELVRPGASVKLSCKASGYTFTDYYINWVKQRPGQGLEWIARIYP |
| | GSGNTYYNEKFKGKATLTAEKSSSTAYMQLSSLTSEDSAVYFCARGYGNSDY |
| | WGQGTTLTVSS |
| 223 | QVQLQQSGAELARPGASVRLSCKASGYTFTSYAIRWVKQRTGQGLEWIGEIYP |
| | RSGNTYYNENFKGKATLTADKSSSTAYMELRSLTSEDSAVYFCARSGGASYT |
| | MDYWGQGTSVTVSS |
| 157 | QVQLQQSGAELARPGASVRLSCKASGYTFTSYGLIWLKQRTGQGLEWIGEIYP |
| | RSGSTYYNEWFKGKATLTADKSSNTAFMELRSLTSEDSAVYFCARRRGTGDG |
| | FDYWGQGTILTVSS |
| 165 | EVQLQQSGPELVKPGASVKMSCKASGYTFTDYYMHWVKQSHGKSLEWIGYIY |
| | PNNGGNGYNQKFKGKATLTVDKSSSTAYMELRSLTSEDSAVYYCAIGDYYGS |
| | LRLTFAYWGQGTLVTVSA |
| 173 | QIQLVQSGPELKKPGETVKISCKASGYTFTTYGVSWVKQAPGKVLKWMGWIN |
| | TYSGVPTYADDFKGRFAFSLETSASTAYLQISNLKNEDTATYFCAPVTTILHWY |
| | FDVWGTGTTVTVSS |
| 175 | QVQLQQSGAELARPGASVRLSCKASGYTFTSYGISWVKQRTGQGLEWIGEIYP |
| | RSGNTYYNEKFKGKATLTADKSSSTAYMELRSLTSEDSAVYFCARNYGSSYPF |
| | AYWGQGTLVTVSA |
| 179 | QVQLQQSGAELARPGASVKLSCKASGYTFTSYGISWVKQRTGQGLEWIGEIYP |
| | RSGNTYYNEKFKGKATLTADKSSSTAYMELRSLTSEDSAVYFCARRRGAGDG |
| | FDYWGQGTTLTVSS |
| 187 | QDQLQQSDAELVKPGASVKISCKVSGYTFTDHTIHWMKQRPEQGLEWIGYIYP |
| | RDGSTKYNEKFKGKATLTADKSSSTAYMQLNSLTSEDSAVYFCASSNWNFDY |
| | WGQGTTLTVSS |
| 194 | QVQLQQSGAELARPGASVKLPCKASGYTFTSYGISWVKQRTGQGLEWIGEIYP |
| | RSGTTYYNEKFKGKATLTADKSSSTAYMELRSLTSEDSAVYFCARRISSGSGV |
| | DYWGQGTTLTVSS |
| 202 | DVQLQESGPGMVKPSQSLSLTCTVTGYSITSGYDWHWIRHFPGNKLEWMGYIS |
| | YSGSTNYNPSLKSRISITHDTSKNHFFLKLNSVTTEDTATYYCARGTPVVAEDY |
| | FDYWGQGTTLTVSS |
| 219 | EVKLVESGGGLVKPGGSLKLSCAASGFTLSSYGMSWVRQIPEKRLEWVASIRS |
| | DGNTYYPDSVKGRFIISRDNARNILYLQMSSLRSEDTAMYYCTRGGYYGSSPY |
| | YWGQGTTLTVSS |
| 217 | DVQLQESGPDLVKPSQSLSVTCTVTGYSITSGYSWHWIRQFPGNKLEWMGYIH |
| | YSGSTNYNPSLKSRISITRDTSKNQFFLQLSSVTTEDTATYYCASGPHRYDGVW |
| | FAYWGQGTLVTVSS |
| 52 | ENVLTQSPAIMAASLGQKVTMTCSASSSVSSSYLHWYQQKSGASPKPLIHRTS |
| | NLASGVPARFSGSGSGTSYSLTISSVEAEDDATYYCQQWSGYPFTFGGGTKLEI |
| | K |
| 60 | DIQMTQSPASQSASLGESVTITCLASQTIGTWLAWYQQKPGKSPQLLIYAATSL |
| | ADGVPSRFSGSGSGTKFSFKISSLQAEDFVSYYCQQLYSTPLTFGAGTKLELK |
| 68 | QIVLTQSPAIMSASPGEKVTMTCSASSSISYIHWYQQKPGTSPKRWIYDTSKLAS |
| | GVPARFSGSGSGTSYSLTISSMEAEDAATYYCHQRSTYPYTFGGGTKLEIK |
| 75 | DIVLTQSPASLAVSLGQRATISCRASKSVSTSGYSYMHWYQQKPGQPPKLLIYL |
| | ASNLESGVPARFSGSGSGTDFTLNIHPVEEEDAATYYCQHSRELPYTFGGGTKL |
| | EIK |
| 83 | DIVMTQSHKFMSTSVGDRVSITCKASQDVSTAVAWYQQKPGQSPKLLIYSASY |
| | RYTGVPDRFTGSGSGTDFTFTISSVQAEDLAVYYCQQHYSTPYTFGGGTKLEIK |
| 87 | ENVLTQSPAIMAASLGQKVTMTCSASSSVSSSYLHWYQQKSGASPKPLIHRTS |
| | NLASGVPARFSGSGSGTSYSLTISSVEAEDDATYYCQQWSGYPWTFGGGTKLE |
| | IK |
| 94 | QIVLTQSPAIMSASPGEKVTMTCSASSSVSYMHWYQQKSGTSPKRWIYDTSKL |
| | ASGVPARFSGSGSGTSYSLTISSMEAEDAATYYCQQWSSNPPTFGSGTKLEIK |
| 102 | NIVMTQSPKSMSMSVGERVTLSCKASENVDTYVSWYQQKPEQSPKLLIYGASN |
| | RYTGVPDRFTGSGSATDFTLTISSVQAEDLADYHCGQSYSYPLTFGAGTKLELI |
| 110 | DVVMTQTPLTLSVTIGQPASISCKSSQSLLDSDGKTYLNWLLQRPGQSPKRLIY |
| | LVSKLDSGVPDRFTGSGSGTDFTLKISRVEAEDLGVYYCWQDTHFPQTFGGGT |
| | KLEIK |
| 117 | DIQMTQSPASLSVSVGETVTITCRASENIYSNLAWYQQKQGKSPQLLVYAATN |
| | LADGVPSRFSGSGSGTQYSLKINSLQSEDFGSYYCQHFRGAPFTFGSGTKLEIK |
| 125 | DIVMTQATPSVPVTPGESVSISCRSSKSLLHSNGNTFLFWFLQRPGQSPQLLIYR |
| | MSDLASGVPDRFSGSGSGTAFTLRISRVEAEDVGIYYCMQHLEYPFTFGSGTKL |
| | EIK |
| 132 | DIVMTQSHKFMSTSVGDRVSITCKASQDVSTAVAWYQEKPGQSPKLLIYWAST |
| | RHTGVPDRFTGSGSGTDYILNISSVQAEDLALYYCQQHYIIPYMFGGGTKLEIK |
| 138 | DVVMTQTPLTLSVTIGQPASISCKSSQSLLDSDGKTYLNWLLQRPGQSPKRLIY |
| | LVSKLDSGVPDRFTGSGSGTDFTLKISRVEAEDLGVYYCWQGTHFPQTFGGGT |
| | KLEIK |
| 142 | DILLTQSPAILSVSPGERVSFSCRASQSIGTRIHWYQQRTNGSPRLLIKYASESI |
| | SGIPSRFSGSGSGTDFTLSINSVESEDIADYYCQQSNSWPYTFGGGTKLEIK |
| 150 | ENVLTQSPAIMAASLGQKVTMTCSASSSVSSSYLHWYQQKSGASPKPLIHRTS |
| | NLASGVPARFSGSGSGTSYSLTISSVEAEDDATYYCQQWSGYPFTFGSGTKLEI |
| | K |
| 158 | DIQMTQTTSSLSASLGDRVTISCSASQGISNYLNWYQQKPDGTVKLLIYYTSSL |
| | HSGVPSRFSGSGSGTDYSLTISNLEPEDIATYYCQQYIELPFTFGSGTKLEIK |
| 166 | DIVMSQSPSSLAVSVGEKVTMSCKSSQSLLYSSNQKNYLAWYQQKPGQSPKLL |
| | IYWASTRESGVPDRFTGSGSGTDFTLTISSVKAEDLAVYYCQQYYSYPLTFGAG |
| | TKLELK |
| 174 | DIQMTQSPSSLSASLGERVSLTCRASQEISGYLSWLQQKPDGTIKRLIYAASTLD |
| | SGVPKRFRGSRSGSDYSLTISSLESEDFADYYCLQYASYPFTFGSGTKLEIK |
| 180 | DIQMTQTTSSLSASLGDRVTISCSASQGISNYLNWYQQKPDGTVKLLIYYTSSL |
| | HSGVPSRFSGSGSGTDYSLTISNLEPEDIATYYCQQYSKLPFTFGSGTKLEIK |
| 188 | DIVMTQSHKFMSTSVGDRVSITCKASQDVGTAVAWYQQKPGQSPKLLIYWAS |
| | TRRTGVPDRFTGSGSGTDFTLTISNVQSEDLADYFCQQYSSYPLTFGAGTKLEL |
| | K |
| 190 | DIVMTQSHKFMSTSVGDRVSITCKASQDVSTAVAWYQQKPGQSPKLLIYSASY |
| | RYTGVPDRFTGSGSGTDFTFTISSVQAEDLAVYYCQQHYSTPWTFGGGTKLEIK |
| 195 | DIQMTQTTSSLSASLGDRVTISCSASQGISNYLNWYQQKPDGTVKLLIYYTSSL |
| | HSGVPSRFSGSGSGTDYSLTISNLEPEDIATYYCQQYSELPWTFGGGTKLEIK |
| 203 | QAVVTQESALSTSPGETVTLTCRSSTGAVTTSNYANWVQEKPDHLFTGLIGAT |
| | NNRAPGVPARFSGSLIGDKAALTITGAQTEDEAIYFCALWYSNHLVFGGGTKL |
| | TVLG |
| 220 | DIVMSQSPSSLPVSVGEKISMTCKSSQSLLYSSNQKNYLAWYQQKPGQSPKLLI |
| | YWASTRDSGVPDRFIGSGSGTDFTLTINSVKAEDLAVYYCQQYYNYLTFGAGT |
| | KLELK |
| 218 | EIVLTQSPTTMAASPGEKITITCSASSSISSNYLHWYQQKPGFSPKLLIYRTSNLA |
| | SGVPARFSGSGSGTSYSLTIGTMEAEDVATYYCQQGTNIPLTFGAGTKLEIK |
| 36 | QVQLVQSGAELKKPGASVKVSCKASGYTFTSYAIRWVRQATGQGLEWMGEIY |
| | PRSGNTYYAQKFQGRATLTADKSISTAYMELSSLRSEDTAVYFCARSGGASYT |
| | MDYWGQGTTVTVSS |
| 37 | ENVLTQSPATLSLSPGERATLSCSASSSVSSSYLHWYQQKPGQSPRPLIHRTSNL |
| | ASGIPARFSGSGSGTDYTLTISSLEPEDFAVYYCQQWSGYPFTFGSGTKLEIK |
| 7 | EVQLVESGGGLVKPGGSLRLSCAASGFTLSSYGMSWVRQAPGKGLEWVASIR |
| | SDGNTYYPDSVKGRFTISRDNAKNSLYLQMSSLRAEDTAVYYCTRGGYYGSSP |
| | YYWGQGTTVTVSS |
| 8 | DIVMTQSPLSLPVTPGEPASISCKSSQSLLYSSNQKNYLAWYQQKPGQSPQLLIY |
| | WASTRDSGVPDRFSGSGSGTDFTLKISRVEAEDVGVYYCQQYYNYLTFGGGT |
| | KVEIK |
| 13 | EVQLVESGGGLVKPGGSLRLSCAASGFTLSSYGMSWVRQAPGKGLEWVASIR |
| | SEGQTYYPDSVKGRFTISRDNAKNTLYLQMSSLRAEDTAVYYCTRGGYYGSSP |
| | YYWGQGTTVTVSS |
| 14 | DIVMTQSPLSLPVTPGEPASISCKSSQSLLYSSNQKNYLAWYQQKPGQSPKLLIY |
| | WASTRESGVPDRFSGSGSGTDFTLKISRVEAEDVGVYYCQQYYNYLTFGGGTK |
| | VEIK |
| 18 | EVQLVESGGGLVKPGGSLRLSCAASGFTLSSYGMSWVRQAPGKGLEWVASIR |
| | SDGQTYYPDSVKGRFTISRDNAKNTLYLQMSSLRAEDTAVYYCTRGGYYGSSP |
| | YYWGQGTTVTVSS |
| 26 | QVTLKESGPALVKPTQTLTLTCTVSGFSLTTYGVHWIRQPPGKALEWLGVMW |
| | PGGRTSYNPSLKSRLTITKDNSKSQVVLTMTNMDPVDTATYYCVRGDYEYDY |
| | YAMDYWGQGTLVTVSS |
| 27 | EIVLTQSPATLSLSPGERATLSCRASSSVSYMHWYQQKPGQAPRPLIYATSNRA |
| | TGIPARFSGSGSGTDYTLTISSLEPEDFAVYYCHQWSSNPYTFGQGTKLEIK |
| 41 | QVQLVQSGAELKKPGASVKVSCKASGYTFTSYAIRWVRQATGQGLEWMGEIY |
| | PRSGQTYYAQSFQGRATLTADKSTSTAYMELSSLRSEDTAVYFCARSGGASYT |
| | MDYWGQGTTVTVSS |
| 43 | ENVLTQSPATLSLSLGERATLSCSASSSVSSSYLHWYQQKPDQSPRPLIHRTSNL |
| | ASGIPSRFSGSGSGTDYTLTISSLEAEDFAVYYCQQWSGYPFTFGSGTKLEIK |
| 210 | EVQLQESGPSLVKPSQTLSLTCSVTGDSITSAYWNWIRKFPGNKLEYMGYISYS |
| | GSTYFNPSLKSRISITRNTSKNQYYLQLNSVTTEDTATYYCARSHYYGYYFDY |
| | WGHGTTLTVSS |
| 211 | DIVLTQSPASLAVSLGQRATISCRASETIDSYGDSLMHWYQQKAGQPPKLLIYR |
| | ASNLESGIPARFSGSGSRTDFTLTINPVEADDVATYYCQQTDEDPYTFGGGTKL |
| | EIK |
| 221 | QVQLKESGPGLVAPSQSLSITCTVSGFSLTTYGVHWVRQSPGKGLEWLGVMW |
| | PGGRTSYNPAPMSRLSISKDNSKSQVFLKMNSLQTDDTAMYYCVRGDYEYDY |
| | YAMDYWGQGTSVTVSS |
| 222 | QIVLSQSPAILSASPGEKVTMTCRASSSVSYMHWYQQKPGSSPKPWIYATSNLA |
| | SGVPARFSGSGSGTSYSLTISRMEAEDAATYYCHQWSSNPYTFGGGTKLEIK |

TABLE 9
Exemplary heavy chain and light chain Sequences

| SEQ ID | |
|---|---|
| NOs | Amino Acid Sequences |
| 9 | EVQLVESGGGLVKPGGSLRLSCAASGFTLSSYGMSWVRQAPGKGLEWVASIR |
| | SDGNTYYPDSVKGRFTISRDNAKNSLYLQMSSLRAEDTAVYYCTRGGYYGSS |
| | PYYWGQGTTVTVSS |
| | ASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKT |
| | HTCPPCPAPELLGGDSVFLFPPKPKDTLMISRTPEVTCVVVDVSHEDPEVKFN |
| | WYVDGVEVHNAKTKPREEQYNSTYRVVSVLTVLHQDWLNGKEYKCKVSNK |
| | ALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEAL |
| | HNHYTQKSLSLSPGK |
| 10 | DIVMTQSPLSLPVTPGEPASISCKSSQSLLYSSNQKNYLAWYQQKPGQSPQLLI |
| | YWASTRDSGVPDRFSGSGSGTDFTLKISRVEAEDVGVYYCQQYYNYLTFGGG |
| | TKVEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |
| 15 | EVQLVESGGGLVKPGGSLRLSCAASGFTLSSYGMSWVRQAPGKGLEWVASIR |
| | SEGQTYYPDSVKGRFTISRDNAKNTLYLQMSSLRAEDTAVYYCTRGGYYGSS |
| | PYYWGQGTTVTVSS |
| | ASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKT |
| | HTCPPCPAPELLGGDSVFLFPPKPKDTLMISRTPEVTCVVVDVSHEDPEVKFN |
| | WYVDGVEVHNAKTKPREEQYNSTYRVVSVLTVLHQDWLNGKEYKCKVSNK |
| | ALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEAL |
| | HNHYTQKSLSLSPGK |
| 16 | DIVMTQSPLSLPVTPGEPASISCKSSQSLLYSSNQKNYLAWYQQKPGQSPKLLI |
| | YWASTRESGVPDRFSGSGSGTDFTLKISRVEAEDVGVYYCQQYYNYLTFGGG |
| | TKVEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |
| 19 | EVQLVESGGGLVKPGGSLRLSCAASGFTLSSYGMSWVRQAPGKGLEWVASIR |
| | SDGQTYYPDSVKGRFTISRDNAKNTLYLQMSSLRAEDTAVYYCTRGGYYGSS |
| | PYYWGQGTTVTVSS |
| | ASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKT |
| | HTCPPCPAPELLGGDSVFLFPPKPKDTLMISRTPEVTCVVVDVSHEDPEVKFN |
| | WYVDGVEVHNAKTKPREEQYNSTYRVVSVLTVLHQDWLNGKEYKCKVSNK |
| | ALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEAL |
| | HNHYTQKSLSLSPGK |
| 28 | QVTLKESGPALVKPTQTLTLTCTVSGFSLTTYGVHWIRQPPGKALEWLGVMW |
| | PGGRTSYNPSLKSRLTITKDNSKSQVVLTMTNMDPVDTATYYCVRGDYEYDY |
| | YAMDYWGQGTLVTVSS |
| | ASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKT |
| | HTCPPCPAPELLGGDSVFLFPPKPKDTLMISRTPEVTCVVVDVSHEDPEVKFN |
| | WYVDGVEVHNAKTKPREEQYNSTYRVVSVLTVLHQDWLNGKEYKCKVSNK |
| | ALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEAL |
| | HNHYTQKSLSLSPGK |
| 29 | EIVLTQSPATLSLSPGERATLSCRASSSVSYMHWYQQKPGQAPRPLIYATSNRA |
| | TGIPARFSGSGSGTDYTLTISSLEPEDFAVYYCHQWSSNPYTFGQGTKLEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |
| 38 | QVQLVQSGAELKKPGASVKVSCKASGYTFTSYAIRWVRQATGQGLEWMGEI |
| | YPRSGNTYYAQKFQGRATLTADKSISTAYMELSSLRSEDTAVYFCARSGGAS |
| | YTMDYWGQGTTVTVSS |
| | ASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKT |
| | HTCPPCPAPELLGGDSVFLFPPKPKDTLMISRTPEVTCVVVDVSHEDPEVKFN |
| | WYVDGVEVHNAKTKPREEQYNSTYRVVSVLTVLHQDWLNGKEYKCKVSNK |
| | ALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEAL |
| | HNHYTQKSLSLSPGK |
| 39 | ENVLTQSPATLSLSPGERATLSCSASSSVSSSYLHWYQQKPGQSPRPLIHRTSN |
| | LASGIPARFSGSGSGTDYTLTISSLEPEDFAVYYCQQWSGYPFTFGSGTKLEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |
| 42 | QVQLVQSGAELKKPGASVKVSCKASGYTFTSYAIRWVRQATGQGLEWMGEI |
| | YPRSGQTYYAQSFQGRATLTADKSTSTAYMELSSLRSEDTAVYFCARSGGAS |
| | YTMDYWGQGTTVTVSS |
| | ASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKT |
| | HTCPPCPAPELLGGDSVFLFPPKPKDTLMISRTPEVTCVVVDVSHEDPEVKFN |
| | WYVDGVEVHNAKTKPREEQYNSTYRVVSVLTVLHQDWLNGKEYKCKVSNK |
| | ALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEAL |
| | HNHYTQKSLSLSPGK |
| 44 | ENVLTQSPATLSLSLGERATLSCSASSSVSSSYLHWYQQKPDQSPRPLIHRTSN |
| | LASGIPSRFSGSGSGTDYTLTISSLEAEDFAVYYCQQWSGYPFTFGSGTKLEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |

Human (Homo sapiens) BTLA polypeptide.
Positions 1-30 is signal sequence,
31-151 is extracellular region, 152-178
is transmembrane region and 179 to end
is intracellular region
SEQ ID NO: 225
MKTLPAMLGTGKLFWVFFLIPYLDIWNIHGKESCD
VQLYIKRQSEHSILAGDPFELECPVKYCANRPHVT
WCKLNGTTCVKLEDRQTSWKEEKNISFFILHFEPV
LPNDNGSYRCSANFQSNLIESHSTTLYVTDVKSAS
ERPSKDEMASRPWLLYRLLPLGGLPLLITTCFCLF
CCLRRHQGKQNELSDTAGREINLVDAHLKSEQTEA
STRQNSQVLLSETGIYDNDPDLCFRMQEGSEVYSN
PCLEENKPGIVYASLNHSVIGPNSRLARNVKEAPT
EYASICVRS
cynomolgus monkey (Macaca fascicularis)
BTLA polypeptide.
SEQ ID NO: 226
MKTLPAMLGSGRLFWVVFLIPYLDIWNIHGKESCD
VQLYIKRQSYHSIFAGDPFKLECPVKYCAHRPQVT
WCKLNGTTCVKLEGRHTSWKQEKNLSFFILHFEPV
LPSDNGSYRCSANFLSAIIESHSTTLYVTDVKSAS
ERPSKDEMASRPWLLYSLLPLGGLPLLITTCFCLF
CFLRRHQGKQNELSDTTGREITLVDVPFKSEQTEA
STRQNSQVLLSETGIYDNEPDFCFRMQEGSEVYSN
PCLEENKPGIIYASLNHSIIGLNSRQARNVKEAPT
EYASICVRS
hIgG1 const region with 238D
SEQ ID NO: 227
ASTKGPSVFPLAPSSKSTSGGTAALGCLVKDYFPE
PVTVSWNSGALTSGVHTFPAVLQSSGLYSLSSVVT
VPSSSLGTQTYICNVNHKPSNTKVDKKVEPKSCDK
THTCPPCPAPELLGGDSVFLFPPKPKDTLMISRTP
EVTCVVVDVSHEDPEVKFNWYVDGVEVHNAKTKPR
EEQYNSTYRVVSVLTVLHQDWLNGKEYKCKVSNKA
LPAPIEKTISKAKGQPREPQVYTLPPSREEMTKNQ
VSLTCLVKGFYPSDIAVEWESNGQPENNYKTTPPV
LDSDGSFFLYSKLTVDKSRWQQGNVFSCSVMHEAL
HNHYTQKSLSLSPGK
hkappa const region
SEQ ID NO: 228
RTVAAPSVFIFPPSDEQLKSGTASVVCLLNNFYPR
EAKVQWKVDNALQSGNSQESVTEQDSKDSTYSLSS
TLTLSKADYEKHKVYACEVTHQGLSSPVTKSFNRG
EC
hHVEM-mFc fusion protein (including
signal peptide and C-terminal His-
tag)
SEQ ID NO: 229
MEPPGDWGPPPWRSTPRTDVLRLVLYLTFLGAPCY
APALPSCKEDEYPVGSECCPKCSPGYRVKEACGEL
TGTVCEPCPPGTYIAHLNGLSKCLQCQMCDPAMGL
RASRNCSRTENAVCGCSPGHFCIVQDGDHCAACRA
YATSSPGQRVQKGGTESQDTLCQNCPPGTFSPNGT
LEECQHQTKCSWLVTKAGAGTSSSHLVPRGSGSKP
SISTVPEVSSVFIFPPKPKDVLTITLTPKVTCVVV
DISKDDPEVQFSWFVDDVEVHTAQTQPREEQFNST
FRSVSELPIMHQDWLNGKEFKCRVNSAAFPAPIEK
TISKTKGRPKAPQVYTIPPPKEQMAKDKVSLTCMI
TDFFPEDITVEWQWNGQPAENYKNTQPIMDTDGSY
FVYSKLNVQKSNWEAGNTFTCSVLHEGLHNHHTEK
SLSHSPGKHHHHHH
Mopc21 hIgG1 P238D isotype control
heavy chain
SEQ ID NO: 230
DVQLVESGGGLVQPGGSRKLSCAASGFTFSSFGMH
WVRQAPEKGLEWVAYISSGSSTLHYADTVKGRFTI
SRDNPKNTLFLQMTSLRSEDTAMYYCARWGNYPYY
AMDYWGQGTSVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKKVEPKSCDKTHTCPPCPAPELLGGDSVFL
FPPKPKDTLMISRTPEVTCVVVDVSHEDPEVKFNW
YVDGVEVHNAKTKPREEQYNSTYRVVSVLTVLHQD
WLNGKEYKCKVSNKALPAPIEKTISKAKGQPREPQ
VYTLPPSREEMTKNQVSLTCLVKGFYPSDIAVEWE
SNGQPENNYKTTPPVLDSDGSFFLYSKLTVDKSRW
QQGNVFSCSVMHEALHNHYTQKSLSLSPGK
Mopc21 hIgG1 P238D isotype
control light chain
SEQ ID NO: 231
NIVMTQSPKSMSMSVGERVTLTCKASENVVTYVSW
YQQKPEQSPKLLIYGASNRYTGVPDRFTGSGSATD
FTLTISSVQAEDLADYHCGQGYSYPYTFGGGTKLE
IKRTVAAPSVFIFPPSDEQLKSGTASVVCLLNNFY
PREAKVQWKVDNALQSGNSQESVTEQDSKDSTYSL
SSTLTISKADYEKHKVYACEVTHQGLSSPVTKSFN
RGEC
SEQ ID NO: 232
GGGGS
SEQ ID NO: 233
KESGSVSSEQLAQFRSLD
SEQ ID NO: 234
EGKSSGSGSESKST
Reference IgG4 constant sequence
containing a P238D and also a S228P
substitution.
SEQ ID NO: 235
ASTKGPSVFPLAPCSRSTSESTAALGCLVKDYFPE
PVTVSWNSGALTSGVHTFPAVLQSSGLYSLSSVVT
VPSSSLGTKTYTCNVDHKPSNTKVDKRVESKYGPP
CPPCPAPEFLGGDSVFLFPPKPKDTLMISRTPEVT
CVVVDVSQEDPEVQFNWYVDGVEVHNAKTKPREEQ
FNSTYRVVSVLTVLHQDWLNGKEYKCKVSNKGLPS
SIEKTISKAKGQPREPQVYTLPPSQEEMTKNQVSL
TCLVKGFYPSDIAVEWESNGQPENNYKTTPPVLDS
DGSFFLYSRLTVDKSRWQEGNVFSCSVMHEALHNH
YTQKSLSLSLGK

What is claimed is:

1. An isolated antibody that specifically binds to human B and T lymphocyte attenuator (BTLA), wherein said antibody comprises a heavy chain and a light chain, wherein said heavy chain comprises an Fc region that comprises a substitution that results in increased binding to FcγR2B compared to a parent molecule that lacks the substitution.

2. The antibody according to claim 1, wherein said heavy chain comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index).

3. The antibody according to claim 1, wherein said heavy chain comprises an Fc region that comprises an aspartic acid at position 238 (EU Index).

4. The antibody according to claim 3, wherein (i) said Fc region binds to FcγR2B with a higher affinity relative to a comparable control antibody that comprises an Fc region with proline at position 238 (EU Index); or (ii) said antibody binds to FcγR2B with an affinity of from about 5 μM to 0.1 μM, as determined by surface plasmon resonance (SPR); or (iii) said Fc region binds to FcγR2A (131R allotype) with a lower or equal affinity relative to a comparable control antibody that comprises an Fc region that comprises a proline at position 238 (EU Index); or (iv) said antibody binds to FcγR2A (131R allotype) with a K_{D} of at least 20 μM, as determined by surface plasmon resonance (SPR); or (v) said antibody binds to FcγR2A (131H allotype) with a lower or equal affinity relative to a comparable control antibody that comprises an Fc region that comprises a proline at position 238 (EU Index); or (vi) said antibody binds to FcγR2A (131H allotype) with a K_{D} of at least 50 μM, as determined by surface plasmon resonance (SPR); or (vii) wherein said antibody exhibits an in vivo half-life of at least 10 days.

5. The antibody of any one of claims 1 to 4, wherein said antibody binds to an epitope of human BTLA selected from the group consisting of:
(i) D52, P53, E55, E57, E83, Q86, E103, L106 and E92; or
(ii) Y39, K41, R42, Q43, E45 and S47; or
(iii) D35, T78, K81, S121 and L123; or
(iv) H68; or
(v) N65 and A64;
wherein each position is in relation to the amino acid sequence disclosed in SEQ ID NO:225.

6. The antibody of any one of claims 1 to 5, wherein said antibody exhibits increased agonism of human BTLA expressed on the surface of a human immune cell, such as measured by a BTLA agonist assay selected from a T cell activation assay or a B cell activation assay.

7. The antibody of any one of claims 1 to 6, wherein the heavy chain comprises a heavy chain variable region comprising three complementarity determining regions (CDRs): CDRH1, CDRH2 and CDRH3 and the light chain comprises a light chain variable region comprising three CDRs: CDRL1, CDRL2, and CDRL3, wherein
(i) CDRH1, CDRH2, CDRH3 have an amino acid sequence as set forth in SEQ ID NO: 1, 17, and 3, respectively, with from 0 to 3 amino acid modifications, and CDRL1, CDRL2, and CDRL3 have an amino acid sequence as set forth in SEQ ID NO: 4, 12, and 6, respectively, with from 0 to 3 amino acid modifications; or
(ii) CDRH1, CDRH2, CDRH3 have an amino acid sequence as set forth in SEQ ID NO: 20, 21, and 22, respectively, with from 0 to 3 amino acid modifications, and CDRL1, CDRL2, and CDRL3 have an amino acid sequence as set forth in SEQ ID NO: 23, 24, and 25, respectively, with from 0 to 3 amino acid modifications; or
(iii) CDRH1, CDRH2, CDRH3 have an amino acid sequence as set forth in SEQ ID NO: 30, 31, and 32, respectively, with from 0 to 3 amino acid modifications, and CDRL1, CDRL2, and CDRL3 have an amino acid sequence as set forth in SEQ ID NO: 33, 34, and 35, respectively, with from 0 to 3 amino acid modifications; or
(iv) CDRH1, CDRH2, CDRH3 have an amino acid sequence as set forth in SEQ ID NO: 45, 46, and 47, respectively, with from 0 to 3 amino acid modifications, and CDRL1, CDRL2, and CDRL3 have an amino acid sequence as set forth in SEQ ID NO: 33, 34, and 35, respectively with from 0 to 3 amino acid modifications; or
(v) CDRH1, CDRH2, CDRH3 have an amino acid sequence as set forth in SEQ ID NO: 53, 54, and 55, respectively, with from 0 to 3 amino acid modifications, and CDRL1, CDRL2, and CDRL3 have an amino acid sequence as set forth in SEQ ID NO: 56, 57, and 58, respectively, with from 0 to 3 amino acid modifications; or
(vi) CDRH1, CDRH2, CDRH3 have an amino acid sequence as set forth in SEQ ID NO: 61, 62, and 63, respectively, with from 0 to 3 amino acid modifications, and CDRL1, CDRL2, and CDRL3 have an amino acid sequence as set forth in SEQ ID NO: 64, 65, and 66, respectively, with from 0 to 3 amino acid modifications; or
(vii) CDRH1, CDRH2, CDRH3 have an amino acid sequence as set forth in SEQ ID NO: 61, 69, and 70, respectively, with from 0 to 3 amino acid modifications, and CDRL1, CDRL2, and CDRL3 have an amino acid sequence as set forth in SEQ ID NO: 71, 72, and 73, respectively, with from 0 to 3 amino acid modifications; or
(viii) CDRH1, CDRH2, CDRH3 have an amino acid sequence as set forth in SEQ ID NO: 76, 77, and 78, respectively, with from 0 to 3 amino acid modifications, and CDRL1, CDRL2, and CDRL3 have an amino acid sequence as set forth in SEQ ID NO: 79, 80, and 81, respectively, with from 0 to 3 amino acid modifications; or
(ix) CDRH1, CDRH2, CDRH3 have an amino acid sequence as set forth in SEQ ID NO: 45, 46, and 84, respectively, with from 0 to 3 amino acid modifications, and CDRL1, CDRL2, and CDRL3 have an amino acid sequence as set forth in SEQ ID NO: 33, 34, and 85, respectively, with from 0 to 3 amino acid modifications; or
(x) CDRH1, CDRH2, CDRH3 have an amino acid sequence as set forth in SEQ ID NO: 88, 89, and 90, respectively, with from 0 to 3 amino acid modifications, and CDRL1, CDRL2, and CDRL3 have an amino acid sequence as set forth in SEQ ID NO: 91, 65, and 92, respectively, with from 0 to 3 amino acid modifications; or
(xi) CDRH1, CDRH2, CDRH3 have an amino acid sequence as set forth in SEQ ID NO: 95, 96, and 97, respectively, with from 0 to 3 amino acid modifications, and CDRL1, CDRL2, and CDRL3 have an amino acid sequence as set forth in SEQ ID NO: 98, 99, and 100, respectively, with from 0 to 3 amino acid modifications; or
(xii) CDRH1, CDRH2, CDRH3 have an amino acid sequence as set forth in SEQ ID NO: 103, 104, and 105, respectively, with from 0 to 3 amino acid modifications, and CDRL1, CDRL2, and CDRL3 have an amino acid sequence as set forth in SEQ ID NO: 106, 107, and 108, respectively, with from 0 to 3 amino acid modifications; or
(xiii) CDRH1, CDRH2, CDRH3 have an amino acid sequence as set forth in SEQ ID NO: 76, 111, and 112, respectively, with from 0 to 3 amino acid modifications, and CDRL1, CDRL2, and CDRL3 have an amino acid sequence as set forth in SEQ ID NO: 113, 114, and 115, respectively, with from 0 to 3 amino acid modifications; or
(xiv) CDRH1, CDRH2, CDRH3 have an amino acid sequence as set forth in SEQ ID NO: 118, 119, and 120, respectively, with from 0 to 3 amino acid modifications, and CDRL1, CDRL2, and CDRL3 have an amino acid sequence as set forth in SEQ ID NO: 121, 122, and 123, respectively, with from 0 to 3 amino acid modifications; or
(xv) CDRH1, CDRH2, CDRH3 have an amino acid sequence as set forth in SEQ ID NO: 126, 127, and 128, respectively, with from 0 to 3 amino acid modifications, and CDRL1, CDRL2, and CDRL3 have an amino acid sequence as set forth in SEQ ID NO: 79, 129, and 130, respectively, with from 0 to 3 amino acid modifications; or
(xvi) CDRH1, CDRH2, CDRH3 have an amino acid sequence as set forth in SEQ ID NO: 133, 134, and 135, respectively, with from 0 to 3 amino acid modifications, and CDRL1, CDRL2, and CDRL3 have an amino acid sequence as set forth in SEQ ID NO: 106, 107, and 136, respectively, with from 0 to 3 amino acid modifications; or
(xvii) CDRH1, CDRH2, CDRH3 have an amino acid sequence as set forth in SEQ ID NO: 103, 134, and 139, respectively, with from 0 to 3 amino acid modifications, and CDRL1, CDRL2, and CDRL3 have an amino acid sequence as set forth in SEQ ID NO: 106, 107, and 136, respectively, with from 0 to 3 amino acid modifications; or
(xviii) CDRH1, CDRH2, CDRH3 have an amino acid sequence as set forth in SEQ ID NO: 143, 144, and 145, respectively, with from 0 to 3 amino acid modifications, and CDRL1, CDRL2, and CDRL3 have an amino acid sequence as set forth in SEQ ID NO: 146, 147, and 148, respectively, with from 0 to 3 amino acid modifications; or
(xix) CDRH1, CDRH2, CDRH3 have an amino acid sequence as set forth in SEQ ID NO: 151, 152, and 153, respectively, with from 0 to 3 amino acid modifications, and CDRL1, CDRL2, and CDRL3 have an amino acid sequence as set forth in SEQ ID NO: 154, 155, and 156, respectively, with from 0 to 3 amino acid modifications; or
(xx) CDRH1, CDRH2, CDRH3 have an amino acid sequence as set forth in SEQ ID NO: 159, 160, and 161, respectively, with from 0 to 3 amino acid modifications, and CDRL1, CDRL2, and CDRL3 have an amino acid sequence as set forth in SEQ ID NO: 4, 12, and 164, respectively, with from 0 to 3 amino acid modifications; or
(xx1) CDRH1, CDRH2, CDRH3 have an amino acid sequence as set forth in SEQ ID NO: 167, 168, and 169, respectively, with from 0 to 3 amino acid modifications, and CDRL1, CDRL2, and CDRL3 have an amino acid sequence as set forth in SEQ ID NO: 170, 171, and 172, respectively, with from 0 to 3 amino acid modifications; or
(xxii) CDRH1, CDRH2, CDRH3 have an amino acid sequence as set forth in SEQ ID NO: 45, 46, and 47, respectively, with from 0 to 3 amino acid modifications, and CDRL1, CDRL2, and CDRL3 have an amino acid sequence as set forth in SEQ ID NO: 170, 171, and 172, respectively, with from 0 to 3 amino acid modifications; or
(xxiii) CDRH1, CDRH2, CDRH3 have an amino acid sequence as set forth in SEQ ID NO: 45, 46, and 177, respectively, with from 0 to 3 amino acid modifications, and CDRL1, CDRL2, and CDRL3 have an amino acid sequence as set forth in SEQ ID NO: 154, 155, and 178, respectively, with from 0 to 3 amino acid modifications; or
(xxiv) CDRH1, CDRH2, CDRH3 have an amino acid sequence as set forth in SEQ ID NO: 181, 182, and 183, respectively, with from 0 to 3 amino acid modifications, and CDRL1, CDRL2, and CDRL3 have an amino acid sequence as set forth in SEQ ID NO: 184, 185, and 186, respectively, with from 0 to 3 amino acid modifications; or
(xxv) CDRH1, CDRH2, CDRH3 have an amino acid sequence as set forth in SEQ ID NO: 76, 77, and 78, respectively, with from 0 to 3 amino acid modifications, and CDRL1, CDRL2, and CDRL3 have an amino acid sequence as set forth in SEQ ID NO: 79, 80, and 189, respectively, with from 0 to 3 amino acid modifications; or
(xxvi) CDRH1, CDRH2, CDRH3 have an amino acid sequence as set forth in SEQ ID NO: 45, 191, and 192, respectively, with from 0 to 3 amino acid modifications, and CDRL1, CDRL2, and CDRL3 have an amino acid sequence as set forth in SEQ ID NO: 154, 155, and 193, respectively, with from 0 to 3 amino acid modifications; or
(xxvii) CDRH1, CDRH2, CDRH3 have an amino acid sequence as set forth in SEQ ID NO: 196, 197, and 198, respectively, with from 0 to 3 amino acid modifications, and CDRL1, CDRL2, and CDRL3 have an amino acid sequence as set forth in SEQ ID NO: 199, 200, and 201, respectively, with from 0 to 3 amino acid modifications; or
(xxviii) CDRH1, CDRH2, CDRH3 have an amino acid sequence as set forth in SEQ ID NO: 204, 205, and 206, respectively, with from 0 to 3 amino acid modifications, and CDRL1, CDRL2, and CDRL3 have an amino acid sequence as set forth in SEQ ID NO: 207, 208, and 209, respectively, with from 0 to 3 amino acid modifications; or
(xxix) CDRH1, CDRH2, CDRH3 have an amino acid sequence as set forth in SEQ ID NO: 212, 213, and 214, respectively, with from 0 to 3 amino acid modifications, and CDRL1, CDRL2, and CDRL3 have an amino acid sequence as set forth in SEQ ID NO: 215, 34, and 216, respectively, with from 0 to 3 amino acid modifications; or
(xxx) CDRH1, CDRH2, CDRH3 have an amino acid sequence as set forth in SEQ ID NO: 1, 2, and 3, respectively, with from 0 to 3 amino acid modifications, and CDRL1, CDRL2, and CDRL3 have an amino acid sequence as set forth in SEQ ID NO: 4, 5, and 6, respectively, with from 0 to 3 amino acid modifications; or
(xxxi) CDRH1, CDRH2, CDRH3 have an amino acid sequence as set forth in SEQ ID NO: 20, 163, and 22, respectively, with from 0 to 3 amino acid modifications, and CDRL1, CDRL2, and CDRL3 have an amino acid sequence as set forth in SEQ ID NO: 23, 176, and 25, respectively, with from 0 to 3 amino acid modifications; or
(xxxii) CDRH1, CDRH2, CDRH3 have an amino acid sequence as set forth in SEQ ID NO: 30, 48, and 32, respectively, with from 0 to 3 amino acid modifications, and CDRL1, CDRL2, and CDRL3 have an amino acid sequence as set forth in SEQ ID NO: 33, 34, and 35, respectively, with from 0 to 3 amino acid modifications; or
(xxxiii) CDRH1, CDRH2, CDRH3 have an amino acid sequence as set forth in SEQ ID NO: 1, 11, and 3, respectively, with from 0 to 3 amino acid modifications, and CDRL1, CDRL2, and CDRL3 have an amino acid sequence as set forth in SEQ ID NO: 4, 12, and 6, respectively, with from 0 to 3 amino acid modifications; or
(xxxiv) CDRH1, CDRH2, CDRH3 have an amino acid sequence as set forth in SEQ ID NO: 1, 11, and 3, respectively, with from 0 to 3 amino acid modifications, and CDRL1, CDRL2, and CDRL3 have an amino acid sequence as set forth in SEQ ID NO: 4, 5, and 6, respectively, with from 0 to 3 amino acid modifications; or
(xxxv) CDRH1, CDRH2, CDRH3 have an amino acid sequence as set forth in SEQ ID NO: 1, 17, and 3, respectively, with from 0 to 3 amino acid modifications, and CDRL1, CDRL2, and CDRL3 have an amino acid sequence as set forth in SEQ ID NO: 4, 12, and 6, respectively, with from 0 to 3 amino acid modifications; or
(xxxvi) CDRH1, CDRH2, CDRH3 have an amino acid sequence as set forth in SEQ ID NO: 20, 21, and 22, respectively, with from 0 to 3 amino acid modifications, and CDRL1, CDRL2, and CDRL3 have an amino acid sequence as set forth in SEQ ID NO: 23, 24, and 25, respectively, with from 0 to 3 amino acid modifications; or
(xxxvii) CDRH1, CDRH2, CDRH3 have an amino acid sequence as set forth in SEQ ID NO: 30, 31, and 32, respectively, with from 0 to 3 amino acid modifications, and CDRL1, CDRL2, and CDRL3 have an amino acid sequence as set forth in SEQ ID NO: 33, 34, and 35, respectively, with from 0 to 3 amino acid modifications; or
(xxxviii) CDRH1, CDRH2, CDRH3 have an amino acid sequence as set forth in SEQ ID NO: 30, 40, and 32, respectively, with from 0 to 3 amino acid modifications, and CDRL1, CDRL2, and CDRL3 have an amino acid sequence as set forth in SEQ ID NO: 33, 34, and 35, respectively, with from 0 to 3 amino acid modifications;
optionally wherein the Fc region comprises an aspartic acid at position 238 (EU Index).

8. An isolated antibody that specifically binds BTLA, comprising a heavy chain and a light chain, wherein (1) the heavy chain comprises a heavy chain variable region comprising an amino acid sequence as set forth in SEQ ID NO: 18, or a sequence with at least 90% identity thereto and an Fc region comprising an aspartic acid at position 238 (EU Index) and the light chain comprises a light chain variable region comprising an amino acid sequence as set forth in SEQ ID NO: 14, or a sequence with at least 90% identity thereto; or (2) the heavy chain comprises a heavy chain variable region comprising an amino acid sequence as set forth in SEQ ID NO: 26, or a sequence with at least 90% identity thereto and an Fc region comprising an aspartic acid at position 238 (EU Index) and the light chain comprises a light chain variable region comprising an amino acid sequence as set forth in SEQ ID NO: 27, or a sequence with at least 90% identity thereto; or (3) the heavy chain comprises a heavy chain variable region comprising an amino acid sequence as set forth in SEQ ID NO: 36, or a sequence with at least 90% identity thereto and an Fc region comprising an aspartic acid at position 238 (EU Index) and the light chain comprises a light chain variable region comprising an amino acid sequence as set forth in SEQ ID NO: 43, or a sequence with at least 90% identity thereto.

9. An isolated antibody that specifically binds BTLA, comprising a heavy chain and a light chain, wherein (1) the heavy chain comprises an amino acid sequence as set forth in SEQ ID NO: 19, or a sequence with at least 90% sequence identity thereto and light chain comprises an amino acid sequence as set forth in SEQ ID NO: 16, or a sequence with at least 90% identity thereto; (2) the heavy chain comprises an amino acid sequence as set forth in SEQ ID NO: 28, or a sequence with at least 90% sequence identity thereto and light chain comprises an amino acid sequence as set forth in SEQ ID NO: 29, or a sequence with at least 90% identity thereto; or (3) the heavy chain comprises an amino acid sequence as set forth in SEQ ID NO: 38, or a sequence with at least 90% sequence identity thereto and light chain comprises an amino acid sequence as set forth in SEQ ID NO: 44, or a sequence with at least 90% identity thereto; wherein the heavy chain comprises an Fc region comprising an aspartic acid at position 238 (EU Index).

10. The antibody of any one of the preceding claims, which is an IgG1, IgG2 or IgG4 antibody.

11. The antibody of any one of the preceding claims, which is selected from the group consisting of: a human antibody, a humanised antibody, a chimeric antibody and a multispecific antibody (such as a bispecific antibody).

12. The antibody of any one of the preceding claims, which is monoclonal.

13. The antibody of any one of the preceding claims, wherein said antibody agonizes human BTLA expressed on the surface of an immune cell, optionally wherein said immune cell is a T cell.

14. The antibody of any one of the preceding claims, wherein binding of said antibody to human BTLA expressed on the surface of an immune cell decreases proliferation of said cell relative to a comparable immune cell not bound by said antibody, optionally wherein said cell is a T cell, optionally wherein said decrease in cell proliferation is at least about 10%, 15%, 20%, 25%, 30%, 40%, or 50%.

15. The antibody of any one of the preceding claims, wherein (i) said antibody specifically binds human B and T Lymphocyte Attenuator (BTLA) with a K_{D} of less than 10 nM; and/or (ii) wherein said antibody binds cynomolgus BTLA with a K_{D} of less than 20 nM; and/or (iii) said antibody does not inhibit binding of BTLA to herpes virus entry mediator (HVEM); and/or (iv) said antibody inhibits proliferation of T cells in vitro, as determined by a mixed lymphocyte reaction assay.

16. The antibody of claim 15, wherein said antibody binds human B and T Lymphocyte Attenuator (BTLA) with an on rate of at least 5.0×10⁵ (l/Ms) at 37° C. and/or with an off rate of less than 3.0×10⁴ (l/s) at 37° C. and/or with a K_{D} of less than 10 nM, as determined by surface plasmon resonance (SPR) at 37° C.

17. An isolated human antibody that specifically binds B and T lymphocyte attenuator (BTLA), comprising a heavy chain and a light chain, wherein
(a) the heavy chain comprises a heavy chain variable region comprising three CDRs: CDRH1, CDRH2 and CDRH3, wherein CDRH1, CDRH2, CDRH3 have an amino acid sequence as set forth in SEQ ID NO: 1, SEQ ID NO: 17, and SEQ ID NO: 3, respectively, and wherein the light chain comprises a light chain variable region comprising three CDRs: CDRL1, CDRL2 and CDRL3, wherein CDRL1 has an amino acid sequence as set forth in SEQ ID NO: 4, CDRL2 has an amino acid sequence as set forth in SEQ ID NO: 12, and CDRL3 has an amino acid sequence as set forth in SEQ ID NO: 6; and/or
(b) the heavy chain comprises a heavy chain variable region comprising an amino acid sequence as set forth in SEQ ID NO: 18; or a sequence with at least 90% identity thereto; and/or
(c) the light chain comprises a light chain variable region comprising an amino acid sequence as set forth in SEQ ID NO: 14, or a sequence with at least 90% identity thereto;
wherein the heavy chain comprises an Fc region that comprises an aspartic act at position 238 (EU Index);
optionally wherein the antibody is an IgG1, IgG2 or IgG4 antibody.

18. A nucleic acid which comprises one or more nucleotide sequences encoding polypeptides capable of forming an antibody in any of claims 1 to 17.

19. An expression vector comprising the nucleic acid molecule of claim 18.

20. A host cell comprising the nucleic acid sequence of claim 18 or 19.

21. A method of producing an antibody that binds to BTLA, comprising the step of culturing the host cell of claim 20 under conditions for production of said antibody, optionally further comprising isolating and/or purifying said antibody.

22. A method for preparing an antibody that specifically binds BTLA, the method comprising the steps of:
(i) providing a host cell comprising one or more nucleic acid molecules encoding the amino acid sequence of a heavy chain and a light chain which when expressed are capable of combining to create an antibody molecule of any one of claims 1 to 17;
(ii) culturing the host cell expressing the encoded amino acid sequence; and
(iii) isolating the antibody.

23. A pharmaceutical composition comprising a therapeutically effective amount of the antibody of any one of claims 1 to 17 and at least one pharmaceutically acceptable excipient.

24. An antibody in accordance with any one of claims 1 to 17, or the pharmaceutical composition in accordance with claim 23, for use in therapy.

25. An antibody in accordance with any one of claims 1 to 17, or the pharmaceutical composition in accordance with claim 23, for use in the treatment or prevention of inflammatory or autoimmune diseases, and disorders of excessive immune cell proliferation.

26. The antibody for use according to claim 25, wherein the inflammatory or autoimmune disease is selected from Addison's disease, allergy, alopecia areata, amyotrophic lateral sclerosis, ankylosing spondylitis, anti-phospholipid syndrome, asthma (including allergic asthma), autoimmune haemolytic anaemia, autoimmune hepatitis, autoimmune pancreatitis, autoimmune polyendocrine syndrome, Behcet's disease, bullous pemphigoid, cerebral malaria, chronic inflammatory demyelinating polyneuropathy, coeliac disease, Crohn's disease, Cushing's Syndrome, dermatomyositis, diabetes mellitus type 1, eosinophilic granulomatosis with polyangiitis, graft versus host disease, Graves' disease, Guillain-Barre syndrome, Hashimoto's thyroiditis, Hidradenitis Suppurativa, inflammatory fibrosis (e.g., scleroderma, lung fibrosis, and cirrhosis), juvenile arthritis, Kawasaki disease, leukemia, lymphoma, lymphoproliferative disorders, multiple sclerosis, myasthenia gravis, myeloma, neuromyelitis optica, pemphigus, polymyositis, primary biliary cholangitis, primary sclerosing cholangitis, psoriasis, psoriatic arthritis, rheumatoid arthritis, sarcoidosis, Sjögren's syndrome, systemic lupus erythematosus, Takayasu's arteritis, temporal arteritis, transplant rejection, transverse myelitis, ulcerative colitis, uveitis, vasculitis, vitiligo and Vogt-Koyanagi-Harada Disease.

27. The antibody for use according to claim 25, wherein the disorder of excessive immune cell proliferation is selected from lymphoma, leukemia, systemic mastocytosis, myeloma, or a lymphoproliferative disorder.

28. A method of treating a BTLA-related disease in a patient, comprising administering to the patient a therapeutically effective amount of the antibody of any one of claims 1-17 or the pharmaceutical composition of claim 23.

29. The method of claim 28, wherein the BTLA-related disease is an inflammatory or autoimmune disease, or an immunoproliferative disease or disorder.

30. The method of claim 29, wherein the inflammatory or autoimmune disease is selected from Addison's disease, allergy, alopecia areata, amyotrophic lateral sclerosis, ankylosing spondylitis, anti-phospholipid syndrome, asthma (including allergic asthma), autoimmune haemolytic anaemia, autoimmune hepatitis, autoimmune pancreatitis, autoimmune polyendocrine syndrome, Behcet's disease, bullous pemphigoid, cerebral malaria, chronic inflammatory demyelinating polyneuropathy, coeliac disease, Crohn's disease, Cushing's Syndrome, dermatomyositis, diabetes mellitus type 1, eosinophilic granulomatosis with polyangiitis, graft versus host disease, Graves' disease, Guillain-Barre syndrome, Hashimoto's thyroiditis, Hidradenitis Suppurativa, inflammatory fibrosis (e.g., scleroderma, lung fibrosis, and cirrhosis), juvenile arthritis, Kawasaki disease, leukemia, lymphoma, lymphoproliferative disorders, multiple sclerosis, myasthenia gravis, myeloma, neuromyelitis optica, pemphigus, polymyositis, primary biliary cholangitis, primary sclerosing cholangitis, psoriasis, psoriatic arthritis, rheumatoid arthritis, sarcoidosis, Sjögren's syndrome, systemic lupus erythematosus, Takayasu's arteritis, temporal arteritis, transplant rejection, transverse myelitis, ulcerative colitis, uveitis, vasculitis, vitiligo and Vogt-Koyanagi-Harada Disease.

31. The method of claim 29, wherein the immunoproliferative disease or disorder is selected from lymphoma, leukemia, systemic mastocytosis, myeloma, or a lymphoproliferative disorder.


SEQUENCE LISTING (235 sequences)

SEQ ID NO: 1 - protein, 5 aa, Artificial Sequence
  CDRH1
SYGMS

SEQ ID NO: 2 - protein, 16 aa, Artificial Sequence
  CDRH2
SIRSDGNTYYPDSVKG

SEQ ID NO: 3 - protein, 10 aa, Artificial Sequence
  CDRH3
GGYYGSSPYY

SEQ ID NO: 4 - protein, 17 aa, Artificial Sequence
  CDRL1
KSSQSLLYSSNQKNYLA

SEQ ID NO: 5 - protein, 7 aa, Artificial Sequence
  CDRL2
WASTRDS

SEQ ID NO: 6 - protein, 8 aa, Artificial Sequence
  CDRL3
QQYYNYLT

SEQ ID NO: 7 - protein, 118 aa, Artificial Sequence
  heavy chain variable region
EVQLVESGGGLVKPGGSLRLSCAASGFTLSSYGMSWVRQAPGKGLEWVASIRSDGNTYYPDSVKGRFTISRDNAKNSLYLQMSSLRAEDTAVYYCTRGGYYGSSPYYWGQGTTVTVSS

SEQ ID NO: 8 - protein, 112 aa, Artificial Sequence
  light chain variable region
DIVMTQSPLSLPVTPGEPASISCKSSQSLLYSSNQKNYLAWYQQKPGQSPQLLIYWASTRDSGVPDRFSGSGSGTDFTLKISRVEAEDVGVYYCQQYYNYLTFGGGTKVEIK

SEQ ID NO: 9 - protein, 448 aa, Artificial Sequence
  heavy chain
EVQLVESGGGLVKPGGSLRLSCAASGFTLSSYGMSWVRQAPGKGLEWVASIRSDGNTYYPDSVKGRFTISRDNAKNSLYLQMSSLRAEDTAVYYCTRGGYYGSSPYYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial Sequence
  light chain
DIVMTQSPLSLPVTPGEPASISCKSSQSLLYSSNQKNYLAWYQQKPGQSPQLLIYWASTRDSGVPDRFSGSGSGTDFTLKISRVEAEDVGVYYCQQYYNYLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 16 aa, Artificial Sequence
  CDRH2 of the humanised antibody 6.2 modified with N56Q and D54E substitutions
SIRSEGQTYYPDSVKG

SEQ ID NO: 12 - protein, 7 aa, Artificial Sequence
  CDRL2 of humanized 6.2 modified with a D61E substitution
WASTRES

SEQ ID NO: 13 - protein, 118 aa, Artificial Sequence
  heavy chain variable region
EVQLVESGGGLVKPGGSLRLSCAASGFTLSSYGMSWVRQAPGKGLEWVASIRSEGQTYYPDSVKGRFTISRDNAKNTLYLQMSSLRAEDTAVYYCTRGGYYGSSPYYWGQGTTVTVSS

SEQ ID NO: 14 - protein, 112 aa, Artificial Sequence
  light chain variable region
DIVMTQSPLSLPVTPGEPASISCKSSQSLLYSSNQKNYLAWYQQKPGQSPKLLIYWASTRESGVPDRFSGSGSGTDFTLKISRVEAEDVGVYYCQQYYNYLTFGGGTKVEIK

SEQ ID NO: 15 - protein, 448 aa, Artificial Sequence
  heavy chain
EVQLVESGGGLVKPGGSLRLSCAASGFTLSSYGMSWVRQAPGKGLEWVASIRSEGQTYYPDSVKGRFTISRDNAKNTLYLQMSSLRAEDTAVYYCTRGGYYGSSPYYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 219 aa, Artificial Sequence
  light chain
DIVMTQSPLSLPVTPGEPASISCKSSQSLLYSSNQKNYLAWYQQKPGQSPKLLIYWASTRESGVPDRFSGSGSGTDFTLKISRVEAEDVGVYYCQQYYNYLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 16 aa, Artificial Sequence
  CDRH2
SIRSDGQTYYPDSVKG

SEQ ID NO: 18 - protein, 118 aa, Artificial Sequence
  heavy chain variable region
EVQLVESGGGLVKPGGSLRLSCAASGFTLSSYGMSWVRQAPGKGLEWVASIRSDGQTYYPDSVKGRFTISRDNAKNTLYLQMSSLRAEDTAVYYCTRGGYYGSSPYYWGQGTTVTVSS

SEQ ID NO: 19 - protein, 448 aa, Artificial Sequence
  heavy chain
EVQLVESGGGLVKPGGSLRLSCAASGFTLSSYGMSWVRQAPGKGLEWVASIRSDGQTYYPDSVKGRFTISRDNAKNTLYLQMSSLRAEDTAVYYCTRGGYYGSSPYYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 5 aa, Artificial Sequence
  CDRH1
TYGVH

SEQ ID NO: 21 - protein, 16 aa, Artificial Sequence
  CDRH2
VMWPGGRTSYNPSLKS

SEQ ID NO: 22 - protein, 12 aa, Artificial Sequence
  CDRH3
GDYEYDYYAMDY

SEQ ID NO: 23 - protein, 10 aa, Artificial Sequence
  CDRL1
RASSSVSYMH

SEQ ID NO: 24 - protein, 7 aa, Artificial Sequence
  CDRL2
ATSNRAT

SEQ ID NO: 25 - protein, 9 aa, Artificial Sequence
  CDRL3
HQWSSNPYT

SEQ ID NO: 26 - protein, 120 aa, Artificial Sequence
  heavy chain variable region
QVTLKESGPALVKPTQTLTLTCTVSGFSLTTYGVHWIRQPPGKALEWLGVMWPGGRTSYNPSLKSRLTITKDNSKSQVVLTMTNMDPVDTATYYCVRGDYEYDYYAMDYWGQGTLVTVSS

SEQ ID NO: 27 - protein, 106 aa, Artificial Sequence
  light chain variable region
EIVLTQSPATLSLSPGERATLSCRASSSVSYMHWYQQKPGQAPRPLIYATSNRATGIPARFSGSGSGTDYTLTISSLEPEDFAVYYCHQWSSNPYTFGQGTKLEIK

SEQ ID NO: 28 - protein, 450 aa, Artificial Sequence
  heavy chain
QVTLKESGPALVKPTQTLTLTCTVSGFSLTTYGVHWIRQPPGKALEWLGVMWPGGRTSYNPSLKSRLTITKDNSKSQVVLTMTNMDPVDTATYYCVRGDYEYDYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 213 aa, Artificial Sequence
  light chain
EIVLTQSPATLSLSPGERATLSCRASSSVSYMHWYQQKPGQAPRPLIYATSNRATGIPARFSGSGSGTDYTLTISSLEPEDFAVYYCHQWSSNPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 5 aa, Artificial Sequence
  CDRH1
SYAIR

SEQ ID NO: 31 - protein, 17 aa, Artificial Sequence
  CDRH2
EIYPRSGNTYYAQKFQG

SEQ ID NO: 32 - protein, 10 aa, Artificial Sequence
  CDRH3
SGGASYTMDY

SEQ ID NO: 33 - protein, 12 aa, Artificial Sequence
  CDRL1
SASSSVSSSYLH

SEQ ID NO: 34 - protein, 7 aa, Artificial Sequence
  CDRL2
RTSNLAS

SEQ ID NO: 35 - protein, 9 aa, Artificial Sequence
  CDRL3
QQWSGYPFT

SEQ ID NO: 36 - protein, 119 aa, Artificial Sequence
  heavy chain variable region
QVQLVQSGAELKKPGASVKVSCKASGYTFTSYAIRWVRQATGQGLEWMGEIYPRSGNTYYAQKFQGRATLTADKSISTAYMELSSLRSEDTAVYFCARSGGASYTMDYWGQGTTVTVSS

SEQ ID NO: 37 - protein, 108 aa, Artificial Sequence
  light chain variable region
ENVLTQSPATLSLSPGERATLSCSASSSVSSSYLHWYQQKPGQSPRPLIHRTSNLASGIPARFSGSGSGTDYTLTISSLEPEDFAVYYCQQWSGYPFTFGSGTKLEIK

SEQ ID NO: 38 - protein, 449 aa, Artificial Sequence
  heavy chain
QVQLVQSGAELKKPGASVKVSCKASGYTFTSYAIRWVRQATGQGLEWMGEIYPRSGNTYYAQKFQGRATLTADKSISTAYMELSSLRSEDTAVYFCARSGGASYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 215 aa, Artificial Sequence
  light chain
ENVLTQSPATLSLSPGERATLSCSASSSVSSSYLHWYQQKPGQSPRPLIHRTSNLASGIPARFSGSGSGTDYTLTISSLEPEDFAVYYCQQWSGYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 17 aa, Artificial Sequence
  VH CDR
EIYPRSGQTYYAQSFQG

SEQ ID NO: 41 - protein, 119 aa, Artificial Sequence
  heavy chain variable region
QVQLVQSGAELKKPGASVKVSCKASGYTFTSYAIRWVRQATGQGLEWMGEIYPRSGQTYYAQSFQGRATLTADKSTSTAYMELSSLRSEDTAVYFCARSGGASYTMDYWGQGTTVTVSS

SEQ ID NO: 42 - protein, 449 aa, Artificial Sequence
  heavy chain
QVQLVQSGAELKKPGASVKVSCKASGYTFTSYAIRWVRQATGQGLEWMGEIYPRSGQTYYAQSFQGRATLTADKSTSTAYMELSSLRSEDTAVYFCARSGGASYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 108 aa, Artificial Sequence
  light chain variable region
ENVLTQSPATLSLSLGERATLSCSASSSVSSSYLHWYQQKPDQSPRPLIHRTSNLASGIPSRFSGSGSGTDYTLTISSLEAEDFAVYYCQQWSGYPFTFGSGTKLEIK

SEQ ID NO: 44 - protein, 215 aa, Artificial Sequence
  light chain
ENVLTQSPATLSLSLGERATLSCSASSSVSSSYLHWYQQKPDQSPRPLIHRTSNLASGIPSRFSGSGSGTDYTLTISSLEAEDFAVYYCQQWSGYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 5 aa, Artificial Sequence
  CDRH1
SYGIS

SEQ ID NO: 46 - protein, 17 aa, Artificial Sequence
  CDRH2
EIYPRSGNTYYNEKFKG

SEQ ID NO: 47 - protein, 10 aa, Artificial Sequence
  CDRH3
NYGSSYPFAY

SEQ ID NO: 48 - protein, 17 aa, Artificial Sequence
  VH CDR
EIYPRSGNTYYNENFKG

SEQ ID NO: 49-50 - skipped (2 entries)

SEQ ID NO: 51 - protein, 119 aa, Artificial Sequence
  VH
QVQLQQSGAELARPGASVKLSCKASGYTFTSYGISWVKQRTGQGLEWIGEIYPRSGNTYYNEKFKGKATLTADKSSSTAYMELRSLTSEDSAVYFCARNYGSSYPFAYWGQGTLVTVSA

SEQ ID NO: 52 - protein, 108 aa, Artificial Sequence
  VL
ENVLTQSPAIMAASLGQKVTMTCSASSSVSSSYLHWYQQKSGASPKPLIHRTSNLASGVPARFSGSGSGTSYSLTISSVEAEDDATYYCQQWSGYPFTFGGGTKLEIK

SEQ ID NO: 53 - protein, 5 aa, Artificial Sequence
  CDRH1
DYYMN

SEQ ID NO: 54 - protein, 17 aa, Artificial Sequence
  CDRH2
DINPNNGGTSYNQKFKG

SEQ ID NO: 55 - protein, 8 aa, Artificial Sequence
  CDRH3
WRQLRSDY

SEQ ID NO: 56 - protein, 11 aa, Artificial Sequence
  CDRL1
LASQTIGTWLA

SEQ ID NO: 57 - protein, 7 aa, Artificial Sequence
  CDRL2
AATSLAD

SEQ ID NO: 58 - protein, 9 aa, Artificial Sequence
  CDRL3
QQLYSTPLT

SEQ ID NO: 59 - protein, 117 aa, Artificial Sequence
  VH
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCARWRQLRSDYWGQGTTLTVSS

SEQ ID NO: 60 - protein, 107 aa, Artificial Sequence
  VL
DIQMTQSPASQSASLGESVTITCLASQTIGTWLAWYQQKPGKSPQLLIYAATSLADGVPSRFSGSGSGTKFSFKISSLQAEDFVSYYCQQLYSTPLTFGAGTKLELK

SEQ ID NO: 61 - protein, 5 aa, Artificial Sequence
  CDRH1
SYWMH

SEQ ID NO: 62 - protein, 17 aa, Artificial Sequence
  CDRH2
MIHPNNGIPNYNEKFKS

SEQ ID NO: 63 - protein, 12 aa, Artificial Sequence
  CDRH3
EGYYGSEGYFDV

SEQ ID NO: 64 - protein, 10 aa, Artificial Sequence
  CDRL1
SASSSISYIH

SEQ ID NO: 65 - protein, 7 aa, Artificial Sequence
  CDRL2
DTSKLAS

SEQ ID NO: 66 - protein, 9 aa, Artificial Sequence
  CDRL3
HQRSTYPYT

SEQ ID NO: 67 - protein, 121 aa, Artificial Sequence
  VH
QVQLQQPGAELVKPRASVKLSCKASGYTFTSYWMHWVKQRPGQGLEWIGMIHPNNGIPNYNEKFKSKATLTVDKSSTTAYMQLSSLTSEDSAVYHCAREGYYGSEGYFDVWGTGTTVTVSS

SEQ ID NO: 68 - protein, 106 aa, Artificial Sequence
  VL
QIVLTQSPAIMSASPGEKVTMTCSASSSISYIHWYQQKPGTSPKRWIYDTSKLASGVPARFSGSGSGTSYSLTISSMEAEDAATYYCHQRSTYPYTFGGGTKLEIK

SEQ ID NO: 69 - protein, 17 aa, Artificial Sequence
  CDRH2
MIHPNSGSTNYNEKFKS

SEQ ID NO: 70 - protein, 8 aa, Artificial Sequence
  CDRH3
KRGGLGDY

SEQ ID NO: 71 - protein, 15 aa, Artificial Sequence
  CDRL1
RASKSVSTSGYSYMH

SEQ ID NO: 72 - protein, 7 aa, Artificial Sequence
  CDRL2
LASNLES

SEQ ID NO: 73 - protein, 9 aa, Artificial Sequence
  CDRL3
QHSRELPYT

SEQ ID NO: 74 - protein, 117 aa, Artificial Sequence
  VH
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARKRGGLGDYWGQGTSVTVSS

SEQ ID NO: 75 - protein, 111 aa, Artificial Sequence
  VL
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIK

SEQ ID NO: 76 - protein, 5 aa, Artificial Sequence
  CDRH1
SSWMN

SEQ ID NO: 77 - protein, 17 aa, Artificial Sequence
  CDRH2
RIYPGDGDTNYNGKFKG

SEQ ID NO: 78 - protein, 8 aa, Artificial Sequence
  CDRH3
RGYGYLAY

SEQ ID NO: 79 - protein, 11 aa, Artificial Sequence
  CDRL1
KASQDVSTAVA

SEQ ID NO: 80 - protein, 7 aa, Artificial Sequence
  CDRL2
SASYRYT

SEQ ID NO: 81 - protein, 9 aa, Artificial Sequence
  CDRL3
QQHYSTPYT

SEQ ID NO: 82 - protein, 117 aa, Artificial Sequence
  VH
QVQLQQSGPELVKPGASVKISCKASGYAFSSSWMNWVKQRPGKGLEWIGRIYPGDGDTNYNGKFKGKATLTADKSSSTAYMQLSSLTSEDSAVYFCARRGYGYLAYWGQGTLVTVSA

SEQ ID NO: 83 - protein, 107 aa, Artificial Sequence
  VL
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPYTFGGGTKLEIK

SEQ ID NO: 84 - protein, 10 aa, Artificial Sequence
  CDRH3
GYGSSYGFAY

SEQ ID NO: 85 - protein, 9 aa, Artificial Sequence
  CDRL3
QQWSGYPWT

SEQ ID NO: 86 - protein, 119 aa, Artificial Sequence
  VH
QVQLQQSGAELARPGASVKLSCKASGYTFTSYGISWVKQRTGQGLEWIGEIYPRSGNTYYNEKFKGKATLTADKSSSTAYMELRSLTSEDSAVYFCARGYGSSYGFAYWGQGTLVTVSA

SEQ ID NO: 87 - protein, 108 aa, Artificial Sequence
  VL
ENVLTQSPAIMAASLGQKVTMTCSASSSVSSSYLHWYQQKSGASPKPLIHRTSNLASGVPARFSGSGSGTSYSLTISSVEAEDDATYYCQQWSGYPWTFGGGTKLEIK

SEQ ID NO: 88 - protein, 6 aa, Artificial Sequence
  CDRH1
SGYYWN

SEQ ID NO: 89 - protein, 16 aa, Artificial Sequence
  CDRH2
YISYDGSNNYNPSLKN

SEQ ID NO: 90 - protein, 10 aa, Artificial Sequence
  CDRH3
IYGNYYAMDY

SEQ ID NO: 91 - protein, 10 aa, Artificial Sequence
  CDRL1
SASSSVSYMH

SEQ ID NO: 92 - protein, 9 aa, Artificial Sequence
  CDRL3
QQWSSNPPT

SEQ ID NO: 93 - protein, 119 aa, Artificial Sequence
  VH
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCASIYGNYYAMDYWGQGTSVTVSS

SEQ ID NO: 94 - protein, 106 aa, Artificial Sequence
  VL
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGSGTKLEIK

SEQ ID NO: 95 - protein, 5 aa, Artificial Sequence
  CDRH1
DYYMI

SEQ ID NO: 96 - protein, 17 aa, Artificial Sequence
  CDRH2
NINPNNGGTTYNQKFKG

SEQ ID NO: 97 - protein, 10 aa, Artificial Sequence
  CDRH3
GGLRPLYFDY

SEQ ID NO: 98 - protein, 11 aa, Artificial Sequence
  CDRL1
KASENVDTYVS

SEQ ID NO: 99 - protein, 7 aa, Artificial Sequence
  CDRL2
GASNRYT

SEQ ID NO: 100 - protein, 9 aa, Artificial Sequence
  CDRL3
GQSYSYPLT

SEQ ID NO: 101 - protein, 119 aa, Artificial Sequence
  VH
EVQLQQSGPELVQPGASVKISCKASGYTFTDYYMIWVKQSHGKSLEWIGNINPNNGGTTYNQKFKGKATLTVDKSSSTAYMGLPSLTSEDSAVYYCARGGLRPLYFDYWGQGTTLTVSS

SEQ ID NO: 102 - protein, 107 aa, Artificial Sequence
  VL
NIVMTQSPKSMSMSVGERVTLSCKASENVDTYVSWYQQKPEQSPKLLIYGASNRYTGVPDRFTGSGSATDFTLTISSVQAEDLADYHCGQSYSYPLTFGAGTKLELI

SEQ ID NO: 103 - protein, 5 aa, Artificial Sequence
  CDRH1
NTYMH

SEQ ID NO: 104 - protein, 17 aa, Artificial Sequence
  CDRH2
RIDPANGNTKYDPKFQG

SEQ ID NO: 105 - protein, 12 aa, Artificial Sequence
  CDRH3
TYYGSSQHYFDY

SEQ ID NO: 106 - protein, 16 aa, Artificial Sequence
  CDRL1
KSSQSLLDSDGKTYLN

SEQ ID NO: 107 - protein, 7 aa, Artificial Sequence
  CDRL2
LVSKLDS

SEQ ID NO: 108 - protein, 9 aa, Artificial Sequence
  CDRL3
WQDTHFPQT

SEQ ID NO: 109 - protein, 121 aa, Artificial Sequence
  VH
EVQLQQSVAELVRPGASVKLSCTASGFNIKNTYMHWVKQRPEQGLEWIGRIDPANGNTKYDPKFQGKATITADTSSNTAYVQLSSLTSEDTAIYYCALTYYGSSQHYFDYWGQGTTLTVSS

SEQ ID NO: 110 - protein, 112 aa, Artificial Sequence
  VL
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQDTHFPQTFGGGTKLEIK

SEQ ID NO: 111 - protein, 17 aa, Artificial Sequence
  CDRH2
RIYPGDGDANYNGKFKG

SEQ ID NO: 112 - protein, 15 aa, Artificial Sequence
  CDRH3
EGHYYGSGYRWYLDV

SEQ ID NO: 113 - protein, 11 aa, Artificial Sequence
  CDRL1
RASENIYSNLA

SEQ ID NO: 114 - protein, 7 aa, Artificial Sequence
  CDRL2
AATNLAD

SEQ ID NO: 115 - protein, 9 aa, Artificial Sequence
  CDRL3
QHFRGAPFT

SEQ ID NO: 116 - protein, 124 aa, Artificial Sequence
  VH
QIQLQQSGPELVKPGASVKISCKASGYAFSSSWMNWVKKRPGKGLEWIGRIYPGDGDANYNGKFKGKATLTADKSSSTAYMQLSSLTSEDSAVYFCAGEGHYYGSGYRWYLDVWGTGTTVTVSS

SEQ ID NO: 117 - protein, 107 aa, Artificial Sequence
  VL
DIQMTQSPASLSVSVGETVTITCRASENIYSNLAWYQQKQGKSPQLLVYAATNLADGVPSRFSGSGSGTQYSLKINSLQSEDFGSYYCQHFRGAPFTFGSGTKLEIK

SEQ ID NO: 118 - protein, 5 aa, Artificial Sequence
  CDRH1
DYEIH

SEQ ID NO: 119 - protein, 17 aa, Artificial Sequence
  CDRH2
PIDPDTGNTAYNQNLKG

SEQ ID NO: 120 - protein, 11 aa, Artificial Sequence
  CDRH3
GGYDSDWGFAY

SEQ ID NO: 121 - protein, 16 aa, Artificial Sequence
  CDRL1
RSSKSLLHSNGNTFLF

SEQ ID NO: 122 - protein, 7 aa, Artificial Sequence
  CDRL2
RMSDLAS

SEQ ID NO: 123 - protein, 9 aa, Artificial Sequence
  CDRL3
MQHLEYPFT

SEQ ID NO: 124 - protein, 120 aa, Artificial Sequence
  VH
QVQLQQSGAELVRPGASVTLSCKASGYTFTDYEIHWVKQTLVHGLEWIGPIDPDTGNTAYNQNLKGKAILTADKSSSTAYMELRSLTSEDSAVYYCTRGGYDSDWGFAYWGQGTLVTVSA

SEQ ID NO: 125 - protein, 112 aa, Artificial Sequence
  VL
DIVMTQATPSVPVTPGESVSISCRSSKSLLHSNGNTFLFWFLQRPGQSPQLLIYRMSDLASGVPDRFSGSGSGTAFTLRISRVEAEDVGIYYCMQHLEYPFTFGSGTKLEIK

SEQ ID NO: 126 - protein, 5 aa, Artificial Sequence
  CDRH1
DYYLN

SEQ ID NO: 127 - protein, 17 aa, Artificial Sequence
  CDRH2
LIDPYNGGSSCNQKFKG

SEQ ID NO: 128 - protein, 6 aa, Artificial Sequence
  CDRH3
GNAMDY

SEQ ID NO: 129 - protein, 7 aa, Artificial Sequence
  CDRL2
WASTRHT

SEQ ID NO: 130 - protein, 9 aa, Artificial Sequence
  CDRL3
QQHYIIPYM

SEQ ID NO: 131 - protein, 115 aa, Artificial Sequence
  VH
EVQLQQSGPVLVKPGASVKMSCKASGYTFTDYYLNWVKQSHGKSLEWIGLIDPYNGGSSCNQKFKGKATLTVDKSSSTAYMDLNSLTSEDSAVYYCARGNAMDYWGQGTSVTVSS

SEQ ID NO: 132 - protein, 107 aa, Artificial Sequence
  VL
DIVMTQSHKFMSTSVGDRVSITCKASQDVSTAVAWYQEKPGQSPKLLIYWASTRHTGVPDRFTGSGSGTDYILNISSVQAEDLALYYCQQHYIIPYMFGGGTKLEIK

SEQ ID NO: 133 - protein, 5 aa, Artificial Sequence
  CDRH1
NTYMY

SEQ ID NO: 134 - protein, 17 aa, Artificial Sequence
  CDRH2
RIDPANGNTKYAPKFQG

SEQ ID NO: 135 - protein, 12 aa, Artificial Sequence
  CDRH3
LYYGSSYDYFDY

SEQ ID NO: 136 - protein, 9 aa, Artificial Sequence
  CDRL3
WQGTHFPQT

SEQ ID NO: 137 - protein, 121 aa, Artificial Sequence
  VH
EVQLQQSVAELVRPGASVKLSCTASGFNIKNTYMYWVKQRPEQGLEWIGRIDPANGNTKYAPKFQGKATITADTSSNTAYLQLSSLTSEDTAIYYCALLYYGSSYDYFDYWGQGTTLTVSS

SEQ ID NO: 138 - protein, 112 aa, Artificial Sequence
  VL
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 139 - protein, 12 aa, Artificial Sequence
  CDRH3
TYYGSSQYYFDY

SEQ ID NO: 140 - skipped

SEQ ID NO: 141 - protein, 121 aa, Artificial Sequence
  VH
EVQLQQSVAELVRPGASVKLSCTASGFNIKNTYMHWVKQRPEQGLEWIGRIDPANGNTKYAPKFQGKATITADTSSNTAYLQLSSLTSEDTAIYYCALTYYGSSQYYFDYWGQGTTLTVSS

SEQ ID NO: 142 - protein, 107 aa, Artificial Sequence
  VL
DILLTQSPAILSVSPGERVSFSCRASQSIGTRIHWYQQRTNGSPRLLIKYASESISGIPSRFSGSGSGTDFTLSINSVESEDIADYYCQQSNSWPYTFGGGTKLEIK

SEQ ID NO: 143 - protein, 5 aa, Artificial Sequence
  CDRH1
DYYIN

SEQ ID NO: 144 - protein, 17 aa, Artificial Sequence
  CDRH2
RIYPGSGNTYYNEKFKG

SEQ ID NO: 145 - protein, 7 aa, Artificial Sequence
  CDRH3
GYGNSDY

SEQ ID NO: 146 - protein, 11 aa, Artificial Sequence
  CDRL1
RASQSIGTRIH

SEQ ID NO: 147 - protein, 7 aa, Artificial Sequence
  CDRL2
YASESIS

SEQ ID NO: 148 - protein, 9 aa, Artificial Sequence
  CDRL3
QQSNSWPYT

SEQ ID NO: 149 - protein, 116 aa, Artificial Sequence
  VH
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGYGNSDYWGQGTTLTVSS

SEQ ID NO: 150 - protein, 108 aa, Artificial Sequence
  VL
ENVLTQSPAIMAASLGQKVTMTCSASSSVSSSYLHWYQQKSGASPKPLIHRTSNLASGVPARFSGSGSGTSYSLTISSVEAEDDATYYCQQWSGYPFTFGSGTKLEIK

SEQ ID NO: 151 - protein, 5 aa, Artificial Sequence
  CDRH1
SYGLI

SEQ ID NO: 152 - protein, 17 aa, Artificial Sequence
  CDRH2
EIYPRSGSTYYNEWFKG

SEQ ID NO: 153 - protein, 10 aa, Artificial Sequence
  CDRH3
RRGTGDGFDY

SEQ ID NO: 154 - protein, 11 aa, Artificial Sequence
  CDRL1
SASQGISNYLN

SEQ ID NO: 155 - protein, 7 aa, Artificial Sequence
  CDRL2
YTSSLHS

SEQ ID NO: 156 - protein, 9 aa, Artificial Sequence
  CDRL3
QQYIELPFT

SEQ ID NO: 157 - protein, 119 aa, Artificial Sequence
  VH
QVQLQQSGAELARPGASVRLSCKASGYTFTSYGLIWLKQRTGQGLEWIGEIYPRSGSTYYNEWFKGKATLTADKSSNTAFMELRSLTSEDSAVYFCARRRGTGDGFDYWGQGTILTVSS

SEQ ID NO: 158 - protein, 107 aa, Artificial Sequence
  VL
DIQMTQTTSSLSASLGDRVTISCSASQGISNYLNWYQQKPDGTVKLLIYYTSSLHSGVPSRFSGSGSGTDYSLTISNLEPEDIATYYCQQYIELPFTFGSGTKLEIK

SEQ ID NO: 159 - protein, 5 aa, Artificial Sequence
  CDRH1
DYYMH

SEQ ID NO: 160 - protein, 17 aa, Artificial Sequence
  CDRH2
YIYPNNGGNGYNQKFKG

SEQ ID NO: 161 - protein, 13 aa, Artificial Sequence
  CDRH3
GDYYGSLRLTFAY

SEQ ID NO: 162 - skipped

SEQ ID NO: 163 - protein, 16 aa, Artificial Sequence
  CDRH2
VMWPGGRTSYNPAPMS

SEQ ID NO: 164 - protein, 9 aa, Artificial Sequence
  CDRL3
QQYYSYPLT

SEQ ID NO: 165 - protein, 122 aa, Artificial Sequence
  VH
EVQLQQSGPELVKPGASVKMSCKASGYTFTDYYMHWVKQSHGKSLEWIGYIYPNNGGNGYNQKFKGKATLTVDKSSSTAYMELRSLTSEDSAVYYCAIGDYYGSLRLTFAYWGQGTLVTVSA

SEQ ID NO: 166 - protein, 113 aa, Artificial Sequence
  VL
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLELK

SEQ ID NO: 167 - protein, 5 aa, Artificial Sequence
  CDRH1
TYGVS

SEQ ID NO: 168 - protein, 17 aa, Artificial Sequence
  CDRH2
WINTYSGVPTYADDFKG

SEQ ID NO: 169 - protein, 11 aa, Artificial Sequence
  CDRH3
VTTILHWYFDV

SEQ ID NO: 170 - protein, 11 aa, Artificial Sequence
  CDRL1
RASQEISGYLS

SEQ ID NO: 171 - protein, 7 aa, Artificial Sequence
  CDRL2
AASTLDS

SEQ ID NO: 172 - protein, 9 aa, Artificial Sequence
  CDRL3
LQYASYPFT

SEQ ID NO: 173 - protein, 120 aa, Artificial Sequence
  VH
QIQLVQSGPELKKPGETVKISCKASGYTFTTYGVSWVKQAPGKVLKWMGWINTYSGVPTYADDFKGRFAFSLETSASTAYLQISNLKNEDTATYFCAPVTTILHWYFDVWGTGTTVTVSS

SEQ ID NO: 174 - protein, 107 aa, Artificial Sequence
  VL
DIQMTQSPSSLSASLGERVSLTCRASQEISGYLSWLQQKPDGTIKRLIYAASTLDSGVPKRFRGSRSGSDYSLTISSLESEDFADYYCLQYASYPFTFGSGTKLEIK

SEQ ID NO: 175 - protein, 119 aa, Artificial Sequence
  VH
QVQLQQSGAELARPGASVRLSCKASGYTFTSYGISWVKQRTGQGLEWIGEIYPRSGNTYYNEKFKGKATLTADKSSSTAYMELRSLTSEDSAVYFCARNYGSSYPFAYWGQGTLVTVSA

SEQ ID NO: 176 - protein, 7 aa, Artificial Sequence
  CDRL2
ATSNLAS

SEQ ID NO: 177 - protein, 10 aa, Artificial Sequence
  CDRH3
RRGAGDGFDY

SEQ ID NO: 178 - protein, 9 aa, Artificial Sequence
  CDRL3
QQYSKLPFT

SEQ ID NO: 179 - protein, 119 aa, Artificial Sequence
  VH
QVQLQQSGAELARPGASVKLSCKASGYTFTSYGISWVKQRTGQGLEWIGEIYPRSGNTYYNEKFKGKATLTADKSSSTAYMELRSLTSEDSAVYFCARRRGAGDGFDYWGQGTTLTVSS

SEQ ID NO: 180 - protein, 107 aa, Artificial Sequence
  VL
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 181 - protein, 5 aa, Artificial Sequence
  CDRH1
DHTIH

SEQ ID NO: 182 - protein, 17 aa, Artificial Sequence
  CDRH2
YIYPRDGSTKYNEKFKG

SEQ ID NO: 183 - protein, 7 aa, Artificial Sequence
  CDRH3
SNWNFDY

SEQ ID NO: 184 - protein, 11 aa, Artificial Sequence
  CDRL1
KASQDVGTAVA

SEQ ID NO: 185 - protein, 7 aa, Artificial Sequence
  CDRL2
WASTRRT

SEQ ID NO: 186 - protein, 9 aa, Artificial Sequence
  CDRL3
QQYSSYPLT

SEQ ID NO: 187 - protein, 116 aa, Artificial Sequence
  VH
QDQLQQSDAELVKPGASVKISCKVSGYTFTDHTIHWMKQRPEQGLEWIGYIYPRDGSTKYNEKFKGKATLTADKSSSTAYMQLNSLTSEDSAVYFCASSNWNFDYWGQGTTLTVSS

SEQ ID NO: 188 - protein, 107 aa, Artificial Sequence
  VL
DIVMTQSHKFMSTSVGDRVSITCKASQDVGTAVAWYQQKPGQSPKLLIYWASTRRTGVPDRFTGSGSGTDFTLTISNVQSEDLADYFCQQYSSYPLTFGAGTKLELK

SEQ ID NO: 189 - protein, 9 aa, Artificial Sequence
  CDRL3
QQHYSTPWT

SEQ ID NO: 190 - protein, 107 aa, Artificial Sequence
  VL
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPWTFGGGTKLEIK

SEQ ID NO: 191 - protein, 17 aa, Artificial Sequence
  CDRH2
EIYPRSGTTYYNEKFKG

SEQ ID NO: 192 - protein, 10 aa, Artificial Sequence
  CDRH3
RISSGSGVDY

SEQ ID NO: 193 - protein, 9 aa, Artificial Sequence
  CDRL3
QQYSELPWT

SEQ ID NO: 194 - protein, 119 aa, Artificial Sequence
  VH
QVQLQQSGAELARPGASVKLPCKASGYTFTSYGISWVKQRTGQGLEWIGEIYPRSGTTYYNEKFKGKATLTADKSSSTAYMELRSLTSEDSAVYFCARRISSGSGVDYWGQGTTLTVSS

SEQ ID NO: 195 - protein, 107 aa, Artificial Sequence
  VL
DIQMTQTTSSLSASLGDRVTISCSASQGISNYLNWYQQKPDGTVKLLIYYTSSLHSGVPSRFSGSGSGTDYSLTISNLEPEDIATYYCQQYSELPWTFGGGTKLEIK

SEQ ID NO: 196 - protein, 6 aa, Artificial Sequence
  CDRH1
SGYDWH

SEQ ID NO: 197 - protein, 16 aa, Artificial Sequence
  CDRH2
YISYSGSTNYNPSLKS

SEQ ID NO: 198 - protein, 12 aa, Artificial Sequence
  CDRH3
GTPVVAEDYFDY

SEQ ID NO: 199 - protein, 14 aa, Artificial Sequence
  CDRL1
RSSTGAVTTSNYAN

SEQ ID NO: 200 - protein, 7 aa, Artificial Sequence
  CDRL2
ATNNRAP

SEQ ID NO: 201 - protein, 9 aa, Artificial Sequence
  CDRL3
ALWYSNHLV

SEQ ID NO: 202 - protein, 121 aa, Artificial Sequence
  VH
DVQLQESGPGMVKPSQSLSLTCTVTGYSITSGYDWHWIRHFPGNKLEWMGYISYSGSTNYNPSLKSRISITHDTSKNHFFLKLNSVTTEDTATYYCARGTPVVAEDYFDYWGQGTTLTVSS

SEQ ID NO: 203 - protein, 110 aa, Artificial Sequence
  VL
QAVVTQESALSTSPGETVTLTCRSSTGAVTTSNYANWVQEKPDHLFTGLIGATNNRAPGVPARFSGSLIGDKAALTITGAQTEDEAIYFCALWYSNHLVFGGGTKLTVLG

SEQ ID NO: 204 - protein, 5 aa, Artificial Sequence
  CDRH1
SAYWN

SEQ ID NO: 205 - protein, 16 aa, Artificial Sequence
  CDRH2
YISYSGSTYFNPSLKS

SEQ ID NO: 206 - protein, 10 aa, Artificial Sequence
  CDRH3
SHYYGYYFDY

SEQ ID NO: 207 - protein, 15 aa, Artificial Sequence
  CDRL1
RASETIDSYGDSLMH

SEQ ID NO: 208 - protein, 7 aa, Artificial Sequence
  CDRL2
RASNLES

SEQ ID NO: 209 - protein, 9 aa, Artificial Sequence
  CDRL3
QQTDEDPYT

SEQ ID NO: 210 - protein, 118 aa, Artificial Sequence
  VH
EVQLQESGPSLVKPSQTLSLTCSVTGDSITSAYWNWIRKFPGNKLEYMGYISYSGSTYFNPSLKSRISITRNTSKNQYYLQLNSVTTEDTATYYCARSHYYGYYFDYWGHGTTLTVSS

SEQ ID NO: 211 - protein, 111 aa, Artificial Sequence
  VL
DIVLTQSPASLAVSLGQRATISCRASETIDSYGDSLMHWYQQKAGQPPKLLIYRASNLESGIPARFSGSGSRTDFTLTINPVEADDVATYYCQQTDEDPYTFGGGTKLEIK

SEQ ID NO: 212 - protein, 6 aa, Artificial Sequence
  CDRH1
SGYSWH

SEQ ID NO: 213 - protein, 16 aa, Artificial Sequence
  CDRH2
YIHYSGSTNYNPSLKS

SEQ ID NO: 214 - protein, 12 aa, Artificial Sequence
  CDRH3
GPHRYDGVWFAY

SEQ ID NO: 215 - protein, 12 aa, Artificial Sequence
  CDRL1
SASSSISSNYLH

SEQ ID NO: 216 - protein, 9 aa, Artificial Sequence
  CDRL3
QQGTNIPLT

SEQ ID NO: 217 - protein, 121 aa, Artificial Sequence
  VH
DVQLQESGPDLVKPSQSLSVTCTVTGYSITSGYSWHWIRQFPGNKLEWMGYIHYSGSTNYNPSLKSRISITRDTSKNQFFLQLSSVTTEDTATYYCASGPHRYDGVWFAYWGQGTLVTVSS

SEQ ID NO: 218 - protein, 108 aa, Artificial Sequence
  VL
EIVLTQSPTTMAASPGEKITITCSASSSISSNYLHWYQQKPGFSPKLLIYRTSNLASGVPARFSGSGSGTSYSLTIGTMEAEDVATYYCQQGTNIPLTFGAGTKLEIK

SEQ ID NO: 219 - protein, 118 aa, Artificial Sequence
  VH
EVKLVESGGGLVKPGGSLKLSCAASGFTLSSYGMSWVRQIPEKRLEWVASIRSDGNTYYPDSVKGRFIISRDNARNILYLQMSSLRSEDTAMYYCTRGGYYGSSPYYWGQGTTLTVSS

SEQ ID NO: 220 - protein, 112 aa, Artificial Sequence
  VL
DIVMSQSPSSLPVSVGEKISMTCKSSQSLLYSSNQKNYLAWYQQKPGQSPKLLIYWASTRDSGVPDRFIGSGSGTDFTLTINSVKAEDLAVYYCQQYYNYLTFGAGTKLELK

SEQ ID NO: 221 - protein, 120 aa, Artificial Sequence
  VH
QVQLKESGPGLVAPSQSLSITCTVSGFSLTTYGVHWVRQSPGKGLEWLGVMWPGGRTSYNPAPMSRLSISKDNSKSQVFLKMNSLQTDDTAMYYCVRGDYEYDYYAMDYWGQGTSVTVSS

SEQ ID NO: 222 - protein, 106 aa, Artificial Sequence
  VL
QIVLSQSPAILSASPGEKVTMTCRASSSVSYMHWYQQKPGSSPKPWIYATSNLASGVPARFSGSGSGTSYSLTISRMEAEDAATYYCHQWSSNPYTFGGGTKLEIK

SEQ ID NO: 223 - protein, 119 aa, Artificial Sequence
  VH
QVQLQQSGAELARPGASVRLSCKASGYTFTSYAIRWVKQRTGQGLEWIGEIYPRSGNTYYNENFKGKATLTADKSSSTAYMELRSLTSEDSAVYFCARSGGASYTMDYWGQGTSVTVSS

SEQ ID NO: 224 - skipped

SEQ ID NO: 225 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 226 - protein, 289 aa, Macaca fascicularis
MKTLPAMLGSGRLFWVVFLIPYLDIWNIHGKESCDVQLYIKRQSYHSIFAGDPFKLECPVKYCAHRPQVTWCKLNGTTCVKLEGRHTSWKQEKNLSFFILHFEPVLPSDNGSYRCSANFLSAIIESHSTTLYVTDVKSASERPSKDEMASRPWLLYSLLPLGGLPLLITTCFCLFCFLRRHQGKQNELSDTTGREITLVDVPFKSEQTEASTRQNSQVLLSETGIYDNEPDFCFRMQEGSEVYSNPCLEENKPGIIYASLNHSIIGLNSRQARNVKEAPTEYASICVRS

SEQ ID NO: 227 - protein, 330 aa, Artificial Sequence
  hIgG1 const region with 238D
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 107 aa, Artificial Sequence [sequence omitted]

SEQ ID NO: 229 - protein, 434 aa, Artificial Sequence
  hHVEM-mFc fusion protein (including signal peptide and C-terminal His-tag)
MEPPGDWGPPPWRSTPRTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHLVPRGSGSKPSISTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKHHHHHH

SEQ ID NO: 230 - protein, 450 aa, Artificial Sequence
  Mopc21 hIgG1 P238D isotype control heavy chain
DVQLVESGGGLVQPGGSRKLSCAASGFTFSSFGMHWVRQAPEKGLEWVAYISSGSSTLHYADTVKGRFTISRDNPKNTLFLQMTSLRSEDTAMYYCARWGNYPYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 214 aa, Artificial Sequence
  Mopc21 hIgG1 P238D isotype control light chain
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 5 aa, Artificial Sequence
  flexible linker
GGGGS

SEQ ID NO: 233 - protein, 18 aa, Artificial Sequence
  linker
KESGSVSSEQLAQFRSLD

SEQ ID NO: 234 - protein, 14 aa, Artificial Sequence
  linker
EGKSSGSGSESKST

SEQ ID NO: 235 - protein, 327 aa, Artificial Sequence
  Reference IgG4 constant sequence containing a P238D and also a S228P substitution.
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGDSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK